SEQUENCE LISTING (2758 sequences)

SEQ ID NO: 1 - DNA, 20 bp, Artificial sequence
  Synthetic
gcatcctaactccaaaggga

SEQ ID NO: 2 - DNA, 20 bp, Artificial sequence
  Synthetic
cttgttgtcttggcagtcgt

SEQ ID NO: 3 - DNA, 20 bp, Artificial sequence
  Synthetic
cgagaagttcaggagcaaca

SEQ ID NO: 4 - DNA, 20 bp, Artificial sequence
  Synthetic
gaccaggttggtgtccttct

SEQ ID NO: 5 - DNA, 21 bp, Artificial sequence
  Synthetic
ggagcgagatccctccaaaat

SEQ ID NO: 6 - DNA, 23 bp, Artificial sequence
  Synthetic
ggctgttgtcatacttctcatgg

SEQ ID NO: 7 - DNA, 28 bp, Artificial sequence
  Synthetic
agtgaattcatgagtgtgaaagaggcag

SEQ ID NO: 8 - DNA, 28 bp, Artificial sequence
  Synthetic
agtgcggccgctcagccattggtctggc

SEQ ID NO: 9 - DNA, 59 bp, Artificial sequence
  Synthetic
agtgcggccgcgcacgtgttcgccagctaccaggttagcatcccgcagtcgtgcgagca

SEQ ID NO: 10 - DNA, 66 bp, Artificial sequence
  Synthetic
gcagtcgtgcgagcagtgcctctcctatatctggctcatggacaaggccctgctctgcagcgtgtg

SEQ ID NO: 11 - DNA, 28 bp, Artificial sequence
  Synthetic
agtgaattcttaccggtaggtgatgtcc

SEQ ID NO: 12 - DNA, 28 bp, Artificial sequence
  Synthetic
agtgaattcatgagtgtgaaagaggcag

SEQ ID NO: 13 - DNA, 28 bp, Artificial sequence
  Synthetic
atgtctagacccgttgtgctcctggaca

SEQ ID NO: 14 - DNA, 28 bp, Artificial sequence
  Synthetic
atggtcgacctgccggagctggacccaa

SEQ ID NO: 15 - DNA, 28 bp, Artificial sequence
  Synthetic
agtgcggccgctcagccattggtctggc

SEQ ID NO: 16 - DNA, 28 bp, Artificial sequence
  Synthetic
ccggaattcccaggcgttgagcctggcc

SEQ ID NO: 17 - DNA, 38 bp, Artificial sequence
  Synthetic
ataagaatgcggccgctcaagcattttgtcgcaggagc

SEQ ID NO: 18 - DNA, 30 bp, Artificial sequence
  Synthetic
ccgcaagtcgggtgaggccaaccgcactcg

SEQ ID NO: 19 - DNA, 30 bp, Artificial sequence
  Synthetic
cgagtgcggttggcctcacccgacttgcgg

SEQ ID NO: 20 - DNA, 33 bp, Artificial sequence
  Synthetic
ctctaccgcaagtcgggtaatgccaaccgcact

SEQ ID NO: 21 - DNA, 33 bp, Artificial sequence
  Synthetic
agtgcggttggcattacccgacttgcggtagag

SEQ ID NO: 22 - DNA, 27 bp, Artificial sequence
  Synthetic
cgcaagtcgggtgttgccaaccgcact

SEQ ID NO: 23 - DNA, 27 bp, Artificial sequence
  Synthetic
agtgcggttggcaacacccgacttgcg

SEQ ID NO: 24 - DNA, 29 bp, Artificial sequence
  Synthetic
gtcgggtgctgccgagcgcactcgggagc

SEQ ID NO: 25 - DNA, 29 bp, Artificial sequence
  Synthetic
gctcccgagtgcgctcggcagcacccgac

SEQ ID NO: 26 - DNA, 31 bp, Artificial sequence
  Synthetic
cgggtgctgccaacgagactcgggagctccg

SEQ ID NO: 27 - DNA, 31 bp, Artificial sequence
  Synthetic
cggagctcccgagtctcgttggcagcacccg

SEQ ID NO: 28 - DNA, 44 bp, Artificial sequence
  Synthetic
ccatcttcatttgaaaacgtgccagaaaagtgggtgcctgagat

SEQ ID NO: 29 - DNA, 44 bp, Artificial sequence
  Synthetic
atctcaggcacccacttttctggcacgttttcaaatgaagatgg

SEQ ID NO: 30 - DNA, 46 bp, Artificial sequence
  Synthetic
ctgtttttcagtggtctctccagattcatttgaaaacgtgccagaa

SEQ ID NO: 31 - DNA, 46 bp, Artificial sequence
  Synthetic
ttctggcacgttttcaaatgaatctggagagaccactgaaaaacag

SEQ ID NO: 32 - DNA, 42 bp, Artificial sequence
  Synthetic
gcatcatttgaaaatgtccctgaaaagtggtatcctgaggtg

SEQ ID NO: 33 - DNA, 42 bp, Artificial sequence
  Synthetic
cacctcaggataccacttttcagggacattttcaaatgatgc

SEQ ID NO: 34 - DNA, 44 bp, Artificial sequence
  Synthetic
gcttttcccttgtgagtcctgattcatttgaaaatgtccctgaa

SEQ ID NO: 35 - DNA, 44 bp, Artificial sequence
  Synthetic
ttcagggacattttcaaatgaatcaggactcacaagggaaaagc

SEQ ID NO: 36 - DNA, 21 bp, Artificial sequence
  Synthetic
cgagaucacgaugcacuggtt

SEQ ID NO: 37 - DNA, 21 bp, Artificial sequence
  Synthetic
agcuguuccucucgaaguctt

SEQ ID NO: 38 - DNA, 21 bp, Artificial sequence
  Synthetic
uucuccgaacgugucacgutt

SEQ ID NO: 39 - DNA, 20 bp, Artificial sequence
  Synthetic
gcatcctaactccaaaggga

SEQ ID NO: 40 - DNA, 20 bp, Artificial sequence
  Synthetic
cttgttgtcttggcagtcgt

SEQ ID NO: 41 - DNA, 20 bp, Artificial sequence
  Synthetic
cgagaagttcaggagcaaca

SEQ ID NO: 42 - DNA, 20 bp, Artificial sequence
  Synthetic
gaccaggttggtgtccttct

SEQ ID NO: 43 - DNA, 21 bp, Artificial sequence
  Synthetic
ggagcgagatccctccaaaat

SEQ ID NO: 44 - DNA, 23 bp, Artificial sequence
  Synthetic
ggctgttgtcatacttctcatgg

SEQ ID NO: 45 - DNA, 28 bp, Artificial sequence
  Synthetic
agtgaattcatgagtgtgaaagaggcag

SEQ ID NO: 46 - DNA, 28 bp, Artificial sequence
  Synthetic
agtgcggccgctcagccattggtctggc

SEQ ID NO: 47 - protein, 202 aa, Homo sapiens
FGVCVDSLTSDKASVPIVLEKLLEHVEMHGLYTEGLYRKSGAANRTRELRQALQTDPAAVKLENFPIHAITGVLKQWLRELPEPLMTFAQYGDFLRAVELPEKQEQLAAIYAVLEHLPEANHNSLERLIFHLVKVALLEDVNRMSPGALAIIFAPCLLRCPDNSDPLTSMKDVLKITTCVEMLIKEQMRKYKVKMEEISQLE

SEQ ID NO: 48 - protein, 202 aa, Mus musculus
FGVCVDSLTSDKASVPIVLEKLLEHVEMHGLYTEGLYRKSGAANRTRELRQALQTDPAAVKLEDFPIHAITGVLKQWLRELPEPLMTFAQYGDFLRAVELPEKQEQLSAIYAVLDHLPEANHTSLERLIFHLVKVALLEDVNRMSPGALAIIFAPCLLRCPDNSDPLTSMKDVLKITTCVEMLIKEQMRKYKMKMEEINHLE

SEQ ID NO: 49 - protein, 202 aa, Rattus norvegicus
FGVCVDSLTSDKASVPIVLEKLLEHVEMHGLYTEGLYRKSGAANRTRELRQALQTDPATVKLEDFPIHAITGVLKQWLRELPEPLMTFAQYGDFLRAVELPEKQEQLAAIYAVLDHLPEANHTSLERLIFHLVKVALLEDVNRMSPGALAIIFAPCLLRCPDNSDPLTSMKDVLKITTCVEMLIKEQMRKYKVKMEEINHLE

SEQ ID NO: 50 - protein, 202 aa, Danio rerio
FGVPLCALVHAADSVPFVLEQMLVHVEMNGLYTEGIYRKSGAACRAKELHQKLEKDPHTVSLDTYPIHTVTGLVKQWLRELPDPLMTYSLYNDFLYAVDLPETSERLRAVYRKLEELPSSNISTLERLIFHLVKVAKEEEHNRMSADSLAIVFAPCILRCPDSSDPLLSMKDINKTTLCVEILIKEQIRRYNQKMEEIQQLE

SEQ ID NO: 51 - protein, 199 aa, Homo sapiens
FGVCVDSLTSDKASVPIVLEKLLEHVEMHGLYTEGLYRKSGAANRTRELRQALQTDPAAVKLENFPIHAITGVLKQWLRELPEPLMTFAQYGDFLRAVELPEKQEQLAAIYAVLEHLPEANHNSLERLIFHLVKVALLEDVNRMSPGALAIIFAPCLLRCPDNSDPLTSMKDVLKITTCVEMLIKEQMRKYKVKMEEIS

SEQ ID NO: 52 - protein, 191 aa, Gallus gallus
YNSNKDNQSEGTAQLDSIGFSIIKKCIHAVETRGINEQGLYRIVGVNSRVQKLLSILMDPKTATETETEICAEWEIKTITSALKTYLRMLPGPLMMYQFQRSFIKAAKLENQESRVSEIHSLVHRLPEKNRQMLHLLMNHLAKVADNHKQNLMTVANLGVVFGPTLLRPQEETVAAIMDIKFQNIVIEILI

SEQ ID NO: 53 - protein, 210 aa, Homo sapiens
FGGDMEKFIQSSGQPVPLVVESCIRFINLNGLQHEGIFRVSGAQLRVSEIRDAFERGEDPLVEGCTAHDLDSVAGVLKLYFRSLEPPLFPPDLFGELLASSELEATAERVEHVSRLLWRLPAPVLVVLRYLFTFLNHLAQYSDENMMDPYNLAVCFGPTLLPVPAGQDPVALQGRVNQLVQTLIVQPDRVFPPLTSLPGPVYEKCMAPPS

SEQ ID NO: 54 - protein, 198 aa, Homo sapiens
FGVSLQHLQEKNPEQEPIPIVLRETVAYLQAHALTTEGIFRRSANTQVVREVQQKYNMGLPVDFDQYNELHLPAVILKTFLRELPEPLLTFDLYPHVVGFLNIDESQRVPATLQVLQTLPEENYQVLRFLTAFLVQISAHSDQNKMTNTNLAVVFGPNLLWAKDAAITLKAINPINTFTKFLLDHQGELFPSPDPSGL

SEQ ID NO: 55 - protein, 196 aa, Homo sapiens
FGSHLHKVCERENSTVPWFVKQCIEAVEKRGLDVDGIYRVSGNLATIQKLRFIVNQEEKLNLDDSQWEDIHVVTGALKMFFRELPEPLFPYSFFEQFVEAIKKQDNNTRIEAVKSLVQKLPPPNRDTMKVLFGHLTKIVAKASKNLMSTQSLGIVFGPTLLRAENETGNMAIHMVYQNQIAELMLSEYSKIFGSEE

SEQ ID NO: 56 - protein, 202 aa, Homo sapiens
FGCDLTEYLESSGQDVPYVLKSCAEFIETHGIVDGIYRLSGVTSNIQRLRQEFGSDQCPDLTREVYLQDIHCVGSLCKLYFRELPNPLLTYELYEKFTEAVSHCPEEGQLARIQNVIQELPPSHYRTLEYLIRHLAHIASFSSKTNMHARNLALVWAPNLLRSKEIEATGCNGDAAFLAVRVQQVVIEFILNHVDQIFNNGA

SEQ ID NO: 57 - protein, 193 aa, Homo sapiens
MAAIRKKLVIVGDGACGKTCLLIVFSKDQFPEVYVPTVFENYVADIEVDGKQVELALWDTAGQEDYDRLRPLSYPDTDVILMCFSIDSPDSLENIPEKWTPEVKHFCPNVPIILVGNKKDLRNDEHTRRELAKMKQEPVKPEEGRDMANRIGAFGYMECSAKTKDGVREVFEMATRAALQARRGKKKSGCLVL

SEQ ID NO: 58 - protein, 190 aa, Homo sapiens
MQTIKCVVVGDGAVGKTCLLISYTTNKFPSEYVPTVFDNYAVTVMIGGEPYTLGLFDTAGQEDYDRLRPLSYPQTDVFLVCFSVVSPSSFENVKEKWVPEITHHCPKTPFLLVGTQIDLRDDPSTIEKLAKNKQKPITPETAEKLARDLKAVKYVECSALTQKGLKNVFDEAILAALEPPEPKKSRRCVL

SEQ ID NO: 59 - protein, 192 aa, Homo sapiens
MQAIKCVVVGDGAVGKTCLLISYTTNAFPGEYIPTVFDNYSANVMVDGKPVNLGLWDTAGQEDYDRLRPLSYPQTDVFLICFSLVSPASFENVRAKWYPEVRHHCPNTPIILVGTKLDLRDDKDTIEKLKEKKLTPITYPQGLAMAKEIGAVKYLECSALTQRGLKTVFDEAIRAVLCPPPVKKRKRKCLLL

SEQ ID NO: 60 - RNA, 1401 nt, Homo sapiens
ggcccccugcgccugcucucccuugccccggcgcgcccaccccgagcccccuccccaccguggccgccccuccacgacgaaggccgucguccuucguaacggucagagugaagaccccccggcggacccccaucaugcccacggccaacaucaagcucccaccaggccugcccucccaccugccucgcugggcaccgggugcccgggaggcggcugccccagugcggcgccgggagccaccugcccgccgcccggaccagauacauuccguguacaucacgcccggggcagaccugccagugcagggcgcccuggagccccuagaagaggauggccagccaccuggggccaagcggagguacucggaucccccaacguacugccugccccccgccucgggccagaccaauggcugagagccacagcugacaaagucugcauguccgaggacggccccugcacuggagcugggcgccagagcugcagagcuaguguucggcccucagagaaggauccagaaucaaaagcucaagagugacgugaggugggcaccggccccaagugcagagucaaggcagggagaggccggcuggagccaggcccccucgcacgcagcccccaaaucauggacgcaccuguggggagcaccacaucuccaccugcggccucacaucuccccacuccccuuuuuguacguuuaacuguuucuuuguacgugguuuacguaacuuuaaacuguaacagccuuaauggaagaccaaaugguuuuuuauauguguauguacaaaguuuucuauuaacgcugcccgucucccuuauaaccuggacgugagcugucagagcagaagccacuaggccacugcgcgucugaggcucagacccugcugugguuggcuugggguggccaaugggcugggacccuccaugagaguuuuggacacuugggucaccugacccgugccucucugacacaugucuccggggggcagccaccuggccaaugugcauuuuugcacaugcuggaaccuuccaugggggucugggcuauuggcuggagccaggacaugagucaggggcaccauggaccucacgugccagggagacuugaauguggcugucacucuuccggacgccaagggcugcaggaggcugcuuuuggcacuacccaccccgugugacagaauaggagccagcgacucaggacugcucacgggucaggagggcaacgccugaagucagaccucccuauaggucaacagggacaaccuggggaucucuggagcagggcccuccucucucaggcuuggcccacucccccagacaccuggacacguggccacaaaucugggacaaggggcccccgcacagcaugaaauaaaaagugccugagaagugugugca

SEQ ID NO: 61 - RNA, 152 nt, Homo sapiens
cggggcggagcggcucgagcagcggcgggcuggcaggcggucguccggccggggacccggcccgggaccggcggcgcgcggcggccgaggccagcuccaggacacgcgcgccccgagccugggaggcaugcugaagccaggcggccggcagg

SEQ ID NO: 62 - DNA, 2000 bp, Homo sapiens
gatcacgaggtcaggagatcgagaccatcctggctaacatggtgaaaccccatctctactaaaaatacaaaaatgagctggggtggtggcatgcgcctgtagtccctcctactcaggagactgacacaggagaactgcttgaacccgggaggcacaggttgcagtgagccaagatcacgccattgcactccagccagggtgacagtgagactccgtctcaataaataaataaataaaaataaaaataaaaataaaaatagaatgaagcctgatgtaataggaaagaagggaccctgagataggaatcgctgactcatcagctgtgggggtgcaggagagggagaatctgggatgactaggggtttcagggtttcagcctgagctggtgagaaaagaagggatccaagaacagacaccagcaacacaggaggggcaagtggatgggcaagtggaggggcagggccaaggccagggcactgtaactcctgctcccatcaccaaaggcggctccactggtccctgcggccacctcctctgaagcaggctcccaggaccccaaagtgagacccagacaatgttggggacacttccaggccaccgtggggagaggcaacttagcaaatgttcgccaggtgccagcagctgcgctcgcccgtaggctcctcctaacccgacagcttcctaactgttcacacctgctttttcgtcgctgcaaaacaaagacgcacaggttttacataggtcacccccggggcagtctcacagccagaaagagacaagagcacagactccctctggggactgtgctagggctgtggcctcaaaccgaatgggctcctgggtgaacctgctgcgacctgaggggggtccctacaacctgtgcgagcctcagctactgcacctgtacctgggaggtgctactgtcaatctatcggttacggcaaggggcggtggagtgattcctcttgctggccaatttcacttcagtttgcacctggcacaatcaacactggcagaattaacaaaactagacaaatatagcctcatgatcattcatgcctagctctcggtcagtattcaacttctttcttgttcatggtgtgttaccctaggcaagtcacttcccctccctgggtctccatcatctcctcagtaaactgggagcacagaataaagcgcgtgggaggattaaaggggacactttggtcgcccaacacttgttcactgcataacgataattccaagagcgacatcacagtaaagacccaggcgccgatctaagcgcgtttcagcctcacgcgacgcctacgaggttgagctgcgtcatcggccccatttcgtagcggaggaaagcgaggcactgggcggtcaggccgcttgccggaggtcgcccagcgtcccagccgaggacgcggaactcagggccggtcgggtgtcagcgctccggagcatgcgcagaggcagcacgccatcggggtcttcaggggcccgcgcagttcctggcggggtcgaacccgaactcaccccgagggtcctaactccccacctccgctgcccatcctctccagccctatggcgtctacaaatccagacctgcggaagccccaactcacccagcgcctcgccccgaggaatccgccattttcccgccttccacctcaaggcccgacccgccggcttttcaaagccggacccgcccccttgcttgcgggtcggaccatttgtggagacgcaggaggggtggctgcgaggcgtgaggcagcgccggctaagtgtggagagcgcggccccagacgcgcgcttcgcctgtcctcccctcgcacttggtctcgcccaccatcggcggtagtgggtaggggcggtgggtgggacctgatctgtcggacagtaggggcggggcttttcgcgatgccccgcccctaggagcgctggcgtcacgcccccgtccccgcctctccggaacccggaagcgggagcgcgcgggggagcggcgggcgcgg

SEQ ID NO: 63 - RNA, 22 nt, Homo sapiens
cuguacagccuccuagcuuucc

SEQ ID NO: 64 - RNA, 21 nt, Homo sapiens
cuauacaaucuacugucuuuc

SEQ ID NO: 65 - RNA, 22 nt, Homo sapiens
ugagguaguagguuguauaguu

SEQ ID NO: 66 - RNA, 22 nt, Homo sapiens
cuauacaaccuacugccuuccc

SEQ ID NO: 67 - RNA, 22 nt, Homo sapiens
ugagguaguagguugugugguu

SEQ ID NO: 68 - RNA, 22 nt, Homo sapiens
ugagguaguagguuguaugguu

SEQ ID NO: 69 - RNA, 22 nt, Homo sapiens
cuguacaaccuucuagcuuucc

SEQ ID NO: 70 - RNA, 22 nt, Homo sapiens
ugagguaguagguuguaugguu

SEQ ID NO: 71 - RNA, 22 nt, Homo sapiens
cuauacgaccugcugccuuucu

SEQ ID NO: 72 - RNA, 22 nt, Homo sapiens
agagguaguagguugcauaguu

SEQ ID NO: 73 - RNA, 22 nt, Homo sapiens
cuauacggccuccuagcuuucc

SEQ ID NO: 74 - RNA, 22 nt, Homo sapiens
ugagguaggagguuguauaguu

SEQ ID NO: 75 - RNA, 22 nt, Homo sapiens
cuauacaaucuauugccuuccc

SEQ ID NO: 76 - RNA, 22 nt, Homo sapiens
cuauacagucuacugucuuucc

SEQ ID NO: 77 - RNA, 22 nt, Homo sapiens
ugagguaguagauuguauaguu

SEQ ID NO: 78 - RNA, 21 nt, Homo sapiens
cuguacaggccacugccuugc

SEQ ID NO: 79 - RNA, 22 nt, Homo sapiens
ugagguaguaguuuguacaguu

SEQ ID NO: 80 - RNA, 22 nt, Homo sapiens
cugcgcaagcuacugccuugcu

SEQ ID NO: 81 - RNA, 22 nt, Homo sapiens
ugagguaguaguuugugcuguu

SEQ ID NO: 82 - RNA, 22 nt, Homo sapiens
uggaauguaaagaaguauguau

SEQ ID NO: 83 - RNA, 22 nt, Homo sapiens
uggaauguaaagaaguauguau

SEQ ID NO: 84 - RNA, 22 nt, Homo sapiens
acauacuucuuuauaugcccau

SEQ ID NO: 85 - RNA, 22 nt, Homo sapiens
caagcuuguaucuauagguaug

SEQ ID NO: 86 - RNA, 22 nt, Homo sapiens
aacccguagauccgaacuugug

SEQ ID NO: 87 - RNA, 21 nt, Homo sapiens
uacaguacugugauaacugaa

SEQ ID NO: 88 - RNA, 22 nt, Homo sapiens
caguuaucacagugcugaugcu

SEQ ID NO: 89 - RNA, 23 nt, Homo sapiens
agcuucuuuacagugcugccuug

SEQ ID NO: 90 - RNA, 23 nt, Homo sapiens
agcagcauuguacagggcuauga

SEQ ID NO: 91 - RNA, 23 nt, Homo sapiens
ucauagcccuguacaaugcugcu

SEQ ID NO: 92 - RNA, 22 nt, Homo sapiens
acggauguuugagcaugugcua

SEQ ID NO: 93 - RNA, 23 nt, Homo sapiens
ucaaaugcucagacuccuguggu

SEQ ID NO: 94 - RNA, 22 nt, Homo sapiens
cugcaauguaagcacuucuuac

SEQ ID NO: 95 - RNA, 23 nt, Homo sapiens
aaaagugcuuacagugcagguag

SEQ ID NO: 96 - RNA, 22 nt, Homo sapiens
ccgcacuguggguacuugcugc

SEQ ID NO: 97 - RNA, 21 nt, Homo sapiens
uaaagugcugacagugcagau

SEQ ID NO: 98 - RNA, 23 nt, Homo sapiens
agcagcauuguacagggcuauca

SEQ ID NO: 99 - RNA, 22 nt, Homo sapiens
caaauucguaucuaggggaaua

SEQ ID NO: 100 - RNA, 23 nt, Homo sapiens
uacccuguagauccgaauuugug

SEQ ID NO: 101 - RNA, 22 nt, Homo sapiens
acagauucgauucuaggggaau

SEQ ID NO: 102 - RNA, 23 nt, Homo sapiens
uacccuguagaaccgaauuugug

SEQ ID NO: 103 - RNA, 21 nt, Homo sapiens
uugcucacuguucuucccuag

SEQ ID NO: 104 - RNA, 18 nt, Homo sapiens
cagggucagcugagcaug

SEQ ID NO: 105 - RNA, 21 nt, Homo sapiens
aagcauucuuucauugguugg

SEQ ID NO: 106 - RNA, 22 nt, Homo sapiens
uuuccggcucgcgugggugugu

SEQ ID NO: 107 - RNA, 22 nt, Homo sapiens
uuuccggcucgcgugggugugu

SEQ ID NO: 108 - RNA, 21 nt, Homo sapiens
ggacccacccggccgggaaua

SEQ ID NO: 109 - RNA, 21 nt, Homo sapiens
ccgucgccgccacccgagccg

SEQ ID NO: 110 - RNA, 23 nt, Homo sapiens
gagggucuugggagggaugugac

SEQ ID NO: 111 - RNA, 27 nt, Homo sapiens
cacuguaggugauggugagagugggca

SEQ ID NO: 112 - RNA, 23 nt, Homo sapiens
ccugcagcgacuugauggcuucc

SEQ ID NO: 113 - RNA, 22 nt, Homo sapiens
auauacagggggagacucuuau

SEQ ID NO: 114 - RNA, 22 nt, Homo sapiens
auauacagggggagacucucau

SEQ ID NO: 115 - RNA, 21 nt, Homo sapiens
agaggauacccuuuguauguu

SEQ ID NO: 116 - RNA, 23 nt, Homo sapiens
gggaugguagaccggugacgugc

SEQ ID NO: 117 - RNA, 21 nt, Homo sapiens
uaggacacauggucuacuucu

SEQ ID NO: 118 - RNA, 21 nt, Homo sapiens
ugcggccggugcucaaccugc

SEQ ID NO: 119 - RNA, 20 nt, Homo sapiens
ccugagcccgggccgcgcag

SEQ ID NO: 120 - RNA, 22 nt, Homo sapiens
cuccugagccauucugagccuc

SEQ ID NO: 121 - RNA, 21 nt, Homo sapiens
gugccagcugcagugggggag

SEQ ID NO: 122 - RNA, 21 nt, Homo sapiens
cccggagccaggaugcagcuc

SEQ ID NO: 123 - RNA, 21 nt, Homo sapiens
ucguggccuggucuccauuau

SEQ ID NO: 124 - RNA, 20 nt, Homo sapiens
ucugcaggguuugcuuugag

SEQ ID NO: 125 - RNA, 21 nt, Homo sapiens
uguucauguagauguuuaagc

SEQ ID NO: 126 - RNA, 18 nt, Homo sapiens
ucagcuggcccucauuuc

SEQ ID NO: 127 - RNA, 21 nt, Homo sapiens
uggcagggaggcugggagggg

SEQ ID NO: 128 - RNA, 20 nt, Homo sapiens
ucacuguucagacaggcgga

SEQ ID NO: 129 - RNA, 22 nt, Homo sapiens
aacgccauuaucacacuaaaua

SEQ ID NO: 130 - RNA, 22 nt, Homo sapiens
uggagugugacaaugguguuug

SEQ ID NO: 131 - RNA, 21 nt, Homo sapiens
ccccaccuccucucuccucag

SEQ ID NO: 132 - RNA, 19 nt, Homo sapiens
gugaggacucgggaggugg

SEQ ID NO: 133 - RNA, 22 nt, Homo sapiens
ugagccccugugccgcccccag

SEQ ID NO: 134 - RNA, 22 nt, Homo sapiens
guggguacggcccagugggggg

SEQ ID NO: 135 - RNA, 22 nt, Homo sapiens
ucaccagcccuguguucccuag

SEQ ID NO: 136 - RNA, 26 nt, Homo sapiens
gugagggcaugcaggccuggaugggg

SEQ ID NO: 137 - RNA, 20 nt, Homo sapiens
cgugccacccuuuuccccag

SEQ ID NO: 138 - RNA, 17 nt, Homo sapiens
guggggccaggcggugg

SEQ ID NO: 139 - RNA, 20 nt, Homo sapiens
ucacaccugccucgcccccc

SEQ ID NO: 140 - RNA, 21 nt, Homo sapiens
gugggcgggggcaggugugug

SEQ ID NO: 141 - RNA, 23 nt, Homo sapiens
cucucaccacugcccucccacag

SEQ ID NO: 142 - RNA, 24 nt, Homo sapiens
guggguaggguuugggggagagcg

SEQ ID NO: 143 - RNA, 20 nt, Homo sapiens
gugucugggcggacagcugc

SEQ ID NO: 144 - RNA, 22 nt, Homo sapiens
agugggaggccagggcacggca

SEQ ID NO: 145 - RNA, 20 nt, Homo sapiens
ugagcccuguccucccgcag

SEQ ID NO: 146 - RNA, 22 nt, Homo sapiens
agugggaggccagggcacggca

SEQ ID NO: 147 - RNA, 22 nt, Homo sapiens
ucggccugaccacccaccccac

SEQ ID NO: 148 - RNA, 21 nt, Homo sapiens
ggggggggggggggggggccg

SEQ ID NO: 149 - RNA, 22 nt, Homo sapiens
ccucuuccccuugucucuccag

SEQ ID NO: 150 - RNA, 22 nt, Homo sapiens
ugagugacaggggaaaugggga

SEQ ID NO: 151 - RNA, 21 nt, Homo sapiens
uccuucugcuccgucccccag

SEQ ID NO: 152 - RNA, 21 nt, Homo sapiens
cgggggcggggccgaagcgcg

SEQ ID NO: 153 - RNA, 20 nt, Homo sapiens
cuuccucgucugucugcccc

SEQ ID NO: 154 - RNA, 23 nt, Homo sapiens
gugagugggagccccagugugug

SEQ ID NO: 155 - RNA, 20 nt, Homo sapiens
uaaggcacgcggugaaugcc

SEQ ID NO: 156 - RNA, 22 nt, Homo sapiens
cguguucacagcggaccuugau

SEQ ID NO: 157 - RNA, 22 nt, Homo sapiens
aacuggaucaauuauaggagug

SEQ ID NO: 158 - RNA, 26 nt, Homo sapiens
aaguaguugguuuguaugagaugguu

SEQ ID NO: 159 - RNA, 21 nt, Homo sapiens
aagugaucuaaaggccuacau

SEQ ID NO: 160 - RNA, 22 nt, Homo sapiens
ucagaugaucuaaaggccuaua

SEQ ID NO: 161 - RNA, 21 nt, Homo sapiens
uaggccuuuagaucacuuaaa

SEQ ID NO: 162 - RNA, 19 nt, Homo sapiens
aauggauuuuuggagcagg

SEQ ID NO: 163 - RNA, 24 nt, Homo sapiens
ccccgggaacgucgagacuggagc

SEQ ID NO: 164 - RNA, 22 nt, Homo sapiens
acccgucccguucguccccgga

SEQ ID NO: 165 - RNA, 27 nt, Homo sapiens
accuucuuguauaagcacugugcuaaa

SEQ ID NO: 166 - RNA, 22 nt, Homo sapiens
acgcccuucccccccuucuuca

SEQ ID NO: 167 - RNA, 22 nt, Homo sapiens
acgcccuucccccccuucuuca

SEQ ID NO: 168 - RNA, 24 nt, Homo sapiens
aggagggaggagaugggccaaguu

SEQ ID NO: 169 - RNA, 21 nt, Homo sapiens
acggugcuggauguggccuuu

SEQ ID NO: 170 - RNA, 19 nt, Homo sapiens
acauuuuccagcccauuca

SEQ ID NO: 171 - RNA, 21 nt, Homo sapiens
acggugcuggauguggccuuu

SEQ ID NO: 172 - RNA, 21 nt, Homo sapiens
acucuagcugccaaaggcgcu

SEQ ID NO: 173 - RNA, 21 nt, Homo sapiens
cgcuuugcucagccaguguag

SEQ ID NO: 174 - RNA, 21 nt, Homo sapiens
acucuagcugccaaaggcgcu

SEQ ID NO: 175 - RNA, 22 nt, Homo sapiens
agaaggaaauugaauucauuua

SEQ ID NO: 176 - RNA, 22 nt, Homo sapiens
caaaugagcuuaauuuccuuuu

SEQ ID NO: 177 - RNA, 22 nt, Homo sapiens
agaaggaaauugaauucauuua

SEQ ID NO: 178 - RNA, 21 nt, Homo sapiens
agagaagaagaucagccugca

SEQ ID NO: 179 - RNA, 24 nt, Homo sapiens
agccuggaagcuggagccugcagu

SEQ ID NO: 180 - RNA, 23 nt, Homo sapiens
aggaugagcaaagaaaguagauu

SEQ ID NO: 181 - RNA, 22 nt, Homo sapiens
aaccacuuucuuugcucaucca

SEQ ID NO: 182 - RNA, 22 nt, Homo sapiens
cggaugagcaaagaaagugguu

SEQ ID NO: 183 - RNA, 22 nt, Homo sapiens
aggcauugacuucucacuagcu

SEQ ID NO: 184 - RNA, 21 nt, Homo sapiens
agugaaugauggguucugacc

SEQ ID NO: 185 - RNA, 21 nt, Homo sapiens
aguuaggauuaggucguggaa

SEQ ID NO: 186 - RNA, 22 nt, Homo sapiens
acaggugagguucuugggagcc

SEQ ID NO: 187 - RNA, 24 nt, Homo sapiens
ucccugagacccuuuaaccuguga

SEQ ID NO: 188 - RNA, 22 nt, Homo sapiens
acggguuaggcucuugggagcu

SEQ ID NO: 189 - RNA, 22 nt, Homo sapiens
ucacaagucaggcucuugggac

SEQ ID NO: 190 - RNA, 22 nt, Homo sapiens
ucccugagacccuaacuuguga

SEQ ID NO: 191 - RNA, 22 nt, Homo sapiens
ucguaccgugaguaauaaugcg

SEQ ID NO: 192 - RNA, 21 nt, Homo sapiens
cauuauuacuuuugguacgcg

SEQ ID NO: 193 - RNA, 18 nt, Homo sapiens
aucccaccucugccacca

SEQ ID NO: 194 - RNA, 19 nt, Homo sapiens
aucccaccacugccaccau

SEQ ID NO: 195 - RNA, 19 nt, Homo sapiens
auggauaaggcuuuggcuu

SEQ ID NO: 196 - RNA, 22 nt, Homo sapiens
augggugaauuuguagaaggau

SEQ ID NO: 197 - RNA, 22 nt, Homo sapiens
augguacccuggcauacugagu

SEQ ID NO: 198 - RNA, 23 nt, Homo sapiens
caagucuuauuugagcaccuguu

SEQ ID NO: 199 - RNA, 22 nt, Homo sapiens
caggauguggucaaguguuguu

SEQ ID NO: 200 - RNA, 23 nt, Homo sapiens
ccucagggcuguagaacagggcu

SEQ ID NO: 201 - RNA, 22 nt, Homo sapiens
cccuguucuaugcccugaggga

SEQ ID NO: 202 - RNA, 23 nt, Homo sapiens
ccucagggcuguagaacagggcu

SEQ ID NO: 203 - RNA, 21 nt, Homo sapiens
ccuguugaaguguaaucccca

SEQ ID NO: 204 - RNA, 18 nt, Homo sapiens
cgggcguggugguggggg

SEQ ID NO: 205 - RNA, 20 nt, Homo sapiens
cgggcgugguggugggggug

SEQ ID NO: 206 - RNA, 22 nt, Homo sapiens
cuggacugagccgugcuacugg

SEQ ID NO: 207 - RNA, 22 nt, Homo sapiens
cuggacugagccaugcuacugg

SEQ ID NO: 208 - RNA, 22 nt, Homo sapiens
ucggauccgucugagcuuggcu

SEQ ID NO: 209 - RNA, 22 nt, Homo sapiens
cugaagcucagagggcucugau

SEQ ID NO: 210 - RNA, 23 nt, Homo sapiens
cuggagauauggaagagcugugu

SEQ ID NO: 211 - RNA, 22 nt, Homo sapiens
agugccugcuaugugccaggca

SEQ ID NO: 212 - RNA, 22 nt, Homo sapiens
cuuggcaccuagcaagcacuca

SEQ ID NO: 213 - RNA, 26 nt, Homo sapiens
gaugaugauggcagcaaauucugaaa

SEQ ID NO: 214 - RNA, 25 nt, Homo sapiens
gggcgacaaagcaagacucuuucuu

SEQ ID NO: 215 - RNA, 22 nt, Homo sapiens
ggcgacaaaacgagacccuguc

SEQ ID NO: 216 - RNA, 25 nt, Homo sapiens
gaacccaugagguugaggcugcagu

SEQ ID NO: 217 - RNA, 22 nt, Homo sapiens
uugcuugaacccaggaagugga

SEQ ID NO: 218 - RNA, 19 nt, Homo sapiens
ggagauggagguugcagug

SEQ ID NO: 219 - RNA, 21 nt, Homo sapiens
accacugcacuccagccugag

SEQ ID NO: 220 - RNA, 22 nt, Homo sapiens
ggugguugaggcugcaguaagu

SEQ ID NO: 221 - RNA, 22 nt, Homo sapiens
cugcagacucgaccucccaggc

SEQ ID NO: 222 - RNA, 21 nt, Homo sapiens
cugggaggucaaggcugcagu

SEQ ID NO: 223 - RNA, 17 nt, Homo sapiens
gugggggagaggcuguc

SEQ ID NO: 224 - RNA, 20 nt, Homo sapiens
uaaagagcccuguggagaca

SEQ ID NO: 225 - RNA, 22 nt, Homo sapiens
uacguagauauauauguauuuu

SEQ ID NO: 226 - RNA, 24 nt, Homo sapiens
aaauauauauauauauguacguau

SEQ ID NO: 227 - RNA, 22 nt, Homo sapiens
uaguacugugcauaucaucuau

SEQ ID NO: 228 - RNA, 17 nt, Homo sapiens
ucauauugcuucuuucu

SEQ ID NO: 229 - RNA, 21 nt, Homo sapiens
ucacagugaaccggucucuuu

SEQ ID NO: 230 - RNA, 23 nt, Homo sapiens
cggggccguagcacugucugaga

SEQ ID NO: 231 - RNA, 23 nt, Homo sapiens
gggggccgauacacuguacgaga

SEQ ID NO: 232 - RNA, 21 nt, Homo sapiens
ucacagugaaccggucucuuu

SEQ ID NO: 233 - RNA, 17 nt, Homo sapiens
ucgccuccuccucuccc

SEQ ID NO: 234 - RNA, 20 nt, Homo sapiens
ucguuugccuuuuucugcuu

SEQ ID NO: 235 - RNA, 22 nt, Homo sapiens
ucuacaaaggaaagcgcuuucu

SEQ ID NO: 236 - RNA, 22 nt, Homo sapiens
ucuauacagacccuggcuuuuc

SEQ ID NO: 237 - RNA, 22 nt, Homo sapiens
ucugggcaacaaagugagaccu

SEQ ID NO: 238 - RNA, 21 nt, Homo sapiens
gaucucacuuuguugcccagg

SEQ ID NO: 239 - RNA, 21 nt, Homo sapiens
ugcaggaccaagaugagcccu

SEQ ID NO: 240 - RNA, 22 nt, Homo sapiens
ugcuggaucagugguucgaguc

SEQ ID NO: 241 - RNA, 22 nt, Homo sapiens
cucuagccacagaugcagugau

SEQ ID NO: 242 - RNA, 22 nt, Homo sapiens
ugcuggaucagugguucgaguc

SEQ ID NO: 243 - RNA, 21 nt, Homo sapiens
uggacugcccugaucuggaga

SEQ ID NO: 244 - RNA, 21 nt, Homo sapiens
uggacugcccugaucuggaga

SEQ ID NO: 245 - RNA, 23 nt, Homo sapiens
gcagaucaggacuguaacucacc

SEQ ID NO: 246 - RNA, 23 nt, Homo sapiens
uggaguccaggaaucugcauuuu

SEQ ID NO: 247 - RNA, 22 nt, Homo sapiens
aagcccuuaccccaaaaaguau

SEQ ID NO: 248 - RNA, 22 nt, Homo sapiens
aagcccuuaccccaaaaagcau

SEQ ID NO: 249 - RNA, 21 nt, Homo sapiens
cuuuuugcggucugggcuugc

SEQ ID NO: 250 - RNA, 19 nt, Homo sapiens
uggauuuuuggaucaggga

SEQ ID NO: 251 - RNA, 24 nt, Homo sapiens
uggcccugacugaagaccagcagu

SEQ ID NO: 252 - RNA, 20 nt, Homo sapiens
ucgcgccccggcucccguuc

SEQ ID NO: 253 - RNA, 25 nt, Homo sapiens
ugggaacggguuccggcagacgcug

SEQ ID NO: 254 - RNA, 22 nt, Homo sapiens
uggguggucuggagauuugugc

SEQ ID NO: 255 - RNA, 22 nt, Homo sapiens
ugugagguuggcauuguugucu

SEQ ID NO: 256 - RNA, 21 nt, Homo sapiens
uuaggccgcagaucuggguga

SEQ ID NO: 257 - RNA, 22 nt, Homo sapiens
aauaggccacggaucugggcaa

SEQ ID NO: 258 - RNA, 21 nt, Homo sapiens
cacccagaucugcggccuaau

SEQ ID NO: 259 - RNA, 22 nt, Homo sapiens
uuagggcccuggcuccaucucc

SEQ ID NO: 260 - RNA, 22 nt, Homo sapiens
gaguggggcuucgacccuaacc

SEQ ID NO: 261 - RNA, 22 nt, Homo sapiens
uuagggcccuggcuccaucucc

SEQ ID NO: 262 - RNA, 17 nt, Homo sapiens
uucaaguaauucaggug

SEQ ID NO: 263 - RNA, 22 nt, Homo sapiens
uucauucggcuguccagaugua

SEQ ID NO: 264 - RNA, 21 nt, Homo sapiens
caucugggcaacugacugaac

SEQ ID NO: 265 - RNA, 22 nt, Homo sapiens
uucauucggcuguccagaugua

SEQ ID NO: 266 - RNA, 22 nt, Homo sapiens
uucuggaauucugugugaggga

SEQ ID NO: 267 - RNA, 24 nt, Homo sapiens
uugcagcugccugggagugacuuc

SEQ ID NO: 268 - RNA, 24 nt, Homo sapiens
uugcagcugccugggagugacuuc

SEQ ID NO: 269 - RNA, 19 nt, Homo sapiens
cgcucuaggcaccgcagca

SEQ ID NO: 270 - RNA, 21 nt, Homo sapiens
uugggacauacuuaugcuaaa

SEQ ID NO: 271 - RNA, 22 nt, Homo sapiens
uuuagagacggggucuugcucu

SEQ ID NO: 272 - RNA, 22 nt, Homo sapiens
ucucacuguagccucgaacccc

SEQ ID NO: 273 - RNA, 22 nt, Homo sapiens
uuugaggcuacagugagaugug

SEQ ID NO: 274 - RNA, 22 nt, Homo sapiens
uuuucaacucuaaugggagaga

SEQ ID NO: 275 - RNA, 18 nt, Homo sapiens
acguuggcucugguggug

SEQ ID NO: 276 - RNA, 22 nt, Homo sapiens
ccaccuccccugcaaacgucca

SEQ ID NO: 277 - RNA, 22 nt, Homo sapiens
acucggcguggcgucggucgug

SEQ ID NO: 278 - RNA, 21 nt, Homo sapiens
ucgaccggaccucgaccggcu

SEQ ID NO: 279 - RNA, 22 nt, Homo sapiens
cagugcaauguuaaaagggcau

SEQ ID NO: 280 - RNA, 22 nt, Homo sapiens
uucacauugugcuacugucugc

SEQ ID NO: 281 - RNA, 22 nt, Homo sapiens
cagugcaaugaugaaagggcau

SEQ ID NO: 282 - RNA, 21 nt, Homo sapiens
acucuuucccuguugcacuac

SEQ ID NO: 283 - RNA, 22 nt, Homo sapiens
uaacagucuacagccauggucg

SEQ ID NO: 284 - RNA, 22 nt, Homo sapiens
accguggcuuucgauuguuacu

SEQ ID NO: 285 - RNA, 18 nt, Homo sapiens
cagggaggugaaugugau

SEQ ID NO: 286 - RNA, 19 nt, Homo sapiens
gaugaugcugcugaugcug

SEQ ID NO: 287 - RNA, 22 nt, Homo sapiens
ucaaaacugaggggcauuuucu

SEQ ID NO: 288 - RNA, 24 nt, Homo sapiens
ccagacagaauucuaugcacuuuc

SEQ ID NO: 289 - RNA, 22 nt, Homo sapiens
uuugguccccuucaaccagcug

SEQ ID NO: 290 - RNA, 22 nt, Homo sapiens
uuugguccccuucaaccagcug

SEQ ID NO: 291 - RNA, 22 nt, Homo sapiens
agcugguaaaauggaaccaaau

SEQ ID NO: 292 - RNA, 22 nt, Homo sapiens
uuugguccccuucaaccagcua

SEQ ID NO: 293 - RNA, 22 nt, Homo sapiens
ugugacugguugaccagagggg

SEQ ID NO: 294 - RNA, 23 nt, Homo sapiens
ccugugggccaccuagucaccaa

SEQ ID NO: 295 - RNA, 22 nt, Homo sapiens
ugugacugguugaccagagggg

SEQ ID NO: 296 - RNA, 22 nt, Homo sapiens
cuccuggggcccgcacucucgc

SEQ ID NO: 297 - RNA, 22 nt, Homo sapiens
cuccuggggcccgcacucucgc

SEQ ID NO: 298 - RNA, 22 nt, Homo sapiens
uggggagcggcccccggguggg

SEQ ID NO: 299 - RNA, 22 nt, Homo sapiens
uauagggauuggagccguggcg

SEQ ID NO: 300 - RNA, 23 nt, Homo sapiens
uauggcuuuuuauuccuauguga

SEQ ID NO: 301 - RNA, 22 nt, Homo sapiens
auguagggcuaaaagccauggg

SEQ ID NO: 302 - RNA, 23 nt, Homo sapiens
uauggcuuuucauuccuauguga

SEQ ID NO: 303 - RNA, 22 nt, Homo sapiens
caucaucgucucaaaugagucu

SEQ ID NO: 304 - RNA, 23 nt, Homo sapiens
acuccauuuguuuugaugaugga

SEQ ID NO: 305 - RNA, 23 nt, Homo sapiens
uuauugcuuaagaauacgcguag

SEQ ID NO: 306 - RNA, 22 nt, Homo sapiens
gcuacuucacaacaccagggcc

SEQ ID NO: 307 - RNA, 22 nt, Homo sapiens
gcuauuucacgacaccaggguu

SEQ ID NO: 308 - RNA, 23 nt, Homo sapiens
agcugguguugugaaucaggccg

SEQ ID NO: 309 - RNA, 23 nt, Homo sapiens
uggagacgcggcccuguuggagu

SEQ ID NO: 310 - RNA, 23 nt, Homo sapiens
ucuacagugcacgugucuccagu

SEQ ID NO: 311 - RNA, 21 nt, Homo sapiens
uaccacaggguagaaccacgg

SEQ ID NO: 312 - RNA, 22 nt, Homo sapiens
cagugguuuuacccuaugguag

SEQ ID NO: 313 - RNA, 22 nt, Homo sapiens
uaacacugucugguaaagaugg

SEQ ID NO: 314 - RNA, 22 nt, Homo sapiens
caucuuccaguacaguguugga

SEQ ID NO: 315 - RNA, 23 nt, Homo sapiens
uguaguguuuccuacuuuaugga

SEQ ID NO: 316 - RNA, 21 nt, Homo sapiens
cauaaaguagaaagcacuacu

SEQ ID NO: 317 - RNA, 21 nt, Homo sapiens
ugagaugaagcacuguagcuc

SEQ ID NO: 318 - RNA, 22 nt, Homo sapiens
ggugcagugcugcaucucuggu

SEQ ID NO: 319 - RNA, 20 nt, Homo sapiens
uacaguauagaugauguacu

SEQ ID NO: 320 - RNA, 22 nt, Homo sapiens
ggauaucaucauauacuguaag

SEQ ID NO: 321 - RNA, 22 nt, Homo sapiens
ggauuccuggaaauacuguucu

SEQ ID NO: 322 - RNA, 23 nt, Homo sapiens
guccaguuuucccaggaaucccu

SEQ ID NO: 323 - RNA, 21 nt, Homo sapiens
cuccguuugccuguuucgcug

SEQ ID NO: 324 - RNA, 22 nt, Homo sapiens
agcaaaauaagcaaauggaaaa

SEQ ID NO: 325 - RNA, 21 nt, Homo sapiens
cuccguuugccuguuucgcug

SEQ ID NO: 326 - RNA, 22 nt, Homo sapiens
cucggcgcggggcgcgggcucc

SEQ ID NO: 327 - RNA, 22 nt, Homo sapiens
ccucugaaauucaguucuucag

SEQ ID NO: 328 - RNA, 22 nt, Homo sapiens
ugagaacugaauuccauggguu

SEQ ID NO: 329 - RNA, 22 nt, Homo sapiens
ugcccuguggacucaguucugg

SEQ ID NO: 330 - RNA, 22 nt, Homo sapiens
ugagaacugaauuccauaggcu

SEQ ID NO: 331 - RNA, 21 nt, Homo sapiens
gcccuccgcccgugcaccccg

SEQ ID NO: 332 - RNA, 22 nt, Homo sapiens
gcccgcguguggagccaggugu

SEQ ID NO: 333 - RNA, 20 nt, Homo sapiens
guguguggaaaugcuucugc

SEQ ID NO: 334 - RNA, 22 nt, Homo sapiens
gugugcggaaaugcuucugcua

SEQ ID NO: 335 - RNA, 22 nt, Homo sapiens
ucagugcacuacagaacuuugu

SEQ ID NO: 336 - RNA, 22 nt, Homo sapiens
aaaguucugagacacuccgacu

SEQ ID NO: 337 - RNA, 22 nt, Homo sapiens
ucagugcaucacagaacuuugu

SEQ ID NO: 338 - RNA, 22 nt, Homo sapiens
aaguucuguuauacacucaggc

SEQ ID NO: 339 - RNA, 21 nt, Homo sapiens
agggagggacgggggcugugc

SEQ ID NO: 340 - RNA, 23 nt, Homo sapiens
ucuggcuccgugucuucacuccc

SEQ ID NO: 341 - RNA, 22 nt, Homo sapiens
cugguacaggccugggggacag

SEQ ID NO: 342 - RNA, 22 nt, Homo sapiens
ucucccaacccuuguaccagug

SEQ ID NO: 343 - RNA, 21 nt, Homo sapiens
cuagacugaagcuccuugagg

SEQ ID NO: 344 - RNA, 21 nt, Homo sapiens
ucgaggagcucacagucuagu

SEQ ID NO: 345 - RNA, 18 nt, Homo sapiens
ucgaggagcucacagucu

SEQ ID NO: 346 - RNA, 21 nt, Homo sapiens
ucagugcaugacagaacuugg

SEQ ID NO: 347 - RNA, 21 nt, Homo sapiens
ucagugcaugacagaacuugg

SEQ ID NO: 348 - RNA, 23 nt, Homo sapiens
agguucugugauacacuccgacu

SEQ ID NO: 349 - RNA, 22 nt, Homo sapiens
uugcauagucacaaaagugauc

SEQ ID NO: 350 - RNA, 22 nt, Homo sapiens
uugcauagucacaaaagugauc

SEQ ID NO: 351 - RNA, 22 nt, Homo sapiens
ucauuuuugugauguugcagcu

SEQ ID NO: 352 - RNA, 22 nt, Homo sapiens
aaaaccgucuaguuacaguugu

SEQ ID NO: 353 - RNA, 22 nt, Homo sapiens
aaaaccgucuaguuacaguugu

SEQ ID NO: 354 - RNA, 22 nt, Homo sapiens
agcuguaauuagucaguuuucu

SEQ ID NO: 355 - RNA, 23 nt, Homo sapiens
cggcccgggcugcugcuguuccu

SEQ ID NO: 356 - RNA, 21 nt, Homo sapiens
uccugcgcgucccagaugccc

SEQ ID NO: 357 - RNA, 22 nt, Homo sapiens
aaucauacacgguugaccuauu

SEQ ID NO: 358 - RNA, 22 nt, Homo sapiens
uagguuauccguguugccuucg

SEQ ID NO: 359 - RNA, 22 nt, Homo sapiens
cuccuacauauuagcauuaaca

SEQ ID NO: 360 - RNA, 23 nt, Homo sapiens
uuaaugcuaaucgugauaggggu

SEQ ID NO: 361 - RNA, 20 nt, Homo sapiens
uugggcugggcuggguuggg

SEQ ID NO: 362 - RNA, 22 nt, Homo sapiens
caggccauauugugcugccuca

SEQ ID NO: 363 - RNA, 22 nt, Homo sapiens
uagcagcacauaaugguuugug

SEQ ID NO: 364 - RNA, 22 nt, Homo sapiens
cgaaucauuauuugcugcucua

SEQ ID NO: 365 - RNA, 22 nt, Homo sapiens
uagcagcacaucaugguuuaca

SEQ ID NO: 366 - RNA, 22 nt, Homo sapiens
ccaguauuaacugugcugcuga

SEQ ID NO: 367 - RNA, 22 nt, Homo sapiens
ccaauauuacugugcugcuuua

SEQ ID NO: 368 - RNA, 22 nt, Homo sapiens
uagcagcacguaaauauuggcg

SEQ ID NO: 369 - RNA, 22 nt, Homo sapiens
acugcagugaaggcacuuguag

SEQ ID NO: 370 - RNA, 23 nt, Homo sapiens
caaagugcuuacagugcagguag

SEQ ID NO: 371 - RNA, 22 nt, Homo sapiens
accacugaccguugacuguacc

SEQ ID NO: 372 - RNA, 22 nt, Homo sapiens
accaucgaccguugauuguacc

SEQ ID NO: 373 - RNA, 23 nt, Homo sapiens
aacauucaacgcugucggugagu

SEQ ID NO: 374 - RNA, 20 nt, Homo sapiens
cucacugaucaaugaaugca

SEQ ID NO: 375 - RNA, 21 nt, Homo sapiens
cucacugaacaaugaaugcaa

SEQ ID NO: 376 - RNA, 23 nt, Homo sapiens
aacauucauugcugucggugggu

SEQ ID NO: 377 - RNA, 22 nt, Homo sapiens
aaccaucgaccguugaguggac

SEQ ID NO: 378 - RNA, 22 nt, Homo sapiens
aacauucaaccugucggugagu

SEQ ID NO: 379 - RNA, 23 nt, Homo sapiens
aacauucauuguugucggugggu

SEQ ID NO: 380 - RNA, 21 nt, Homo sapiens
ccaccgggggaugaaugucac

SEQ ID NO: 381 - RNA, 23 nt, Homo sapiens
aacauucauuguugucggugggu

SEQ ID NO: 382 - RNA, 21 nt, Homo sapiens
ugguucuagacuugccaacua

SEQ ID NO: 383 - RNA, 24 nt, Homo sapiens
uuuggcaaugguagaacucacacu

SEQ ID NO: 384 - RNA, 18 nt, Homo sapiens
uccagugcccuccucucc

SEQ ID NO: 385 - RNA, 18 nt, Homo sapiens
ugaggcaguagauugaau

SEQ ID NO: 386 - RNA, 22 nt, Homo sapiens
gugaauuaccgaagggccauaa

SEQ ID NO: 387 - RNA, 22 nt, Homo sapiens
uauggcacugguagaauucacu

SEQ ID NO: 388 - RNA, 22 nt, Homo sapiens
uggacggagaacugauaagggu

SEQ ID NO: 389 - RNA, 22 nt, Homo sapiens
aggggcuggcuuuccucugguc

SEQ ID NO: 390 - RNA, 22 nt, Homo sapiens
uggagagaaaggcaguuccuga

SEQ ID NO: 391 - RNA, 22 nt, Homo sapiens
gcccaaaggugaauuuuuuggg

SEQ ID NO: 392 - RNA, 22 nt, Homo sapiens
caaagaauucuccuuuugggcu

SEQ ID NO: 393 - RNA, 22 nt, Homo sapiens
ucgugucuuguguugcagccgg

SEQ ID NO: 394 - RNA, 22 nt, Homo sapiens
ggcuacaacacaggacccgggc

SEQ ID NO: 395 - RNA, 21 nt, Homo sapiens
cucccacaugcaggguuugca

SEQ ID NO: 396 - RNA, 21 nt, Homo sapiens
caucccuugcaugguggaggg

SEQ ID NO: 397 - RNA, 23 nt, Homo sapiens
acugcccuaagugcuccuucugg

SEQ ID NO: 398 - RNA, 23 nt, Homo sapiens
uaaggugcaucuagugcagauag

SEQ ID NO: 399 - RNA, 22 nt, Homo sapiens
ugcccuaaaugccccuucuggc

SEQ ID NO: 400 - RNA, 23 nt, Homo sapiens
uaaggugcaucuagugcaguuag

SEQ ID NO: 401 - RNA, 21 nt, Homo sapiens
cggcggggacggcgauugguc

SEQ ID NO: 402 - RNA, 21 nt, Homo sapiens
ccggccgccggcuccgccccg

SEQ ID NO: 403 - RNA, 21 nt, Homo sapiens
cggcggggacggcgauugguc

SEQ ID NO: 404 - RNA, 22 nt, Homo sapiens
cgcaggggccgggugcucaccg

SEQ ID NO: 405 - RNA, 21 nt, Homo sapiens
ugagugccggugccugcccug

SEQ ID NO: 406 - RNA, 22 nt, Homo sapiens
ugauauguuugauauauuaggu

SEQ ID NO: 407 - RNA, 21 nt, Homo sapiens
cuauauaucaaacauauuccu

SEQ ID NO: 408 - RNA, 22 nt, Homo sapiens
ugauauguuugauauauuaggu

SEQ ID NO: 409 - RNA, 21 nt, Homo sapiens
ugauauguuugauauuggguu

SEQ ID NO: 410 - RNA, 22 nt, Homo sapiens
gcugcgcuuggauuucgucccc

SEQ ID NO: 411 - RNA, 23 nt, Homo sapiens
caacggaaucccaaaagcagcug

SEQ ID NO: 412 - RNA, 21 nt, Homo sapiens
ccaguccugugccugccgccu

SEQ ID NO: 413 - RNA, 20 nt, Homo sapiens
gaggcagaagcaggaugaca

SEQ ID NO: 414 - RNA, 21 nt, Homo sapiens
ccaguccugugccugccgccu

SEQ ID NO: 415 - RNA, 20 nt, Homo sapiens
caccaggcauuguggucucc

SEQ ID NO: 416 - RNA, 23 nt, Homo sapiens
ugaguaccgccaugucuguuggg

SEQ ID NO: 417 - RNA, 22 nt, Homo sapiens
uacccagagcaugcagugugaa

SEQ ID NO: 418 - RNA, 22 nt, Homo sapiens
ucugcccccuccgcugcugcca

SEQ ID NO: 419 - RNA, 22 nt, Homo sapiens
ggaggggucccgcacugggagg

SEQ ID NO: 420 - RNA, 22 nt, Homo sapiens
cccugugcccggcccacuucug

SEQ ID NO: 421 - RNA, 20 nt, Homo sapiens
ccccagggcgacgcggcggg

SEQ ID NO: 422 - RNA, 22 nt, Homo sapiens
accuugccuugcugcccgggcc

SEQ ID NO: 423 - RNA, 22 nt, Homo sapiens
cugccaauuccauaggucacag

SEQ ID NO: 424 - RNA, 21 nt, Homo sapiens
cugaccuaugaauugacagcc

SEQ ID NO: 425 - RNA, 22 nt, Homo sapiens
aacuggccuacaaagucccagu

SEQ ID NO: 426 - RNA, 22 nt, Homo sapiens
ugggucuuugcgggcgagauga

SEQ ID NO: 427 - RNA, 22 nt, Homo sapiens
aacuggcccucaaagucccgcu

SEQ ID NO: 428 - RNA, 22 nt, Homo sapiens
cgggguuuugagggcgagauga

SEQ ID NO: 429 - RNA, 22 nt, Homo sapiens
ccaguggggcugcuguuaucug

SEQ ID NO: 430 - RNA, 22 nt, Homo sapiens
uguaacagcaacuccaugugga

SEQ ID NO: 431 - RNA, 22 nt, Homo sapiens
ccaauauuggcugugcugcucc

SEQ ID NO: 432 - RNA, 21 nt, Homo sapiens
uagcagcacagaaauauuggc

SEQ ID NO: 433 - RNA, 22 nt, Homo sapiens
cggcaacaagaaacugccugag

SEQ ID NO: 434 - RNA, 22 nt, Homo sapiens
uagguaguuucauguuguuggg

SEQ ID NO: 435 - RNA, 22 nt, Homo sapiens
ucgacagcacgacacugccuuc

SEQ ID NO: 436 - RNA, 22 nt, Homo sapiens
uagguaguuuccuguuguuggg

SEQ ID NO: 437 - RNA, 22 nt, Homo sapiens
uucaccaccuucuccacccagc

SEQ ID NO: 438 - RNA, 23 nt, Homo sapiens
cggguagagagggcagugggagg

SEQ ID NO: 439 - RNA, 22 nt, Homo sapiens
ucaggccaggcacaguggcuca

SEQ ID NO: 440 - RNA, 19 nt, Homo sapiens
accgugcaaagguagcaua

SEQ ID NO: 441 - RNA, 20 nt, Homo sapiens
ccuccugcccuccuugcugu

SEQ ID NO: 442 - RNA, 22 nt, Homo sapiens
gguccagaggggagauagguuc

SEQ ID NO: 443 - RNA, 22 nt, Homo sapiens
acaguagucugcacauugguua

SEQ ID NO: 444 - RNA, 23 nt, Homo sapiens
cccaguguucagacuaccuguuc

SEQ ID NO: 445 - RNA, 22 nt, Homo sapiens
acaguagucugcacauugguua

SEQ ID NO: 446 - RNA, 23 nt, Homo sapiens
cccaguguuuagacuaucuguuc

SEQ ID NO: 447 - RNA, 23 nt, Homo sapiens
ugugcaaaucuaugcaaaacuga

SEQ ID NO: 448 - RNA, 22 nt, Homo sapiens
aguuuugcauaguugcacuaca

SEQ ID NO: 449 - RNA, 23 nt, Homo sapiens
aguuuugcagguuugcauccagc

SEQ ID NO: 450 - RNA, 22 nt, Homo sapiens
aguuuugcagguuugcauuuca

SEQ ID NO: 451 - RNA, 23 nt, Homo sapiens
ugugcaaauccaugcaaaacuga

SEQ ID NO: 452 - RNA, 22 nt, Homo sapiens
uaacacugucugguaacgaugu

SEQ ID NO: 453 - RNA, 22 nt, Homo sapiens
caucuuaccggacagugcugga

SEQ ID NO: 454 - RNA, 22 nt, Homo sapiens
uaauacugccugguaaugauga

SEQ ID NO: 455 - RNA, 22 nt, Homo sapiens
caucuuacugggcagcauugga

SEQ ID NO: 456 - RNA, 23 nt, Homo sapiens
uaauacugccggguaaugaugga

SEQ ID NO: 457 - RNA, 22 nt, Homo sapiens
cgucuuacccagcaguguuugg

SEQ ID NO: 458 - RNA, 20 nt, Homo sapiens
agagguauagggcaugggaa

SEQ ID NO: 459 - RNA, 22 nt, Homo sapiens
uuccuaugcauauacuucuuug

SEQ ID NO: 460 - RNA, 22 nt, Homo sapiens
gugaaauguuuaggaccacuag

SEQ ID NO: 461 - RNA, 22 nt, Homo sapiens
gugaaauguuuaggaccacuag

SEQ ID NO: 462 - RNA, 25 nt, Homo sapiens
agugguucuuaacaguucaacaguu

SEQ ID NO: 463 - RNA, 23 nt, Homo sapiens
uugaacuguuaagaaccacugga

SEQ ID NO: 464 - RNA, 22 nt, Homo sapiens
uagugguccuaaacauuucaca

SEQ ID NO: 465 - RNA, 21 nt, Homo sapiens
gcugggaaggcaaagggacgu

SEQ ID NO: 466 - RNA, 22 nt, Homo sapiens
uucccuuugucauccuaugccu

SEQ ID NO: 467 - RNA, 21 nt, Homo sapiens
gauuucaguggagugaaguuc

SEQ ID NO: 468 - RNA, 22 nt, Homo sapiens
uccuucauuccaccggagucug

SEQ ID NO: 469 - RNA, 20 nt, Homo sapiens
uguuuugauaacaguaaugu

SEQ ID NO: 470 - RNA, 23 nt, Homo sapiens
guguuaauuaaaccucuauuuac

SEQ ID NO: 471 - RNA, 23 nt, Homo sapiens
cuguaauauaaauuuaauuuauu

SEQ ID NO: 472 - RNA, 22 nt, Homo sapiens
uggaauguaaggaagugugugg

SEQ ID NO: 473 - RNA, 22 nt, Homo sapiens
auaagacgagcaaaaagcuugu

SEQ ID NO: 474 - RNA, 22 nt, Homo sapiens
auaagacgagcaaaaagcuugu

SEQ ID NO: 475 - RNA, 22 nt, Homo sapiens
gagcuuuuggcccggguuauac

SEQ ID NO: 476 - RNA, 22 nt, Homo sapiens
auaagacgaacaaaagguuugu

SEQ ID NO: 477 - RNA, 22 nt, Homo sapiens
auaagacgaacaaaagguuugu

SEQ ID NO: 478 - RNA, 22 nt, Homo sapiens
aagcuuuuugcucgaauuaugu

SEQ ID NO: 479 - RNA, 22 nt, Homo sapiens
acugcauuaugagcacuuaaag

SEQ ID NO: 480 - RNA, 23 nt, Homo sapiens
uaaagugcuuauagugcagguag

SEQ ID NO: 481 - RNA, 22 nt, Homo sapiens
acuguaguaugggcacuuccag

SEQ ID NO: 482 - RNA, 23 nt, Homo sapiens
caaagugcucauagugcagguag

SEQ ID NO: 483 - RNA, 21 nt, Homo sapiens
caacaccagucgaugggcugu

SEQ ID NO: 484 - RNA, 22 nt, Homo sapiens
uagcuuaucagacugauguuga

SEQ ID NO: 485 - RNA, 22 nt, Homo sapiens
cugugcgugugacagcggcuga

SEQ ID NO: 486 - RNA, 22 nt, Homo sapiens
cugugcgugugacagcggcuga

SEQ ID NO: 487 - RNA, 22 nt, Homo sapiens
agccccugcccaccgcacacug

SEQ ID NO: 488 - RNA, 21 nt, Homo sapiens
gcagggacagcaaaggggugc

SEQ ID NO: 489 - RNA, 22 nt, Homo sapiens
uucccuuugucauccuucgccu

SEQ ID NO: 490 - RNA, 22 nt, Homo sapiens
uuggggaaacggccgcugagug

SEQ ID NO: 491 - RNA, 21 nt, Homo sapiens
auuugugcuuggcucugucac

SEQ ID NO: 492 - RNA, 21 nt, Homo sapiens
cgagccucaagcaagggacuu

SEQ ID NO: 493 - RNA, 22 nt, Homo sapiens
uagucccuuccuugaagcgguc

SEQ ID NO: 494 - RNA, 22 nt, Homo sapiens
caucagaauucauggaggcuag

SEQ ID NO: 495 - RNA, 22 nt, Homo sapiens
agcuuccaugacuccugaugga

SEQ ID NO: 496 - RNA, 21 nt, Homo sapiens
ccucccaugccaagaacuccc

SEQ ID NO: 497 - RNA, 21 nt, Homo sapiens
gguucuuagcauaggaggucu

SEQ ID NO: 498 - RNA, 21 nt, Homo sapiens
uguucucuuugccaaggacag

SEQ ID NO: 499 - RNA, 21 nt, Homo sapiens
uaacagucuccagucacggcc

SEQ ID NO: 500 - RNA, 23 nt, Homo sapiens
accuuggcucuagacugcuuacu

SEQ ID NO: 501 - RNA, 22 nt, Homo sapiens
acagcaggcacagacaggcagu

SEQ ID NO: 502 - RNA, 22 nt, Homo sapiens
ugccugucuacacuugcugugc

SEQ ID NO: 503 - RNA, 21 nt, Homo sapiens
augaccuaugaauugacagac

SEQ ID NO: 504 - RNA, 23 nt, Homo sapiens
ucugucauuucuuuaggccaaua

SEQ ID NO: 505 - RNA, 21 nt, Homo sapiens
augaccuaugaauugacagac

SEQ ID NO: 506 - RNA, 22 nt, Homo sapiens
ucacaguggucucugggauuau

SEQ ID NO: 507 - RNA, 22 nt, Homo sapiens
uaaucucagcuggcaacuguga

SEQ ID NO: 508 - RNA, 22 nt, Homo sapiens
aaaucucugcaggcaaauguga

SEQ ID NO: 509 - RNA, 24 nt, Homo sapiens
acacacuuacccguagagauucua

SEQ ID NO: 510 - RNA, 22 nt, Homo sapiens
aaaucucugcaggcaaauguga

SEQ ID NO: 511 - RNA, 23 nt, Homo sapiens
uacugcaucaggaacugauugga

SEQ ID NO: 512 - RNA, 22 nt, Homo sapiens
augguuccgucaagcaccaugg

SEQ ID NO: 513 - RNA, 22 nt, Homo sapiens
caugguucugucaagcaccgcg

SEQ ID NO: 514 - RNA, 21 nt, Homo sapiens
uugugcuugaucuaaccaugu

SEQ ID NO: 515 - RNA, 22 nt, Homo sapiens
agaguugagucuggacgucccg

SEQ ID NO: 516 - RNA, 22 nt, Homo sapiens
agaauuguggcuggacaucugu

SEQ ID NO: 517 - RNA, 21 nt, Homo sapiens
ugauuguccaaacgcaauucu

SEQ ID NO: 518 - RNA, 22 nt, Homo sapiens
agaguugagucuggacgucccg

SEQ ID NO: 519 - RNA, 22 nt, Homo sapiens
agaauuguggcuggacaucugu

SEQ ID NO: 520 - RNA, 21 nt, Homo sapiens
ugauuguccaaacgcaauucu

SEQ ID NO: 521 - RNA, 23 nt, Homo sapiens
agaauugcguuuggacaaucagu

SEQ ID NO: 522 - RNA, 22 nt, Homo sapiens
agauguccagccacaauucucg

SEQ ID NO: 523 - RNA, 22 nt, Homo sapiens
aagcugccaguugaagaacugu

SEQ ID NO: 524 - RNA, 22 nt, Homo sapiens
aguucuucaguggcaagcuuua

SEQ ID NO: 525 - RNA, 23 nt, Homo sapiens
agcuacauugucugcuggguuuc

SEQ ID NO: 526 - RNA, 22 nt, Homo sapiens
accuggcauacaauguagauuu

SEQ ID NO: 527 - RNA, 21 nt, Homo sapiens
agcuacaucuggcuacugggu

SEQ ID NO: 528 - RNA, 22 nt, Homo sapiens
cucaguagccaguguagauccu

SEQ ID NO: 529 - RNA, 22 nt, Homo sapiens
ugucaguuugucaaauacccca

SEQ ID NO: 530 - RNA, 22 nt, Homo sapiens
cguguauuugacaagcugaguu

SEQ ID NO: 531 - RNA, 23 nt, Homo sapiens
aaaauggugcccuagugacuaca

SEQ ID NO: 532 - RNA, 21 nt, Homo sapiens
caagucacuagugguuccguu

SEQ ID NO: 533 - RNA, 22 nt, Homo sapiens
ucugcaagugucagaggcgagg

SEQ ID NO: 534 - RNA, 22 nt, Homo sapiens
ucugcaagugucagaggcgagg

SEQ ID NO: 535 - RNA, 22 nt, Homo sapiens
gcccucugucaccuugcagacg

SEQ ID NO: 536 - RNA, 21 nt, Homo sapiens
ugacagcgcccugccuggcuc

SEQ ID NO: 537 - RNA, 24 nt, Homo sapiens
agcgcgggcugagcgcugccaguc

SEQ ID NO: 538 - RNA, 22 nt, Homo sapiens
gagagcaguguguguugccugg

SEQ ID NO: 539 - RNA, 22 nt, Homo sapiens
auuguccuugcuguuuggagau

SEQ ID NO: 540 - RNA, 21 nt, Homo sapiens
auccccagauacaauggacaa

SEQ ID NO: 541 - RNA, 20 nt, Homo sapiens
uaggaugggggugagaggug

SEQ ID NO: 542 - RNA, 21 nt, Homo sapiens
aucacauugccagggauuucc

SEQ ID NO: 543 - RNA, 22 nt, Homo sapiens
gggguuccuggggaugggauuu

SEQ ID NO: 544 - RNA, 21 nt, Homo sapiens
aucacauugccagggauuacc

SEQ ID NO: 545 - RNA, 22 nt, Homo sapiens
uggguuccuggcaugcugauuu

SEQ ID NO: 546 - RNA, 22 nt, Homo sapiens
aucacauugccagugauuaccc

SEQ ID NO: 547 - RNA, 22 nt, Homo sapiens
ugccuacugagcugauaucagu

SEQ ID NO: 548 - RNA, 22 nt, Homo sapiens
ugccuacugagcugaaacacag

SEQ ID NO: 549 - RNA, 22 nt, Homo sapiens
uggcucaguucagcaggaacag

SEQ ID NO: 550 - RNA, 22 nt, Homo sapiens
agcagaggcagagaggcucagg

SEQ ID NO: 551 - RNA, 23 nt, Homo sapiens
ugaggcucuguuagccuuggcuc

SEQ ID NO: 552 - RNA, 22 nt, Homo sapiens
cauugcacuugucucggucuga

SEQ ID NO: 553 - RNA, 21 nt, Homo sapiens
aggcggagacuugggcaauug

SEQ ID NO: 554 - RNA, 22 nt, Homo sapiens
uaucauggaguugguaaagcac

SEQ ID NO: 555 - RNA, 22 nt, Homo sapiens
guuuuaccaccuccaggagacu

SEQ ID NO: 556 - RNA, 22 nt, Homo sapiens
cgccucuucagcgcugucuucc

SEQ ID NO: 557 - RNA, 23 nt, Homo sapiens
caggcagugacuguucagacguc

SEQ ID NO: 558 - RNA, 22 nt, Homo sapiens
ccuauucuugguuacuugcacg

SEQ ID NO: 559 - RNA, 22 nt, Homo sapiens
ccuauucuugauuacuuguuuc

SEQ ID NO: 560 - RNA, 22 nt, Homo sapiens
uucaaguaauccaggauaggcu

SEQ ID NO: 561 - RNA, 22 nt, Homo sapiens
ccuguucuccauuacuuggcuc

SEQ ID NO: 562 - RNA, 21 nt, Homo sapiens
uucaaguaauucaggauaggu

SEQ ID NO: 563 - RNA, 21 nt, Homo sapiens
uucacaguggcuaaguuccgc

SEQ ID NO: 564 - RNA, 22 nt, Homo sapiens
agggcuuagcugcuugugagca

SEQ ID NO: 565 - RNA, 21 nt, Homo sapiens
uucacaguggcuaaguucugc

SEQ ID NO: 566 - RNA, 22 nt, Homo sapiens
agagcuuagcugauuggugaac

SEQ ID NO: 567 - RNA, 22 nt, Homo sapiens
cacuagauugugagcuccugga

SEQ ID NO: 568 - RNA, 22 nt, Homo sapiens
aaggagcucacagucuauugag

SEQ ID NO: 569 - RNA, 19 nt, Homo sapiens
ggggccuggcggugggcgg

SEQ ID NO: 570 - RNA, 21 nt, Homo sapiens
guuagggccaacaucucuugg

SEQ ID NO: 571 - RNA, 22 nt, Homo sapiens
gaggguuggguggaggcucucc

SEQ ID NO: 572 - RNA, 21 nt, Homo sapiens
agggcccccccucaauccugu

SEQ ID NO: 573 - RNA, 23 nt, Homo sapiens
agaauugcguuuggacaaucagu

SEQ ID NO: 574 - RNA, 22 nt, Homo sapiens
agauguccagccacaauucucg

SEQ ID NO: 575 - RNA, 21 nt, Homo sapiens
auguaugugugcaugugcaug

SEQ ID NO: 576 - RNA, 24 nt, Homo sapiens
agcagaagcagggagguucuccca

SEQ ID NO: 577 - RNA, 22 nt, Homo sapiens
uaugugggaugguaaaccgcuu

SEQ ID NO: 578 - RNA, 22 nt, Homo sapiens
ugguuuaccgucccacauacau

SEQ ID NO: 579 - RNA, 22 nt, Homo sapiens
uagcaccaucugaaaucgguua

SEQ ID NO: 580 - RNA, 22 nt, Homo sapiens
acugauuucuuuugguguucag

SEQ ID NO: 581 - RNA, 24 nt, Homo sapiens
gcugguuucauauggugguuuaga

SEQ ID NO: 582 - RNA, 22 nt, Homo sapiens
cugguuucacaugguggcuuag

SEQ ID NO: 583 - RNA, 23 nt, Homo sapiens
uagcaccauuugaaaucaguguu

SEQ ID NO: 584 - RNA, 22 nt, Homo sapiens
uagcaccauuugaaaucgguua

SEQ ID NO: 585 - RNA, 22 nt, Homo sapiens
ugaccgauuucuccugguguuc

SEQ ID NO: 586 - RNA, 22 nt, Homo sapiens
uauacaagggcagacucucucu

SEQ ID NO: 587 - RNA, 23 nt, Homo sapiens
cagugcaauaguauugucaaagc

SEQ ID NO: 588 - RNA, 22 nt, Homo sapiens
gcucugacuuuauugcacuacu

SEQ ID NO: 589 - RNA, 23 nt, Homo sapiens
cagugcaaugauauugucaaagc

SEQ ID NO: 590 - RNA, 23 nt, Homo sapiens
cagugcaaugauauugucaaagc

SEQ ID NO: 591 - RNA, 22 nt, Homo sapiens
gcucugacgagguugcacuacu

SEQ ID NO: 592 - RNA, 23 nt, Homo sapiens
uaagugcuuccauguuuugguga

SEQ ID NO: 593 - RNA, 23 nt, Homo sapiens
acuuaaacguggauguacuugcu

SEQ ID NO: 594 - RNA, 23 nt, Homo sapiens
uaagugcuuccauguuuuaguag

SEQ ID NO: 595 - RNA, 22 nt, Homo sapiens
acuuuaacauggaagugcuuuc

SEQ ID NO: 596 - RNA, 23 nt, Homo sapiens
uaagugcuuccauguuucagugg

SEQ ID NO: 597 - RNA, 22 nt, Homo sapiens
uuuaacauggggguaccugcug

SEQ ID NO: 598 - RNA, 23 nt, Homo sapiens
uaagugcuuccauguuugagugu

SEQ ID NO: 599 - RNA, 22 nt, Homo sapiens
acuuuaacauggaggcacuugc

SEQ ID NO: 600 - RNA, 17 nt, Homo sapiens
uaagugcuuccaugcuu

SEQ ID NO: 601 - RNA, 17 nt, Homo sapiens
uaauugcuuccauguuu

SEQ ID NO: 602 - RNA, 23 nt, Homo sapiens
uugccacacugcaacaccuuaca

SEQ ID NO: 603 - RNA, 21 nt, Homo sapiens
ucuggcuguuguggugugcaa

SEQ ID NO: 604 - RNA, 23 nt, Homo sapiens
ucagcaccaggauauuguuggag

SEQ ID NO: 605 - RNA, 23 nt, Homo sapiens
ucaacaaaaucacugaugcugga

SEQ ID NO: 606 - RNA, 22 nt, Homo sapiens
gauaucagcucaguaggcaccg

SEQ ID NO: 607 - RNA, 21 nt, Homo sapiens
guuccugcugaacugagccag

SEQ ID NO: 608 - RNA, 22 nt, Homo sapiens
cuuucagucggauguuugcagc

SEQ ID NO: 609 - RNA, 22 nt, Homo sapiens
uguaaacauccucgacuggaag

SEQ ID NO: 610 - RNA, 22 nt, Homo sapiens
cugggagguggauguuuacuuc

SEQ ID NO: 611 - RNA, 22 nt, Homo sapiens
uguaaacauccuacacucagcu

SEQ ID NO: 612 - RNA, 22 nt, Homo sapiens
cugggagaggguuguuuacucc

SEQ ID NO: 613 - RNA, 22 nt, Homo sapiens
cugggagaaggcuguuuacucu

SEQ ID NO: 614 - RNA, 23 nt, Homo sapiens
uguaaacauccuacacucucagc

SEQ ID NO: 615 - RNA, 22 nt, Homo sapiens
cuuucagucagauguuugcugc

SEQ ID NO: 616 - RNA, 22 nt, Homo sapiens
uguaaacauccccgacuggaag

SEQ ID NO: 617 - RNA, 22 nt, Homo sapiens
cuuucagucggauguuuacagc

SEQ ID NO: 618 - RNA, 22 nt, Homo sapiens
uguaaacauccuugacuggaag

SEQ ID NO: 619 - RNA, 22 nt, Homo sapiens
ugcuaugccaacauauugccau

SEQ ID NO: 620 - RNA, 21 nt, Homo sapiens
aggcaagaugcuggcauagcu

SEQ ID NO: 621 - RNA, 20 nt, Homo sapiens
auauggguuuacuaguuggu

SEQ ID NO: 622 - RNA, 22 nt, Homo sapiens
ugccuggaacauaguagggacu

SEQ ID NO: 623 - RNA, 21 nt, Homo sapiens
auaggacucauauagugccag

SEQ ID NO: 624 - RNA, 20 nt, Homo sapiens
agacacuauacgagucauau

SEQ ID NO: 625 - RNA, 23 nt, Homo sapiens
ugugacugcauuaugaaaauucu

SEQ ID NO: 626 - RNA, 20 nt, Homo sapiens
uggcuuuuaacuuugauggc

SEQ ID NO: 627 - RNA, 21 nt, Homo sapiens
cacagcaaguguagacaggca

SEQ ID NO: 628 - RNA, 21 nt, Homo sapiens
ccugucugugccugcuguaca

SEQ ID NO: 629 - RNA, 22 nt, Homo sapiens
uaaauagaguaggcaaaggaca

SEQ ID NO: 630 - RNA, 22 nt, Homo sapiens
uccuuugccuauucuauuuaag

SEQ ID NO: 631 - RNA, 22 nt, Homo sapiens
guugggacaagaggacggucuu

SEQ ID NO: 632 - RNA, 17 nt, Homo sapiens
cagagaauuguuuaauc

SEQ ID NO: 633 - RNA, 22 nt, Homo sapiens
acuuuccucacucccgugaagu

SEQ ID NO: 634 - RNA, 21 nt, Homo sapiens
uucgcgggcgaaggcaaaguc

SEQ ID NO: 635 - RNA, 20 nt, Homo sapiens
uagaggaagcuguggagaga

SEQ ID NO: 636 - RNA, 22 nt, Homo sapiens
caucuggcauccgucacacaga

SEQ ID NO: 637 - RNA, 22 nt, Homo sapiens
ugagggacagaugccagaagca

SEQ ID NO: 638 - RNA, 22 nt, Homo sapiens
uccccuucugcaggccugcugg

SEQ ID NO: 639 - RNA, 23 nt, Homo sapiens
aucagggcuuguggaaugggaag

SEQ ID NO: 640 - RNA, 23 nt, Homo sapiens
ucuggcaaguaaaaaacucucau

SEQ ID NO: 641 - RNA, 22 nt, Homo sapiens
aaacuaaucucuacacugcugc

SEQ ID NO: 642 - RNA, 22 nt, Homo sapiens
gcaguaguguagagauugguuu

SEQ ID NO: 643 - RNA, 21 nt, Homo sapiens
gcugcaccggagacuggguaa

SEQ ID NO: 644 - RNA, 21 nt, Homo sapiens
uacccagucuccggugcagcc

SEQ ID NO: 645 - RNA, 23 nt, Homo sapiens
ucgaggacugguggaagggccuu

SEQ ID NO: 646 - RNA, 24 nt, Homo sapiens
uggguagagaaggagcucagagga

SEQ ID NO: 647 - RNA, 22 nt, Homo sapiens
uaaagaacucuuaaaacccaau

SEQ ID NO: 648 - RNA, 23 nt, Homo sapiens
ugauggauaaaagacuacauauu

SEQ ID NO: 649 - RNA, 22 nt, Homo sapiens
ugccuaggcugagacugcagug

SEQ ID NO: 650 - RNA, 22 nt, Homo sapiens
ggcuggagcgagugcaguggug

SEQ ID NO: 651 - RNA, 22 nt, Homo sapiens
uggcccaaccuauucaguuagu

SEQ ID NO: 652 - RNA, 23 nt, Homo sapiens
cugacugaauagguagggucauu

SEQ ID NO: 653 - RNA, 24 nt, Homo sapiens
ucuguagccugggagcaauggggu

SEQ ID NO: 654 - RNA, 24 nt, Homo sapiens
uguggacagugagguagagggagu

SEQ ID NO: 655 - RNA, 23 nt, Homo sapiens
uaggagcucaacagaugccuguu

SEQ ID NO: 656 - RNA, 22 nt, Homo sapiens
agcuuuugggaauucagguagu

SEQ ID NO: 657 - RNA, 21 nt, Homo sapiens
accugaauuaccaaaagcuuu

SEQ ID NO: 658 - RNA, 19 nt, Homo sapiens
gagggcggguggaggagga

SEQ ID NO: 659 - RNA, 22 nt, Homo sapiens
aaggccuuucugaaccuucaga

SEQ ID NO: 660 - RNA, 25 nt, Homo sapiens
auaacauuguaaagcgcuucuuucg

SEQ ID NO: 661 - RNA, 22 nt, Homo sapiens
auauaccuguucggucucuuua

SEQ ID NO: 662 - RNA, 22 nt, Homo sapiens
aggggaccaaagagauauauag

SEQ ID NO: 663 - RNA, 24 nt, Homo sapiens
agauauuuugaguguuuggaauug

SEQ ID NO: 664 - RNA, 22 nt, Homo sapiens
aacuccaaacacucaaaacuca

SEQ ID NO: 665 - RNA, 22 nt, Homo sapiens
caugcuaggauagaaagaaugg

SEQ ID NO: 666 - RNA, 24 nt, Homo sapiens
gguugggcagugaggaggguguga

SEQ ID NO: 667 - RNA, 22 nt, Homo sapiens
uggaaaaaacuggugugugcuu

SEQ ID NO: 668 - RNA, 23 nt, Homo sapiens
uuuguauggauauguguguguau

SEQ ID NO: 669 - RNA, 22 nt, Homo sapiens
cuggggagauccucgagguugg

SEQ ID NO: 670 - RNA, 22 nt, Homo sapiens
caaccucgacgaucuccucagc

SEQ ID NO: 671 - RNA, 21 nt, Homo sapiens
ugaggagaucgucgagguugg

SEQ ID NO: 672 - RNA, 22 nt, Homo sapiens
caaccucgaggaucuccccagc

SEQ ID NO: 673 - RNA, 21 nt, Homo sapiens
gguggggcaaugggaucaggu

SEQ ID NO: 674 - RNA, 20 nt, Homo sapiens
ccugaucccacagcccaccu

SEQ ID NO: 675 - RNA, 21 nt, Homo sapiens
gguggggcaaugggaucaggu

SEQ ID NO: 676 - RNA, 22 nt, Homo sapiens
uguguuagaauaggggcaauaa

SEQ ID NO: 677 - RNA, 22 nt, Homo sapiens
auugccucuguucuaacacaag

SEQ ID NO: 678 - RNA, 23 nt, Homo sapiens
ggggaaagcgaguagggacauuu

SEQ ID NO: 679 - RNA, 22 nt, Homo sapiens
cagaaggggaguugggagcaga

SEQ ID NO: 680 - RNA, 21 nt, Homo sapiens
ccaggcucugcagugggaacu

SEQ ID NO: 681 - RNA, 18 nt, Homo sapiens
ccaggcucugcaguggga

SEQ ID NO: 682 - RNA, 21 nt, Homo sapiens
cucccacuuccagaucuuucu

SEQ ID NO: 683 - RNA, 22 nt, Homo sapiens
aaagaucuggaagugggagaca

SEQ ID NO: 684 - RNA, 22 nt, Homo sapiens
cugcccuagucuagcugaagcu

SEQ ID NO: 685 - RNA, 22 nt, Homo sapiens
uucagccaggcuagugcagucu

SEQ ID NO: 686 - RNA, 22 nt, Homo sapiens
aagggcuuccucucugcaggac

SEQ ID NO: 687 - RNA, 21 nt, Homo sapiens
ccugcagagaggaagcccuuc

SEQ ID NO: 688 - RNA, 22 nt, Homo sapiens
uaggauuacaagugucggccac

SEQ ID NO: 689 - RNA, 22 nt, Homo sapiens
agagcugagacuagaaagccca

SEQ ID NO: 690 - RNA, 22 nt, Homo sapiens
ggcuuucuagucucagcucucc

SEQ ID NO: 691 - RNA, 23 nt, Homo sapiens
cugauaagaacagaggcccagau

SEQ ID NO: 692 - RNA, 21 nt, Homo sapiens
ucccuaccccuccacucccca

SEQ ID NO: 693 - RNA, 23 nt, Homo sapiens
uuagggaguagaaggguggggag

SEQ ID NO: 694 - RNA, 22 nt, Homo sapiens
uauaaaaugagggcaguaagac

SEQ ID NO: 695 - RNA, 22 nt, Homo sapiens
ugugacuuuaagggaaauggcg

SEQ ID NO: 696 - RNA, 22 nt, Homo sapiens
agguggaugcaaugugaccuca

SEQ ID NO: 697 - RNA, 23 nt, Homo sapiens
cgcagacaaugccuacuggccua

SEQ ID NO: 698 - RNA, 22 nt, Homo sapiens
aggauuucagaaauacuggugu

SEQ ID NO: 699 - RNA, 17 nt, Homo sapiens
gaguucuacagucagac

SEQ ID NO: 700 - RNA, 22 nt, Homo sapiens
uaggacugugcuuggcacauag

SEQ ID NO: 701 - RNA, 22 nt, Homo sapiens
cugggguucugagacagacagu

SEQ ID NO: 702 - RNA, 24 nt, Homo sapiens
agauguauggaaucuguauauauc

SEQ ID NO: 703 - RNA, 22 nt, Homo sapiens
aaaggaggaaauaggcaggcca

SEQ ID NO: 704 - RNA, 22 nt, Homo sapiens
ugcccugccuguuuucuccuuu

SEQ ID NO: 705 - RNA, 23 nt, Homo sapiens
uagugaguuagagaugcagagcc

SEQ ID NO: 706 - RNA, 22 nt, Homo sapiens
cggggagagaacgcagugacgu

SEQ ID NO: 707 - RNA, 19 nt, Homo sapiens
acuggccugggacuaccgg

SEQ ID NO: 708 - RNA, 21 nt, Homo sapiens
ugcacggcacuggggacacgu

SEQ ID NO: 709 - RNA, 23 nt, Homo sapiens
uguguacacacgugccaggcgcu

SEQ ID NO: 710 - RNA, 17 nt, Homo sapiens
ggggcgcggccggaucg

SEQ ID NO: 711 - RNA, 22 nt, Homo sapiens
agaaggggugaaauuuaaacgu

SEQ ID NO: 712 - RNA, 19 nt, Homo sapiens
uggggcggagcuuccggag

SEQ ID NO: 713 - RNA, 22 nt, Homo sapiens
uggggcggagcuuccggaggcc

SEQ ID NO: 714 - RNA, 25 nt, Homo sapiens
cuuccagacgcuccgccccacgucg

SEQ ID NO: 715 - RNA, 19 nt, Homo sapiens
aucgggcccucggcgccgg

SEQ ID NO: 716 - RNA, 17 nt, Homo sapiens
gcuucuguaguguaguc

SEQ ID NO: 717 - RNA, 22 nt, Homo sapiens
gccucucucggagucgcucgga

SEQ ID NO: 718 - RNA, 23 nt, Homo sapiens
aaagucucgcucucugccccuca

SEQ ID NO: 719 - RNA, 24 nt, Homo sapiens
ugaggggccucagaccgagcuuuu

SEQ ID NO: 720 - RNA, 23 nt, Homo sapiens
agaagaaggcggucggucugcgg

SEQ ID NO: 721 - RNA, 21 nt, Homo sapiens
ucacgcggagagauggcuuug

SEQ ID NO: 722 - RNA, 22 nt, Homo sapiens
caggcgucugucuacguggcuu

SEQ ID NO: 723 - RNA, 20 nt, Homo sapiens
uuggccauggggcugcgcgg

SEQ ID NO: 724 - RNA, 23 nt, Homo sapiens
ccugggcagcguguggcugaagg

SEQ ID NO: 725 - RNA, 23 nt, Homo sapiens
agaggcuuugugcggauacgggg

SEQ ID NO: 726 - RNA, 21 nt, Homo sapiens
cccuugggucugaugggguag

SEQ ID NO: 727 - RNA, 25 nt, Homo sapiens
ugccccaucugugcccuggguagga

SEQ ID NO: 728 - RNA, 23 nt, Homo sapiens
uguggaagguagacggccagaga

SEQ ID NO: 729 - RNA, 20 nt, Homo sapiens
ucuggccagcuacgucccca

SEQ ID NO: 730 - RNA, 23 nt, Homo sapiens
uggggacguagcuggccagacag

SEQ ID NO: 731 - RNA, 22 nt, Homo sapiens
cucucuggccgucuaccuucca

SEQ ID NO: 732 - RNA, 23 nt, Homo sapiens
ucugggagguuguagcaguggaa

SEQ ID NO: 733 - RNA, 21 nt, Homo sapiens
cucugaucgcccucucagcuc

SEQ ID NO: 734 - RNA, 23 nt, Homo sapiens
ucugggagguuguagcaguggaa

SEQ ID NO: 735 - RNA, 22 nt, Homo sapiens
uccugcguaggaucugaggagu

SEQ ID NO: 736 - RNA, 22 nt, Homo sapiens
agcucugcugcucacuggcagu

SEQ ID NO: 737 - RNA, 21 nt, Homo sapiens
ggccagccaccaggagggcug

SEQ ID NO: 738 - RNA, 17 nt, Homo sapiens
cgcgccgggcccggguu

SEQ ID NO: 739 - RNA, 18 nt, Homo sapiens
cggggcggcaggggccuc

SEQ ID NO: 740 - RNA, 23 nt, Homo sapiens
ggaggcgcaggcucggaaaggcg

SEQ ID NO: 741 - RNA, 22 nt, Homo sapiens
guggaguccuggggaauggaga

SEQ ID NO: 742 - RNA, 23 nt, Homo sapiens
agggacugccuuaggagaaaguu

SEQ ID NO: 743 - RNA, 22 nt, Homo sapiens
caauuuagugugugugauauuu

SEQ ID NO: 744 - RNA, 22 nt, Homo sapiens
uauugcacauuacuaaguugca

SEQ ID NO: 745 - RNA, 22 nt, Homo sapiens
caccuugcgcuacucaggucug

SEQ ID NO: 746 - RNA, 22 nt, Homo sapiens
aaucugagaaggcgcacaaggu

SEQ ID NO: 747 - RNA, 17 nt, Homo sapiens
gggauaugaagaaaaau

SEQ ID NO: 748 - RNA, 22 nt, Homo sapiens
uggaagggagaagagcuuuaau

SEQ ID NO: 749 - RNA, 22 nt, Homo sapiens
aaaagcuggguugagagggcga

SEQ ID NO: 750 - RNA, 22 nt, Homo sapiens
aaaagcuggguugagagggcaa

SEQ ID NO: 751 - RNA, 20 nt, Homo sapiens
aaaagcuggguugagagggu

SEQ ID NO: 752 - RNA, 19 nt, Homo sapiens
aaaagcuggguugagagga

SEQ ID NO: 753 - RNA, 18 nt, Homo sapiens
aaagcuggguugagaagg

SEQ ID NO: 754 - RNA, 21 nt, Homo sapiens
cacauuacacggucgaccucu

SEQ ID NO: 755 - RNA, 22 nt, Homo sapiens
aggugguccguggcgcguucgc

SEQ ID NO: 756 - RNA, 22 nt, Homo sapiens
cccaauacacggucgaccucuu

SEQ ID NO: 757 - RNA, 23 nt, Homo sapiens
agguuguccguggugaguucgca

SEQ ID NO: 758 - RNA, 20 nt, Homo sapiens
acugccccaggugcugcugg

SEQ ID NO: 759 - RNA, 23 nt, Homo sapiens
cgcauccccuagggcauuggugu

SEQ ID NO: 760 - RNA, 23 nt, Homo sapiens
ccuaguagguguccaguaagugu

SEQ ID NO: 761 - RNA, 20 nt, Homo sapiens
ccucugggcccuuccuccag

SEQ ID NO: 762 - RNA, 22 nt, Homo sapiens
cuggcccucucugcccuuccgu

SEQ ID NO: 763 - RNA, 22 nt, Homo sapiens
cuggcccucucugcccuuccgu

SEQ ID NO: 764 - RNA, 23 nt, Homo sapiens
gggggggcaggaggggcucaggg

SEQ ID NO: 765 - RNA, 22 nt, Homo sapiens
aacacaccugguuaaccucuuu

SEQ ID NO: 766 - RNA, 22 nt, Homo sapiens
aacacaccugguuaaccucuuu

SEQ ID NO: 767 - RNA, 23 nt, Homo sapiens
gagguuuucuggguuucuguuuc

SEQ ID NO: 768 - RNA, 23 nt, Homo sapiens
gcaaagcacacggccugcagaga

SEQ ID NO: 769 - RNA, 22 nt, Homo sapiens
ucucugggccugugucuuaggc

SEQ ID NO: 770 - RNA, 21 nt, Homo sapiens
gccccugggccuauccuagaa

SEQ ID NO: 771 - RNA, 22 nt, Homo sapiens
cuagguauggucccagggaucc

SEQ ID NO: 772 - RNA, 22 nt, Homo sapiens
uuuuucauuauugcuccugacc

SEQ ID NO: 773 - RNA, 23 nt, Homo sapiens
ucaagagcaauaacgaaaaaugu

SEQ ID NO: 774 - RNA, 22 nt, Homo sapiens
cuccuauaugaugccuuucuuc

SEQ ID NO: 775 - RNA, 21 nt, Homo sapiens
gaacggcuucauacaggaguu

SEQ ID NO: 776 - RNA, 22 nt, Homo sapiens
uccagcaucagugauuuuguug

SEQ ID NO: 777 - RNA, 22 nt, Homo sapiens
aacaauauccuggugcugagug

SEQ ID NO: 778 - RNA, 23 nt, Homo sapiens
ugagcgccucgacgacagagccg

SEQ ID NO: 779 - RNA, 23 nt, Homo sapiens
ucccuguccuccaggagcucacg

SEQ ID NO: 780 - RNA, 22 nt, Homo sapiens
caauguuuccacagugcaucac

SEQ ID NO: 781 - RNA, 21 nt, Homo sapiens
gugcauuguaguugcauugca

SEQ ID NO: 782 - RNA, 22 nt, Homo sapiens
cagugccucggcagugcagccc

SEQ ID NO: 783 - RNA, 20 nt, Homo sapiens
gugcauugcuguugcauugc

SEQ ID NO: 784 - RNA, 22 nt, Homo sapiens
uccgucucaguuacuuuauagc

SEQ ID NO: 785 - RNA, 22 nt, Homo sapiens
uuauaaagcaaugagacugauu

SEQ ID NO: 786 - RNA, 23 nt, Homo sapiens
ucucacacagaaaucgcacccgu

SEQ ID NO: 787 - RNA, 21 nt, Homo sapiens
aggggugcuaucugugauuga

SEQ ID NO: 788 - RNA, 22 nt, Homo sapiens
gcccugaacgaggggucuggag

SEQ ID NO: 789 - RNA, 22 nt, Homo sapiens
gcugacuccuaguccagggcuc

SEQ ID NO: 790 - RNA, 23 nt, Homo sapiens
ugucugcccgcaugccugccucu

SEQ ID NO: 791 - RNA, 22 nt, Homo sapiens
caaucagcaaguauacugcccu

SEQ ID NO: 792 - RNA, 22 nt, Homo sapiens
uggcagugucuuagcugguugu

SEQ ID NO: 793 - RNA, 22 nt, Homo sapiens
caaucacuaacuccacugccau

SEQ ID NO: 794 - RNA, 23 nt, Homo sapiens
uaggcagugucauuagcugauug

SEQ ID NO: 795 - RNA, 22 nt, Homo sapiens
aaucacuaaccacacggccagg

SEQ ID NO: 796 - RNA, 23 nt, Homo sapiens
aggcaguguaguuagcugauugc

SEQ ID NO: 797 - RNA, 24 nt, Homo sapiens
aacaacaaaaucacuagucuucca

SEQ ID NO: 798 - RNA, 22 nt, Homo sapiens
agguagacugggauuuguuguu

SEQ ID NO: 799 - RNA, 22 nt, Homo sapiens
aaacaccauugucacacuccac

SEQ ID NO: 800 - RNA, 23 nt, Homo sapiens
uuuagugugauaauggcguuuga

SEQ ID NO: 801 - RNA, 23 nt, Homo sapiens
ccuccguguuaccuguccucuag

SEQ ID NO: 802 - RNA, 23 nt, Homo sapiens
ugaggauggauagcaaggaagcc

SEQ ID NO: 803 - RNA, 21 nt, Homo sapiens
aaaauuucuuucacuacuuag

SEQ ID NO: 804 - RNA, 21 nt, Homo sapiens
uuagugaaggcuauuuuaauu

SEQ ID NO: 805 - RNA, 20 nt, Homo sapiens
acuguaaacgcuuucugaug

SEQ ID NO: 806 - RNA, 22 nt, Homo sapiens
gcaugugaugaagcaaaucagu

SEQ ID NO: 807 - RNA, 24 nt, Homo sapiens
caaagugaugaguaauacuggcug

SEQ ID NO: 808 - RNA, 23 nt, Homo sapiens
ucccccaggugugauucugauuu

SEQ ID NO: 809 - RNA, 22 nt, Homo sapiens
uuaucagaaucuccagggguac

SEQ ID NO: 810 - RNA, 20 nt, Homo sapiens
gaaucggaaaggaggcgccg

SEQ ID NO: 811 - RNA, 21 nt, Homo sapiens
uugugaagaaagaaauucuua

SEQ ID NO: 812 - RNA, 22 nt, Homo sapiens
aggaggcaucuugagaaaugga

SEQ ID NO: 813 - RNA, 24 nt, Homo sapiens
acaaaaaaaaaagcccaacccuuc

SEQ ID NO: 814 - RNA, 22 nt, Homo sapiens
uguuguacuuuuuuuuuuguuc

SEQ ID NO: 815 - RNA, 23 nt, Homo sapiens
uagccuucagaucuugguguuuu

SEQ ID NO: 816 - RNA, 23 nt, Homo sapiens
ccacuuggaucugaaggcugccc

SEQ ID NO: 817 - RNA, 21 nt, Homo sapiens
ucucucggcuccucgcggcuc

SEQ ID NO: 818 - RNA, 23 nt, Homo sapiens
cgagggcauuucaugaugcaggc

SEQ ID NO: 819 - RNA, 22 nt, Homo sapiens
augaagugcacucaugauaugu

SEQ ID NO: 820 - RNA, 23 nt, Homo sapiens
caucagcacccuauguccuuucu

SEQ ID NO: 821 - RNA, 22 nt, Homo sapiens
aaagacauaguugcaagauggg

SEQ ID NO: 822 - RNA, 22 nt, Homo sapiens
ugucuacauuaaugaaaagagc

SEQ ID NO: 823 - RNA, 22 nt, Homo sapiens
gggaccauccugccugcugugg

SEQ ID NO: 824 - RNA, 22 nt, Homo sapiens
ucagcaggcaggcuggugcagc

SEQ ID NO: 825 - RNA, 22 nt, Homo sapiens
aacacaccuauucaaggauuca

SEQ ID NO: 826 - RNA, 24 nt, Homo sapiens
aauccuuggaaccuaggugugagu

SEQ ID NO: 827 - RNA, 22 nt, Homo sapiens
ucacccugcaucccgcacccag

SEQ ID NO: 828 - RNA, 22 nt, Homo sapiens
gugggcugggcugggcugggcc

SEQ ID NO: 829 - RNA, 20 nt, Homo sapiens
cgcgggucggggucugcagg

SEQ ID NO: 830 - RNA, 22 nt, Homo sapiens
ucaccugaccucccaugccugu

SEQ ID NO: 831 - RNA, 22 nt, Homo sapiens
caggcacgggagcucaggugag

SEQ ID NO: 832 - RNA, 21 nt, Homo sapiens
ucaccugagcucccgugccug

SEQ ID NO: 833 - RNA, 20 nt, Homo sapiens
aggcaugggaggucagguga

SEQ ID NO: 834 - RNA, 22 nt, Homo sapiens
aauugcacgguauccaucugua

SEQ ID NO: 835 - RNA, 22 nt, Homo sapiens
cggguggaucacgaugcaauuu

SEQ ID NO: 836 - RNA, 22 nt, Homo sapiens
aaaaugaaaugagcccagccca

SEQ ID NO: 837 - RNA, 21 nt, Homo sapiens
agccgcggggaucgccgaggg

SEQ ID NO: 838 - RNA, 19 nt, Homo sapiens
agggaccugagugucuaag

SEQ ID NO: 839 - RNA, 19 nt, Homo sapiens
aggugugucuguagagucc

SEQ ID NO: 840 - RNA, 24 nt, Homo sapiens
cauagcccggucgcugguacauga

SEQ ID NO: 841 - RNA, 18 nt, Homo sapiens
cggcuggaggugugagga

SEQ ID NO: 842 - RNA, 18 nt, Homo sapiens
cuaagaaguugacugaag

SEQ ID NO: 843 - RNA, 18 nt, Homo sapiens
cuaagaaguugacugaag

SEQ ID NO: 844 - RNA, 20 nt, Homo sapiens
ccuccugaugauucuucuuc

SEQ ID NO: 845 - RNA, 19 nt, Homo sapiens
gacuggacaagcugaggaa

SEQ ID NO: 846 - RNA, 21 nt, Homo sapiens
gcuugucgcugcgguguugcu

SEQ ID NO: 847 - RNA, 17 nt, Homo sapiens
ggcgggugcgggggugg

SEQ ID NO: 848 - RNA, 21 nt, Homo sapiens
ugugucccauuauuggugauu

SEQ ID NO: 849 - RNA, 22 nt, Homo sapiens
uuuaagaaaacaccauggagau

SEQ ID NO: 850 - RNA, 21 nt, Homo sapiens
ugaguguugucuacgagggca

SEQ ID NO: 851 - RNA, 22 nt, Homo sapiens
uaaugccccuaaaaauccuuau

SEQ ID NO: 852 - RNA, 23 nt, Homo sapiens
agggacuuuugggggcagaugug

SEQ ID NO: 853 - RNA, 22 nt, Homo sapiens
uaaugccccuaaaaauccuuau

SEQ ID NO: 854 - RNA, 22 nt, Homo sapiens
agggacuuucaggggcagcugu

SEQ ID NO: 855 - RNA, 21 nt, Homo sapiens
acugacaggagagcauuuuga

SEQ ID NO: 856 - RNA, 22 nt, Homo sapiens
ugaccugggacucggacagcug

SEQ ID NO: 857 - RNA, 24 nt, Homo sapiens
gaaaaugaugaguagugacugaug

SEQ ID NO: 858 - RNA, 23 nt, Homo sapiens
ugagcaccacacaggccgggcgc

SEQ ID NO: 859 - RNA, 21 nt, Homo sapiens
gcuggucugcguggugcucgg

SEQ ID NO: 860 - RNA, 22 nt, Homo sapiens
ucucaggaguaaagacagaguu

SEQ ID NO: 861 - RNA, 22 nt, Homo sapiens
aacucugucuucacucaugagu

SEQ ID NO: 862 - RNA, 18 nt, Homo sapiens
agcaggugcggggcggcg

SEQ ID NO: 863 - RNA, 21 nt, Homo sapiens
cagugcaaguguagaugccga

SEQ ID NO: 864 - RNA, 22 nt, Homo sapiens
accuuccucuccaugggucuuu

SEQ ID NO: 865 - RNA, 22 nt, Homo sapiens
aaagacccauugaggagaaggu

SEQ ID NO: 866 - RNA, 21 nt, Homo sapiens
aauguagagauugaucaaaau

SEQ ID NO: 867 - RNA, 23 nt, Homo sapiens
acggaauauguauacggaauaua

SEQ ID NO: 868 - RNA, 22 nt, Homo sapiens
aauugcacuuuagcaaugguga

SEQ ID NO: 869 - RNA, 22 nt, Homo sapiens
acuguugcuaauaugcaacucu

SEQ ID NO: 870 - RNA, 24 nt, Homo sapiens
agagcucacagcuguccuucucua

SEQ ID NO: 871 - RNA, 22 nt, Homo sapiens
aucaaauaaggacuagucugca

SEQ ID NO: 872 - RNA, 23 nt, Homo sapiens
augagacucauguaaaacaucuu

SEQ ID NO: 873 - RNA, 21 nt, Homo sapiens
auggaauguauauacggaaua

SEQ ID NO: 874 - RNA, 22 nt, Homo sapiens
auuguagaaccuaagauuggcc

SEQ ID NO: 875 - RNA, 22 nt, Homo sapiens
caucucuaaggaacucccccaa

SEQ ID NO: 876 - RNA, 23 nt, Homo sapiens
uauggggcuucuguagagauuuc

SEQ ID NO: 877 - RNA, 20 nt, Homo sapiens
ccguguuucccccacgcuuu

SEQ ID NO: 878 - RNA, 15 nt, Homo sapiens
aggagauccuggguu

SEQ ID NO: 879 - RNA, 22 nt, Homo sapiens
cucgugggcucuggccacggcc

SEQ ID NO: 880 - RNA, 22 nt, Homo sapiens
caguggccagagcccugcagug

SEQ ID NO: 881 - RNA, 22 nt, Homo sapiens
cugcagaguuuguacggaccgg

SEQ ID NO: 882 - RNA, 20 nt, Homo sapiens
uccguacaaacucugcugug

SEQ ID NO: 883 - RNA, 22 nt, Homo sapiens
cuuccccccaguaaucuucauc

SEQ ID NO: 884 - RNA, 23 nt, Homo sapiens
ugaggauauggcagggaagggga

SEQ ID NO: 885 - RNA, 23 nt, Homo sapiens
uuuugcaugacccugggaguagg

SEQ ID NO: 886 - RNA, 22 nt, Homo sapiens
gacucacucacaggauugugca

SEQ ID NO: 887 - RNA, 22 nt, Homo sapiens
acacagugcuucauccacuacu

SEQ ID NO: 888 - RNA, 22 nt, Homo sapiens
uaguggaugaugcacucugugc

SEQ ID NO: 889 - RNA, 21 nt, Homo sapiens
ugaugauacagguggagguag

SEQ ID NO: 890 - RNA, 22 nt, Homo sapiens
cuacuucuaccuguguuaucau

SEQ ID NO: 891 - RNA, 22 nt, Homo sapiens
ugcgacauuggaaguaguauca

SEQ ID NO: 892 - RNA, 21 nt, Homo sapiens
uuagaccuaguacacguccuu

SEQ ID NO: 893 - RNA, 22 nt, Homo sapiens
uuuccuacccuaccugaagacu

SEQ ID NO: 894 - RNA, 22 nt, Homo sapiens
aucuguaagagaaaguaaauga

SEQ ID NO: 895 - RNA, 24 nt, Homo sapiens
cccggacaggcguucgugcgacgu

SEQ ID NO: 896 - RNA, 22 nt, Homo sapiens
uauggaaagacuuugccacucu

SEQ ID NO: 897 - RNA, 21 nt, Homo sapiens
aguggcaaagucuuuccauau

SEQ ID NO: 898 - RNA, 22 nt, Homo sapiens
cugggaggugugauaucguggu

SEQ ID NO: 899 - RNA, 22 nt, Homo sapiens
ugugauaucaugguuccuggga

SEQ ID NO: 900 - RNA, 22 nt, Homo sapiens
cugggaggugugauauuguggu

SEQ ID NO: 901 - RNA, 22 nt, Homo sapiens
ugugauaucaugguuccuggga

SEQ ID NO: 902 - RNA, 22 nt, Homo sapiens
cugggaggugugauauuguggu

SEQ ID NO: 903 - RNA, 22 nt, Homo sapiens
gggaggugugaucucacacucg

SEQ ID NO: 904 - RNA, 22 nt, Homo sapiens
ugugauaucaugguuccuggga

SEQ ID NO: 905 - RNA, 22 nt, Homo sapiens
ugugauaucgugcuuccuggga

SEQ ID NO: 906 - RNA, 21 nt, Homo sapiens
aauaauacaugguugaucuuu

SEQ ID NO: 907 - RNA, 22 nt, Homo sapiens
agaucgaccguguuauauucgc

SEQ ID NO: 908 - RNA, 23 nt, Homo sapiens
accuggacccagcguagacaaag

SEQ ID NO: 909 - RNA, 22 nt, Homo sapiens
accaagucugcgucauccucuc

SEQ ID NO: 910 - RNA, 23 nt, Homo sapiens
aguggaugauggagacucgguac

SEQ ID NO: 911 - RNA, 24 nt, Homo sapiens
guuccacacugacacugcagaagu

SEQ ID NO: 912 - RNA, 24 nt, Homo sapiens
ccugcuggucaggaguggauacug

SEQ ID NO: 913 - RNA, 22 nt, Homo sapiens
gccugcugggguggaaccuggu

SEQ ID NO: 914 - RNA, 22 nt, Homo sapiens
gccugcugggguggaaccuggu

SEQ ID NO: 915 - RNA, 22 nt, Homo sapiens
caggucacgucucugcaguuac

SEQ ID NO: 916 - RNA, 20 nt, Homo sapiens
gguauccguuuggggauggu

SEQ ID NO: 917 - RNA, 22 nt, Homo sapiens
gaaggcagcagugcuccccugu

SEQ ID NO: 918 - RNA, 23 nt, Homo sapiens
aagugccgccaucuuuugagugu

SEQ ID NO: 919 - RNA, 20 nt, Homo sapiens
acucaaacugugggggcacu

SEQ ID NO: 920 - RNA, 23 nt, Homo sapiens
aagugcccccacaguuugagugc

SEQ ID NO: 921 - RNA, 22 nt, Homo sapiens
acucaaaagauggcggcacuuu

SEQ ID NO: 922 - RNA, 23 nt, Homo sapiens
aaagugcugcgacauuugagcgu

SEQ ID NO: 923 - RNA, 23 nt, Homo sapiens
aaagugcugcgacauuugagcgu

SEQ ID NO: 924 - RNA, 23 nt, Homo sapiens
ccucaaauguggagcacuauucu

SEQ ID NO: 925 - RNA, 23 nt, Homo sapiens
gaagugcuucgauuuuggggugu

SEQ ID NO: 926 - RNA, 22 nt, Homo sapiens
acucaaaaugggggcgcuuucc

SEQ ID NO: 927 - RNA, 22 nt, Homo sapiens
cuuaucagauuguauuguaauu

SEQ ID NO: 928 - RNA, 22 nt, Homo sapiens
uuauaauacaaccugauaagug

SEQ ID NO: 929 - RNA, 22 nt, Homo sapiens
cuuagcagguuguauuaucauu

SEQ ID NO: 930 - RNA, 22 nt, Homo sapiens
auauaauacaaccugcuaagug

SEQ ID NO: 931 - RNA, 22 nt, Homo sapiens
cacuuagcagguuguauuauau

SEQ ID NO: 932 - RNA, 22 nt, Homo sapiens
auaauacaaccugcuaagugcu

SEQ ID NO: 933 - RNA, 22 nt, Homo sapiens
uuuguucguucggcucgcguga

SEQ ID NO: 934 - RNA, 21 nt, Homo sapiens
gguagauuuuccuucuauggu

SEQ ID NO: 935 - RNA, 21 nt, Homo sapiens
aucauagaggaaaauccacgu

SEQ ID NO: 936 - RNA, 22 nt, Homo sapiens
guagauucuccuucuaugagua

SEQ ID NO: 937 - RNA, 22 nt, Homo sapiens
aucauagaggaaaauccauguu

SEQ ID NO: 938 - RNA, 22 nt, Homo sapiens
cguggauauuccuucuauguuu

SEQ ID NO: 939 - RNA, 21 nt, Homo sapiens
aacauagaggaaauuccacgu

SEQ ID NO: 940 - RNA, 21 nt, Homo sapiens
gguggauauuccuucuauguu

SEQ ID NO: 941 - RNA, 22 nt, Homo sapiens
aucacacaaaggcaacuuuugu

SEQ ID NO: 942 - RNA, 22 nt, Homo sapiens
agagguugcccuuggugaauuc

SEQ ID NO: 943 - RNA, 22 nt, Homo sapiens
acuggacuuggagucagaaggc

SEQ ID NO: 944 - RNA, 21 nt, Homo sapiens
acuggacuuggagucagaagg

SEQ ID NO: 945 - RNA, 22 nt, Homo sapiens
cuccugacuccagguccugugu

SEQ ID NO: 946 - RNA, 19 nt, Homo sapiens
acuggacuuggaggcagaa

SEQ ID NO: 947 - RNA, 25 nt, Homo sapiens
acuggacuuggagucagaagagugg

SEQ ID NO: 948 - RNA, 20 nt, Homo sapiens
acuggacuuggagucagaaa

SEQ ID NO: 949 - RNA, 19 nt, Homo sapiens
acuggacuuggagucagga

SEQ ID NO: 950 - RNA, 20 nt, Homo sapiens
acuggacuuggagccagaag

SEQ ID NO: 951 - RNA, 20 nt, Homo sapiens
acugggcuuggagucagaag

SEQ ID NO: 952 - RNA, 21 nt, Homo sapiens
acuggacuuggugucagaugg

SEQ ID NO: 953 - RNA, 21 nt, Homo sapiens
acuggacuaggagucagaagg

SEQ ID NO: 954 - RNA, 19 nt, Homo sapiens
acuggauuuggagccagaa

SEQ ID NO: 955 - RNA, 22 nt, Homo sapiens
uauguaacaugguccacuaacu

SEQ ID NO: 956 - RNA, 21 nt, Homo sapiens
ugguagacuauggaacguagg

SEQ ID NO: 957 - RNA, 22 nt, Homo sapiens
uauguaauaugguccacaucuu

SEQ ID NO: 958 - RNA, 22 nt, Homo sapiens
ugguugaccauagaacaugcgc

SEQ ID NO: 959 - RNA, 22 nt, Homo sapiens
uauacaagggcaagcucucugu

SEQ ID NO: 960 - RNA, 22 nt, Homo sapiens
agcgagguugcccuuuguauau

SEQ ID NO: 961 - RNA, 21 nt, Homo sapiens
aaucauucacggacaacacuu

SEQ ID NO: 962 - RNA, 22 nt, Homo sapiens
gaaguuguucgugguggauucg

SEQ ID NO: 963 - RNA, 22 nt, Homo sapiens
agaucagaaggugauuguggcu

SEQ ID NO: 964 - RNA, 19 nt, Homo sapiens
acagcacugccuggucaga

SEQ ID NO: 965 - RNA, 22 nt, Homo sapiens
agaucagaaggugauuguggcu

SEQ ID NO: 966 - RNA, 20 nt, Homo sapiens
auuccuagaaauuguucaua

SEQ ID NO: 967 - RNA, 22 nt, Homo sapiens
aggugcuccaggcuggcucaca

SEQ ID NO: 968 - RNA, 22 nt, Homo sapiens
gagcaauguagguagacuguuu

SEQ ID NO: 969 - RNA, 22 nt, Homo sapiens
uguccucuagggccugcagucu

SEQ ID NO: 970 - RNA, 20 nt, Homo sapiens
aaaggcauaaaaccaagaca

SEQ ID NO: 971 - RNA, 22 nt, Homo sapiens
uguguggauccuggaggaggca

SEQ ID NO: 972 - RNA, 21 nt, Homo sapiens
uaacgcauaauauggacaugu

SEQ ID NO: 973 - RNA, 21 nt, Homo sapiens
uaacgcauaauauggacaugu

SEQ ID NO: 974 - RNA, 21 nt, Homo sapiens
auguccauauuauggguuagu

SEQ ID NO: 975 - RNA, 22 nt, Homo sapiens
agacaucaagaucagucccaaa

SEQ ID NO: 976 - RNA, 22 nt, Homo sapiens
uuugggacugaucuugaugucu

SEQ ID NO: 977 - RNA, 22 nt, Homo sapiens
aaggaaccagaaaaugagaagu

SEQ ID NO: 978 - RNA, 22 nt, Homo sapiens
uugaggaaaagauggucuuauu

SEQ ID NO: 979 - RNA, 26 nt, Homo sapiens
aagaggaagaaauggcugguucucag

SEQ ID NO: 980 - RNA, 20 nt, Homo sapiens
gcucggacugagcagguggg

SEQ ID NO: 981 - RNA, 21 nt, Homo sapiens
acagggccgcagauggagacu

SEQ ID NO: 982 - RNA, 21 nt, Homo sapiens
gcagagaacaaaggacucagu

SEQ ID NO: 983 - RNA, 22 nt, Homo sapiens
acugauuaucuuaacucucuga

SEQ ID NO: 984 - RNA, 23 nt, Homo sapiens
ucucugaguaccauaugccuugu

SEQ ID NO: 985 - RNA, 22 nt, Homo sapiens
ucuggccuugacuugacucuuu

SEQ ID NO: 986 - RNA, 23 nt, Homo sapiens
ucaaggccagaggucccacagca

SEQ ID NO: 987 - RNA, 22 nt, Homo sapiens
aacuaguaauguuggauuaggg

SEQ ID NO: 988 - RNA, 22 nt, Homo sapiens
auauguauaugugacugcuacu

SEQ ID NO: 989 - RNA, 21 nt, Homo sapiens
acuccaguuuuaguucucuug

SEQ ID NO: 990 - RNA, 22 nt, Homo sapiens
aagagaacugaaaguggagccu

SEQ ID NO: 991 - RNA, 21 nt, Homo sapiens
uggccaaaaagcaggcagaga

SEQ ID NO: 992 - RNA, 22 nt, Homo sapiens
cagguagauauuugauaggcau

SEQ ID NO: 993 - RNA, 21 nt, Homo sapiens
gccuaucacauaucugccugu

SEQ ID NO: 994 - RNA, 22 nt, Homo sapiens
ggaggaaccuuggagcuucggc

SEQ ID NO: 995 - RNA, 22 nt, Homo sapiens
ggaggaaccuuggagcuucggc

SEQ ID NO: 996 - RNA, 23 nt, Homo sapiens
ugaagcucuaagguuccgccugc

SEQ ID NO: 997 - RNA, 23 nt, Homo sapiens
gaggcugaugugaguagaccacu

SEQ ID NO: 998 - RNA, 22 nt, Homo sapiens
ugcucagguugcacagcuggga

SEQ ID NO: 999 - RNA, 22 nt, Homo sapiens
ucagguguggaaacugaggcag

SEQ ID NO: 1000 - RNA, 22 nt, Homo sapiens
uguagauacgagcaccagccac

SEQ ID NO: 1001 - RNA, 22 nt, Homo sapiens
uaagggguguauggcagaugca

SEQ ID NO: 1002 - RNA, 23 nt, Homo sapiens
acaggcggcuguagcaauggggg

SEQ ID NO: 1003 - RNA, 22 nt, Homo sapiens
aauucccuuguagauaacccgg

SEQ ID NO: 1004 - RNA, 22 nt, Homo sapiens
uacgcgcagaccacaggauguc

SEQ ID NO: 1005 - RNA, 22 nt, Homo sapiens
cagcccggaucccagcccacuu

SEQ ID NO: 1006 - RNA, 20 nt, Homo sapiens
guggguuggggcgggcucug

SEQ ID NO: 1007 - RNA, 22 nt, Homo sapiens
uuacacacaacugaggaucaua

SEQ ID NO: 1008 - RNA, 21 nt, Homo sapiens
uuucagauaacaguauuacau

SEQ ID NO: 1009 - RNA, 22 nt, Homo sapiens
aagcaauacuguuaccugaaau

SEQ ID NO: 1010 - RNA, 23 nt, Homo sapiens
uagcccccaggcuucacuuggcg

SEQ ID NO: 1011 - RNA, 23 nt, Homo sapiens
uucgggcuggccugcugcuccgg

SEQ ID NO: 1012 - RNA, 21 nt, Homo sapiens
ugugcagcaggccaaccgaga

SEQ ID NO: 1013 - RNA, 23 nt, Homo sapiens
agggcauaggagaggguugauau

SEQ ID NO: 1014 - RNA, 20 nt, Homo sapiens
ggcggcggcggaggcggggg

SEQ ID NO: 1015 - RNA, 22 nt, Homo sapiens
cugccagccccguuccagggca

SEQ ID NO: 1016 - RNA, 23 nt, Homo sapiens
acaaaguacagcauuagccuuag

SEQ ID NO: 1017 - RNA, 23 nt, Homo sapiens
aaaggucauuguaagguuaaugc

SEQ ID NO: 1018 - RNA, 22 nt, Homo sapiens
ugaggcuaaugcacuacuucac

SEQ ID NO: 1019 - RNA, 24 nt, Homo sapiens
uauagagagcaggaagauuaaugu

SEQ ID NO: 1020 - RNA, 23 nt, Homo sapiens
gugcuucaucguaauuaaccuua

SEQ ID NO: 1021 - RNA, 24 nt, Homo sapiens
guggaaagcaugcauccagggugu

SEQ ID NO: 1022 - RNA, 22 nt, Homo sapiens
gaauguugcucggugaaccccu

SEQ ID NO: 1023 - RNA, 23 nt, Homo sapiens
agguuacccgagcaacuuugcau

SEQ ID NO: 1024 - RNA, 21 nt, Homo sapiens
aauauaacacagauggccugu

SEQ ID NO: 1025 - RNA, 21 nt, Homo sapiens
aauauaacacagauggccugu

SEQ ID NO: 1026 - RNA, 21 nt, Homo sapiens
agguugucugugaugaguucg

SEQ ID NO: 1027 - RNA, 22 nt, Homo sapiens
uauguaacacgguccacuaacc

SEQ ID NO: 1028 - RNA, 21 nt, Homo sapiens
uaguagaccguauagcguacg

SEQ ID NO: 1029 - RNA, 23 nt, Homo sapiens
acuucaccugguccacuagccgu

SEQ ID NO: 1030 - RNA, 23 nt, Homo sapiens
acuucaccugguccacuagccgu

SEQ ID NO: 1031 - RNA, 23 nt, Homo sapiens
uggucgaccaguuggaaaguaau

SEQ ID NO: 1032 - RNA, 23 nt, Homo sapiens
aucaacagacauuaauugggcgc

SEQ ID NO: 1033 - RNA, 22 nt, Homo sapiens
acuggacuuagggucagaaggc

SEQ ID NO: 1034 - RNA, 23 nt, Homo sapiens
agcucggucugaggccccucagu

SEQ ID NO: 1035 - RNA, 23 nt, Homo sapiens
ugaggggcagagagcgagacuuu

SEQ ID NO: 1036 - RNA, 21 nt, Homo sapiens
caaaacgugaggcgcugcuau

SEQ ID NO: 1037 - RNA, 22 nt, Homo sapiens
cagcagcaauucauguuuugaa

SEQ ID NO: 1038 - RNA, 22 nt, Homo sapiens
aucgggaaugucguguccgccc

SEQ ID NO: 1039 - RNA, 23 nt, Homo sapiens
aaugacacgaucacucccguuga

SEQ ID NO: 1040 - RNA, 17 nt, Homo sapiens
ccugagaaaagggccaa

SEQ ID NO: 1041 - RNA, 19 nt, Homo sapiens
ggccacugagucagcacca

SEQ ID NO: 1042 - RNA, 18 nt, Homo sapiens
agggcauguccagggggu

SEQ ID NO: 1043 - RNA, 23 nt, Homo sapiens
gccuggagcuacuccaccaucuc

SEQ ID NO: 1044 - RNA, 17 nt, Homo sapiens
caguguucagagaugga

SEQ ID NO: 1045 - RNA, 18 nt, Homo sapiens
aucugaccugaugaaggu

SEQ ID NO: 1046 - RNA, 18 nt, Homo sapiens
ccagagguggggacugag

SEQ ID NO: 1047 - RNA, 17 nt, Homo sapiens
ccccgccaccgccuugg

SEQ ID NO: 1048 - RNA, 22 nt, Homo sapiens
caguugggucuaggggucagga

SEQ ID NO: 1049 - RNA, 19 nt, Homo sapiens
cuuggggcauggaguccca

SEQ ID NO: 1050 - RNA, 16 nt, Homo sapiens
aggaaacagggaccca

SEQ ID NO: 1051 - RNA, 17 nt, Homo sapiens
gacauucagacuaccug

SEQ ID NO: 1052 - RNA, 18 nt, Homo sapiens
auucuaagugccuuggcc

SEQ ID NO: 1053 - RNA, 17 nt, Homo sapiens
acucagucauggucauu

SEQ ID NO: 1054 - RNA, 19 nt, Homo sapiens
cugugggcucagcucuggg

SEQ ID NO: 1055 - RNA, 16 nt, Homo sapiens
cuaggaggccuuggcc

SEQ ID NO: 1056 - RNA, 16 nt, Homo sapiens
uccagcucgguggcac

SEQ ID NO: 1057 - RNA, 21 nt, Homo sapiens
ggcuccuccucucaggaugug

SEQ ID NO: 1058 - RNA, 21 nt, Homo sapiens
gcaggcacagacagcccuggc

SEQ ID NO: 1059 - RNA, 20 nt, Homo sapiens
ucagggagucaggggagggc

SEQ ID NO: 1060 - RNA, 19 nt, Homo sapiens
gggggaagaaaaggugggg

SEQ ID NO: 1061 - RNA, 18 nt, Homo sapiens
cauucaacuagugauugu

SEQ ID NO: 1062 - RNA, 18 nt, Homo sapiens
guguucucugauggacag

SEQ ID NO: 1063 - RNA, 18 nt, Homo sapiens
cagcagucccucccccug

SEQ ID NO: 1064 - RNA, 17 nt, Homo sapiens
ccaauuaccacuucuuu

SEQ ID NO: 1065 - RNA, 17 nt, Homo sapiens
cucagugacucaugugc

SEQ ID NO: 1066 - RNA, 21 nt, Homo sapiens
gcaguucugagcacaguacac

SEQ ID NO: 1067 - RNA, 18 nt, Homo sapiens
cuaggggguuugcccuug

SEQ ID NO: 1068 - RNA, 16 nt, Homo sapiens
cucuccucccggcuuc

SEQ ID NO: 1069 - RNA, 21 nt, Homo sapiens
gaguguaguucugagcagagc

SEQ ID NO: 1070 - RNA, 18 nt, Homo sapiens
gggucccggggagggggg

SEQ ID NO: 1071 - RNA, 18 nt, Homo sapiens
uaaaauuugcauccagga

SEQ ID NO: 1072 - RNA, 17 nt, Homo sapiens
uggggcucagcgaguuu

SEQ ID NO: 1073 - RNA, 18 nt, Homo sapiens
gggcucacaucaccccau

SEQ ID NO: 1074 - RNA, 18 nt, Homo sapiens
gcggcgaguccgacucau

SEQ ID NO: 1075 - RNA, 17 nt, Homo sapiens
accccacuccugguacc

SEQ ID NO: 1076 - RNA, 19 nt, Homo sapiens
ucucccuugagggcacuuu

SEQ ID NO: 1077 - RNA, 17 nt, Homo sapiens
uugucugcugaguuucc

SEQ ID NO: 1078 - RNA, 19 nt, Homo sapiens
gcauugugcagggcuauca

SEQ ID NO: 1079 - RNA, 22 nt, Homo sapiens
uaauacugucugguaaaaccgu

SEQ ID NO: 1080 - RNA, 19 nt, Homo sapiens
ugcccuccuuucuucccuc

SEQ ID NO: 1081 - RNA, 16 nt, Homo sapiens
uucagcaggaacagcu

SEQ ID NO: 1082 - RNA, 18 nt, Homo sapiens
ccccugggccggccuugg

SEQ ID NO: 1083 - RNA, 17 nt, Homo sapiens
cagccugacaggaacag

SEQ ID NO: 1084 - RNA, 17 nt, Homo sapiens
gggagucuacagcaggg

SEQ ID NO: 1085 - RNA, 18 nt, Homo sapiens
cagugcaauguuuuccuu

SEQ ID NO: 1086 - RNA, 17 nt, Homo sapiens
augugggcucaggcuca

SEQ ID NO: 1087 - RNA, 16 nt, Homo sapiens
ugccuuccugucugug

SEQ ID NO: 1088 - RNA, 22 nt, Homo sapiens
cugggacaggaggaggaggcag

SEQ ID NO: 1089 - RNA, 18 nt, Homo sapiens
gcuggugacaugagaggc

SEQ ID NO: 1090 - RNA, 18 nt, Homo sapiens
ugggagcuggacuacuuc

SEQ ID NO: 1091 - RNA, 20 nt, Homo sapiens
ucccacuacuucacuuguga

SEQ ID NO: 1092 - RNA, 18 nt, Homo sapiens
ccaguguggcucagcgag

SEQ ID NO: 1093 - RNA, 17 nt, Homo sapiens
uucugagcugaggacag

SEQ ID NO: 1094 - RNA, 17 nt, Homo sapiens
ccggcauguccagggca

SEQ ID NO: 1095 - RNA, 18 nt, Homo sapiens
ccuagacaccuccaguuc

SEQ ID NO: 1096 - RNA, 17 nt, Homo sapiens
uggagagaaaggcagua

SEQ ID NO: 1097 - RNA, 19 nt, Homo sapiens
aauguuuuuuccuguuucc

SEQ ID NO: 1098 - RNA, 18 nt, Homo sapiens
ucccuggaguuucuucuu

SEQ ID NO: 1099 - RNA, 18 nt, Homo sapiens
cuggagucuaggauucca

SEQ ID NO: 1100 - RNA, 22 nt, Homo sapiens
caggucgucuugcagggcuucu

SEQ ID NO: 1101 - RNA, 21 nt, Homo sapiens
ugucuugcaggccgucaugca

SEQ ID NO: 1102 - RNA, 16 nt, Homo sapiens
gcagcauucauguccc

SEQ ID NO: 1103 - RNA, 18 nt, Homo sapiens
gaaagagagcugagugug

SEQ ID NO: 1104 - RNA, 19 nt, Homo sapiens
ggccuuguuccugucccca

SEQ ID NO: 1105 - RNA, 20 nt, Homo sapiens
agcccccuggccccaaaccc

SEQ ID NO: 1106 - RNA, 18 nt, Homo sapiens
cucugggaaaugggacag

SEQ ID NO: 1107 - RNA, 18 nt, Homo sapiens
ccgcuuucugagcuggac

SEQ ID NO: 1108 - RNA, 17 nt, Homo sapiens
ggugaggcuagcuggug

SEQ ID NO: 1109 - RNA, 17 nt, Homo sapiens
acauugccagggaguuu

SEQ ID NO: 1110 - RNA, 17 nt, Homo sapiens
cacuguggguacaugcu

SEQ ID NO: 1111 - RNA, 17 nt, Homo sapiens
ucccugagcaaagccac

SEQ ID NO: 1112 - RNA, 21 nt, Homo sapiens
cuggauggcuccuccaugucu

SEQ ID NO: 1113 - RNA, 23 nt, Homo sapiens
ucuuggaguaggucauugggugg

SEQ ID NO: 1114 - RNA, 18 nt, Homo sapiens
gggauucuguagcuuccu

SEQ ID NO: 1115 - RNA, 21 nt, Homo sapiens
uuagcgguggaccgcccugcg

SEQ ID NO: 1116 - RNA, 21 nt, Homo sapiens
cugugggcucagcgcgugggg

SEQ ID NO: 1117 - RNA, 18 nt, Homo sapiens
cagccccacagccucaga

SEQ ID NO: 1118 - RNA, 20 nt, Homo sapiens
cccugagacccuaaccuuaa

SEQ ID NO: 1119 - RNA, 18 nt, Homo sapiens
uugcacuugucucaguga

SEQ ID NO: 1120 - RNA, 20 nt, Homo sapiens
uguuccucugucucccagac

SEQ ID NO: 1121 - RNA, 19 nt, Homo sapiens
ggcuugcaugggggacugg

SEQ ID NO: 1122 - RNA, 17 nt, Homo sapiens
ccaguuuucccaggauu

SEQ ID NO: 1123 - RNA, 19 nt, Homo sapiens
ccugagacccuaguuccac

SEQ ID NO: 1124 - RNA, 22 nt, Homo sapiens
aucaugaugggcuccucggugu

SEQ ID NO: 1125 - RNA, 22 nt, Homo sapiens
aucaugaugggcuccucggugu

SEQ ID NO: 1126 - RNA, 22 nt, Homo sapiens
uacggugagccugucauuauuc

SEQ ID NO: 1127 - RNA, 19 nt, Homo sapiens
ccucagaucagagccuugc

SEQ ID NO: 1128 - RNA, 18 nt, Homo sapiens
ggugggcuucccggaggg

SEQ ID NO: 1129 - RNA, 18 nt, Homo sapiens
cacugcaggacucagcag

SEQ ID NO: 1130 - RNA, 17 nt, Homo sapiens
ugagggaggagacugca

SEQ ID NO: 1131 - RNA, 18 nt, Homo sapiens
gaggcugaaggaagaugg

SEQ ID NO: 1132 - RNA, 22 nt, Homo sapiens
gucacugaugucuguagcugag

SEQ ID NO: 1133 - RNA, 22 nt, Homo sapiens
accugucuguggaaaggagcua

SEQ ID NO: 1134 - RNA, 21 nt, Homo sapiens
aaaagcaucaggaaguaccca

SEQ ID NO: 1135 - RNA, 21 nt, Homo sapiens
auaggcaccaaaaagcaacaa

SEQ ID NO: 1136 - RNA, 22 nt, Homo sapiens
aguugccuuuuuguucccaugc

SEQ ID NO: 1137 - RNA, 22 nt, Homo sapiens
agaguuaacucaaaauggacua

SEQ ID NO: 1138 - RNA, 22 nt, Homo sapiens
uguugggauucagcaggaccau

SEQ ID NO: 1139 - RNA, 17 nt, Homo sapiens
gaagauggacguacuuu

SEQ ID NO: 1140 - RNA, 21 nt, Homo sapiens
ucugaauagagucugaagagu

SEQ ID NO: 1141 - RNA, 22 nt, Homo sapiens
caaggagacgggaacauggagc

SEQ ID NO: 1142 - RNA, 20 nt, Homo sapiens
aaaagcugggcugagaggcg

SEQ ID NO: 1143 - RNA, 18 nt, Homo sapiens
aggcuggagugagcggag

SEQ ID NO: 1144 - RNA, 22 nt, Homo sapiens
gcgacucugaaaacuagaaggu

SEQ ID NO: 1145 - RNA, 20 nt, Homo sapiens
aaagacucugcaagaugccu

SEQ ID NO: 1146 - RNA, 21 nt, Homo sapiens
acaggagugggggugggacau

SEQ ID NO: 1147 - RNA, 21 nt, Homo sapiens
cgucccaccccccacuccugu

SEQ ID NO: 1148 - RNA, 21 nt, Homo sapiens
acaggagugggggugggacau

SEQ ID NO: 1149 - RNA, 21 nt, Homo sapiens
cgucccaccccccacuccugu

SEQ ID NO: 1150 - RNA, 21 nt, Homo sapiens
caggaguggggggugggacgu

SEQ ID NO: 1151 - RNA, 21 nt, Homo sapiens
augucccacccccacuccugu

SEQ ID NO: 1152 - RNA, 18 nt, Homo sapiens
aggagaaguaaaguagaa

SEQ ID NO: 1153 - RNA, 22 nt, Homo sapiens
auggccagagcucacacagagg

SEQ ID NO: 1154 - RNA, 21 nt, Homo sapiens
gcaggacaggcagaaguggau

SEQ ID NO: 1155 - RNA, 22 nt, Homo sapiens
cagggcaggaagaaguggacaa

SEQ ID NO: 1156 - RNA, 22 nt, Homo sapiens
guccacuucugccugcccugcc

SEQ ID NO: 1157 - RNA, 21 nt, Homo sapiens
ugggcucaggguacaaagguu

SEQ ID NO: 1158 - RNA, 21 nt, Homo sapiens
cacaggcuuagaaaagacagu

SEQ ID NO: 1159 - RNA, 22 nt, Homo sapiens
gugacugauaccuuggaggcau

SEQ ID NO: 1160 - RNA, 22 nt, Homo sapiens
ugucguggggcuugcuggcuug

SEQ ID NO: 1161 - RNA, 17 nt, Homo sapiens
acagggaggagauugua

SEQ ID NO: 1162 - RNA, 17 nt, Homo sapiens
gccggacaagagggagg

SEQ ID NO: 1163 - RNA, 17 nt, Homo sapiens
uuggaggcguggguuuu

SEQ ID NO: 1164 - RNA, 18 nt, Homo sapiens
cucgaguuggaagaggcg

SEQ ID NO: 1165 - RNA, 21 nt, Homo sapiens
cacggcaaaagaaacaaucca

SEQ ID NO: 1166 - RNA, 22 nt, Homo sapiens
agauuguuucuuuugccgugca

SEQ ID NO: 1167 - RNA, 22 nt, Homo sapiens
cagggcuggcagugacaugggu

SEQ ID NO: 1168 - RNA, 22 nt, Homo sapiens
auuucccugccauucccuuggc

SEQ ID NO: 1169 - RNA, 17 nt, Homo sapiens
ggugggggcuguuguuu

SEQ ID NO: 1170 - RNA, 20 nt, Homo sapiens
ggcuccuuggucuaggggua

SEQ ID NO: 1171 - RNA, 22 nt, Homo sapiens
cgucccggggcugcgcgaggca

SEQ ID NO: 1172 - RNA, 22 nt, Homo sapiens
uggggauuuggagaagugguga

SEQ ID NO: 1173 - RNA, 17 nt, Homo sapiens
ugguagagcugaggaca

SEQ ID NO: 1174 - RNA, 23 nt, Homo sapiens
uugaauucuuggccuuaagugau

SEQ ID NO: 1175 - RNA, 20 nt, Homo sapiens
gagcuuggucuguagcgguu

SEQ ID NO: 1176 - RNA, 20 nt, Homo sapiens
ggauccgagucacggcacca

SEQ ID NO: 1177 - RNA, 17 nt, Homo sapiens
aggguguguguguuuuu

SEQ ID NO: 1178 - RNA, 17 nt, Homo sapiens
ccugguggcuuccuuuu

SEQ ID NO: 1179 - RNA, 22 nt, Homo sapiens
ucacaagguauugacuggcgua

SEQ ID NO: 1180 - RNA, 19 nt, Homo sapiens
agagguagguguggaagaa

SEQ ID NO: 1181 - RNA, 22 nt, Homo sapiens
ccaggaggcggaggagguggag

SEQ ID NO: 1182 - RNA, 22 nt, Homo sapiens
auagugguugugaauuuaccuu

SEQ ID NO: 1183 - RNA, 23 nt, Homo sapiens
gauugagacuaguagggcuaggc

SEQ ID NO: 1184 - RNA, 23 nt, Homo sapiens
ugacacggaggguggcuugggaa

SEQ ID NO: 1185 - RNA, 17 nt, Homo sapiens
gagacugggguggggcc

SEQ ID NO: 1186 - RNA, 21 nt, Homo sapiens
aagguuuggauagaugcaaua

SEQ ID NO: 1187 - RNA, 22 nt, Homo sapiens
cucaaguagucugaccagggga

SEQ ID NO: 1188 - RNA, 18 nt, Homo sapiens
gggugcgggccggcgggg

SEQ ID NO: 1189 - RNA, 22 nt, Homo sapiens
uggcggcgguaguuaugggcuu

SEQ ID NO: 1190 - RNA, 18 nt, Homo sapiens
agagcagaaggaugagau

SEQ ID NO: 1191 - RNA, 22 nt, Homo sapiens
gcucccucuagggucgcucgga

SEQ ID NO: 1192 - RNA, 21 nt, Homo sapiens
uggcaaacguggaagccgaga

SEQ ID NO: 1193 - RNA, 22 nt, Homo sapiens
ugggaacuuaguagagguuuaa

SEQ ID NO: 1194 - RNA, 18 nt, Homo sapiens
ggugggggguguuguuuu

SEQ ID NO: 1195 - RNA, 22 nt, Homo sapiens
cuagugcucuccguuacaagua

SEQ ID NO: 1196 - RNA, 22 nt, Homo sapiens
uuguggcuggucaugaggcuaa

SEQ ID NO: 1197 - RNA, 21 nt, Homo sapiens
uuagucucaugaucagacaca

SEQ ID NO: 1198 - RNA, 22 nt, Homo sapiens
caagggaccaagcauucauuau

SEQ ID NO: 1199 - RNA, 22 nt, Homo sapiens
caggaaggauuuagggacaggc

SEQ ID NO: 1200 - RNA, 22 nt, Homo sapiens
cuauuaaggacauuugugauuc

SEQ ID NO: 1201 - RNA, 22 nt, Homo sapiens
auuaaggacauuugugauugau

SEQ ID NO: 1202 - RNA, 17 nt, Homo sapiens
gaggcugagcugaggag

SEQ ID NO: 1203 - RNA, 22 nt, Homo sapiens
cgcgcggccgugcucggagcag

SEQ ID NO: 1204 - RNA, 22 nt, Homo sapiens
uugcauauguaggaugucccau

SEQ ID NO: 1205 - RNA, 21 nt, Homo sapiens
agccaaguggaaguuacuuua

SEQ ID NO: 1206 - RNA, 17 nt, Homo sapiens
ggagugggcuggugguu

SEQ ID NO: 1207 - RNA, 22 nt, Homo sapiens
uuucuauuucucaguggggcuc

SEQ ID NO: 1208 - RNA, 22 nt, Homo sapiens
aacccagugggcuauggaaaug

SEQ ID NO: 1209 - RNA, 17 nt, Homo sapiens
gggguggucuguuguug

SEQ ID NO: 1210 - RNA, 20 nt, Homo sapiens
aaaaggcgggagaagcccca

SEQ ID NO: 1211 - RNA, 20 nt, Homo sapiens
uaacggccgcgguacccuaa

SEQ ID NO: 1212 - RNA, 20 nt, Homo sapiens
uaacggccgcgguacccuaa

SEQ ID NO: 1213 - RNA, 16 nt, Homo sapiens
accgccugcccaguga

SEQ ID NO: 1214 - RNA, 17 nt, Homo sapiens
gcugggcgaggcuggca

SEQ ID NO: 1215 - RNA, 19 nt, Homo sapiens
agagcuggcugaagggcag

SEQ ID NO: 1216 - RNA, 18 nt, Homo sapiens
agggggcgggcuccggcg

SEQ ID NO: 1217 - RNA, 21 nt, Homo sapiens
uggggcuagugaugcaggacg

SEQ ID NO: 1218 - RNA, 22 nt, Homo sapiens
ucugguaagagauuugggcaua

SEQ ID NO: 1219 - RNA, 22 nt, Homo sapiens
aauguggacuggugugaccaaa

SEQ ID NO: 1220 - RNA, 17 nt, Homo sapiens
ggggcugggcgcgcgcc

SEQ ID NO: 1221 - RNA, 21 nt, Homo sapiens
agaaggccuuuccaucucugu

SEQ ID NO: 1222 - RNA, 22 nt, Homo sapiens
ccagacuguggcugaccagagg

SEQ ID NO: 1223 - RNA, 21 nt, Homo sapiens
aauguaaacaggcuuuuugcu

SEQ ID NO: 1224 - RNA, 22 nt, Homo sapiens
gaggaaacugaagcugagaggg

SEQ ID NO: 1225 - RNA, 17 nt, Homo sapiens
cuccgggacggcugggc

SEQ ID NO: 1226 - RNA, 22 nt, Homo sapiens
ugggcuggcagggcaagugcug

SEQ ID NO: 1227 - RNA, 17 nt, Homo sapiens
aagacugagaggaggga

SEQ ID NO: 1228 - RNA, 22 nt, Homo sapiens
uggcaguguauuguuagcuggu

SEQ ID NO: 1229 - RNA, 22 nt, Homo sapiens
cagccacaacuacccugccacu

SEQ ID NO: 1230 - RNA, 22 nt, Homo sapiens
aggcaguguauuguuagcuggc

SEQ ID NO: 1231 - RNA, 23 nt, Homo sapiens
uugcuaguugcacuccucucugu

SEQ ID NO: 1232 - RNA, 25 nt, Homo sapiens
uaggcaguguauugcuagcggcugu

SEQ ID NO: 1233 - RNA, 17 nt, Homo sapiens
ugagguaguaguuucuu

SEQ ID NO: 1234 - RNA, 21 nt, Homo sapiens
uaugugaccucggaugaauca

SEQ ID NO: 1235 - RNA, 22 nt, Homo sapiens
gcugaugaugauggugcugaag

SEQ ID NO: 1236 - RNA, 22 nt, Homo sapiens
uuuaagcaggaaauagaauuua

SEQ ID NO: 1237 - RNA, 22 nt, Homo sapiens
ugugacaauagagaugaacaug

SEQ ID NO: 1238 - RNA, 18 nt, Homo sapiens
aggcugggcugggacgga

SEQ ID NO: 1239 - RNA, 20 nt, Homo sapiens
aaauggguggucugaggcaa

SEQ ID NO: 1240 - RNA, 20 nt, Homo sapiens
cuggguugggcugggcuggg

SEQ ID NO: 1241 - RNA, 17 nt, Homo sapiens
gcggggcugggcgcgcg

SEQ ID NO: 1242 - RNA, 22 nt, Homo sapiens
acuaaaggauauagaagguuuu

SEQ ID NO: 1243 - RNA, 22 nt, Homo sapiens
auugggaacauuuugcauguau

SEQ ID NO: 1244 - RNA, 22 nt, Homo sapiens
auuggggacauuuugcauucau

SEQ ID NO: 1245 - RNA, 22 nt, Homo sapiens
auuggggacauuuugcauucau

SEQ ID NO: 1246 - RNA, 22 nt, Homo sapiens
uuuugcgauguguuccuaauau

SEQ ID NO: 1247 - RNA, 22 nt, Homo sapiens
uugggaucauuuugcauccaua

SEQ ID NO: 1248 - RNA, 22 nt, Homo sapiens
uuuugcaauauguuccugaaua

SEQ ID NO: 1249 - RNA, 22 nt, Homo sapiens
ugagggaguaggauguaugguu

SEQ ID NO: 1250 - RNA, 22 nt, Homo sapiens
gaagaacuguugcauuugcccu

SEQ ID NO: 1251 - RNA, 22 nt, Homo sapiens
cagggccucacuguaucgccca

SEQ ID NO: 1252 - RNA, 22 nt, Homo sapiens
agacugacggcuggaggcccau

SEQ ID NO: 1253 - RNA, 18 nt, Homo sapiens
acaggcaggauuggggaa

SEQ ID NO: 1254 - RNA, 22 nt, Homo sapiens
aggacuggacucccggcagccc

SEQ ID NO: 1255 - RNA, 17 nt, Homo sapiens
gggagaagggucggggc

SEQ ID NO: 1256 - RNA, 25 nt, Homo sapiens
aaauaugaugaaacucacagcugag

SEQ ID NO: 1257 - RNA, 26 nt, Homo sapiens
gcucagggaugauaacugugcugaga

SEQ ID NO: 1258 - RNA, 18 nt, Homo sapiens
cagcagugcgcagggcug

SEQ ID NO: 1259 - RNA, 22 nt, Homo sapiens
aaaccguuaccauuacugaguu

SEQ ID NO: 1260 - RNA, 22 nt, Homo sapiens
uagcaagagaaccauuaccauu

SEQ ID NO: 1261 - RNA, 22 nt, Homo sapiens
cucaucugcaaagaaguaagug

SEQ ID NO: 1262 - RNA, 22 nt, Homo sapiens
aacuguuugcagaggaaacuga

SEQ ID NO: 1263 - RNA, 22 nt, Homo sapiens
uuuggacagaaaacacgcaggu

SEQ ID NO: 1264 - RNA, 22 nt, Homo sapiens
uuggacagaaaacacgcaggaa

SEQ ID NO: 1265 - RNA, 20 nt, Homo sapiens
ccugcguguuuucuguccaa

SEQ ID NO: 1266 - RNA, 22 nt, Homo sapiens
uuggacagaaaacacgcaggaa

SEQ ID NO: 1267 - RNA, 20 nt, Homo sapiens
ccugcguguuuucuguccaa

SEQ ID NO: 1268 - RNA, 22 nt, Homo sapiens
uuuggacagaaaacacgcaggu

SEQ ID NO: 1269 - RNA, 20 nt, Homo sapiens
ccugcguguuuucuguccaa

SEQ ID NO: 1270 - RNA, 22 nt, Homo sapiens
gcuaaggaaguccugugcucag

SEQ ID NO: 1271 - RNA, 21 nt, Homo sapiens
ugacucugccuguaggccggu

SEQ ID NO: 1272 - RNA, 21 nt, Homo sapiens
gaccgagagggccucggcugu

SEQ ID NO: 1273 - RNA, 22 nt, Homo sapiens
ugagacaggcuuaugcugcuau

SEQ ID NO: 1274 - RNA, 22 nt, Homo sapiens
auagcagcaugaaccugucuca

SEQ ID NO: 1275 - RNA, 20 nt, Homo sapiens
gagacagguucaugcugcua

SEQ ID NO: 1276 - RNA, 21 nt, Homo sapiens
auagcagcauaagccugucuc

SEQ ID NO: 1277 - RNA, 21 nt, Homo sapiens
ggggggaugugcaugcugguu

SEQ ID NO: 1278 - RNA, 22 nt, Homo sapiens
gcugacagcagggcuggccgcu

SEQ ID NO: 1279 - RNA, 22 nt, Homo sapiens
uggucugcaaagagaugacugu

SEQ ID NO: 1280 - RNA, 22 nt, Homo sapiens
ucauuauauguaugaucuggac

SEQ ID NO: 1281 - RNA, 21 nt, Homo sapiens
auuggacugcugauggcccgu

SEQ ID NO: 1282 - RNA, 22 nt, Homo sapiens
aggccaucagcaguccaaugaa

SEQ ID NO: 1283 - RNA, 18 nt, Homo sapiens
cccagcaggacgggagcg

SEQ ID NO: 1284 - RNA, 17 nt, Homo sapiens
auggagaaggcuucuga

SEQ ID NO: 1285 - RNA, 17 nt, Homo sapiens
ccccggggagcccggcg

SEQ ID NO: 1286 - RNA, 22 nt, Homo sapiens
uggaaggagguugccggacgcu

SEQ ID NO: 1287 - RNA, 17 nt, Homo sapiens
ggauggaggaggggucu

SEQ ID NO: 1288 - RNA, 17 nt, Homo sapiens
guggaccuggcugggac

SEQ ID NO: 1289 - RNA, 21 nt, Homo sapiens
ucgugcauauaucuaccacau

SEQ ID NO: 1290 - RNA, 21 nt, Homo sapiens
ugugguagauauaugcacgau

SEQ ID NO: 1291 - RNA, 22 nt, Homo sapiens
ugagccgagcugagcuuagcug

SEQ ID NO: 1292 - RNA, 22 nt, Homo sapiens
gagcuuggaugagcugggcuga

SEQ ID NO: 1293 - RNA, 22 nt, Homo sapiens
gcugaacugggcugagcugggc

SEQ ID NO: 1294 - RNA, 23 nt, Homo sapiens
uagugcaauauugcuuauagggu

SEQ ID NO: 1295 - RNA, 22 nt, Homo sapiens
acccuaucaauauugucucugc

SEQ ID NO: 1296 - RNA, 21 nt, Homo sapiens
uuaguccugccuguagguuua

SEQ ID NO: 1297 - RNA, 21 nt, Homo sapiens
gcaguccaugggcauauacac

SEQ ID NO: 1298 - RNA, 22 nt, Homo sapiens
uaugugccuuuggacuacaucg

SEQ ID NO: 1299 - RNA, 22 nt, Homo sapiens
ugccgcccucucgcugcucuag

SEQ ID NO: 1300 - RNA, 23 nt, Homo sapiens
gagggcagcguggguguggcgga

SEQ ID NO: 1301 - RNA, 22 nt, Homo sapiens
aggagcuagccaggcauaugca

SEQ ID NO: 1302 - RNA, 20 nt, Homo sapiens
auaugccuggcuagcuccuc

SEQ ID NO: 1303 - RNA, 19 nt, Homo sapiens
cggcgcgaccggcccgggg

SEQ ID NO: 1304 - RNA, 21 nt, Homo sapiens
ucuugaagucagaacccgcaa

SEQ ID NO: 1305 - RNA, 22 nt, Homo sapiens
aacucguguucaaagccuuuag

SEQ ID NO: 1306 - RNA, 22 nt, Homo sapiens
uacuaacugcagauucaaguga

SEQ ID NO: 1307 - RNA, 23 nt, Homo sapiens
ccuggacaccgcucagccggccg

SEQ ID NO: 1308 - RNA, 21 nt, Homo sapiens
acucggcugcgguggacaagu

SEQ ID NO: 1309 - RNA, 20 nt, Homo sapiens
ucacucucaccuugcuuugc

SEQ ID NO: 1310 - RNA, 22 nt, Homo sapiens
uugcuaaguaggcugagauuga

SEQ ID NO: 1311 - RNA, 22 nt, Homo sapiens
cacccccuguuuccuggcccac

SEQ ID NO: 1312 - RNA, 23 nt, Homo sapiens
ugggccagggagcagcugguggg

SEQ ID NO: 1313 - RNA, 24 nt, Homo sapiens
ugcccaugccauacuuuugccuca

SEQ ID NO: 1314 - RNA, 22 nt, Homo sapiens
auggcaucguccccugguggcu

SEQ ID NO: 1315 - RNA, 22 nt, Homo sapiens
gacacaugaccauaaaugcuaa

SEQ ID NO: 1316 - RNA, 23 nt, Homo sapiens
uggagagagaaaagagacagaag

SEQ ID NO: 1317 - RNA, 22 nt, Homo sapiens
agacaguaguucuugccugguu

SEQ ID NO: 1318 - RNA, 19 nt, Homo sapiens
accaggcaagaaauauugu

SEQ ID NO: 1319 - RNA, 21 nt, Homo sapiens
auugucccucucccuucccag

SEQ ID NO: 1320 - RNA, 22 nt, Homo sapiens
acugggaagaggagcugaggga

SEQ ID NO: 1321 - RNA, 23 nt, Homo sapiens
gaagauggugcugugcugaggaa

SEQ ID NO: 1322 - RNA, 20 nt, Homo sapiens
ugugggacugcaaaugggag

SEQ ID NO: 1323 - RNA, 21 nt, Homo sapiens
ucugaggccugccucucccca

SEQ ID NO: 1324 - RNA, 24 nt, Homo sapiens
ugggcgaggggugggcucucagag

SEQ ID NO: 1325 - RNA, 21 nt, Homo sapiens
agguagaaugaggccugacau

SEQ ID NO: 1326 - RNA, 19 nt, Homo sapiens
ucaggccucuuucuaccuu

SEQ ID NO: 1327 - RNA, 20 nt, Homo sapiens
cggggugggugaggucgggc

SEQ ID NO: 1328 - RNA, 22 nt, Homo sapiens
guucuguuaacccauccccuca

SEQ ID NO: 1329 - RNA, 22 nt, Homo sapiens
aggggacugguuaauagaacua

SEQ ID NO: 1330 - RNA, 23 nt, Homo sapiens
uggaguuaaggguugcuuggaga

SEQ ID NO: 1331 - RNA, 22 nt, Homo sapiens
ucucugagcaaggcuuaacacc

SEQ ID NO: 1332 - RNA, 22 nt, Homo sapiens
ugugggaucuggaggcaucugg

SEQ ID NO: 1333 - RNA, 21 nt, Homo sapiens
acccucgucagguccccgggg

SEQ ID NO: 1334 - RNA, 22 nt, Homo sapiens
caccggggauggcagagggucg

SEQ ID NO: 1335 - RNA, 23 nt, Homo sapiens
ugggcugagggcaggaggccugu

SEQ ID NO: 1336 - RNA, 23 nt, Homo sapiens
aauguggaaguggucugaggcau

SEQ ID NO: 1337 - RNA, 23 nt, Homo sapiens
gugaguguggauccuggaggaau

SEQ ID NO: 1338 - RNA, 22 nt, Homo sapiens
uuucuucuuagacauggcaacg

SEQ ID NO: 1339 - RNA, 22 nt, Homo sapiens
cugccaugucuaagaagaaaac

SEQ ID NO: 1340 - RNA, 22 nt, Homo sapiens
uuucuucuuagacauggcagcu

SEQ ID NO: 1341 - RNA, 19 nt, Homo sapiens
uugccaugucuaagaagaa

SEQ ID NO: 1342 - RNA, 23 nt, Homo sapiens
auacacauacacgcaacacacau

SEQ ID NO: 1343 - RNA, 23 nt, Homo sapiens
ugcagcucugguggaaaauggag

SEQ ID NO: 1344 - RNA, 22 nt, Homo sapiens
caggauccacagagcuagucca

SEQ ID NO: 1345 - RNA, 22 nt, Homo sapiens
aacuagcucuguggauccugac

SEQ ID NO: 1346 - RNA, 22 nt, Homo sapiens
aaagauagacaauuggcuaaau

SEQ ID NO: 1347 - RNA, 22 nt, Homo sapiens
uuagccaauuguccaucuuuag

SEQ ID NO: 1348 - RNA, 22 nt, Homo sapiens
aaagauggacaauuggcuaaau

SEQ ID NO: 1349 - RNA, 24 nt, Homo sapiens
agcugagcuccauggacgugcagu

SEQ ID NO: 1350 - RNA, 22 nt, Homo sapiens
cuuccggucugugagccccguc

SEQ ID NO: 1351 - RNA, 22 nt, Homo sapiens
uggggugcccacuccgcaaguu

SEQ ID NO: 1352 - RNA, 26 nt, Homo sapiens
cucggccgcggcgcguagcccccgcc

SEQ ID NO: 1353 - RNA, 23 nt, Homo sapiens
cugggggacgcgugagcgcgagc

SEQ ID NO: 1354 - RNA, 21 nt, Homo sapiens
cauacaaucugacauguauuu

SEQ ID NO: 1355 - RNA, 21 nt, Homo sapiens
auacaugucagauuguaugcc

SEQ ID NO: 1356 - RNA, 23 nt, Homo sapiens
uugcaugucagauuguaauuccc

SEQ ID NO: 1357 - RNA, 21 nt, Homo sapiens
ucccuccuucuguccccacag

SEQ ID NO: 1358 - RNA, 22 nt, Homo sapiens
acuggggagcagaaggagaacc

SEQ ID NO: 1359 - RNA, 23 nt, Homo sapiens
gaaaauccuuuuuguuuuuccag

SEQ ID NO: 1360 - RNA, 23 nt, Homo sapiens
agggaaaaaaaaaaggauuuguc

SEQ ID NO: 1361 - RNA, 22 nt, Homo sapiens
uguguccgggaaguggaggagg

SEQ ID NO: 1362 - RNA, 22 nt, Homo sapiens
ugaaguuacaucauggucgcuu

SEQ ID NO: 1363 - RNA, 22 nt, Homo sapiens
aagcgaccaugauguaacuuca

SEQ ID NO: 1364 - RNA, 21 nt, Homo sapiens
uuagugcauagucuuuggucu

SEQ ID NO: 1365 - RNA, 22 nt, Homo sapiens
accgaagacugugcgcuaaucu

SEQ ID NO: 1366 - RNA, 21 nt, Homo sapiens
uuacacagcuggacagaggca

SEQ ID NO: 1367 - RNA, 22 nt, Homo sapiens
uccaggcaggagccggacugga

SEQ ID NO: 1368 - RNA, 21 nt, Homo sapiens
cugggcucgggacgcgcggcu

SEQ ID NO: 1369 - RNA, 22 nt, Homo sapiens
ggggcugugauugaccagcagg

SEQ ID NO: 1370 - RNA, 22 nt, Homo sapiens
cacuguuucaccacuggcucuu

SEQ ID NO: 1371 - RNA, 21 nt, Homo sapiens
gagccaguggugagacaguga

SEQ ID NO: 1372 - RNA, 22 nt, Homo sapiens
ucugugagaccaaagaacuacu

SEQ ID NO: 1373 - RNA, 22 nt, Homo sapiens
uuguucuuuggucuuucagcca

SEQ ID NO: 1374 - RNA, 22 nt, Homo sapiens
aagguauuguucagacuuauga

SEQ ID NO: 1375 - RNA, 22 nt, Homo sapiens
ucugugauagagauucuuugcu

SEQ ID NO: 1376 - RNA, 21 nt, Homo sapiens
ucugaauuguaagaguuguua

SEQ ID NO: 1377 - RNA, 23 nt, Homo sapiens
agaacucuugcagucuuagaugu

SEQ ID NO: 1378 - RNA, 22 nt, Homo sapiens
aacgggaaugcaggcuguaucu

SEQ ID NO: 1379 - RNA, 23 nt, Homo sapiens
ucugaguuccuggagccuggucu

SEQ ID NO: 1380 - RNA, 23 nt, Homo sapiens
uggagauccagugcucgcccgau

SEQ ID NO: 1381 - RNA, 22 nt, Homo sapiens
uguugcaagucgguggagacgu

SEQ ID NO: 1382 - RNA, 22 nt, Homo sapiens
cucucuacugacuugcaacaua

SEQ ID NO: 1383 - RNA, 22 nt, Homo sapiens
ucucccuuccugcccuggcuag

SEQ ID NO: 1384 - RNA, 26 nt, Homo sapiens
cccagggcuuggaguggggcaagguu

SEQ ID NO: 1385 - RNA, 23 nt, Homo sapiens
uaucugcugggcuuucugguguu

SEQ ID NO: 1386 - RNA, 21 nt, Homo sapiens
uggcuguuggagggggcaggc

SEQ ID NO: 1387 - RNA, 22 nt, Homo sapiens
cagcccuccucccgcacccaaa

SEQ ID NO: 1388 - RNA, 22 nt, Homo sapiens
uaggggcagcagaggaccuggg

SEQ ID NO: 1389 - RNA, 22 nt, Homo sapiens
uugaggagacauggugggggcc

SEQ ID NO: 1390 - RNA, 21 nt, Homo sapiens
gcagcccagcugaggccucug

SEQ ID NO: 1391 - RNA, 22 nt, Homo sapiens
gagcaggcgaggcugggcugaa

SEQ ID NO: 1392 - RNA, 23 nt, Homo sapiens
ccagccacggacugagagugcau

SEQ ID NO: 1393 - RNA, 23 nt, Homo sapiens
guccuccaggccaugagcugcgg

SEQ ID NO: 1394 - RNA, 22 nt, Homo sapiens
ucaggcaguguggguaucagau

SEQ ID NO: 1395 - RNA, 23 nt, Homo sapiens
ugagaguggaauucacaguauuu

SEQ ID NO: 1396 - RNA, 23 nt, Homo sapiens
auacugugaauuucacugucaca

SEQ ID NO: 1397 - RNA, 21 nt, Homo sapiens
caaauggacaggauaacaccu

SEQ ID NO: 1398 - RNA, 22 nt, Homo sapiens
agguguuauccuauccauuugc

SEQ ID NO: 1399 - RNA, 22 nt, Homo sapiens
ugaucucaccgcugccuccuuc

SEQ ID NO: 1400 - RNA, 22 nt, Homo sapiens
caggaggcagugggcgagcagg

SEQ ID NO: 1401 - RNA, 22 nt, Homo sapiens
ugcaagacggauacugucaucu

SEQ ID NO: 1402 - RNA, 24 nt, Homo sapiens
ugucagugacuccugccccuuggu

SEQ ID NO: 1403 - RNA, 22 nt, Homo sapiens
agggggcgcagucacugacgug

SEQ ID NO: 1404 - RNA, 23 nt, Homo sapiens
ucaaaauguagaggaagacccca

SEQ ID NO: 1405 - RNA, 22 nt, Homo sapiens
aauuuacucugcaaucuucucc

SEQ ID NO: 1406 - RNA, 22 nt, Homo sapiens
agaagauugcagaguaaguucc

SEQ ID NO: 1407 - RNA, 26 nt, Homo sapiens
cacaggacugacuccucaccccagug

SEQ ID NO: 1408 - RNA, 22 nt, Homo sapiens
ucuggggaugaggacagugugu

SEQ ID NO: 1409 - RNA, 20 nt, Homo sapiens
augggugauggguguggugu

SEQ ID NO: 1410 - RNA, 22 nt, Homo sapiens
uuggccaccacaccuaccccuu

SEQ ID NO: 1411 - RNA, 22 nt, Homo sapiens
uguaguuguauuguauugccac

SEQ ID NO: 1412 - RNA, 23 nt, Homo sapiens
uagcaauacaguacaaauauagu

SEQ ID NO: 1413 - RNA, 22 nt, Homo sapiens
ucagucacauaucuagugucua

SEQ ID NO: 1414 - RNA, 22 nt, Homo sapiens
gacacuaggcaugugagugauu

SEQ ID NO: 1415 - RNA, 22 nt, Homo sapiens
ucaaucacuugguaauugcugu

SEQ ID NO: 1416 - RNA, 25 nt, Homo sapiens
agcggggaggaagugggcgcugcuu

SEQ ID NO: 1417 - RNA, 22 nt, Homo sapiens
agcccgccccagccgagguucu

SEQ ID NO: 1418 - RNA, 23 nt, Homo sapiens
gccccggcgcgggcggguucugg

SEQ ID NO: 1419 - RNA, 22 nt, Homo sapiens
agcaaggcggcaucucucugau

SEQ ID NO: 1420 - RNA, 21 nt, Homo sapiens
agagaugccgccuugcuccuu

SEQ ID NO: 1421 - RNA, 23 nt, Homo sapiens
uugaagaggaggugcucuguagc

SEQ ID NO: 1422 - RNA, 22 nt, Homo sapiens
acaacagugacuugcucuccaa

SEQ ID NO: 1423 - RNA, 18 nt, Homo sapiens
gggugagggcaggugguu

SEQ ID NO: 1424 - RNA, 18 nt, Homo sapiens
cgugucuucuggcuugau

SEQ ID NO: 1425 - RNA, 23 nt, Homo sapiens
ugcaucaggccagaagacaugag

SEQ ID NO: 1426 - RNA, 21 nt, Homo sapiens
aaugagagaccuguacuguau

SEQ ID NO: 1427 - RNA, 22 nt, Homo sapiens
uccaguacaggucucucauuuc

SEQ ID NO: 1428 - RNA, 22 nt, Homo sapiens
ugggauccagacagugggagaa

SEQ ID NO: 1429 - RNA, 22 nt, Homo sapiens
uucucccacuaccaggcuccca

SEQ ID NO: 1430 - RNA, 22 nt, Homo sapiens
ccaaccuagguggucagaguug

SEQ ID NO: 1431 - RNA, 22 nt, Homo sapiens
aacucugaccccuuagguugau

SEQ ID NO: 1432 - RNA, 23 nt, Homo sapiens
gugccaccuuaacugcagccaau

SEQ ID NO: 1433 - RNA, 22 nt, Homo sapiens
aaguuggcugcaguuaaggugg

SEQ ID NO: 1434 - RNA, 22 nt, Homo sapiens
aagggggaaggaaacauggaga

SEQ ID NO: 1435 - RNA, 22 nt, Homo sapiens
uccauguuuccuucccccuucu

SEQ ID NO: 1436 - RNA, 21 nt, Homo sapiens
acacauggguggcuguggccu

SEQ ID NO: 1437 - RNA, 22 nt, Homo sapiens
uaggccacagccacccaugugu

SEQ ID NO: 1438 - RNA, 21 nt, Homo sapiens
agcuguaccugaaaccaagca

SEQ ID NO: 1439 - RNA, 22 nt, Homo sapiens
ucacaaaucuauaauaugcagg

SEQ ID NO: 1440 - RNA, 21 nt, Homo sapiens
ugcuuaaguuguaccaaguau

SEQ ID NO: 1441 - RNA, 22 nt, Homo sapiens
ccuggcauauuugguauaacuu

SEQ ID NO: 1442 - RNA, 22 nt, Homo sapiens
ugagggcuccaggugacggugg

SEQ ID NO: 1443 - RNA, 22 nt, Homo sapiens
accugccagcaccucccugcag

SEQ ID NO: 1444 - RNA, 23 nt, Homo sapiens
ggcaggagggcugugccagguug

SEQ ID NO: 1445 - RNA, 22 nt, Homo sapiens
cccucucuggcuccuccccaaa

SEQ ID NO: 1446 - RNA, 24 nt, Homo sapiens
ugggggagccaugagauaagagca

SEQ ID NO: 1447 - RNA, 21 nt, Homo sapiens
guaccuucugguucagcuagu

SEQ ID NO: 1448 - RNA, 23 nt, Homo sapiens
aacugaaccaggagugagcuucg

SEQ ID NO: 1449 - RNA, 22 nt, Homo sapiens
uggggaaggcgucagugucggg

SEQ ID NO: 1450 - RNA, 21 nt, Homo sapiens
agacccugcagccuucccacc

SEQ ID NO: 1451 - RNA, 22 nt, Homo sapiens
acccagguucccucuggccgca

SEQ ID NO: 1452 - RNA, 23 nt, Homo sapiens
agggccagaggagccuggagugg

SEQ ID NO: 1453 - RNA, 22 nt, Homo sapiens
auagugggaagcuggcagauuc

SEQ ID NO: 1454 - RNA, 21 nt, Homo sapiens
aucugccagcuuccacagugg

SEQ ID NO: 1455 - RNA, 25 nt, Homo sapiens
caugcugaccucccuccugccccag

SEQ ID NO: 1456 - RNA, 23 nt, Homo sapiens
ugggaggggagaggcagcaagca

SEQ ID NO: 1457 - RNA, 22 nt, Homo sapiens
ucauuuaucuguugggaagcua

SEQ ID NO: 1458 - RNA, 23 nt, Homo sapiens
cuggcggagcccauuccaugcca

SEQ ID NO: 1459 - RNA, 22 nt, Homo sapiens
cacacaaguggcccccaacacu

SEQ ID NO: 1460 - RNA, 22 nt, Homo sapiens
ugcugggggccacaugagugug

SEQ ID NO: 1461 - RNA, 21 nt, Homo sapiens
gcccugaccuguccuguucug

SEQ ID NO: 1462 - RNA, 23 nt, Homo sapiens
uguagagcagggagcaggaagcu

SEQ ID NO: 1463 - RNA, 22 nt, Homo sapiens
ccaccaggucuagcauugggau

SEQ ID NO: 1464 - RNA, 22 nt, Homo sapiens
aaucccaaugcuagacccggug

SEQ ID NO: 1465 - RNA, 22 nt, Homo sapiens
gcugcgggcugcggucagggcg

SEQ ID NO: 1466 - RNA, 21 nt, Homo sapiens
aaaggugcucaaauuagacau

SEQ ID NO: 1467 - RNA, 22 nt, Homo sapiens
ccuaauuugaacaccuucggua

SEQ ID NO: 1468 - RNA, 19 nt, Homo sapiens
aggcagguuaucugggcug

SEQ ID NO: 1469 - RNA, 20 nt, Homo sapiens
augcgaggaugcugacagug

SEQ ID NO: 1470 - RNA, 22 nt, Homo sapiens
ugaaacuggagcgccuggagga

SEQ ID NO: 1471 - RNA, 22 nt, Homo sapiens
accagcgcguuuucaguuucau

SEQ ID NO: 1472 - RNA, 25 nt, Homo sapiens
aagggaggaggagcggaggggcccu

SEQ ID NO: 1473 - RNA, 22 nt, Homo sapiens
gcccgagaggauccgucccugc

SEQ ID NO: 1474 - RNA, 22 nt, Homo sapiens
aggacugauccucucgggcagg

SEQ ID NO: 1475 - RNA, 23 nt, Homo sapiens
cgggcuguccggaggggucggcu

SEQ ID NO: 1476 - RNA, 23 nt, Homo sapiens
ucuguauucuccuuugccugcag

SEQ ID NO: 1477 - RNA, 23 nt, Homo sapiens
ucaggcaaagggauauuuacaga

SEQ ID NO: 1478 - RNA, 21 nt, Homo sapiens
uuucugucuuuucugguccag

SEQ ID NO: 1479 - RNA, 23 nt, Homo sapiens
uggccggaugggacaggaggcau

SEQ ID NO: 1480 - RNA, 23 nt, Homo sapiens
ucuaaagacuagacuucgcuaug

SEQ ID NO: 1481 - RNA, 23 nt, Homo sapiens
uggcccggcgacgucucacgguc

SEQ ID NO: 1482 - RNA, 23 nt, Homo sapiens
ugaguggggcucccgggacggcg

SEQ ID NO: 1483 - RNA, 21 nt, Homo sapiens
agcggugcuccugcgggccga

SEQ ID NO: 1484 - RNA, 23 nt, Homo sapiens
ccggucccaggagaaccugcaga

SEQ ID NO: 1485 - RNA, 22 nt, Homo sapiens
aaggcccgggcuuuccucccag

SEQ ID NO: 1486 - RNA, 22 nt, Homo sapiens
agggaaggaggcuuggucuuag

SEQ ID NO: 1487 - RNA, 21 nt, Homo sapiens
gagguuuggggaggauuugcu

SEQ ID NO: 1488 - RNA, 20 nt, Homo sapiens
cgccccuccugcccccacag

SEQ ID NO: 1489 - RNA, 22 nt, Homo sapiens
ugcggggacaggccagggcauc

SEQ ID NO: 1490 - RNA, 22 nt, Homo sapiens
ccugacccacccccucccgcag

SEQ ID NO: 1491 - RNA, 22 nt, Homo sapiens
cucgggcggaggugguugagug

SEQ ID NO: 1492 - RNA, 24 nt, Homo sapiens
agaggacccguagcugcuagaagg

SEQ ID NO: 1493 - RNA, 22 nt, Homo sapiens
uuguggaucucaaggaugugcu

SEQ ID NO: 1494 - RNA, 22 nt, Homo sapiens
uucucuuucuuuagccuugugu

SEQ ID NO: 1495 - RNA, 22 nt, Homo sapiens
caaggccaaaggaagagaacag

SEQ ID NO: 1496 - RNA, 23 nt, Homo sapiens
augcggaccuggguuagcggagu

SEQ ID NO: 1497 - RNA, 22 nt, Homo sapiens
agccaggcucugaagggaaagu

SEQ ID NO: 1498 - RNA, 22 nt, Homo sapiens
uuucccuucagagccuggcuuu

SEQ ID NO: 1499 - RNA, 22 nt, Homo sapiens
ccagagaugguugccuuccuau

SEQ ID NO: 1500 - RNA, 23 nt, Homo sapiens
cagggaggcgcucacucucugcu

SEQ ID NO: 1501 - RNA, 22 nt, Homo sapiens
caugacgucacagaggcuucgc

SEQ ID NO: 1502 - RNA, 23 nt, Homo sapiens
aggccucugugacgucacggugu

SEQ ID NO: 1503 - RNA, 23 nt, Homo sapiens
ugccccaccugcugaccacccuc

SEQ ID NO: 1504 - RNA, 23 nt, Homo sapiens
gugagugggagccgguggggcug

SEQ ID NO: 1505 - RNA, 21 nt, Homo sapiens
uaggacuagauguuggaauua

SEQ ID NO: 1506 - RNA, 22 nt, Homo sapiens
aaauucauguucaaucuaaacc

SEQ ID NO: 1507 - RNA, 22 nt, Homo sapiens
uuuagauugaacaugaaguuag

SEQ ID NO: 1508 - RNA, 21 nt, Homo sapiens
gagggcaugcgcacuuugucc

SEQ ID NO: 1509 - RNA, 21 nt, Homo sapiens
acaaggugugcaugccugacc

SEQ ID NO: 1510 - RNA, 22 nt, Homo sapiens
cuucugaucaagauuuguggug

SEQ ID NO: 1511 - RNA, 21 nt, Homo sapiens
ccaaaucuugaucagaagccu

SEQ ID NO: 1512 - RNA, 24 nt, Homo sapiens
aggcaggggcuggugcugggcggg

SEQ ID NO: 1513 - RNA, 21 nt, Homo sapiens
cgccugcccagcccuccugcu

SEQ ID NO: 1514 - RNA, 22 nt, Homo sapiens
uuaacuccuuucacacccaugg

SEQ ID NO: 1515 - RNA, 21 nt, Homo sapiens
uggauguggaaggaguuaucu

SEQ ID NO: 1516 - RNA, 22 nt, Homo sapiens
ugagugauugauagcuauguuc

SEQ ID NO: 1517 - RNA, 21 nt, Homo sapiens
auagcaauugcucuuuuggaa

SEQ ID NO: 1518 - RNA, 21 nt, Homo sapiens
ucugaaagagcaguugguguu

SEQ ID NO: 1519 - RNA, 23 nt, Homo sapiens
cgcgggcgcuccuggccgccgcc

SEQ ID NO: 1520 - RNA, 20 nt, Homo sapiens
ccaggagauccagagagaau

SEQ ID NO: 1521 - RNA, 22 nt, Homo sapiens
auucucucuggaucccauggau

SEQ ID NO: 1522 - RNA, 22 nt, Homo sapiens
ucugccauccucccuccccuac

SEQ ID NO: 1523 - RNA, 24 nt, Homo sapiens
ggugggauggagagaagguaugag

SEQ ID NO: 1524 - RNA, 18 nt, Homo sapiens
ugagaugacacuguagcu

SEQ ID NO: 1525 - RNA, 21 nt, Homo sapiens
agcagacuugaccuacaauua

SEQ ID NO: 1526 - RNA, 22 nt, Homo sapiens
ccugcaacuuugccugaucaga

SEQ ID NO: 1527 - RNA, 22 nt, Homo sapiens
ugaucaggcaaaauugcagacu

SEQ ID NO: 1528 - RNA, 22 nt, Homo sapiens
cagaacaggagcauagaaaggc

SEQ ID NO: 1529 - RNA, 21 nt, Homo sapiens
auugccuaacaugugccagaa

SEQ ID NO: 1530 - RNA, 22 nt, Homo sapiens
ucugguauguaguagguaauaa

SEQ ID NO: 1531 - RNA, 22 nt, Homo sapiens
uuaauuuuuuguuucggucacu

SEQ ID NO: 1532 - RNA, 23 nt, Homo sapiens
cuugccauccugguccacugcau

SEQ ID NO: 1533 - RNA, 22 nt, Homo sapiens
guggaccaggauggcaagggcu

SEQ ID NO: 1534 - RNA, 22 nt, Homo sapiens
auaccucaucuagaaugcugua

SEQ ID NO: 1535 - RNA, 22 nt, Homo sapiens
uucuagaugagagauauauaua

SEQ ID NO: 1536 - RNA, 22 nt, Homo sapiens
ucuucuuccuuugcagaguuga

SEQ ID NO: 1537 - RNA, 22 nt, Homo sapiens
aauucuguaaaggaagaagagg

SEQ ID NO: 1538 - RNA, 22 nt, Homo sapiens
uaggagggaauaguaaaagcag

SEQ ID NO: 1539 - RNA, 22 nt, Homo sapiens
acccuugagccugaucccuagc

SEQ ID NO: 1540 - RNA, 22 nt, Homo sapiens
aauguuggaauccucgcuagag

SEQ ID NO: 1541 - RNA, 21 nt, Homo sapiens
uagcggggauuccaauauugg

SEQ ID NO: 1542 - RNA, 22 nt, Homo sapiens
ugauugucuucauaucuagaac

SEQ ID NO: 1543 - RNA, 22 nt, Homo sapiens
uucuggauaugaagacaaucaa

SEQ ID NO: 1544 - RNA, 23 nt, Homo sapiens
ccccgguguuggggcgcgucugc

SEQ ID NO: 1545 - RNA, 21 nt, Homo sapiens
ggcgcgcccagcucccgggcu

SEQ ID NO: 1546 - RNA, 20 nt, Homo sapiens
ugaggagaugcugggacuga

SEQ ID NO: 1547 - RNA, 21 nt, Homo sapiens
agagucggcgacgccgccagc

SEQ ID NO: 1548 - RNA, 22 nt, Homo sapiens
ugaagccagcucuggucugggc

SEQ ID NO: 1549 - RNA, 22 nt, Homo sapiens
ugagaccaggacuggaugcacc

SEQ ID NO: 1550 - RNA, 24 nt, Homo sapiens
gaugcgccgcccacugccccgcgc

SEQ ID NO: 1551 - RNA, 22 nt, Homo sapiens
gcggggguggcggcggcauccc

SEQ ID NO: 1552 - RNA, 22 nt, Homo sapiens
uuacggaccagcuaagggaggc

SEQ ID NO: 1553 - RNA, 21 nt, Homo sapiens
cacacauagcagguguauaua

SEQ ID NO: 1554 - RNA, 22 nt, Homo sapiens
guauacaccugauauguguaug

SEQ ID NO: 1555 - RNA, 22 nt, Homo sapiens
ugaaugguaaagcgaugucaca

SEQ ID NO: 1556 - RNA, 20 nt, Homo sapiens
aucgcuuuaccauucauguu

SEQ ID NO: 1557 - RNA, 18 nt, Homo sapiens
uggauaugaugacugaaa

SEQ ID NO: 1558 - RNA, 18 nt, Homo sapiens
cggugagcgcucgcuggc

SEQ ID NO: 1559 - RNA, 23 nt, Homo sapiens
ucugcacugugaguuggcuggcu

SEQ ID NO: 1560 - RNA, 24 nt, Homo sapiens
acauccugcuccacagggcagagg

SEQ ID NO: 1561 - RNA, 22 nt, Homo sapiens
ucuggcuaucucacgagacugu

SEQ ID NO: 1562 - RNA, 22 nt, Homo sapiens
auauuauuagccacuucuggau

SEQ ID NO: 1563 - RNA, 21 nt, Homo sapiens
agaaguggcuaauaauauuga

SEQ ID NO: 1564 - RNA, 22 nt, Homo sapiens
uaaaguggcagaguauagacac

SEQ ID NO: 1565 - RNA, 22 nt, Homo sapiens
ugucuauacucugucacuuuac

SEQ ID NO: 1566 - RNA, 21 nt, Homo sapiens
ucucaguaaguggcacucugu

SEQ ID NO: 1567 - RNA, 21 nt, Homo sapiens
gacagagugccacuuacugaa

SEQ ID NO: 1568 - RNA, 22 nt, Homo sapiens
aacucacgaaguauaccgaagu

SEQ ID NO: 1569 - RNA, 22 nt, Homo sapiens
uucgguauacuuugugaauugg

SEQ ID NO: 1570 - RNA, 22 nt, Homo sapiens
acuggcaugcugcauuuauaua

SEQ ID NO: 1571 - RNA, 22 nt, Homo sapiens
aucuaaaugcagcaugccaguc

SEQ ID NO: 1572 - RNA, 19 nt, Homo sapiens
cauccguccgucuguccac

SEQ ID NO: 1573 - RNA, 21 nt, Homo sapiens
aguggaccgaggaaggaagga

SEQ ID NO: 1574 - RNA, 22 nt, Homo sapiens
uacacaagaaaaccaaggcuca

SEQ ID NO: 1575 - RNA, 23 nt, Homo sapiens
uacauggauggaaaccuucaagc

SEQ ID NO: 1576 - RNA, 22 nt, Homo sapiens
uauggagguucuagaccauguu

SEQ ID NO: 1577 - RNA, 21 nt, Homo sapiens
uaacauaauaguguggauuga

SEQ ID NO: 1578 - RNA, 21 nt, Homo sapiens
ugcuuaaccuugcccucgaaa

SEQ ID NO: 1579 - RNA, 21 nt, Homo sapiens
uuggacgguaagguuaagcaa

SEQ ID NO: 1580 - RNA, 21 nt, Homo sapiens
ucacuccucuccucccgucuu

SEQ ID NO: 1581 - RNA, 22 nt, Homo sapiens
aagacgggaggaaagaagggag

SEQ ID NO: 1582 - RNA, 22 nt, Homo sapiens
ucaggcucaguccccucccgau

SEQ ID NO: 1583 - RNA, 22 nt, Homo sapiens
gucauacacggcucuccucucu

SEQ ID NO: 1584 - RNA, 22 nt, Homo sapiens
agaggcuggccgugaugaauuc

SEQ ID NO: 1585 - RNA, 21 nt, Homo sapiens
cggggcagcucaguacaggau

SEQ ID NO: 1586 - RNA, 22 nt, Homo sapiens
uccuguacugagcugccccgag

SEQ ID NO: 1587 - RNA, 22 nt, Homo sapiens
aaucauacagggacauccaguu

SEQ ID NO: 1588 - RNA, 22 nt, Homo sapiens
aaucauacagggacauccaguu

SEQ ID NO: 1589 - RNA, 22 nt, Homo sapiens
gugguuaucccugcuguguucg

SEQ ID NO: 1590 - RNA, 22 nt, Homo sapiens
aaucguacagggucauccacuu

SEQ ID NO: 1591 - RNA, 22 nt, Homo sapiens
aaucguacagggucauccacuu

SEQ ID NO: 1592 - RNA, 22 nt, Homo sapiens
gugguuaucccuguccuguucg

SEQ ID NO: 1593 - RNA, 21 nt, Homo sapiens
uugaaaggcuauuucuugguc

SEQ ID NO: 1594 - RNA, 21 nt, Homo sapiens
cccagauaauggcacucucaa

SEQ ID NO: 1595 - RNA, 22 nt, Homo sapiens
gugacaucacauauacggcagc

SEQ ID NO: 1596 - RNA, 22 nt, Homo sapiens
gugacaucacauauacggcagc

SEQ ID NO: 1597 - RNA, 22 nt, Homo sapiens
ggucguaugugugacgccauuu

SEQ ID NO: 1598 - RNA, 22 nt, Homo sapiens
caaccuggaggacuccaugcug

SEQ ID NO: 1599 - RNA, 20 nt, Homo sapiens
ccauggaucuccaggugggu

SEQ ID NO: 1600 - RNA, 22 nt, Homo sapiens
cuuaugcaagauucccuucuac

SEQ ID NO: 1601 - RNA, 22 nt, Homo sapiens
aguggggaacccuuccaugagg

SEQ ID NO: 1602 - RNA, 23 nt, Homo sapiens
aggaccugcgggacaagauucuu

SEQ ID NO: 1603 - RNA, 22 nt, Homo sapiens
ugaaggucuacugugugccagg

SEQ ID NO: 1604 - RNA, 22 nt, Homo sapiens
uuguacaugguaggcuuucauu

SEQ ID NO: 1605 - RNA, 22 nt, Homo sapiens
ugaaacauacacgggaaaccuc

SEQ ID NO: 1606 - RNA, 22 nt, Homo sapiens
ugaaacauacacgggaaaccuc

SEQ ID NO: 1607 - RNA, 23 nt, Homo sapiens
agguuguccguguugucuucucu

SEQ ID NO: 1608 - RNA, 22 nt, Homo sapiens
aaacaaacauggugcacuucuu

SEQ ID NO: 1609 - RNA, 22 nt, Homo sapiens
gaaguugcccauguuauuuucg

SEQ ID NO: 1610 - RNA, 22 nt, Homo sapiens
ugaguauuacauggccaaucuc

SEQ ID NO: 1611 - RNA, 22 nt, Homo sapiens
caaaccacacugugguguuaga

SEQ ID NO: 1612 - RNA, 21 nt, Homo sapiens
cagcagcacacugugguuugu

SEQ ID NO: 1613 - RNA, 23 nt, Homo sapiens
uuucaagccagggggcguuuuuc

SEQ ID NO: 1614 - RNA, 20 nt, Homo sapiens
ucacuaccugacaauacagu

SEQ ID NO: 1615 - RNA, 21 nt, Homo sapiens
ugcuguauugucagguaguga

SEQ ID NO: 1616 - RNA, 22 nt, Homo sapiens
aacaucacagcaagucugugcu

SEQ ID NO: 1617 - RNA, 21 nt, Homo sapiens
uuaagacuugcagugauguuu

SEQ ID NO: 1618 - RNA, 22 nt, Homo sapiens
aacaucacugcaagucuuaaca

SEQ ID NO: 1619 - RNA, 21 nt, Homo sapiens
acagacuugcugugauguuca

SEQ ID NO: 1620 - RNA, 22 nt, Homo sapiens
ucaggacacuucugaacuugga

SEQ ID NO: 1621 - RNA, 22 nt, Homo sapiens
caguucagaaguguuccugagu

SEQ ID NO: 1622 - RNA, 22 nt, Homo sapiens
uucugccucuguccagguccuu

SEQ ID NO: 1623 - RNA, 24 nt, Homo sapiens
agggcuggacucagcggcggagcu

SEQ ID NO: 1624 - RNA, 21 nt, Homo sapiens
ugacugccucacugaccacuu

SEQ ID NO: 1625 - RNA, 24 nt, Homo sapiens
aauuugguuucugaggcacuuagu

SEQ ID NO: 1626 - RNA, 21 nt, Homo sapiens
uacuuuucuagguuguugggg

SEQ ID NO: 1627 - RNA, 23 nt, Homo sapiens
ucacaacaaccuugcaggguaga

SEQ ID NO: 1628 - RNA, 22 nt, Homo sapiens
cuuggauuuuccugggccucag

SEQ ID NO: 1629 - RNA, 22 nt, Homo sapiens
ugaggacagggcaaauucacga

SEQ ID NO: 1630 - RNA, 21 nt, Homo sapiens
uuucccuuuccauccuggcag

SEQ ID NO: 1631 - RNA, 21 nt, Homo sapiens
uugccagggcaggagguggaa

SEQ ID NO: 1632 - RNA, 22 nt, Homo sapiens
aucauaugaaccaaacucuaau

SEQ ID NO: 1633 - RNA, 22 nt, Homo sapiens
uagagucuggcugauaugguuu

SEQ ID NO: 1634 - RNA, 21 nt, Homo sapiens
ccugugcucccagggccucgc

SEQ ID NO: 1635 - RNA, 22 nt, Homo sapiens
ugaggcccuuggggcacagugg

SEQ ID NO: 1636 - RNA, 22 nt, Homo sapiens
uccuaaaucugaaaguccaaaa

SEQ ID NO: 1637 - RNA, 24 nt, Homo sapiens
uuggacuuuuucagauuuggggau

SEQ ID NO: 1638 - RNA, 22 nt, Homo sapiens
augcaccugggcaaggauucug

SEQ ID NO: 1639 - RNA, 23 nt, Homo sapiens
uaauccuugcuaccugggugaga

SEQ ID NO: 1640 - RNA, 18 nt, Homo sapiens
aauccuugcuaccugggu

SEQ ID NO: 1641 - RNA, 20 nt, Homo sapiens
gcacccaggcaaggauucug

SEQ ID NO: 1642 - RNA, 18 nt, Homo sapiens
aauccuugcuaccugggu

SEQ ID NO: 1643 - RNA, 22 nt, Homo sapiens
aaugcacccgggcaaggauucu

SEQ ID NO: 1644 - RNA, 22 nt, Homo sapiens
aauccuuugucccugggugaga

SEQ ID NO: 1645 - RNA, 22 nt, Homo sapiens
uuuugugucucccauuccccag

SEQ ID NO: 1646 - RNA, 22 nt, Homo sapiens
agggggauggcagagcaaaauu

SEQ ID NO: 1647 - RNA, 22 nt, Homo sapiens
gugcauggcuguauauauaaca

SEQ ID NO: 1648 - RNA, 21 nt, Homo sapiens
uauauauacagccaugcacuc

SEQ ID NO: 1649 - RNA, 22 nt, Homo sapiens
aaugcaccugggcaaggauuca

SEQ ID NO: 1650 - RNA, 21 nt, Homo sapiens
auccuugcuaucugggugcua

SEQ ID NO: 1651 - RNA, 23 nt, Homo sapiens
gggguauuguuuccgcugccagg

SEQ ID NO: 1652 - RNA, 23 nt, Homo sapiens
uagcagcgggaacaguucugcag

SEQ ID NO: 1653 - RNA, 22 nt, Homo sapiens
agacccuggucugcacucuauc

SEQ ID NO: 1654 - RNA, 21 nt, Homo sapiens
gggagugcagggcaggguuuc

SEQ ID NO: 1655 - RNA, 22 nt, Homo sapiens
agacccuggucugcacucuauc

SEQ ID NO: 1656 - RNA, 21 nt, Homo sapiens
uugcagcugcgguuguaaggu

SEQ ID NO: 1657 - RNA, 22 nt, Homo sapiens
cgucaacacuugcugguuuccu

SEQ ID NO: 1658 - RNA, 22 nt, Homo sapiens
gggagccaggaaguauugaugu

SEQ ID NO: 1659 - RNA, 21 nt, Homo sapiens
uaaggcacccuucugaguaga

SEQ ID NO: 1660 - RNA, 22 nt, Homo sapiens
uauucaggaagguguuacuuaa

SEQ ID NO: 1661 - RNA, 21 nt, Homo sapiens
uuuugcaccuuuuggagugaa

SEQ ID NO: 1662 - RNA, 23 nt, Homo sapiens
ugauuguagccuuuuggaguaga

SEQ ID NO: 1663 - RNA, 23 nt, Homo sapiens
uacuccagagggcgucacucaug

SEQ ID NO: 1664 - RNA, 23 nt, Homo sapiens
ggguuuguagcuuugcuggcaug

SEQ ID NO: 1665 - RNA, 23 nt, Homo sapiens
cagggcucagggauuggauggag

SEQ ID NO: 1666 - RNA, 21 nt, Homo sapiens
ucccuucuuccugggcccuca

SEQ ID NO: 1667 - RNA, 24 nt, Homo sapiens
cagggcucagggauuggauggagg

SEQ ID NO: 1668 - RNA, 23 nt, Homo sapiens
augcuacucggaaaucccacuga

SEQ ID NO: 1669 - RNA, 21 nt, Homo sapiens
gugggauuucugaguagcauc

SEQ ID NO: 1670 - RNA, 22 nt, Homo sapiens
uacugcagacguggcaaucaug

SEQ ID NO: 1671 - RNA, 22 nt, Homo sapiens
ugauugguacgucuguggguag

SEQ ID NO: 1672 - RNA, 21 nt, Homo sapiens
uacugcagacaguggcaauca

SEQ ID NO: 1673 - RNA, 23 nt, Homo sapiens
ccggggcagauugguguagggug

SEQ ID NO: 1674 - RNA, 23 nt, Homo sapiens
acggagacgacaagacugugcug

SEQ ID NO: 1675 - RNA, 22 nt, Homo sapiens
aauccacgcugagcuuggcauc

SEQ ID NO: 1676 - RNA, 23 nt, Homo sapiens
aggaaaugaggcuggcuaggagc

SEQ ID NO: 1677 - RNA, 22 nt, Homo sapiens
aaucagugaaugccuugaaccu

SEQ ID NO: 1678 - RNA, 21 nt, Homo sapiens
uuacaggcgugaaccaccgcg

SEQ ID NO: 1679 - RNA, 21 nt, Homo sapiens
guuucaccauguuggucaggc

SEQ ID NO: 1680 - RNA, 22 nt, Homo sapiens
uacucaggagaguggcaaucac

SEQ ID NO: 1681 - RNA, 21 nt, Homo sapiens
auugaaaccucuaagagugga

SEQ ID NO: 1682 - RNA, 22 nt, Homo sapiens
uacucaggagaguggcaaucac

SEQ ID NO: 1683 - RNA, 22 nt, Homo sapiens
uucagaucccagcggugccucu

SEQ ID NO: 1684 - RNA, 21 nt, Homo sapiens
gugucuuuugcucugcaguca

SEQ ID NO: 1685 - RNA, 20 nt, Homo sapiens
aauguguagcaaaagacaga

SEQ ID NO: 1686 - RNA, 21 nt, Homo sapiens
gugucuuuugcucugcaguca

SEQ ID NO: 1687 - RNA, 22 nt, Homo sapiens
aagugcugucauagcugagguc

SEQ ID NO: 1688 - RNA, 23 nt, Homo sapiens
cacucagccuugagggcacuuuc

SEQ ID NO: 1689 - RNA, 23 nt, Homo sapiens
uaaauuucaccuuucugagaagg

SEQ ID NO: 1690 - RNA, 18 nt, Homo sapiens
uucacagggaggugucau

SEQ ID NO: 1691 - RNA, 22 nt, Homo sapiens
uucacaaggaggugucauuuau

SEQ ID NO: 1692 - RNA, 22 nt, Homo sapiens
aaaugucaccuuuuugagagga

SEQ ID NO: 1693 - RNA, 22 nt, Homo sapiens
uucacaaggaggugucauuuau

SEQ ID NO: 1694 - RNA, 23 nt, Homo sapiens
uaaauuucaccuuucugagaaga

SEQ ID NO: 1695 - RNA, 22 nt, Homo sapiens
uucucaaggaggugucguuuau

SEQ ID NO: 1696 - RNA, 21 nt, Homo sapiens
auugacacuucugugaguaga

SEQ ID NO: 1697 - RNA, 23 nt, Homo sapiens
uacucuggagagugacaaucaug

SEQ ID NO: 1698 - RNA, 21 nt, Homo sapiens
auugacaccucugugagugga

SEQ ID NO: 1699 - RNA, 22 nt, Homo sapiens
uucucaagagggaggcaaucau

SEQ ID NO: 1700 - RNA, 22 nt, Homo sapiens
gagugccuucuuuuggagcguu

SEQ ID NO: 1701 - RNA, 24 nt, Homo sapiens
uucuccaaaagaaagcacuuucug

SEQ ID NO: 1702 - RNA, 18 nt, Homo sapiens
ugcuuccuuucagagggu

SEQ ID NO: 1703 - RNA, 23 nt, Homo sapiens
uucucgaggaaagaagcacuuuc

SEQ ID NO: 1704 - RNA, 18 nt, Homo sapiens
ugcuuccuuucagagggu

SEQ ID NO: 1705 - RNA, 22 nt, Homo sapiens
aucuggagguaagaagcacuuu

SEQ ID NO: 1706 - RNA, 22 nt, Homo sapiens
ccucuagauggaagcacugucu

SEQ ID NO: 1707 - RNA, 22 nt, Homo sapiens
aucgugcaucccuuuagagugu

SEQ ID NO: 1708 - RNA, 22 nt, Homo sapiens
aucgugcaucccuuuagagugu

SEQ ID NO: 1709 - RNA, 22 nt, Homo sapiens
aucgugcauccuuuuagagugu

SEQ ID NO: 1710 - RNA, 21 nt, Homo sapiens
agagauugguagaaaucaggu

SEQ ID NO: 1711 - RNA, 21 nt, Homo sapiens
acugaauccucuuuuccucag

SEQ ID NO: 1712 - RNA, 22 nt, Homo sapiens
ugggaugagggauugaagugga

SEQ ID NO: 1713 - RNA, 23 nt, Homo sapiens
aaucggacccauuuaaaccggag

SEQ ID NO: 1714 - RNA, 24 nt, Homo sapiens
ucugggcacaggcggauggacagg

SEQ ID NO: 1715 - RNA, 21 nt, Homo sapiens
ugccaaccgucagagcccaga

SEQ ID NO: 1716 - RNA, 24 nt, Homo sapiens
ucugggcacaggcggauggacagg

SEQ ID NO: 1717 - RNA, 22 nt, Homo sapiens
gaaagcgcuucccuuugcugga

SEQ ID NO: 1718 - RNA, 20 nt, Homo sapiens
cugcaaagggaagcccuuuc

SEQ ID NO: 1719 - RNA, 22 nt, Homo sapiens
caaagcgcuccccuuuagaggu

SEQ ID NO: 1720 - RNA, 23 nt, Homo sapiens
caaagcgcuucucuuuagagugu

SEQ ID NO: 1721 - RNA, 23 nt, Homo sapiens
ucucuggagggaagcacuuucug

SEQ ID NO: 1722 - RNA, 21 nt, Homo sapiens
caaagcgcuucccuuuggagc

SEQ ID NO: 1723 - RNA, 22 nt, Homo sapiens
cucuagagggaagcacuuucug

SEQ ID NO: 1724 - RNA, 21 nt, Homo sapiens
aaagcgcuucccuucagagug

SEQ ID NO: 1725 - RNA, 22 nt, Homo sapiens
cucuagagggaagcgcuuucug

SEQ ID NO: 1726 - RNA, 21 nt, Homo sapiens
gaaagcgcuucucuuuagagg

SEQ ID NO: 1727 - RNA, 22 nt, Homo sapiens
cucuagagggaagcacuuucuc

SEQ ID NO: 1728 - RNA, 21 nt, Homo sapiens
ccagugacugagcuggagcca

SEQ ID NO: 1729 - RNA, 22 nt, Homo sapiens
aggauaggaagaaugaagugcu

SEQ ID NO: 1730 - RNA, 22 nt, Homo sapiens
aggagaguggauuccagguggu

SEQ ID NO: 1731 - RNA, 22 nt, Homo sapiens
uccuccucuaccucaucccagu

SEQ ID NO: 1732 - RNA, 22 nt, Homo sapiens
ugagggguuuggaaugggaugg

SEQ ID NO: 1733 - RNA, 21 nt, Homo sapiens
auccaguucucugagggggcu

SEQ ID NO: 1734 - RNA, 22 nt, Homo sapiens
aaccccuaaggcaacuggaugg

SEQ ID NO: 1735 - RNA, 21 nt, Homo sapiens
ucauccucgucucccucccag

SEQ ID NO: 1736 - RNA, 22 nt, Homo sapiens
agggaaggggacgaggguuggg

SEQ ID NO: 1737 - RNA, 23 nt, Homo sapiens
aagaagagacugagucaucgaau

SEQ ID NO: 1738 - RNA, 23 nt, Homo sapiens
caauggcacaaacucauucuuga

SEQ ID NO: 1739 - RNA, 22 nt, Homo sapiens
aaagugcauccuuuuagagugu

SEQ ID NO: 1740 - RNA, 22 nt, Homo sapiens
cucuagagggaagcgcuuucug

SEQ ID NO: 1741 - RNA, 22 nt, Homo sapiens
aaagugcauccuuuuagagguu

SEQ ID NO: 1742 - RNA, 22 nt, Homo sapiens
cucuagagggaagcgcuuucug

SEQ ID NO: 1743 - RNA, 22 nt, Homo sapiens
aaagugcaucuuuuuagaggau

SEQ ID NO: 1744 - RNA, 22 nt, Homo sapiens
cucuagagggaagcgcuuucug

SEQ ID NO: 1745 - RNA, 22 nt, Homo sapiens
caaagugccucccuuuagagug

SEQ ID NO: 1746 - RNA, 22 nt, Homo sapiens
caaagugccucccuuuagagug

SEQ ID NO: 1747 - RNA, 25 nt, Homo sapiens
ccuccaaagggaagcgcuuucuguu

SEQ ID NO: 1748 - RNA, 22 nt, Homo sapiens
aagugccuccuuuuagaguguu

SEQ ID NO: 1749 - RNA, 22 nt, Homo sapiens
uucuccaaaagggagcacuuuc

SEQ ID NO: 1750 - RNA, 22 nt, Homo sapiens
aaagugcuucccuuuggacugu

SEQ ID NO: 1751 - RNA, 21 nt, Homo sapiens
cuccagagggaaguacuuucu

SEQ ID NO: 1752 - RNA, 21 nt, Homo sapiens
aaagugcuuccuuuuagaggg

SEQ ID NO: 1753 - RNA, 22 nt, Homo sapiens
aaagugcuuccuuuuagagggu

SEQ ID NO: 1754 - RNA, 22 nt, Homo sapiens
cucuagagggaagcacuuucug

SEQ ID NO: 1755 - RNA, 22 nt, Homo sapiens
aaagugcuucucuuuggugggu

SEQ ID NO: 1756 - RNA, 20 nt, Homo sapiens
cuacaaagggaagcccuuuc

SEQ ID NO: 1757 - RNA, 21 nt, Homo sapiens
aaagugcuuccuuuuugaggg

SEQ ID NO: 1758 - RNA, 22 nt, Homo sapiens
aagugcuuccuuuuagaggguu

SEQ ID NO: 1759 - RNA, 22 nt, Homo sapiens
aagugcuuccuuuuagaggguu

SEQ ID NO: 1760 - RNA, 22 nt, Homo sapiens
ccucuaaagggaagcgcuuucu

SEQ ID NO: 1761 - RNA, 24 nt, Homo sapiens
acaaagugcuucccuuuagagugu

SEQ ID NO: 1762 - RNA, 24 nt, Homo sapiens
acaaagugcuucccuuuagagugu

SEQ ID NO: 1763 - RNA, 23 nt, Homo sapiens
ucuagaggaagcacuuucuguuu

SEQ ID NO: 1764 - RNA, 22 nt, Homo sapiens
acaaagugcuucccuuuagagu

SEQ ID NO: 1765 - RNA, 22 nt, Homo sapiens
aacgcacuucccuuuagagugu

SEQ ID NO: 1766 - RNA, 22 nt, Homo sapiens
aaaaugguucccuuuagagugu

SEQ ID NO: 1767 - RNA, 22 nt, Homo sapiens
cucuagagggaagcgcuuucug

SEQ ID NO: 1768 - RNA, 23 nt, Homo sapiens
gaacgcgcuucccuauagagggu

SEQ ID NO: 1769 - RNA, 22 nt, Homo sapiens
cucuagagggaagcgcuuucug

SEQ ID NO: 1770 - RNA, 21 nt, Homo sapiens
gaaggcgcuucccuuuggagu

SEQ ID NO: 1771 - RNA, 22 nt, Homo sapiens
cuacaaagggaagcacuuucuc

SEQ ID NO: 1772 - RNA, 22 nt, Homo sapiens
gaaggcgcuucccuuuagagcg

SEQ ID NO: 1773 - RNA, 21 nt, Homo sapiens
cuccagagggaugcacuuucu

SEQ ID NO: 1774 - RNA, 22 nt, Homo sapiens
cucuagagggaagcacuuucug

SEQ ID NO: 1775 - RNA, 22 nt, Homo sapiens
gaaagugcuuccuuuuagaggc

SEQ ID NO: 1776 - RNA, 23 nt, Homo sapiens
cucuugagggaagcacuuucugu

SEQ ID NO: 1777 - RNA, 20 nt, Homo sapiens
cugcaaagggaagcccuuuc

SEQ ID NO: 1778 - RNA, 22 nt, Homo sapiens
ccucccacacccaaggcuugca

SEQ ID NO: 1779 - RNA, 22 nt, Homo sapiens
caugccuugaguguaggaccgu

SEQ ID NO: 1780 - RNA, 22 nt, Homo sapiens
aucauacaaggacaauuucuuu

SEQ ID NO: 1781 - RNA, 22 nt, Homo sapiens
ggagaaauuauccuuggugugu

SEQ ID NO: 1782 - RNA, 22 nt, Homo sapiens
uggugggcacagaaucuggacu

SEQ ID NO: 1783 - RNA, 25 nt, Homo sapiens
aaaggauucugcugucggucccacu

SEQ ID NO: 1784 - RNA, 22 nt, Homo sapiens
ugugacagauugauaacugaaa

SEQ ID NO: 1785 - RNA, 23 nt, Homo sapiens
ucggggaucaucaugucacgaga

SEQ ID NO: 1786 - RNA, 22 nt, Homo sapiens
aaacauucgcggugcacuucuu

SEQ ID NO: 1787 - RNA, 22 nt, Homo sapiens
auucugcauuuuuagcaaguuc

SEQ ID NO: 1788 - RNA, 22 nt, Homo sapiens
accugagguugugcauuucuaa

SEQ ID NO: 1789 - RNA, 22 nt, Homo sapiens
ucagcaaacauuuauugugugc

SEQ ID NO: 1790 - RNA, 22 nt, Homo sapiens
ucaguaaauguuuauuagauga

SEQ ID NO: 1791 - RNA, 22 nt, Homo sapiens
caaaacuggcaauuacuuuugc

SEQ ID NO: 1792 - RNA, 22 nt, Homo sapiens
aaaaguaauugcgaguuuuacc

SEQ ID NO: 1793 - RNA, 25 nt, Homo sapiens
aaaaaccacaauuacuuuugcacca

SEQ ID NO: 1794 - RNA, 22 nt, Homo sapiens
aaaaguaauuguggauuuugcu

SEQ ID NO: 1795 - RNA, 22 nt, Homo sapiens
caaaaaccggcaauuacuuuug

SEQ ID NO: 1796 - RNA, 22 nt, Homo sapiens
gaaaacgacaaugacuuuugca

SEQ ID NO: 1797 - RNA, 22 nt, Homo sapiens
gaaaacgacaaugacuuuugca

SEQ ID NO: 1798 - RNA, 20 nt, Homo sapiens
aaaaguaauugugguuuuug

SEQ ID NO: 1799 - RNA, 21 nt, Homo sapiens
caaaaacugcaauuacuuuca

SEQ ID NO: 1800 - RNA, 21 nt, Homo sapiens
caaaaacugcaauuacuuuca

SEQ ID NO: 1801 - RNA, 20 nt, Homo sapiens
aaaaguaauugugguuuuug

SEQ ID NO: 1802 - RNA, 21 nt, Homo sapiens
aaagguaauugugguuucugc

SEQ ID NO: 1803 - RNA, 22 nt, Homo sapiens
caaaaacugcaguuacuuuugc

SEQ ID NO: 1804 - RNA, 20 nt, Homo sapiens
aaaagugauugcaguguuug

SEQ ID NO: 1805 - RNA, 22 nt, Homo sapiens
aaagguaauugcaguuuuuccc

SEQ ID NO: 1806 - RNA, 21 nt, Homo sapiens
uaaaaacugcaauuacuuuua

SEQ ID NO: 1807 - RNA, 23 nt, Homo sapiens
ugcaaaaguaauugcaguuuuug

SEQ ID NO: 1808 - RNA, 21 nt, Homo sapiens
aaaaguaacugcgguuuuuga

SEQ ID NO: 1809 - RNA, 22 nt, Homo sapiens
aacggcaaugacuuuuguacca

SEQ ID NO: 1810 - RNA, 22 nt, Homo sapiens
caaaaacugcaguuacuuuugu

SEQ ID NO: 1811 - RNA, 22 nt, Homo sapiens
aaaaguaauugcgguuuuugcc

SEQ ID NO: 1812 - RNA, 20 nt, Homo sapiens
aaaaggcauugugguuuuug

SEQ ID NO: 1813 - RNA, 22 nt, Homo sapiens
aaagaccgugacuacuuuugca

SEQ ID NO: 1814 - RNA, 22 nt, Homo sapiens
agaaguaacuacgguuuuugca

SEQ ID NO: 1815 - RNA, 19 nt, Homo sapiens
aaaaaccacaauuacuuuu

SEQ ID NO: 1816 - RNA, 19 nt, Homo sapiens
aaaaguaauugcggucuuu

SEQ ID NO: 1817 - RNA, 22 nt, Homo sapiens
caaaaacugcaauuacuuuugc

SEQ ID NO: 1818 - RNA, 22 nt, Homo sapiens
gaaaguaauugcuguuuuugcc

SEQ ID NO: 1819 - RNA, 21 nt, Homo sapiens
uaaaacugcaguuauuuuugc

SEQ ID NO: 1820 - RNA, 21 nt, Homo sapiens
aaaaguaauugcaguuuuugc

SEQ ID NO: 1821 - RNA, 22 nt, Homo sapiens
uaaaacccacaauuauguuugu

SEQ ID NO: 1822 - RNA, 22 nt, Homo sapiens
aaaaguaauugcggguuuugcc

SEQ ID NO: 1823 - RNA, 21 nt, Homo sapiens
caaaaccgcaguaacuuuugu

SEQ ID NO: 1824 - RNA, 22 nt, Homo sapiens
aaaaguuauugcgguuuuggcu

SEQ ID NO: 1825 - RNA, 21 nt, Homo sapiens
uggcaguuacuuuugcaccag

SEQ ID NO: 1826 - RNA, 21 nt, Homo sapiens
aaaaguaauugcgguuuuugc

SEQ ID NO: 1827 - RNA, 20 nt, Homo sapiens
aaaacugcaguuacuuuugc

SEQ ID NO: 1828 - RNA, 18 nt, Homo sapiens
aaaaguacuugcggauuu

SEQ ID NO: 1829 - RNA, 20 nt, Homo sapiens
gugcaaaagucaucacgguu

SEQ ID NO: 1830 - RNA, 22 nt, Homo sapiens
agaaguaauugcgguuuugcca

SEQ ID NO: 1831 - RNA, 22 nt, Homo sapiens
caaaaccgcgauuacucuugca

SEQ ID NO: 1832 - RNA, 21 nt, Homo sapiens
aaaaguaauugugguuuuugc

SEQ ID NO: 1833 - RNA, 21 nt, Homo sapiens
aaaaacugcaaucacuuuugc

SEQ ID NO: 1834 - RNA, 22 nt, Homo sapiens
caaaagugauugugguuuuugc

SEQ ID NO: 1835 - RNA, 22 nt, Homo sapiens
caagaaccucaguugcuuuugu

SEQ ID NO: 1836 - RNA, 22 nt, Homo sapiens
aaaaguaauugugguuuuggcc

SEQ ID NO: 1837 - RNA, 22 nt, Homo sapiens
aaagguaacugugauuuuugcu

SEQ ID NO: 1838 - RNA, 22 nt, Homo sapiens
caaaaaccauaguuacuuuugc

SEQ ID NO: 1839 - RNA, 22 nt, Homo sapiens
aaaaguaacuaugguuuuugcc

SEQ ID NO: 1840 - RNA, 22 nt, Homo sapiens
caaaaaucucaauuacuuuugc

SEQ ID NO: 1841 - RNA, 22 nt, Homo sapiens
aaaaguaauugcgguuuuugcc

SEQ ID NO: 1842 - RNA, 22 nt, Homo sapiens
caaaaaccacaguuucuuuugc

SEQ ID NO: 1843 - RNA, 22 nt, Homo sapiens
aaaaguaauugugguuuuugcc

SEQ ID NO: 1844 - RNA, 22 nt, Homo sapiens
aaaaacugagacuacuuuugca

SEQ ID NO: 1845 - RNA, 22 nt, Homo sapiens
aaaaacugagacuacuuuugca

SEQ ID NO: 1846 - RNA, 22 nt, Homo sapiens
caaaagcaaucgcgguuuuugc

SEQ ID NO: 1847 - RNA, 19 nt, Homo sapiens
aaaaacuguaauuacuuuu

SEQ ID NO: 1848 - RNA, 19 nt, Homo sapiens
aaaaacuguaauuacuuuu

SEQ ID NO: 1849 - RNA, 22 nt, Homo sapiens
ugcaaaaguaaucacaguuuuu

SEQ ID NO: 1850 - RNA, 22 nt, Homo sapiens
aaaacuguaauuacuuuuguac

SEQ ID NO: 1851 - RNA, 23 nt, Homo sapiens
ugcaaaaguaauugcaguuuuug

SEQ ID NO: 1852 - RNA, 23 nt, Homo sapiens
caaaaaccgcaauuacuuuugca

SEQ ID NO: 1853 - RNA, 22 nt, Homo sapiens
aaaaguaaucgcgguuuuuguc

SEQ ID NO: 1854 - RNA, 22 nt, Homo sapiens
aaaaguaauugcggauuuugcc

SEQ ID NO: 1855 - RNA, 22 nt, Homo sapiens
aaaaguaauugcggucuuuggu

SEQ ID NO: 1856 - RNA, 21 nt, Homo sapiens
caaaaacugcauuacuuuugc

SEQ ID NO: 1857 - RNA, 22 nt, Homo sapiens
aaaaguaauugcggucuuuggu

SEQ ID NO: 1858 - RNA, 22 nt, Homo sapiens
aaaaguacuugcggauuuugcu

SEQ ID NO: 1859 - RNA, 22 nt, Homo sapiens
aaaaguauuugcggguuuuguc

SEQ ID NO: 1860 - RNA, 21 nt, Homo sapiens
caaagguauuugugguuuuug

SEQ ID NO: 1861 - RNA, 22 nt, Homo sapiens
caaaaguaauuguggauuuugu

SEQ ID NO: 1862 - RNA, 22 nt, Homo sapiens
ccaaaacugcaguuacuuuugc

SEQ ID NO: 1863 - RNA, 22 nt, Homo sapiens
aaaaguaauugcgguuuuugcc

SEQ ID NO: 1864 - RNA, 22 nt, Homo sapiens
uagcaaaaacugcaguuacuuu

SEQ ID NO: 1865 - RNA, 22 nt, Homo sapiens
gcuggugcaaaaguaauggcgg

SEQ ID NO: 1866 - RNA, 23 nt, Homo sapiens
auggccaaaacugcaguuauuuu

SEQ ID NO: 1867 - RNA, 25 nt, Homo sapiens
aaaaaccacaauuacuuuugcacca

SEQ ID NO: 1868 - RNA, 21 nt, Homo sapiens
caaaagugaucgugguuuuug

SEQ ID NO: 1869 - RNA, 23 nt, Homo sapiens
caaagacugcaauuacuuuugcg

SEQ ID NO: 1870 - RNA, 22 nt, Homo sapiens
agcuacaguuacuuuugcacca

SEQ ID NO: 1871 - RNA, 23 nt, Homo sapiens
aaaaguaacugcgguuuuugccu

SEQ ID NO: 1872 - RNA, 20 nt, Homo sapiens
uaaaaacugcaauuacuuuc

SEQ ID NO: 1873 - RNA, 23 nt, Homo sapiens
ugcaaaaguaauugcaguuuuug

SEQ ID NO: 1874 - RNA, 22 nt, Homo sapiens
aaaaguaaucacuguuuuugcc

SEQ ID NO: 1875 - RNA, 23 nt, Homo sapiens
caaaaaccgcaauuacuuuugca

SEQ ID NO: 1876 - RNA, 21 nt, Homo sapiens
ugacaacuauggaugagcucu

SEQ ID NO: 1877 - RNA, 20 nt, Homo sapiens
agugccugagggaguaagag

SEQ ID NO: 1878 - RNA, 22 nt, Homo sapiens
ugucuuacucccucaggcacau

SEQ ID NO: 1879 - RNA, 23 nt, Homo sapiens
agugccugagggaguaagagccc

SEQ ID NO: 1880 - RNA, 22 nt, Homo sapiens
augugccugagggaguaagaca

SEQ ID NO: 1881 - RNA, 20 nt, Homo sapiens
ucuuacucccucaggcacug

SEQ ID NO: 1882 - RNA, 21 nt, Homo sapiens
gcgacccacucuugguuucca

SEQ ID NO: 1883 - RNA, 21 nt, Homo sapiens
gcgacccauacuugguuucag

SEQ ID NO: 1884 - RNA, 22 nt, Homo sapiens
gaaaucaagcgugggugagacc

SEQ ID NO: 1885 - RNA, 21 nt, Homo sapiens
aacaggugacugguuagacaa

SEQ ID NO: 1886 - RNA, 21 nt, Homo sapiens
aacaggugacugguuagacaa

SEQ ID NO: 1887 - RNA, 21 nt, Homo sapiens
guuuaaccuuuugccuguugg

SEQ ID NO: 1888 - RNA, 21 nt, Homo sapiens
aaaacggugagauuuuguuuu

SEQ ID NO: 1889 - RNA, 21 nt, Homo sapiens
gcuaguccugacucagccagu

SEQ ID NO: 1890 - RNA, 21 nt, Homo sapiens
aggguaagcugaaccucugau

SEQ ID NO: 1891 - RNA, 22 nt, Homo sapiens
auauuaccauuagcucaucuuu

SEQ ID NO: 1892 - RNA, 22 nt, Homo sapiens
gaugagcucauuguaauaugag

SEQ ID NO: 1893 - RNA, 23 nt, Homo sapiens
guuugcacgggugggccuugucu

SEQ ID NO: 1894 - RNA, 19 nt, Homo sapiens
guccuaggaggcuccucug

SEQ ID NO: 1895 - RNA, 21 nt, Homo sapiens
caauucucaaaggagccuccc

SEQ ID NO: 1896 - RNA, 21 nt, Homo sapiens
guuggggugcaggggucugcu

SEQ ID NO: 1897 - RNA, 22 nt, Homo sapiens
uuagcuuaaggaguaccagauc

SEQ ID NO: 1898 - RNA, 21 nt, Homo sapiens
uaugguacuccuuaagcuaac

SEQ ID NO: 1899 - RNA, 19 nt, Homo sapiens
ugagcugcuguaccaaaau

SEQ ID NO: 1900 - RNA, 22 nt, Homo sapiens
cacauaugaagugagccagcac

SEQ ID NO: 1901 - RNA, 22 nt, Homo sapiens
ugcuggcucauuucauaugugu

SEQ ID NO: 1902 - RNA, 22 nt, Homo sapiens
uuccaugccuccuagaaguucc

SEQ ID NO: 1903 - RNA, 22 nt, Homo sapiens
agccuuccaggagaaauggaga

SEQ ID NO: 1904 - RNA, 22 nt, Homo sapiens
uaaaacuuuaagugugccuagg

SEQ ID NO: 1905 - RNA, 22 nt, Homo sapiens
uaggcacacuuaaaguuauagc

SEQ ID NO: 1906 - RNA, 22 nt, Homo sapiens
gaauauggguauauuaguuugg

SEQ ID NO: 1907 - RNA, 22 nt, Homo sapiens
aaacuaauauacccauauucug

SEQ ID NO: 1908 - RNA, 22 nt, Homo sapiens
uaguucuucccuuugcccaauu

SEQ ID NO: 1909 - RNA, 22 nt, Homo sapiens
cagggaaaugggaagaacuaga

SEQ ID NO: 1910 - RNA, 22 nt, Homo sapiens
cugaauagcugggacuacaggu

SEQ ID NO: 1911 - RNA, 22 nt, Homo sapiens
ugaaguaccagcuacucgagag

SEQ ID NO: 1912 - RNA, 22 nt, Homo sapiens
cagagugacaagcugguuaaag

SEQ ID NO: 1913 - RNA, 22 nt, Homo sapiens
uauccagcuuguuacuauaugc

SEQ ID NO: 1914 - RNA, 21 nt, Homo sapiens
gccccgggcagugugaucauc

SEQ ID NO: 1915 - RNA, 18 nt, Homo sapiens
auggucaccuccgggacu

SEQ ID NO: 1916 - RNA, 19 nt, Homo sapiens
aagucccacuaaugccagc

SEQ ID NO: 1917 - RNA, 21 nt, Homo sapiens
acuggcauuagugggacuuuu

SEQ ID NO: 1918 - RNA, 23 nt, Homo sapiens
ugcacauggcaaccuagcuccca

SEQ ID NO: 1919 - RNA, 21 nt, Homo sapiens
ggcugggugcucuugugcagu

SEQ ID NO: 1920 - RNA, 21 nt, Homo sapiens
uaaaguaaauaugcaccaaaa

SEQ ID NO: 1921 - RNA, 21 nt, Homo sapiens
aauaaaguucauguauggcaa

SEQ ID NO: 1922 - RNA, 21 nt, Homo sapiens
uugccauacauagacuuuauu

SEQ ID NO: 1923 - RNA, 21 nt, Homo sapiens
auacccauagcuuagcuccca

SEQ ID NO: 1924 - RNA, 21 nt, Homo sapiens
ugggagcuaagcuauggguau

SEQ ID NO: 1925 - RNA, 22 nt, Homo sapiens
caaaguuuaagauccuugaagu

SEQ ID NO: 1926 - RNA, 22 nt, Homo sapiens
aucaaggaucuuaaacuuugcc

SEQ ID NO: 1927 - RNA, 20 nt, Homo sapiens
aaaguagcuguaccauuugc

SEQ ID NO: 1928 - RNA, 19 nt, Homo sapiens
agguugacauacguuuccc

SEQ ID NO: 1929 - RNA, 19 nt, Homo sapiens
aggcacggugucagcaggc

SEQ ID NO: 1930 - RNA, 19 nt, Homo sapiens
gggcgccugugaucccaac

SEQ ID NO: 1931 - RNA, 23 nt, Homo sapiens
aguauguucuuccaggacagaac

SEQ ID NO: 1932 - RNA, 20 nt, Homo sapiens
auguauaaauguauacacac

SEQ ID NO: 1933 - RNA, 22 nt, Homo sapiens
gagaaaugcuggacuaaucugc

SEQ ID NO: 1934 - RNA, 22 nt, Homo sapiens
agaaaggguggcaauaccucuu

SEQ ID NO: 1935 - RNA, 22 nt, Homo sapiens
agguauugccacccuuucuagu

SEQ ID NO: 1936 - RNA, 22 nt, Homo sapiens
guagcaccuugcaggauaaggu

SEQ ID NO: 1937 - RNA, 24 nt, Homo sapiens
uacagaugcagauucucugacuuc

SEQ ID NO: 1938 - RNA, 20 nt, Homo sapiens
aacucuagccugagcaacag

SEQ ID NO: 1939 - RNA, 22 nt, Homo sapiens
acagcccagcaguuaucacggg

SEQ ID NO: 1940 - RNA, 21 nt, Homo sapiens
uaucguaucguauuguauugu

SEQ ID NO: 1941 - RNA, 22 nt, Homo sapiens
uuagaacguuuuagggucaaau

SEQ ID NO: 1942 - RNA, 22 nt, Homo sapiens
uaacaaacaccuguaaaacagc

SEQ ID NO: 1943 - RNA, 22 nt, Homo sapiens
agcauacaccuguaguccuaga

SEQ ID NO: 1944 - RNA, 21 nt, Homo sapiens
aguuaaugaauccuggaaagu

SEQ ID NO: 1945 - RNA, 21 nt, Homo sapiens
ucagcuacuaccucuauuagg

SEQ ID NO: 1946 - RNA, 22 nt, Homo sapiens
uugcucugagcuccgagaaagc

SEQ ID NO: 1947 - RNA, 22 nt, Homo sapiens
caaauaauaccacagugggugu

SEQ ID NO: 1948 - RNA, 20 nt, Homo sapiens
aauaauaucacaguaggugu

SEQ ID NO: 1949 - RNA, 22 nt, Homo sapiens
aauaauaucacaguagguguac

SEQ ID NO: 1950 - RNA, 22 nt, Homo sapiens
gcaguggcucugaaaugaacuc

SEQ ID NO: 1951 - RNA, 21 nt, Homo sapiens
cagaucaugggacugucucag

SEQ ID NO: 1952 - RNA, 22 nt, Homo sapiens
acuccaagaagaaucuagacag

SEQ ID NO: 1953 - RNA, 22 nt, Homo sapiens
cucauuuaaguagucugaugcc

SEQ ID NO: 1954 - RNA, 22 nt, Homo sapiens
ucaaguaguuucaugauaaagg

SEQ ID NO: 1955 - RNA, 22 nt, Homo sapiens
ugggggagugcagugauugugg

SEQ ID NO: 1956 - RNA, 22 nt, Homo sapiens
uccugucuuuccuuguuggagc

SEQ ID NO: 1957 - RNA, 22 nt, Homo sapiens
uccugucuuuccuuguuggagc

SEQ ID NO: 1958 - RNA, 22 nt, Homo sapiens
ugccccaacaaggaaggacaag

SEQ ID NO: 1959 - RNA, 22 nt, Homo sapiens
cgaaaacagcaauuaccuuugc

SEQ ID NO: 1960 - RNA, 22 nt, Homo sapiens
aaagguaauugcaguuuuuccc

SEQ ID NO: 1961 - RNA, 22 nt, Homo sapiens
uaaugcauuaaauuauugaagg

SEQ ID NO: 1962 - RNA, 19 nt, Homo sapiens
uuauugucacguucugauu

SEQ ID NO: 1963 - RNA, 22 nt, Homo sapiens
ugagucagcaacauaucccaug

SEQ ID NO: 1964 - RNA, 18 nt, Homo sapiens
aggagaagucgggaaggu

SEQ ID NO: 1965 - RNA, 22 nt, Homo sapiens
uuaggccaucaucccauuaugc

SEQ ID NO: 1966 - RNA, 23 nt, Homo sapiens
uguuucggggcucauggccugug

SEQ ID NO: 1967 - RNA, 22 nt, Homo sapiens
uucuggauaacaugcugaagcu

SEQ ID NO: 1968 - RNA, 22 nt, Homo sapiens
acguuugaaugcuguacaaggc

SEQ ID NO: 1969 - RNA, 20 nt, Homo sapiens
augagcgacugugccugacc

SEQ ID NO: 1970 - RNA, 21 nt, Homo sapiens
ugaguuggccaucugagugag

SEQ ID NO: 1971 - RNA, 20 nt, Homo sapiens
guccgcucggcgguggccca

SEQ ID NO: 1972 - RNA, 24 nt, Homo sapiens
cugaagugauguguaacugaucag

SEQ ID NO: 1973 - RNA, 20 nt, Homo sapiens
gcggagagagaauggggagc

SEQ ID NO: 1974 - RNA, 22 nt, Homo sapiens
cacgcucaugcacacacccaca

SEQ ID NO: 1975 - RNA, 23 nt, Homo sapiens
ugagugugugugugugagugugu

SEQ ID NO: 1976 - RNA, 19 nt, Homo sapiens
gagccaguuggacaggagc

SEQ ID NO: 1977 - RNA, 22 nt, Homo sapiens
aagauguggaaaaauuggaauc

SEQ ID NO: 1978 - RNA, 22 nt, Homo sapiens
auucuaauuucuccacgucuuu

SEQ ID NO: 1979 - RNA, 21 nt, Homo sapiens
uagauaaaauauugguaccug

SEQ ID NO: 1980 - RNA, 21 nt, Homo sapiens
cuucuugugcucuaggauugu

SEQ ID NO: 1981 - RNA, 20 nt, Homo sapiens
gggcuggggcgcggggaggu

SEQ ID NO: 1982 - RNA, 23 nt, Homo sapiens
uucauuugguauaaaccgcgauu

SEQ ID NO: 1983 - RNA, 23 nt, Homo sapiens
uucauuugguauaaaccgcgauu

SEQ ID NO: 1984 - RNA, 22 nt, Homo sapiens
ucgcgguuugugccagaugacg

SEQ ID NO: 1985 - RNA, 22 nt, Homo sapiens
uugagaaugaugaaucauuagg

SEQ ID NO: 1986 - RNA, 22 nt, Homo sapiens
uugagaaugaugaaucauuagg

SEQ ID NO: 1987 - RNA, 22 nt, Homo sapiens
uaaugauucaucagacucagau

SEQ ID NO: 1988 - RNA, 21 nt, Homo sapiens
ucuuguguucucuagaucagu

SEQ ID NO: 1989 - RNA, 22 nt, Homo sapiens
uaacugguugaacaacugaacc

SEQ ID NO: 1990 - RNA, 23 nt, Homo sapiens
uuacaguuguucaaccaguuacu

SEQ ID NO: 1991 - RNA, 21 nt, Homo sapiens
caaagaggaaggucccauuac

SEQ ID NO: 1992 - RNA, 22 nt, Homo sapiens
ucaguuccaggccaaccaggcu

SEQ ID NO: 1993 - RNA, 22 nt, Homo sapiens
uuaugguuugccugggacugag

SEQ ID NO: 1994 - RNA, 19 nt, Homo sapiens
ugggcguaucuguaugcua

SEQ ID NO: 1995 - RNA, 19 nt, Homo sapiens
ugggcguaucuguaugcua

SEQ ID NO: 1996 - RNA, 22 nt, Homo sapiens
cuagcacacagauacgcccaga

SEQ ID NO: 1997 - RNA, 22 nt, Homo sapiens
uaugcauuguauuuuuaggucc

SEQ ID NO: 1998 - RNA, 21 nt, Homo sapiens
uuuccauaggugaugagucac

SEQ ID NO: 1999 - RNA, 21 nt, Homo sapiens
uuggccacaauggguuagaac

SEQ ID NO: 2000 - RNA, 24 nt, Homo sapiens
ucagaacaaaugccgguucccaga

SEQ ID NO: 2001 - RNA, 22 nt, Homo sapiens
ugagaaccacgucugcucugag

SEQ ID NO: 2002 - RNA, 21 nt, Homo sapiens
uaauuuuauguauaagcuagu

SEQ ID NO: 2003 - RNA, 22 nt, Homo sapiens
gagcuuauucauaaaagugcag

SEQ ID NO: 2004 - RNA, 20 nt, Homo sapiens
agaccauggguucucauugu

SEQ ID NO: 2005 - RNA, 22 nt, Homo sapiens
uugugucaauaugcgaugaugu

SEQ ID NO: 2006 - RNA, 19 nt, Homo sapiens
ugucucugcugggguuucu

SEQ ID NO: 2007 - RNA, 25 nt, Homo sapiens
aggcaccagccaggcauugcucagc

SEQ ID NO: 2008 - RNA, 21 nt, Homo sapiens
gaagugugccguggugugucu

SEQ ID NO: 2009 - RNA, 21 nt, Homo sapiens
aagccugcccggcuccucggg

SEQ ID NO: 2010 - RNA, 22 nt, Homo sapiens
ugugucacucgaugaccacugu

SEQ ID NO: 2011 - RNA, 23 nt, Homo sapiens
ugguucucuuguggcucaagcgu

SEQ ID NO: 2012 - RNA, 22 nt, Homo sapiens
ugugucacucgaugaccacugu

SEQ ID NO: 2013 - RNA, 22 nt, Homo sapiens
uacgucaucguugucaucguca

SEQ ID NO: 2014 - RNA, 22 nt, Homo sapiens
uacgucaucguugucaucguca

SEQ ID NO: 2015 - RNA, 23 nt, Homo sapiens
gcggugaucccgauggugugagc

SEQ ID NO: 2016 - RNA, 20 nt, Homo sapiens
guugugucaguuuaucaaac

SEQ ID NO: 2017 - RNA, 23 nt, Homo sapiens
acuuacagacaagagccuugcuc

SEQ ID NO: 2018 - RNA, 22 nt, Homo sapiens
uggucuaggauuguuggaggag

SEQ ID NO: 2019 - RNA, 23 nt, Homo sapiens
gacacgggcgacagcugcggccc

SEQ ID NO: 2020 - RNA, 22 nt, Homo sapiens
cacacacugcaauuacuuuugc

SEQ ID NO: 2021 - RNA, 19 nt, Homo sapiens
aggcugcggaauucaggac

SEQ ID NO: 2022 - RNA, 23 nt, Homo sapiens
uaaaucccauggugccuucuccu

SEQ ID NO: 2023 - RNA, 22 nt, Homo sapiens
agaaggcacuaugagauuuaga

SEQ ID NO: 2024 - RNA, 23 nt, Homo sapiens
uaaaucccauggugccuucuccu

SEQ ID NO: 2025 - RNA, 21 nt, Homo sapiens
aaacuacugaaaaucaaagau

SEQ ID NO: 2026 - RNA, 21 nt, Homo sapiens
ccugcgagucuccggcggugg

SEQ ID NO: 2027 - RNA, 21 nt, Homo sapiens
gggcuagggccugcugccccc

SEQ ID NO: 2028 - RNA, 21 nt, Homo sapiens
guucaaauccagaucuauaac

SEQ ID NO: 2029 - RNA, 19 nt, Homo sapiens
ccgguuccagucccuggag

SEQ ID NO: 2030 - RNA, 19 nt, Homo sapiens
uucugcugccggccaaggc

SEQ ID NO: 2031 - RNA, 22 nt, Homo sapiens
uccucaucacacugcaccuuag

SEQ ID NO: 2032 - RNA, 20 nt, Homo sapiens
gguagugaguuaucagcuac

SEQ ID NO: 2033 - RNA, 20 nt, Homo sapiens
gauauucagaggcuaggugg

SEQ ID NO: 2034 - RNA, 21 nt, Homo sapiens
acggcccaggcggcauuggug

SEQ ID NO: 2035 - RNA, 21 nt, Homo sapiens
agcaugacagaggagaggugg

SEQ ID NO: 2036 - RNA, 21 nt, Homo sapiens
gggaagagcuguacggccuuc

SEQ ID NO: 2037 - RNA, 19 nt, Homo sapiens
ccgccugagcuagcugugg

SEQ ID NO: 2038 - RNA, 24 nt, Homo sapiens
uuggaagcuuggaccaacuagcug

SEQ ID NO: 2039 - RNA, 25 nt, Homo sapiens
aggggugguguugggacagcuccgu

SEQ ID NO: 2040 - RNA, 19 nt, Homo sapiens
ucuagugcgggcguucccg

SEQ ID NO: 2041 - RNA, 24 nt, Homo sapiens
aggagcagugccggccaaggcgcc

SEQ ID NO: 2042 - RNA, 19 nt, Homo sapiens
gaauacgucugguugaucc

SEQ ID NO: 2043 - RNA, 20 nt, Homo sapiens
cuuauaucagaggcuguggg

SEQ ID NO: 2044 - RNA, 20 nt, Homo sapiens
uuccgccagucgguggccgg

SEQ ID NO: 2045 - RNA, 19 nt, Homo sapiens
aaggggcugggggagcaca

SEQ ID NO: 2046 - RNA, 20 nt, Homo sapiens
ggagguugggaagggcagag

SEQ ID NO: 2047 - RNA, 18 nt, Homo sapiens
ugaggcgggggggcgagc

SEQ ID NO: 2048 - RNA, 20 nt, Homo sapiens
agagaugaagcgggggggcg

SEQ ID NO: 2049 - RNA, 24 nt, Homo sapiens
ggaggccgggguggggcggggcgg

SEQ ID NO: 2050 - RNA, 20 nt, Homo sapiens
aggguguuucucucaucucu

SEQ ID NO: 2051 - RNA, 19 nt, Homo sapiens
ggggagcgaggggcggggc

SEQ ID NO: 2052 - RNA, 21 nt, Homo sapiens
ugagcuaaaugugugcuggga

SEQ ID NO: 2053 - RNA, 23 nt, Homo sapiens
gcgaggaccccucggggucugac

SEQ ID NO: 2054 - RNA, 25 nt, Homo sapiens
gcugggcagggcuucugagcuccuu

SEQ ID NO: 2055 - RNA, 20 nt, Homo sapiens
gggaaaaggaagggggagga

SEQ ID NO: 2056 - RNA, 20 nt, Homo sapiens
gcggaaggcggagcggcgga

SEQ ID NO: 2057 - RNA, 18 nt, Homo sapiens
gugaaggcccggcggaga

SEQ ID NO: 2058 - RNA, 19 nt, Homo sapiens
ugagggagugggugggagg

SEQ ID NO: 2059 - RNA, 19 nt, Homo sapiens
acuggaauuggagucaaaa

SEQ ID NO: 2060 - RNA, 19 nt, Homo sapiens
ugagggaguuggguguaua

SEQ ID NO: 2061 - RNA, 20 nt, Homo sapiens
aggaauguuccuucuuugcc

SEQ ID NO: 2062 - RNA, 19 nt, Homo sapiens
ugagggaguggauuguaug

SEQ ID NO: 2063 - RNA, 19 nt, Homo sapiens
ggcuggucagaugggagug

SEQ ID NO: 2064 - RNA, 19 nt, Homo sapiens
agcagggcuggggauugca

SEQ ID NO: 2065 - RNA, 19 nt, Homo sapiens
ugagggaggagguugggua

SEQ ID NO: 2066 - RNA, 19 nt, Homo sapiens
ugaggugguaggauguaga

SEQ ID NO: 2067 - RNA, 23 nt, Homo sapiens
gaacgccuguucuugccaggugg

SEQ ID NO: 2068 - RNA, 22 nt, Homo sapiens
uccgagccugggucucccucuu

SEQ ID NO: 2069 - RNA, 22 nt, Homo sapiens
ggggguccccggugcucggauc

SEQ ID NO: 2070 - RNA, 22 nt, Homo sapiens
agucauuggaggguuugagcag

SEQ ID NO: 2071 - RNA, 22 nt, Homo sapiens
acucaaaacccuucagugacuu

SEQ ID NO: 2072 - RNA, 19 nt, Homo sapiens
cagcaggaggugaggggag

SEQ ID NO: 2073 - RNA, 22 nt, Homo sapiens
agacuucccauuugaagguggc

SEQ ID NO: 2074 - RNA, 23 nt, Homo sapiens
aaacucuacuuguccuucugagu

SEQ ID NO: 2075 - RNA, 24 nt, Homo sapiens
gaccuggacauguuugugcccagu

SEQ ID NO: 2076 - RNA, 24 nt, Homo sapiens
gaccuggacauguuugugcccagu

SEQ ID NO: 2077 - RNA, 22 nt, Homo sapiens
gcugggauuacaggcaugagcc

SEQ ID NO: 2078 - RNA, 20 nt, Homo sapiens
auggagauagauauagaaau

SEQ ID NO: 2079 - RNA, 21 nt, Homo sapiens
ggcuagcaacagcgcuuaccu

SEQ ID NO: 2080 - RNA, 21 nt, Homo sapiens
acagucugcugagguuggagc

SEQ ID NO: 2081 - RNA, 23 nt, Homo sapiens
aucccuugcaggggcuguugggu

SEQ ID NO: 2082 - RNA, 21 nt, Homo sapiens
cacaagguauugguauuaccu

SEQ ID NO: 2083 - RNA, 22 nt, Homo sapiens
uaguaccaguaccuuguguuca

SEQ ID NO: 2084 - RNA, 22 nt, Homo sapiens
gacuauagaacuuucccccuca

SEQ ID NO: 2085 - RNA, 21 nt, Homo sapiens
agggggaaaguucuauagucc

SEQ ID NO: 2086 - RNA, 19 nt, Homo sapiens
agcugucugaaaaugucuu

SEQ ID NO: 2087 - RNA, 22 nt, Homo sapiens
gugagucucuaagaaaagagga

SEQ ID NO: 2088 - RNA, 20 nt, Homo sapiens
ucuuuucuuugagacucacu

SEQ ID NO: 2089 - RNA, 22 nt, Homo sapiens
gugagucucuaagaaaagagga

SEQ ID NO: 2090 - RNA, 21 nt, Homo sapiens
ucuaguaagaguggcagucga

SEQ ID NO: 2091 - RNA, 22 nt, Homo sapiens
augcugacauauuuacuagagg

SEQ ID NO: 2092 - RNA, 22 nt, Homo sapiens
guucucccaacguaagcccagc

SEQ ID NO: 2093 - RNA, 21 nt, Homo sapiens
uggguuuacguugggagaacu

SEQ ID NO: 2094 - RNA, 22 nt, Homo sapiens
aguauucuguaccagggaaggu

SEQ ID NO: 2095 - RNA, 21 nt, Homo sapiens
agaccuggcccagaccucagc

SEQ ID NO: 2096 - RNA, 19 nt, Homo sapiens
gugucugcuuccuguggga

SEQ ID NO: 2097 - RNA, 23 nt, Homo sapiens
cuaauaguaucuaccacaauaaa

SEQ ID NO: 2098 - RNA, 22 nt, Homo sapiens
aaccagcaccccaacuuuggac

SEQ ID NO: 2099 - RNA, 23 nt, Homo sapiens
acuugggcacugaaacaaugucc

SEQ ID NO: 2100 - RNA, 23 nt, Homo sapiens
ugugcuugcucgucccgcccgca

SEQ ID NO: 2101 - RNA, 24 nt, Homo sapiens
acugggggcuuucgggcucugcgu

SEQ ID NO: 2102 - RNA, 25 nt, Homo sapiens
agggaucgcgggcggguggcggccu

SEQ ID NO: 2103 - RNA, 23 nt, Homo sapiens
aucgcugcgguugcgagcgcugu

SEQ ID NO: 2104 - RNA, 21 nt, Homo sapiens
augauccaggaaccugccucu

SEQ ID NO: 2105 - RNA, 24 nt, Homo sapiens
aaagacauaggauagagucaccuc

SEQ ID NO: 2106 - RNA, 22 nt, Homo sapiens
agacacauuuggagagggaacc

SEQ ID NO: 2107 - RNA, 22 nt, Homo sapiens
gucccucuccaaaugugucuug

SEQ ID NO: 2108 - RNA, 22 nt, Homo sapiens
agacacauuuggagagggaccc

SEQ ID NO: 2109 - RNA, 22 nt, Homo sapiens
gguucccucuccaaaugugucu

SEQ ID NO: 2110 - RNA, 22 nt, Homo sapiens
acuuguaugcuagcucagguag

SEQ ID NO: 2111 - RNA, 19 nt, Homo sapiens
aguguggcuuucuuagagc

SEQ ID NO: 2112 - RNA, 19 nt, Homo sapiens
ucuaggcugguacugcuga

SEQ ID NO: 2113 - RNA, 19 nt, Homo sapiens
aagcagcugccucugaggc

SEQ ID NO: 2114 - RNA, 21 nt, Homo sapiens
guggcugcacucacuuccuuc

SEQ ID NO: 2115 - RNA, 19 nt, Homo sapiens
aagugugcagggcacuggu

SEQ ID NO: 2116 - RNA, 22 nt, Homo sapiens
aaaccuguguuguucaagaguc

SEQ ID NO: 2117 - RNA, 22 nt, Homo sapiens
agcaguguuuguuuugcccaca

SEQ ID NO: 2118 - RNA, 22 nt, Homo sapiens
ucgggcgcaagagcacugcagu

SEQ ID NO: 2119 - RNA, 21 nt, Homo sapiens
aggaggcagcgcucucaggac

SEQ ID NO: 2120 - RNA, 21 nt, Homo sapiens
acacuuguugggaugaccugc

SEQ ID NO: 2121 - RNA, 24 nt, Homo sapiens
aggagcuauccacuccaggugucc

SEQ ID NO: 2122 - RNA, 23 nt, Homo sapiens
ccagagcagccugcgguaacagu

SEQ ID NO: 2123 - RNA, 22 nt, Homo sapiens
aguugccagggcugccuuuggu

SEQ ID NO: 2124 - RNA, 21 nt, Homo sapiens
uagaccaucuuucuagaguau

SEQ ID NO: 2125 - RNA, 22 nt, Homo sapiens
agcucuagaaagauuguugacc

SEQ ID NO: 2126 - RNA, 21 nt, Homo sapiens
gggacuaggaugcagaccucc

SEQ ID NO: 2127 - RNA, 23 nt, Homo sapiens
aggucugcauucaaauccccaga

SEQ ID NO: 2128 - RNA, 20 nt, Homo sapiens
cauuacagcacagccauucu

SEQ ID NO: 2129 - RNA, 21 nt, Homo sapiens
ucuggcugugcuguaaugcag

SEQ ID NO: 2130 - RNA, 21 nt, Homo sapiens
ugacuucuaccucuuccaaag

SEQ ID NO: 2131 - RNA, 22 nt, Homo sapiens
uuggaauaggggauaucucagc

SEQ ID NO: 2132 - RNA, 22 nt, Homo sapiens
ucguaucagagauuccagacac

SEQ ID NO: 2133 - RNA, 23 nt, Homo sapiens
acugggaugucacugaauauggu

SEQ ID NO: 2134 - RNA, 22 nt, Homo sapiens
caaaguccuuccuauuuuuccc

SEQ ID NO: 2135 - RNA, 21 nt, Homo sapiens
gaagaauaggagggacuuugu

SEQ ID NO: 2136 - RNA, 22 nt, Homo sapiens
ugggccaugcauuucuagaacu

SEQ ID NO: 2137 - RNA, 21 nt, Homo sapiens
ucuagaaaugcaugacccacc

SEQ ID NO: 2138 - RNA, 22 nt, Homo sapiens
uuccacugccacuaccuaauuu

SEQ ID NO: 2139 - RNA, 21 nt, Homo sapiens
auuagguaguggcaguggaac

SEQ ID NO: 2140 - RNA, 22 nt, Homo sapiens
uuuaggauaagcuugacuuuug

SEQ ID NO: 2141 - RNA, 22 nt, Homo sapiens
aaaggaaaguguauccuaaaag

SEQ ID NO: 2142 - RNA, 22 nt, Homo sapiens
uuuaggauaagcuugacuuuug

SEQ ID NO: 2143 - RNA, 21 nt, Homo sapiens
caccgacucugucuccugcag

SEQ ID NO: 2144 - RNA, 22 nt, Homo sapiens
cagcaggggagagagaggaguc

SEQ ID NO: 2145 - RNA, 22 nt, Homo sapiens
ccucaccaucccuucugccugc

SEQ ID NO: 2146 - RNA, 23 nt, Homo sapiens
caggcagaaguggggcugacagg

SEQ ID NO: 2147 - RNA, 23 nt, Homo sapiens
ccucaccaccccuucugccugca

SEQ ID NO: 2148 - RNA, 24 nt, Homo sapiens
cugcaggcagaaguggggcugaca

SEQ ID NO: 2149 - RNA, 22 nt, Homo sapiens
uuccagcccuucuaaugguagg

SEQ ID NO: 2150 - RNA, 22 nt, Homo sapiens
uaccauuagaagagcuggaaga

SEQ ID NO: 2151 - RNA, 21 nt, Homo sapiens
ucaagugucaucugucccuag

SEQ ID NO: 2152 - RNA, 23 nt, Homo sapiens
uuugggauugacgccacaugucu

SEQ ID NO: 2153 - RNA, 21 nt, Homo sapiens
cugccuguucuuccacuccag

SEQ ID NO: 2154 - RNA, 23 nt, Homo sapiens
uauggaguggacuuucagcuggc

SEQ ID NO: 2155 - RNA, 20 nt, Homo sapiens
ucucuucaucuaccccccag

SEQ ID NO: 2156 - RNA, 21 nt, Homo sapiens
uuggaggguguggaagacauc

SEQ ID NO: 2157 - RNA, 22 nt, Homo sapiens
aucauguaugauacugcaaaca

SEQ ID NO: 2158 - RNA, 22 nt, Homo sapiens
uuugcaguaacaggugugagca

SEQ ID NO: 2159 - RNA, 21 nt, Homo sapiens
aauggcgccacuaggguugug

SEQ ID NO: 2160 - RNA, 25 nt, Homo sapiens
caacccuaggagagggugccauuca

SEQ ID NO: 2161 - RNA, 21 nt, Homo sapiens
guguugaaacaaucucuacug

SEQ ID NO: 2162 - RNA, 22 nt, Homo sapiens
uucacuggaguuuguuucaaua

SEQ ID NO: 2163 - RNA, 21 nt, Homo sapiens
guguugaaacaaucucuacug

SEQ ID NO: 2164 - RNA, 22 nt, Homo sapiens
uaugucugcugaccaucaccuu

SEQ ID NO: 2165 - RNA, 22 nt, Homo sapiens
uggugggccgcagaacaugugc

SEQ ID NO: 2166 - RNA, 22 nt, Homo sapiens
auaauacaugguuaaccucuuu

SEQ ID NO: 2167 - RNA, 22 nt, Homo sapiens
auaauacaugguuaaccucuuu

SEQ ID NO: 2168 - RNA, 22 nt, Homo sapiens
agagguuauccguguuauguuc

SEQ ID NO: 2169 - RNA, 21 nt, Homo sapiens
aauauuauacagucaaccucu

SEQ ID NO: 2170 - RNA, 21 nt, Homo sapiens
aauauuauacagucaaccucu

SEQ ID NO: 2171 - RNA, 21 nt, Homo sapiens
agguugccugugagguguuca

SEQ ID NO: 2172 - RNA, 23 nt, Homo sapiens
ggcagguucucacccucucuagg

SEQ ID NO: 2173 - RNA, 25 nt, Homo sapiens
ggcggagggaaguagguccguuggu

SEQ ID NO: 2174 - RNA, 22 nt, Homo sapiens
cuugguucagggagggucccca

SEQ ID NO: 2175 - RNA, 22 nt, Homo sapiens
aggaccuucccugaaccaagga

SEQ ID NO: 2176 - RNA, 21 nt, Homo sapiens
accuccugugugcauggauua

SEQ ID NO: 2177 - RNA, 22 nt, Homo sapiens
uacccauugcauaucggaguug

SEQ ID NO: 2178 - RNA, 24 nt, Homo sapiens
ugccugggucucuggccugcgcgu

SEQ ID NO: 2179 - RNA, 21 nt, Homo sapiens
ucccacguuguggcccagcag

SEQ ID NO: 2180 - RNA, 22 nt, Homo sapiens
aggcggggcgccgcgggaccgc

SEQ ID NO: 2181 - RNA, 22 nt, Homo sapiens
gguggcccggccgugccugagg

SEQ ID NO: 2182 - RNA, 23 nt, Homo sapiens
uauucauuuauccccagccuaca

SEQ ID NO: 2183 - RNA, 24 nt, Homo sapiens
acuggcuagggaaaaugauuggau

SEQ ID NO: 2184 - RNA, 22 nt, Homo sapiens
uucauuugccucccagccuaca

SEQ ID NO: 2185 - RNA, 24 nt, Homo sapiens
ugggcuaagggagaugauugggua

SEQ ID NO: 2186 - RNA, 20 nt, Homo sapiens
accaggaggcugaggccccu

SEQ ID NO: 2187 - RNA, 23 nt, Homo sapiens
ugucacucggcucggcccacuac

SEQ ID NO: 2188 - RNA, 23 nt, Homo sapiens
ugucacucggcucggcccacuac

SEQ ID NO: 2189 - RNA, 19 nt, Homo sapiens
ugcgccucgggugagcaug

SEQ ID NO: 2190 - RNA, 20 nt, Homo sapiens
gucccugaguguauguggug

SEQ ID NO: 2191 - RNA, 21 nt, Homo sapiens
uuuccucauauucauucagga

SEQ ID NO: 2192 - RNA, 20 nt, Homo sapiens
gucccugaguguauguggug

SEQ ID NO: 2193 - RNA, 21 nt, Homo sapiens
uccgguucucagggcuccacc

SEQ ID NO: 2194 - RNA, 23 nt, Homo sapiens
aggaagcccuggaggggcuggag

SEQ ID NO: 2195 - RNA, 21 nt, Homo sapiens
ccaaaccagucgugccugugg

SEQ ID NO: 2196 - RNA, 22 nt, Homo sapiens
cucaaaccggcugugccugugg

SEQ ID NO: 2197 - RNA, 21 nt, Homo sapiens
acaggcacgacugguuuggca

SEQ ID NO: 2198 - RNA, 22 nt, Homo sapiens
uccgaacucuccauuccucugc

SEQ ID NO: 2199 - RNA, 20 nt, Homo sapiens
ugggaauggggguaagggcc

SEQ ID NO: 2200 - RNA, 22 nt, Homo sapiens
aggcgauguggggauguagaga

SEQ ID NO: 2201 - RNA, 21 nt, Homo sapiens
uaguggucagagggcuuauga

SEQ ID NO: 2202 - RNA, 22 nt, Homo sapiens
ucugacaucagugauucuccug

SEQ ID NO: 2203 - RNA, 22 nt, Homo sapiens
cgcgccugcaggaacugguaga

SEQ ID NO: 2204 - RNA, 23 nt, Homo sapiens
uuccagcccugguaggcgccgcg

SEQ ID NO: 2205 - RNA, 23 nt, Homo sapiens
ugggcaggggcuuauuguaggag

SEQ ID NO: 2206 - RNA, 22 nt, Homo sapiens
ugcaggggucgggugggccagg

SEQ ID NO: 2207 - RNA, 20 nt, Homo sapiens
aggcgcacccgaccacaugc

SEQ ID NO: 2208 - RNA, 22 nt, Homo sapiens
auaguccgaguaacgucggggc

SEQ ID NO: 2209 - RNA, 23 nt, Homo sapiens
cugggcccgcggcgggcgugggg

SEQ ID NO: 2210 - RNA, 20 nt, Homo sapiens
cucgcccugucucccgcuag

SEQ ID NO: 2211 - RNA, 21 nt, Homo sapiens
cgggagcuggggucugcaggu

SEQ ID NO: 2212 - RNA, 20 nt, Homo sapiens
uccugccaccuccuccgcag

SEQ ID NO: 2213 - RNA, 23 nt, Homo sapiens
cucggggcaggcggcugggagcg

SEQ ID NO: 2214 - RNA, 21 nt, Homo sapiens
ucucugcucugcucuccccag

SEQ ID NO: 2215 - RNA, 23 nt, Homo sapiens
uugggaugguaggaccagagggg

SEQ ID NO: 2216 - RNA, 21 nt, Homo sapiens
ucaucccccucgcccucucag

SEQ ID NO: 2217 - RNA, 22 nt, Homo sapiens
ugggcgagggcggcugagcggc

SEQ ID NO: 2218 - RNA, 22 nt, Homo sapiens
ccugacaccccaucugcccuca

SEQ ID NO: 2219 - RNA, 23 nt, Homo sapiens
agaaagguggagggguugucaga

SEQ ID NO: 2220 - RNA, 21 nt, Homo sapiens
ucuauuccccacucuccccag

SEQ ID NO: 2221 - RNA, 22 nt, Homo sapiens
ugggagagcaggguauugugga

SEQ ID NO: 2222 - RNA, 23 nt, Homo sapiens
uaacccuguccucucccucccag

SEQ ID NO: 2223 - RNA, 20 nt, Homo sapiens
uaggggguggcaggcuggcc

SEQ ID NO: 2224 - RNA, 20 nt, Homo sapiens
ucagugucuggauuuccuag

SEQ ID NO: 2225 - RNA, 22 nt, Homo sapiens
ugggaaagacaaacucagaguu

SEQ ID NO: 2226 - RNA, 23 nt, Homo sapiens
cccuucccucacucuucucucag

SEQ ID NO: 2227 - RNA, 23 nt, Homo sapiens
uugaggggagaaugagguggaga

SEQ ID NO: 2228 - RNA, 22 nt, Homo sapiens
aggccuguggcuccucccucag

SEQ ID NO: 2229 - RNA, 25 nt, Homo sapiens
cagggcagagggcacaggaaucuga

SEQ ID NO: 2230 - RNA, 21 nt, Homo sapiens
ucagcuccucucuacccacag

SEQ ID NO: 2231 - RNA, 21 nt, Homo sapiens
cugggugagggcaucuguggu

SEQ ID NO: 2232 - RNA, 22 nt, Homo sapiens
ucugugcuucaccccuacccag

SEQ ID NO: 2233 - RNA, 21 nt, Homo sapiens
uugggguggucggcccuggag

SEQ ID NO: 2234 - RNA, 24 nt, Homo sapiens
cuucugccugcauucuacucccag

SEQ ID NO: 2235 - RNA, 23 nt, Homo sapiens
cgagggguagaagagcacagggg

SEQ ID NO: 2236 - RNA, 21 nt, Homo sapiens
auuguucugucuuucucccag

SEQ ID NO: 2237 - RNA, 22 nt, Homo sapiens
ugggaaagagaaagaacaagua

SEQ ID NO: 2238 - RNA, 22 nt, Homo sapiens
ugucuucucuccucccaaacag

SEQ ID NO: 2239 - RNA, 22 nt, Homo sapiens
aguuugggauggagagaggaga

SEQ ID NO: 2240 - RNA, 22 nt, Homo sapiens
ucggcucucucccucacccuag

SEQ ID NO: 2241 - RNA, 22 nt, Homo sapiens
gugggugcuggugggagccgug

SEQ ID NO: 2242 - RNA, 20 nt, Homo sapiens
accuggguuguccccucuag

SEQ ID NO: 2243 - RNA, 22 nt, Homo sapiens
aguggggugggacccagcuguu

SEQ ID NO: 2244 - RNA, 23 nt, Homo sapiens
agccgcucuucucccugcccaca

SEQ ID NO: 2245 - RNA, 22 nt, Homo sapiens
aaggggcagggacggguggccc

SEQ ID NO: 2246 - RNA, 23 nt, Homo sapiens
gggccucucuugucauccugcag

SEQ ID NO: 2247 - RNA, 19 nt, Homo sapiens
uggaugacaguggaggccu

SEQ ID NO: 2248 - RNA, 21 nt, Homo sapiens
uggguggaagaaggucugguu

SEQ ID NO: 2249 - RNA, 22 nt, Homo sapiens
cagccgccgccugucuccacag

SEQ ID NO: 2250 - RNA, 22 nt, Homo sapiens
ccgggagaaggagguggccugg

SEQ ID NO: 2251 - RNA, 21 nt, Homo sapiens
uccugccuuccucugcaccag

SEQ ID NO: 2252 - RNA, 24 nt, Homo sapiens
agggguguggaaagaggcagaaca

SEQ ID NO: 2253 - RNA, 21 nt, Homo sapiens
uccugucccugucuccuacag

SEQ ID NO: 2254 - RNA, 22 nt, Homo sapiens
ugugggugggaaggacuggauu

SEQ ID NO: 2255 - RNA, 21 nt, Homo sapiens
cuccuccccugccuggcccag

SEQ ID NO: 2256 - RNA, 22 nt, Homo sapiens
ucgggccugggguugggggagc

SEQ ID NO: 2257 - RNA, 20 nt, Homo sapiens
cuguaugcccucaccgcuca

SEQ ID NO: 2258 - RNA, 23 nt, Homo sapiens
uggugcggagagggcccacagug

SEQ ID NO: 2259 - RNA, 21 nt, Homo sapiens
gaacucacccucugcucccag

SEQ ID NO: 2260 - RNA, 24 nt, Homo sapiens
cagggaacagcugggugagcugcu

SEQ ID NO: 2261 - RNA, 21 nt, Homo sapiens
acugagccucucucucuccag

SEQ ID NO: 2262 - RNA, 23 nt, Homo sapiens
uugggggugagguuggugucugg

SEQ ID NO: 2263 - RNA, 21 nt, Homo sapiens
ucccugcccccauacucccag

SEQ ID NO: 2264 - RNA, 22 nt, Homo sapiens
gggggguguggagccagggggc

SEQ ID NO: 2265 - RNA, 22 nt, Homo sapiens
uggucugucucugcccuggcac

SEQ ID NO: 2266 - RNA, 22 nt, Homo sapiens
caccagggcagagcagggcuga

SEQ ID NO: 2267 - RNA, 22 nt, Homo sapiens
ucuucaccugccucugccugca

SEQ ID NO: 2268 - RNA, 22 nt, Homo sapiens
ccagggaggcugguuuggagga

SEQ ID NO: 2269 - RNA, 22 nt, Homo sapiens
uguugucauguuuuuucccuag

SEQ ID NO: 2270 - RNA, 22 nt, Homo sapiens
uaggguagacacugacaacguu

SEQ ID NO: 2271 - RNA, 20 nt, Homo sapiens
uccccuuccucccugcccag

SEQ ID NO: 2272 - RNA, 23 nt, Homo sapiens
aggguggggcuggagguggggcu

SEQ ID NO: 2273 - RNA, 22 nt, Homo sapiens
aacacuggccuugcuaucccca

SEQ ID NO: 2274 - RNA, 22 nt, Homo sapiens
uagggaugggaggccaggauga

SEQ ID NO: 2275 - RNA, 20 nt, Homo sapiens
acucauucuccucuguccag

SEQ ID NO: 2276 - RNA, 23 nt, Homo sapiens
uagagaggggaaggaugugaugu

SEQ ID NO: 2277 - RNA, 22 nt, Homo sapiens
ugaccuuugccucuccccucag

SEQ ID NO: 2278 - RNA, 22 nt, Homo sapiens
uugugggugggcagaagucugu

SEQ ID NO: 2279 - RNA, 21 nt, Homo sapiens
cuguccuaagguuguugaguu

SEQ ID NO: 2280 - RNA, 21 nt, Homo sapiens
ucuucaaccucaggacuugca

SEQ ID NO: 2281 - RNA, 21 nt, Homo sapiens
acacuguccccuucuccccag

SEQ ID NO: 2282 - RNA, 23 nt, Homo sapiens
cagggagaagguggaagugcaga

SEQ ID NO: 2283 - RNA, 22 nt, Homo sapiens
uccuacgcugcucucucacucc

SEQ ID NO: 2284 - RNA, 21 nt, Homo sapiens
ucugagagagcucgauggcag

SEQ ID NO: 2285 - RNA, 23 nt, Homo sapiens
uggcugcuucccuuggucuccag

SEQ ID NO: 2286 - RNA, 23 nt, Homo sapiens
cggggccauggagcagccugugu

SEQ ID NO: 2287 - RNA, 21 nt, Homo sapiens
cuccccggccucugcccccag

SEQ ID NO: 2288 - RNA, 19 nt, Homo sapiens
cuggggaguggcuggggag

SEQ ID NO: 2289 - RNA, 21 nt, Homo sapiens
ucucuggucuuuccuugacag

SEQ ID NO: 2290 - RNA, 22 nt, Homo sapiens
ucccagggucuggucagaguug

SEQ ID NO: 2291 - RNA, 21 nt, Homo sapiens
ucaccuggcuggcccgcccag

SEQ ID NO: 2292 - RNA, 25 nt, Homo sapiens
gugaggcggggccaggagggugugu

SEQ ID NO: 2293 - RNA, 22 nt, Homo sapiens
ugauugucuucccccacccuca

SEQ ID NO: 2294 - RNA, 24 nt, Homo sapiens
cgggugggagcagaucuuauugag

SEQ ID NO: 2295 - RNA, 22 nt, Homo sapiens
ccacgugcuucucuuuccgcag

SEQ ID NO: 2296 - RNA, 23 nt, Homo sapiens
ucgcagacagggacacauggaga

SEQ ID NO: 2297 - RNA, 24 nt, Homo sapiens
caaaggccacauucuccugugcac

SEQ ID NO: 2298 - RNA, 23 nt, Homo sapiens
cacacaggaaaagcggggcccug

SEQ ID NO: 2299 - RNA, 21 nt, Homo sapiens
gagccccucucugcucuccag

SEQ ID NO: 2300 - RNA, 21 nt, Homo sapiens
agguggguauggaggagcccu

SEQ ID NO: 2301 - RNA, 21 nt, Homo sapiens
cccucucugucccacccauag

SEQ ID NO: 2302 - RNA, 23 nt, Homo sapiens
ugguggguggggaggagaagugc

SEQ ID NO: 2303 - RNA, 21 nt, Homo sapiens
cuggcggcugugucuucacag

SEQ ID NO: 2304 - RNA, 24 nt, Homo sapiens
ugagaaggcacagcuugcacguga

SEQ ID NO: 2305 - RNA, 21 nt, Homo sapiens
caaaccccugucuacccgcag

SEQ ID NO: 2306 - RNA, 22 nt, Homo sapiens
cucgggagggcaugggccaggc

SEQ ID NO: 2307 - RNA, 23 nt, Homo sapiens
uugcuccugacucugugcccaca

SEQ ID NO: 2308 - RNA, 22 nt, Homo sapiens
uggguguaggcuggagcugagg

SEQ ID NO: 2309 - RNA, 22 nt, Homo sapiens
acugucacuucucugcccauag

SEQ ID NO: 2310 - RNA, 23 nt, Homo sapiens
uugggcccaggaguaaacaggau

SEQ ID NO: 2311 - RNA, 21 nt, Homo sapiens
ucgugucccucuuguccacag

SEQ ID NO: 2312 - RNA, 25 nt, Homo sapiens
acuugggcaggagggacccuguaug

SEQ ID NO: 2313 - RNA, 21 nt, Homo sapiens
aggcccuguccucugccccag

SEQ ID NO: 2314 - RNA, 25 nt, Homo sapiens
ucggggcaugggggagggaggcugg

SEQ ID NO: 2315 - RNA, 23 nt, Homo sapiens
caaccaccacugucucuccccag

SEQ ID NO: 2316 - RNA, 19 nt, Homo sapiens
ucugggugcaguggggguu

SEQ ID NO: 2317 - RNA, 20 nt, Homo sapiens
uccacucuccuggcccccag

SEQ ID NO: 2318 - RNA, 23 nt, Homo sapiens
acggggagucaggcaguggugga

SEQ ID NO: 2319 - RNA, 21 nt, Homo sapiens
ugccucccugacauuccacag

SEQ ID NO: 2320 - RNA, 22 nt, Homo sapiens
agugggaggacaggaggcaggu

SEQ ID NO: 2321 - RNA, 21 nt, Homo sapiens
aagcccugucuccucccaucu

SEQ ID NO: 2322 - RNA, 21 nt, Homo sapiens
cugggaggggcuggguuuggc

SEQ ID NO: 2323 - RNA, 21 nt, Homo sapiens
cuccucuguuuucuuuccuag

SEQ ID NO: 2324 - RNA, 23 nt, Homo sapiens
uugggagggaagacagcuggaga

SEQ ID NO: 2325 - RNA, 21 nt, Homo sapiens
ucccuugucuccuuucccuag

SEQ ID NO: 2326 - RNA, 23 nt, Homo sapiens
uggggaaggcuuggcagggaaga

SEQ ID NO: 2327 - RNA, 22 nt, Homo sapiens
ugccucuuuuccacggccucag

SEQ ID NO: 2328 - RNA, 21 nt, Homo sapiens
cgggccggaggucaagggcgu

SEQ ID NO: 2329 - RNA, 23 nt, Homo sapiens
caccuuuguguccccauccugca

SEQ ID NO: 2330 - RNA, 25 nt, Homo sapiens
uaggggugggggaauucaggggugu

SEQ ID NO: 2331 - RNA, 22 nt, Homo sapiens
uuccugggcuucuccucuguag

SEQ ID NO: 2332 - RNA, 22 nt, Homo sapiens
uaggggaaaaguccugauccgg

SEQ ID NO: 2333 - RNA, 22 nt, Homo sapiens
ucucaccccaacucugccccag

SEQ ID NO: 2334 - RNA, 20 nt, Homo sapiens
gccggggcuuugggugaggg

SEQ ID NO: 2335 - RNA, 21 nt, Homo sapiens
acaucgccccaccuuccccag

SEQ ID NO: 2336 - RNA, 22 nt, Homo sapiens
ugggagggcguggaugauggug

SEQ ID NO: 2337 - RNA, 23 nt, Homo sapiens
ugacgccccuucugauucugccu

SEQ ID NO: 2338 - RNA, 21 nt, Homo sapiens
gcgguggggccggaggggcgu

SEQ ID NO: 2339 - RNA, 22 nt, Homo sapiens
ucucagcugcugcccucuccag

SEQ ID NO: 2340 - RNA, 22 nt, Homo sapiens
uggcggggguagagcuggcugc

SEQ ID NO: 2341 - RNA, 22 nt, Homo sapiens
uucgccacuucccucccugcag

SEQ ID NO: 2342 - RNA, 21 nt, Homo sapiens
cugggagaagaguggugaaga

SEQ ID NO: 2343 - RNA, 21 nt, Homo sapiens
cggcgcccgugucuccuccag

SEQ ID NO: 2344 - RNA, 24 nt, Homo sapiens
guaggggcgucccgggcgcgcggg

SEQ ID NO: 2345 - RNA, 21 nt, Homo sapiens
cgaccucggcgaccccucacu

SEQ ID NO: 2346 - RNA, 23 nt, Homo sapiens
gugaguguggauuuggcgggguu

SEQ ID NO: 2347 - RNA, 21 nt, Homo sapiens
ugccuccuuggucuccggcag

SEQ ID NO: 2348 - RNA, 22 nt, Homo sapiens
ccccuggggcugggcaggcgga

SEQ ID NO: 2349 - RNA, 22 nt, Homo sapiens
cuccuccacagccccugcucau

SEQ ID NO: 2350 - RNA, 21 nt, Homo sapiens
guaagcaggggcucuggguga

SEQ ID NO: 2351 - RNA, 20 nt, Homo sapiens
uccccaaccccugcccgcag

SEQ ID NO: 2352 - RNA, 22 nt, Homo sapiens
uguggguucuggguugggguga

SEQ ID NO: 2353 - RNA, 20 nt, Homo sapiens
cucacucucagucccucccu

SEQ ID NO: 2354 - RNA, 20 nt, Homo sapiens
cagggggacugggggugagc

SEQ ID NO: 2355 - RNA, 21 nt, Homo sapiens
accccucguuucuucccccag

SEQ ID NO: 2356 - RNA, 24 nt, Homo sapiens
uggggggacaggaugagaggcugu

SEQ ID NO: 2357 - RNA, 21 nt, Homo sapiens
gaagcucuccccuccccgcag

SEQ ID NO: 2358 - RNA, 23 nt, Homo sapiens
uugugggguuggagagcuggcug

SEQ ID NO: 2359 - RNA, 22 nt, Homo sapiens
ugcaugacccuucccuccccac

SEQ ID NO: 2360 - RNA, 25 nt, Homo sapiens
aggagggaaggggcugagaacagga

SEQ ID NO: 2361 - RNA, 21 nt, Homo sapiens
cuaccccccaucccccuguag

SEQ ID NO: 2362 - RNA, 23 nt, Homo sapiens
ccagggggaugggcgagcuuggg

SEQ ID NO: 2363 - RNA, 23 nt, Homo sapiens
ugcccugcaugguguccccacag

SEQ ID NO: 2364 - RNA, 20 nt, Homo sapiens
ggggaggugugcagggcugg

SEQ ID NO: 2365 - RNA, 21 nt, Homo sapiens
caccucuccuggcaucgcccc

SEQ ID NO: 2366 - RNA, 21 nt, Homo sapiens
guaggugacagucaggggcgg

SEQ ID NO: 2367 - RNA, 20 nt, Homo sapiens
accccugccacucacuggcc

SEQ ID NO: 2368 - RNA, 23 nt, Homo sapiens
uggucagaggcagcaggaaauga

SEQ ID NO: 2369 - RNA, 22 nt, Homo sapiens
uucaccccucucaccuaagcag

SEQ ID NO: 2370 - RNA, 20 nt, Homo sapiens
cuagguggggggcuugaagc

SEQ ID NO: 2371 - RNA, 22 nt, Homo sapiens
ucccucgccuucucacccucag

SEQ ID NO: 2372 - RNA, 22 nt, Homo sapiens
cuggggguggggggcugggcgu

SEQ ID NO: 2373 - RNA, 22 nt, Homo sapiens
cgcaccugccucucacccacag

SEQ ID NO: 2374 - RNA, 21 nt, Homo sapiens
ugagggugucagcaggugacg

SEQ ID NO: 2375 - RNA, 23 nt, Homo sapiens
uugcucugcucccccgcccccag

SEQ ID NO: 2376 - RNA, 22 nt, Homo sapiens
uagggggcggcuuguggagugu

SEQ ID NO: 2377 - RNA, 22 nt, Homo sapiens
ugaagcucugacauuccugcag

SEQ ID NO: 2378 - RNA, 24 nt, Homo sapiens
uguaggcaugaggcagggcccagg

SEQ ID NO: 2379 - RNA, 23 nt, Homo sapiens
cacugcauuccugcuuggcccag

SEQ ID NO: 2380 - RNA, 22 nt, Homo sapiens
gugagccaguggaauggagagg

SEQ ID NO: 2381 - RNA, 21 nt, Homo sapiens
gugugaccaccguuccugcag

SEQ ID NO: 2382 - RNA, 22 nt, Homo sapiens
caggcagggaggugggaccaug

SEQ ID NO: 2383 - RNA, 21 nt, Homo sapiens
cuucucuucucuccuucccag

SEQ ID NO: 2384 - RNA, 22 nt, Homo sapiens
uggcaaggaaagaagaggauca

SEQ ID NO: 2385 - RNA, 23 nt, Homo sapiens
uccccugcucccuuguuccccag

SEQ ID NO: 2386 - RNA, 23 nt, Homo sapiens
auggggacagggaucagcauggc

SEQ ID NO: 2387 - RNA, 21 nt, Homo sapiens
agccugugcuugucccugcag

SEQ ID NO: 2388 - RNA, 22 nt, Homo sapiens
augcaggccuguguacagcacu

SEQ ID NO: 2389 - RNA, 21 nt, Homo sapiens
ccgcucuuccccugaccccag

SEQ ID NO: 2390 - RNA, 25 nt, Homo sapiens
auggggugagauggggaggagcagc

SEQ ID NO: 2391 - RNA, 21 nt, Homo sapiens
aaccuuggccccucuccccag

SEQ ID NO: 2392 - RNA, 23 nt, Homo sapiens
caggggcugggguuucagguucu

SEQ ID NO: 2393 - RNA, 22 nt, Homo sapiens
acucgcauccuucccuuggcag

SEQ ID NO: 2394 - RNA, 22 nt, Homo sapiens
ucccaagggugagaugcugcca

SEQ ID NO: 2395 - RNA, 21 nt, Homo sapiens
uggcuucucuugcacacccag

SEQ ID NO: 2396 - RNA, 23 nt, Homo sapiens
uagguggcgccggaggagucauu

SEQ ID NO: 2397 - RNA, 18 nt, Homo sapiens
gaaggaccugcaccuucg

SEQ ID NO: 2398 - RNA, 21 nt, Homo sapiens
uggggcggggcaggucccugc

SEQ ID NO: 2399 - RNA, 19 nt, Homo sapiens
ucucucugacuccauggca

SEQ ID NO: 2400 - RNA, 24 nt, Homo sapiens
ucugccauaggaagcuuggagugg

SEQ ID NO: 2401 - RNA, 22 nt, Homo sapiens
uugucucuuguuccucacacag

SEQ ID NO: 2402 - RNA, 22 nt, Homo sapiens
uugugugaguacagagagcauc

SEQ ID NO: 2403 - RNA, 21 nt, Homo sapiens
aagccucuguccccaccccag

SEQ ID NO: 2404 - RNA, 22 nt, Homo sapiens
uugggguggagggccaaggagc

SEQ ID NO: 2405 - RNA, 22 nt, Homo sapiens
ugugacuucuccccugccacag

SEQ ID NO: 2406 - RNA, 19 nt, Homo sapiens
ugcggcagagcugggguca

SEQ ID NO: 2407 - RNA, 21 nt, Homo sapiens
ugaccucuccgcuccgcacag

SEQ ID NO: 2408 - RNA, 23 nt, Homo sapiens
gugcgugguggcucgaggcgggg

SEQ ID NO: 2409 - RNA, 24 nt, Homo sapiens
aggcucuaacuggcuuucccugca

SEQ ID NO: 2410 - RNA, 20 nt, Homo sapiens
cagggaaccaguuggggcuu

SEQ ID NO: 2411 - RNA, 21 nt, Homo sapiens
ugagccucuccuucccuccag

SEQ ID NO: 2412 - RNA, 21 nt, Homo sapiens
ucaggguugguagggguugcu

SEQ ID NO: 2413 - RNA, 21 nt, Homo sapiens
ucucuggucuugccaccccag

SEQ ID NO: 2414 - RNA, 22 nt, Homo sapiens
guaggggagguugggccaggga

SEQ ID NO: 2415 - RNA, 22 nt, Homo sapiens
gcgcugacccgccuucuccgca

SEQ ID NO: 2416 - RNA, 22 nt, Homo sapiens
uggggagguguggagucagcau

SEQ ID NO: 2417 - RNA, 22 nt, Homo sapiens
cuccccucucuuuccuguucag

SEQ ID NO: 2418 - RNA, 23 nt, Homo sapiens
ucaauaggaaagaggugggaccu

SEQ ID NO: 2419 - RNA, 21 nt, Homo sapiens
accgucucuucuguuccccag

SEQ ID NO: 2420 - RNA, 22 nt, Homo sapiens
ugggagccaugagggucugugc

SEQ ID NO: 2421 - RNA, 20 nt, Homo sapiens
aucugcucucuuguucccag

SEQ ID NO: 2422 - RNA, 22 nt, Homo sapiens
aggaagcaagagaacccugugg

SEQ ID NO: 2423 - RNA, 20 nt, Homo sapiens
ugccuccuccguggccucag

SEQ ID NO: 2424 - RNA, 20 nt, Homo sapiens
ugggcugcugagaaggggca

SEQ ID NO: 2425 - RNA, 23 nt, Homo sapiens
ugucuuucuucucucccuugcag

SEQ ID NO: 2426 - RNA, 22 nt, Homo sapiens
ccaaggaaggaggcuggacauc

SEQ ID NO: 2427 - RNA, 21 nt, Homo sapiens
ugacuaacucccacucuacag

SEQ ID NO: 2428 - RNA, 24 nt, Homo sapiens
uagguagagugugaggaggagguc

SEQ ID NO: 2429 - RNA, 20 nt, Homo sapiens
acccuuuuucucuuucccag

SEQ ID NO: 2430 - RNA, 23 nt, Homo sapiens
aguagagaggaaaaguuaggguc

SEQ ID NO: 2431 - RNA, 21 nt, Homo sapiens
uuucucucuccacuuccucag

SEQ ID NO: 2432 - RNA, 22 nt, Homo sapiens
guguggaagaugggaggagaaa

SEQ ID NO: 2433 - RNA, 20 nt, Homo sapiens
uaugucccaucccuccauca

SEQ ID NO: 2434 - RNA, 21 nt, Homo sapiens
gugagggacugggauuugugg

SEQ ID NO: 2435 - RNA, 23 nt, Homo sapiens
aaaagcacuuuucugucucccag

SEQ ID NO: 2436 - RNA, 22 nt, Homo sapiens
aggggguagaaaguggcugaag

SEQ ID NO: 2437 - RNA, 21 nt, Homo sapiens
augccucccccggccccgcag

SEQ ID NO: 2438 - RNA, 22 nt, Homo sapiens
cgcagggcccuggcgcaggcau

SEQ ID NO: 2439 - RNA, 23 nt, Homo sapiens
ccuucacugugacucugcugcag

SEQ ID NO: 2440 - RNA, 21 nt, Homo sapiens
accagggccagcagggaaugu

SEQ ID NO: 2441 - RNA, 20 nt, Homo sapiens
aaguccugcuucuguugcag

SEQ ID NO: 2442 - RNA, 22 nt, Homo sapiens
aagcagcaguggcaagacuccu

SEQ ID NO: 2443 - RNA, 22 nt, Homo sapiens
uuggguuuucucuucaauccag

SEQ ID NO: 2444 - RNA, 22 nt, Homo sapiens
ucuggauugaagagacgaccca

SEQ ID NO: 2445 - RNA, 22 nt, Homo sapiens
gcccaggacuuugugcggggug

SEQ ID NO: 2446 - RNA, 24 nt, Homo sapiens
acccccgggcaaagaccugcagau

SEQ ID NO: 2447 - RNA, 21 nt, Homo sapiens
accuugcaucugcauccccag

SEQ ID NO: 2448 - RNA, 22 nt, Homo sapiens
uaggguacucagagcaaguugu

SEQ ID NO: 2449 - RNA, 22 nt, Homo sapiens
uuggcuggucucugcuccgcag

SEQ ID NO: 2450 - RNA, 22 nt, Homo sapiens
uggggguggucucuagccaagg

SEQ ID NO: 2451 - RNA, 21 nt, Homo sapiens
auggucuccuguucucugcag

SEQ ID NO: 2452 - RNA, 21 nt, Homo sapiens
uucuuuguuuuuaauucacag

SEQ ID NO: 2453 - RNA, 21 nt, Homo sapiens
ccucuccucccugugccccag

SEQ ID NO: 2454 - RNA, 19 nt, Homo sapiens
cggggccagagcagagagc

SEQ ID NO: 2455 - RNA, 21 nt, Homo sapiens
ugaccccuucugucucccuag

SEQ ID NO: 2456 - RNA, 22 nt, Homo sapiens
ugggggcuggaugggguagagu

SEQ ID NO: 2457 - RNA, 22 nt, Homo sapiens
ggcucaugugucuguccucuuc

SEQ ID NO: 2458 - RNA, 23 nt, Homo sapiens
acagaggacaguggagugugagc

SEQ ID NO: 2459 - RNA, 19 nt, Homo sapiens
guggucucuuggcccccag

SEQ ID NO: 2460 - RNA, 23 nt, Homo sapiens
ugggggcugggaugggccauggu

SEQ ID NO: 2461 - RNA, 22 nt, Homo sapiens
accagccuguguccaccuccag

SEQ ID NO: 2462 - RNA, 23 nt, Homo sapiens
gaguggauaggggagugugugga

SEQ ID NO: 2463 - RNA, 20 nt, Homo sapiens
cccggccggaacgccgcacu

SEQ ID NO: 2464 - RNA, 22 nt, Homo sapiens
gugcggaacgcuggccggggcg

SEQ ID NO: 2465 - RNA, 21 nt, Homo sapiens
uggcccuuuguaccccuccag

SEQ ID NO: 2466 - RNA, 23 nt, Homo sapiens
aggaggugguacuaggggccagc

SEQ ID NO: 2467 - RNA, 17 nt, Homo sapiens
uguccucuguuccucag

SEQ ID NO: 2468 - RNA, 21 nt, Homo sapiens
cccugggguucugaggacaug

SEQ ID NO: 2469 - RNA, 22 nt, Homo sapiens
uguucauuggaacccugcgcag

SEQ ID NO: 2470 - RNA, 24 nt, Homo sapiens
agcgugggauguccaugaagucag

SEQ ID NO: 2471 - RNA, 21 nt, Homo sapiens
ugcguuucuccucuugagcag

SEQ ID NO: 2472 - RNA, 24 nt, Homo sapiens
aagcucagguuugagaacugcuga

SEQ ID NO: 2473 - RNA, 22 nt, Homo sapiens
agacugaccuucaaccccacag

SEQ ID NO: 2474 - RNA, 25 nt, Homo sapiens
uugggguuuggggugcagacauugc

SEQ ID NO: 2475 - RNA, 22 nt, Homo sapiens
uacagcccugugaucuuuccag

SEQ ID NO: 2476 - RNA, 24 nt, Homo sapiens
aagagaggagcaguggugcugugg

SEQ ID NO: 2477 - RNA, 21 nt, Homo sapiens
ugacugagcuucuccccacag

SEQ ID NO: 2478 - RNA, 22 nt, Homo sapiens
uuggggauugggucaggccagu

SEQ ID NO: 2479 - RNA, 22 nt, Homo sapiens
cagccagccccugcucaccccu

SEQ ID NO: 2480 - RNA, 22 nt, Homo sapiens
gugaggaggggcuggcagggac

SEQ ID NO: 2481 - RNA, 23 nt, Homo sapiens
ugacccccaugucgccucuguag

SEQ ID NO: 2482 - RNA, 23 nt, Homo sapiens
gagaggaacaugggcucaggaca

SEQ ID NO: 2483 - RNA, 22 nt, Homo sapiens
acugggcagggcuguggugagu

SEQ ID NO: 2484 - RNA, 18 nt, Homo sapiens
uggaccucuccuccccag

SEQ ID NO: 2485 - RNA, 22 nt, Homo sapiens
acuggguagguggggcuccagg

SEQ ID NO: 2486 - RNA, 24 nt, Homo sapiens
ccucacccagcucucuggcccucu

SEQ ID NO: 2487 - RNA, 22 nt, Homo sapiens
cgggcaugcugggagagacuuu

SEQ ID NO: 2488 - RNA, 23 nt, Homo sapiens
uagacguggugaaggauugagug

SEQ ID NO: 2489 - RNA, 21 nt, Homo sapiens
gugagacuucucucccuucag

SEQ ID NO: 2490 - RNA, 24 nt, Homo sapiens
uugaagggacaagucagauaugcc

SEQ ID NO: 2491 - RNA, 21 nt, Homo sapiens
acacccucuuucccuaccgcc

SEQ ID NO: 2492 - RNA, 23 nt, Homo sapiens
uagguggcagaggagggacuuca

SEQ ID NO: 2493 - RNA, 22 nt, Homo sapiens
gaucccuuuaucuguccucuag

SEQ ID NO: 2494 - RNA, 23 nt, Homo sapiens
uuagaggcuggaauagagauucu

SEQ ID NO: 2495 - RNA, 21 nt, Homo sapiens
cucucccucuuuacccacuag

SEQ ID NO: 2496 - RNA, 23 nt, Homo sapiens
uguguguguagaggaagaaggga

SEQ ID NO: 2497 - RNA, 21 nt, Homo sapiens
uuccuucuguugucugugcag

SEQ ID NO: 2498 - RNA, 22 nt, Homo sapiens
acuggcagaacacugaagcagc

SEQ ID NO: 2499 - RNA, 21 nt, Homo sapiens
cgccgcgcgcaucggcucagc

SEQ ID NO: 2500 - RNA, 22 nt, Homo sapiens
gugaguaguggcgcgcggcggc

SEQ ID NO: 2501 - RNA, 22 nt, Homo sapiens
gcucauccccaucuccuuucag

SEQ ID NO: 2502 - RNA, 19 nt, Homo sapiens
ugggggagauggggguuga

SEQ ID NO: 2503 - RNA, 22 nt, Homo sapiens
cagcacccuguggcucccacag

SEQ ID NO: 2504 - RNA, 22 nt, Homo sapiens
caugggaguucggggugguugc

SEQ ID NO: 2505 - RNA, 21 nt, Homo sapiens
cccaugccuccugccgcgguc

SEQ ID NO: 2506 - RNA, 20 nt, Homo sapiens
ucucgcaucaggaggcaagg

SEQ ID NO: 2507 - RNA, 23 nt, Homo sapiens
uucucucugucuuucucucucag

SEQ ID NO: 2508 - RNA, 22 nt, Homo sapiens
cagagggaauacagagggcaau

SEQ ID NO: 2509 - RNA, 23 nt, Homo sapiens
caguucugcuguucugacucuag

SEQ ID NO: 2510 - RNA, 23 nt, Homo sapiens
auggagcuggaaccagaucaggc

SEQ ID NO: 2511 - RNA, 22 nt, Homo sapiens
auucuuccugcccuggcuccau

SEQ ID NO: 2512 - RNA, 21 nt, Homo sapiens
ugagggacccaggacaggaga

SEQ ID NO: 2513 - RNA, 24 nt, Homo sapiens
agcugucuguguuuuccuucucag

SEQ ID NO: 2514 - RNA, 22 nt, Homo sapiens
caggaaggagacaggcaguuca

SEQ ID NO: 2515 - RNA, 21 nt, Homo sapiens
cagccucugcccuuggccucc

SEQ ID NO: 2516 - RNA, 22 nt, Homo sapiens
agggccgaaggguggaagcugc

SEQ ID NO: 2517 - RNA, 21 nt, Homo sapiens
cuggccucuucuuucuccuag

SEQ ID NO: 2518 - RNA, 23 nt, Homo sapiens
agggagaaagcuagaagcugaag

SEQ ID NO: 2519 - RNA, 21 nt, Homo sapiens
ugucacccgcuccuugcccag

SEQ ID NO: 2520 - RNA, 22 nt, Homo sapiens
cagggcagggaaggugggagag

SEQ ID NO: 2521 - RNA, 21 nt, Homo sapiens
ccgccuucucuccucccccag

SEQ ID NO: 2522 - RNA, 22 nt, Homo sapiens
ugguggaggaagagggcagcuc

SEQ ID NO: 2523 - RNA, 22 nt, Homo sapiens
auccucuuucguccuucccacu

SEQ ID NO: 2524 - RNA, 22 nt, Homo sapiens
ugggguaaggauaggaggguca

SEQ ID NO: 2525 - RNA, 24 nt, Homo sapiens
ugcugccucuccucuugccugcag

SEQ ID NO: 2526 - RNA, 22 nt, Homo sapiens
uacaagucaggagcugaagcag

SEQ ID NO: 2527 - RNA, 22 nt, Homo sapiens
uucccuaucucacucuccucag

SEQ ID NO: 2528 - RNA, 22 nt, Homo sapiens
agggagggugugguauggaugu

SEQ ID NO: 2529 - RNA, 23 nt, Homo sapiens
cccaucaccuuuccgucuccccu

SEQ ID NO: 2530 - RNA, 22 nt, Homo sapiens
agaggcugagaaggugauguug

SEQ ID NO: 2531 - RNA, 21 nt, Homo sapiens
cuuugcuuccugcuccccuag

SEQ ID NO: 2532 - RNA, 25 nt, Homo sapiens
aggggggcacugcgcaagcaaagcc

SEQ ID NO: 2533 - RNA, 21 nt, Homo sapiens
ugcccuucucuccuccugccu

SEQ ID NO: 2534 - RNA, 21 nt, Homo sapiens
cccgcaggugagaugagggcu

SEQ ID NO: 2535 - RNA, 21 nt, Homo sapiens
uccccuccacuuuccuccuag

SEQ ID NO: 2536 - RNA, 23 nt, Homo sapiens
uggggggacagauggagaggaca

SEQ ID NO: 2537 - RNA, 21 nt, Homo sapiens
aucugucucgauuguuuccag

SEQ ID NO: 2538 - RNA, 20 nt, Homo sapiens
aaggagaugcucaggcagau

SEQ ID NO: 2539 - RNA, 20 nt, Homo sapiens
ucugugccccuacuucccag

SEQ ID NO: 2540 - RNA, 23 nt, Homo sapiens
ucggggagucugggguccggaau

SEQ ID NO: 2541 - RNA, 20 nt, Homo sapiens
ccacugccuaugccccacag

SEQ ID NO: 2542 - RNA, 21 nt, Homo sapiens
caugggguagggcagaguagg

SEQ ID NO: 2543 - RNA, 21 nt, Homo sapiens
cccucaucuuccccuccuuuc

SEQ ID NO: 2544 - RNA, 19 nt, Homo sapiens
uaaggagggggaugagggg

SEQ ID NO: 2545 - RNA, 21 nt, Homo sapiens
ucccucucccaccccuugcag

SEQ ID NO: 2546 - RNA, 21 nt, Homo sapiens
guaagggaccggagaguagga

SEQ ID NO: 2547 - RNA, 22 nt, Homo sapiens
cccugcugccuucaccugccag

SEQ ID NO: 2548 - RNA, 21 nt, Homo sapiens
caggcagguguaggguggagc

SEQ ID NO: 2549 - RNA, 21 nt, Homo sapiens
uugccugcccucuuccuccag

SEQ ID NO: 2550 - RNA, 24 nt, Homo sapiens
aggaggauggagagcugggccaga

SEQ ID NO: 2551 - RNA, 22 nt, Homo sapiens
ugucucucgcccuuggccuuag

SEQ ID NO: 2552 - RNA, 21 nt, Homo sapiens
cagggccaggcacagaguaag

SEQ ID NO: 2553 - RNA, 22 nt, Homo sapiens
caacaaaucacagucugccaua

SEQ ID NO: 2554 - RNA, 22 nt, Homo sapiens
caacaaaucccagucuaccuaa

SEQ ID NO: 2555 - RNA, 23 nt, Homo sapiens
uggaagacuagugauuuuguugu

SEQ ID NO: 2556 - RNA, 22 nt, Homo sapiens
caacuagacugugagcuucuag

SEQ ID NO: 2557 - RNA, 23 nt, Homo sapiens
aaggagcuuacaaucuagcuggg

SEQ ID NO: 2558 - RNA, 23 nt, Homo sapiens
agcucccugaaucccugucccag

SEQ ID NO: 2559 - RNA, 20 nt, Homo sapiens
ugggaggaggggaucuuggg

SEQ ID NO: 2560 - RNA, 27 nt, Homo sapiens
uggucuguucauucucucuuuuuggcc

SEQ ID NO: 2561 - RNA, 22 nt, Homo sapiens
ucggccuggggaggaggaaggg

SEQ ID NO: 2562 - RNA, 20 nt, Homo sapiens
acccgcccgucuccccacag

SEQ ID NO: 2563 - RNA, 21 nt, Homo sapiens
guguggccggcaggcgggugg

SEQ ID NO: 2564 - RNA, 22 nt, Homo sapiens
caagccucuccugcccuuccag

SEQ ID NO: 2565 - RNA, 21 nt, Homo sapiens
cuggggggaggagacccugcu

SEQ ID NO: 2566 - RNA, 22 nt, Homo sapiens
gggacccagggagagacguaag

SEQ ID NO: 2567 - RNA, 22 nt, Homo sapiens
ucucucucccacuucccugcag

SEQ ID NO: 2568 - RNA, 21 nt, Homo sapiens
uggggguguggggagagagag

SEQ ID NO: 2569 - RNA, 22 nt, Homo sapiens
auccucucuucccuccucccag

SEQ ID NO: 2570 - RNA, 22 nt, Homo sapiens
ugggggaggaaggacaggccau

SEQ ID NO: 2571 - RNA, 23 nt, Homo sapiens
ugcaucacagccuuuggcccuag

SEQ ID NO: 2572 - RNA, 22 nt, Homo sapiens
acgggcagggcagugcacccug

SEQ ID NO: 2573 - RNA, 23 nt, Homo sapiens
ccucccugcccgccucucugcag

SEQ ID NO: 2574 - RNA, 21 nt, Homo sapiens
uccagggagacagugugugag

SEQ ID NO: 2575 - RNA, 21 nt, Homo sapiens
ugacccaccccucuccaccag

SEQ ID NO: 2576 - RNA, 21 nt, Homo sapiens
ucuguggaguggggugccugu

SEQ ID NO: 2577 - RNA, 18 nt, Homo sapiens
cuggcagggggagaggua

SEQ ID NO: 2578 - RNA, 21 nt, Homo sapiens
cuacaggcuggaaugggcuca

SEQ ID NO: 2579 - RNA, 23 nt, Homo sapiens
gauccaucucugccuguauuggc

SEQ ID NO: 2580 - RNA, 20 nt, Homo sapiens
ucugguccuggacaggaggc

SEQ ID NO: 2581 - RNA, 22 nt, Homo sapiens
uuuccuguccuccaaccagacc

SEQ ID NO: 2582 - RNA, 18 nt, Homo sapiens
caccauggacgguuuacc

SEQ ID NO: 2583 - RNA, 23 nt, Homo sapiens
ugagaacugacaaaugugguagg

SEQ ID NO: 2584 - RNA, 20 nt, Homo sapiens
aggaggacaaguugugggau

SEQ ID NO: 2585 - RNA, 23 nt, Homo sapiens
uucaugaacugggucuagcuugg

SEQ ID NO: 2586 - RNA, 19 nt, Homo sapiens
uggcccaagaccucagacc

SEQ ID NO: 2587 - RNA, 19 nt, Homo sapiens
ucuggggucuugggccauc

SEQ ID NO: 2588 - RNA, 23 nt, Homo sapiens
cugcagccacuuggggaacuggu

SEQ ID NO: 2589 - RNA, 23 nt, Homo sapiens
uuguucucaaacuggcugucaga

SEQ ID NO: 2590 - RNA, 22 nt, Homo sapiens
ucugugcuacuggaugaagagu

SEQ ID NO: 2591 - RNA, 23 nt, Homo sapiens
ucagcauucauuggcaccagaga

SEQ ID NO: 2592 - RNA, 21 nt, Homo sapiens
cugaacuagagauugggccca

SEQ ID NO: 2593 - RNA, 24 nt, Homo sapiens
ggcucaaucucugguccugcagcc

SEQ ID NO: 2594 - RNA, 19 nt, Homo sapiens
uuucuauguuaguuggaag

SEQ ID NO: 2595 - RNA, 21 nt, Homo sapiens
uucaacaaggguguaggaugg

SEQ ID NO: 2596 - RNA, 21 nt, Homo sapiens
cagggcccuggcuuuagcaga

SEQ ID NO: 2597 - RNA, 21 nt, Homo sapiens
ugcugagguccgggcugugcc

SEQ ID NO: 2598 - RNA, 28 nt, Homo sapiens
uagaucuuugacucuggcagucuccagg

SEQ ID NO: 2599 - RNA, 23 nt, Homo sapiens
uaaagacuguagaggcaacuggu

SEQ ID NO: 2600 - RNA, 19 nt, Homo sapiens
ucugagguggaacagcagc

SEQ ID NO: 2601 - RNA, 18 nt, Homo sapiens
ugcuuccuuucucagcug

SEQ ID NO: 2602 - RNA, 21 nt, Homo sapiens
cuuccgccccgccgggcgucg

SEQ ID NO: 2603 - RNA, 22 nt, Homo sapiens
cuguugccacuaaccucaaccu

SEQ ID NO: 2604 - RNA, 22 nt, Homo sapiens
ugcggggcuagggcuaacagca

SEQ ID NO: 2605 - RNA, 18 nt, Homo sapiens
agaagggaagauggugac

SEQ ID NO: 2606 - RNA, 22 nt, Homo sapiens
uuugugaccugguccacuaacc

SEQ ID NO: 2607 - RNA, 22 nt, Homo sapiens
gaugguugaccagagagcacac

SEQ ID NO: 2608 - RNA, 22 nt, Homo sapiens
gcagagugcaaacaauuuugac

SEQ ID NO: 2609 - RNA, 20 nt, Homo sapiens
cggcucugggucugugggga

SEQ ID NO: 2610 - RNA, 22 nt, Homo sapiens
gcagcagggugaaacugacaca

SEQ ID NO: 2611 - RNA, 22 nt, Homo sapiens
ggggcuggggccggggccgagc

SEQ ID NO: 2612 - RNA, 22 nt, Homo sapiens
gcaggugcucacuuguccuccu

SEQ ID NO: 2613 - RNA, 19 nt, Homo sapiens
uugaucucggaagcuaagc

SEQ ID NO: 2614 - RNA, 21 nt, Homo sapiens
uggaggagaaggaaggugaug

SEQ ID NO: 2615 - RNA, 22 nt, Homo sapiens
acuccagccccacagccucagc

SEQ ID NO: 2616 - RNA, 22 nt, Homo sapiens
aggaggaauuggugcuggucuu

SEQ ID NO: 2617 - RNA, 23 nt, Homo sapiens
ucugcucauaccccaugguuucu

SEQ ID NO: 2618 - RNA, 23 nt, Homo sapiens
ugcaccaugguugucugagcaug

SEQ ID NO: 2619 - RNA, 23 nt, Homo sapiens
cugggaucuccggggucuugguu

SEQ ID NO: 2620 - RNA, 22 nt, Homo sapiens
ugagaccucuggguucugagcu

SEQ ID NO: 2621 - RNA, 23 nt, Homo sapiens
uccaguaccacgugucagggcca

SEQ ID NO: 2622 - RNA, 22 nt, Homo sapiens
cuuagacugccagacucccuga

SEQ ID NO: 2623 - RNA, 23 nt, Homo sapiens
uugcacucuggccuucucccagg

SEQ ID NO: 2624 - RNA, 19 nt, Homo sapiens
cggggucggcggcgacgug

SEQ ID NO: 2625 - RNA, 23 nt, Homo sapiens
aauagcucagaaugucaguucug

SEQ ID NO: 2626 - RNA, 24 nt, Homo sapiens
ugaagcgccugugcucugccgaga

SEQ ID NO: 2627 - RNA, 22 nt, Homo sapiens
augaagccuucucugccuuacg

SEQ ID NO: 2628 - RNA, 22 nt, Homo sapiens
gagggcagagccagcuuccuga

SEQ ID NO: 2629 - RNA, 22 nt, Homo sapiens
aaaacuaggacuguguggugua

SEQ ID NO: 2630 - RNA, 21 nt, Homo sapiens
aagggacagggagggucgugg

SEQ ID NO: 2631 - RNA, 21 nt, Homo sapiens
cagcggagccuggagagaagg

SEQ ID NO: 2632 - RNA, 21 nt, Homo sapiens
cguggaggacgaggaggaggc

SEQ ID NO: 2633 - RNA, 23 nt, Homo sapiens
cuacccucggucugcuuaccaca

SEQ ID NO: 2634 - RNA, 22 nt, Homo sapiens
gacaauuguugaucuugggccu

SEQ ID NO: 2635 - RNA, 21 nt, Homo sapiens
guuuggacauaguguggcugg

SEQ ID NO: 2636 - RNA, 22 nt, Homo sapiens
uaccugggagacugagguugga

SEQ ID NO: 2637 - RNA, 22 nt, Homo sapiens
uauguaguagucaaaggcauuu

SEQ ID NO: 2638 - RNA, 21 nt, Homo sapiens
ucaaaugcagauccugacuuc

SEQ ID NO: 2639 - RNA, 21 nt, Homo sapiens
ugaggugaccgcagaugggaa

SEQ ID NO: 2640 - RNA, 22 nt, Homo sapiens
uuggugaggaccccaagcucgg

SEQ ID NO: 2641 - RNA, 21 nt, Homo sapiens
uuuuaaggacacugagggauc

SEQ ID NO: 2642 - RNA, 20 nt, Homo sapiens
ugugacccuagaauaauuac

SEQ ID NO: 2643 - RNA, 24 nt, Homo sapiens
aggcugugaugcucuccugagccc

SEQ ID NO: 2644 - RNA, 18 nt, Homo sapiens
auccuagucacggcacca

SEQ ID NO: 2645 - RNA, 19 nt, Homo sapiens
ugcccugagacuuuugcuc

SEQ ID NO: 2646 - RNA, 18 nt, Homo sapiens
uucccagccaacgcacca

SEQ ID NO: 2647 - RNA, 21 nt, Homo sapiens
ucugguguauagcguugcuca

SEQ ID NO: 2648 - RNA, 23 nt, Homo sapiens
caguaacaaagauucauccuugu

SEQ ID NO: 2649 - RNA, 22 nt, Homo sapiens
cgggacuguagagggcaugagc

SEQ ID NO: 2650 - RNA, 24 nt, Homo sapiens
uggcgauuuuggaacucaauggca

SEQ ID NO: 2651 - RNA, 21 nt, Homo sapiens
gaaaguacagaucggaugggu

SEQ ID NO: 2652 - RNA, 23 nt, Homo sapiens
cuuugagcacaugagcagacgga

SEQ ID NO: 2653 - RNA, 20 nt, Homo sapiens
cguggauugucuggaugcau

SEQ ID NO: 2654 - RNA, 21 nt, Homo sapiens
guggcucuguaguaagaugga

SEQ ID NO: 2655 - RNA, 22 nt, Homo sapiens
cuggacuuugaucuugccauaa

SEQ ID NO: 2656 - RNA, 22 nt, Homo sapiens
ggggaacuguagaugaaaaggc

SEQ ID NO: 2657 - RNA, 24 nt, Homo sapiens
ccaugaagcaguggguaggaggac

SEQ ID NO: 2658 - RNA, 21 nt, Homo sapiens
cuuagauuagaggauauuguu

SEQ ID NO: 2659 - RNA, 21 nt, Homo sapiens
cagugauuugaggauuauugc

SEQ ID NO: 2660 - RNA, 22 nt, Homo sapiens
ucaaaaucaggagucggggcuu

SEQ ID NO: 2661 - RNA, 20 nt, Homo sapiens
agcacacugagcgagcggac

SEQ ID NO: 2662 - RNA, 24 nt, Homo sapiens
uguaggaacaguugaauuuuggcu

SEQ ID NO: 2663 - RNA, 21 nt, Homo sapiens
caaugugaucuuuuggaugua

SEQ ID NO: 2664 - RNA, 22 nt, Homo sapiens
ccuagaaacuguaaacuuaguc

SEQ ID NO: 2665 - RNA, 22 nt, Homo sapiens
uguuuguuguaaggaucguugu

SEQ ID NO: 2666 - RNA, 23 nt, Homo sapiens
ggaugguugggggcggucggcgu

SEQ ID NO: 2667 - RNA, 22 nt, Homo sapiens
augugauugacggcugacucca

SEQ ID NO: 2668 - RNA, 20 nt, Homo sapiens
cgguggacuggagugggugg

SEQ ID NO: 2669 - RNA, 20 nt, Homo sapiens
ggcggcggggagguaggcag

SEQ ID NO: 2670 - RNA, 22 nt, Homo sapiens
accuggcagcagggagcgucgu

SEQ ID NO: 2671 - RNA, 24 nt, Homo sapiens
cuauggcgagacuggcauguacuc

SEQ ID NO: 2672 - RNA, 24 nt, Homo sapiens
ugcugauggcagaugucgggucug

SEQ ID NO: 2673 - RNA, 24 nt, Homo sapiens
uauauggacuuuucugauacaaug

SEQ ID NO: 2674 - RNA, 22 nt, Homo sapiens
ggcugagugggguucugacucc

SEQ ID NO: 2675 - RNA, 23 nt, Homo sapiens
ggucuaggcccggugagagacuc

SEQ ID NO: 2676 - RNA, 20 nt, Homo sapiens
cagugaucgucucugcuggc

SEQ ID NO: 2677 - RNA, 23 nt, Homo sapiens
gaaggacacuggugucaacggcu

SEQ ID NO: 2678 - RNA, 23 nt, Homo sapiens
cuugagucgugccuuucugaaug

SEQ ID NO: 2679 - RNA, 22 nt, Homo sapiens
ugauggagcugggaauacucug

SEQ ID NO: 2680 - RNA, 21 nt, Homo sapiens
caggacuugacggcugcaacu

SEQ ID NO: 2681 - RNA, 23 nt, Homo sapiens
gaauacuaaguaaaaaaucagua

SEQ ID NO: 2682 - RNA, 21 nt, Homo sapiens
ugggagagaggacugugaggc

SEQ ID NO: 2683 - RNA, 22 nt, Homo sapiens
ugcuagucuggacugauauggu

SEQ ID NO: 2684 - RNA, 23 nt, Homo sapiens
gaagacuucuuggauuacagggg

SEQ ID NO: 2685 - RNA, 21 nt, Homo sapiens
ccucgguacuggaaaggggua

SEQ ID NO: 2686 - RNA, 24 nt, Homo sapiens
ccuggggacaggggauuggggcag

SEQ ID NO: 2687 - RNA, 21 nt, Homo sapiens
cacacacacacacacacguau

SEQ ID NO: 2688 - RNA, 22 nt, Homo sapiens
ggagacugaugaguucccggga

SEQ ID NO: 2689 - RNA, 21 nt, Homo sapiens
gcaggaacuugugagucuccu

SEQ ID NO: 2690 - RNA, 22 nt, Homo sapiens
cugcccuggcccgagggaccga

SEQ ID NO: 2691 - RNA, 22 nt, Homo sapiens
cugcccuggcccgagggaccga

SEQ ID NO: 2692 - RNA, 23 nt, Homo sapiens
cggccccacgcaccaggguaaga

SEQ ID NO: 2693 - RNA, 21 nt, Homo sapiens
ccuggaaacacugagguugug

SEQ ID NO: 2694 - RNA, 22 nt, Homo sapiens
uauaccucaguuuuaucaggug

SEQ ID NO: 2695 - RNA, 22 nt, Homo sapiens
uggugguuuacaaaguaauuca

SEQ ID NO: 2696 - RNA, 22 nt, Homo sapiens
uggauuucuuugugaaucacca

SEQ ID NO: 2697 - RNA, 21 nt, Homo sapiens
uccucuucucccuccucccag

SEQ ID NO: 2698 - RNA, 20 nt, Homo sapiens
guagaggagauggcgcaggg

SEQ ID NO: 2699 - RNA, 22 nt, Homo sapiens
aggcagcgggguguaguggaua

SEQ ID NO: 2700 - RNA, 22 nt, Homo sapiens
uccauuacacuacccugccucu

SEQ ID NO: 2701 - RNA, 22 nt, Homo sapiens
gugaacgggcgccaucccgagg

SEQ ID NO: 2702 - RNA, 22 nt, Homo sapiens
gugaacgggcgccaucccgagg

SEQ ID NO: 2703 - RNA, 21 nt, Homo sapiens
cuugggagcccuguuagacuc

SEQ ID NO: 2704 - RNA, 22 nt, Homo sapiens
gacugacaccucuuugggugaa

SEQ ID NO: 2705 - RNA, 21 nt, Homo sapiens
uacucaaaaagcugucaguca

SEQ ID NO: 2706 - RNA, 21 nt, Homo sapiens
uuaauaucggacaaccauugu

SEQ ID NO: 2707 - RNA, 21 nt, Homo sapiens
uuaauaucggacaaccauugu

SEQ ID NO: 2708 - RNA, 22 nt, Homo sapiens
aauggcuguccguaguaugguc

SEQ ID NO: 2709 - RNA, 21 nt, Homo sapiens
uacuuggaaaggcaucaguug

SEQ ID NO: 2710 - RNA, 22 nt, Homo sapiens
ugcaacgaaccugagccacuga

SEQ ID NO: 2711 - RNA, 22 nt, Homo sapiens
aguggcacauguuuguugugag

SEQ ID NO: 2712 - RNA, 22 nt, Homo sapiens
ugcaacgaaccugagccacuga

SEQ ID NO: 2713 - RNA, 22 nt, Homo sapiens
ugcaacuuaccugagucauuga

SEQ ID NO: 2714 - RNA, 21 nt, Homo sapiens
cacuguguccuuucugcguag

SEQ ID NO: 2715 - RNA, 22 nt, Homo sapiens
cacuggcuccuuucuggguaga

SEQ ID NO: 2716 - RNA, 22 nt, Homo sapiens
cacuguuuccuuucugagugga

SEQ ID NO: 2717 - RNA, 21 nt, Homo sapiens
uauucagaaaggugccaguca

SEQ ID NO: 2718 - RNA, 22 nt, Homo sapiens
auaaagcuagauaaccgaaagu

SEQ ID NO: 2719 - RNA, 23 nt, Homo sapiens
ucuuugguuaucuagcuguauga

SEQ ID NO: 2720 - RNA, 20 nt, Homo sapiens
ggggagcuguggaagcagua

SEQ ID NO: 2721 - RNA, 25 nt, Homo sapiens
cuagugagggacagaaccaggauuc

SEQ ID NO: 2722 - RNA, 23 nt, Homo sapiens
gcagcagagaauaggacuacguc

SEQ ID NO: 2723 - RNA, 20 nt, Homo sapiens
agagucuugugaugucuugc

SEQ ID NO: 2724 - RNA, 23 nt, Homo sapiens
agguugggaucgguugcaaugcu

SEQ ID NO: 2725 - RNA, 22 nt, Homo sapiens
ggguggggauuuguugcauuac

SEQ ID NO: 2726 - RNA, 22 nt, Homo sapiens
uauugcacuugucccggccugu

SEQ ID NO: 2727 - RNA, 22 nt, Homo sapiens
uauugcacucgucccggccucc

SEQ ID NO: 2728 - RNA, 22 nt, Homo sapiens
agggacgggacgcggugcagug

SEQ ID NO: 2729 - RNA, 22 nt, Homo sapiens
acugcugagcuagcacuucccg

SEQ ID NO: 2730 - RNA, 23 nt, Homo sapiens
caaagugcuguucgugcagguag

SEQ ID NO: 2731 - RNA, 22 nt, Homo sapiens
ugugcgcagggagaccucuccc

SEQ ID NO: 2732 - RNA, 22 nt, Homo sapiens
ugucuacuacuggagacacugg

SEQ ID NO: 2733 - RNA, 23 nt, Homo sapiens
ccaguuaccgcuuccgcuaccgc

SEQ ID NO: 2734 - RNA, 22 nt, Homo sapiens
acaguagagggaggaaucgcag

SEQ ID NO: 2735 - RNA, 22 nt, Homo sapiens
auccgcgcucugacucucugcc

SEQ ID NO: 2736 - RNA, 20 nt, Homo sapiens
gugagucaggguggggcugg

SEQ ID NO: 2737 - RNA, 22 nt, Homo sapiens
ugcccuuaaaggugaacccagu

SEQ ID NO: 2738 - RNA, 21 nt, Homo sapiens
cccugggccucugcuccccag

SEQ ID NO: 2739 - RNA, 24 nt, Homo sapiens
uggggagcugaggcucugggggug

SEQ ID NO: 2740 - RNA, 21 nt, Homo sapiens
aaggcagggcccccgcucccc

SEQ ID NO: 2741 - RNA, 23 nt, Homo sapiens
cacccggcugugugcacaugugc

SEQ ID NO: 2742 - RNA, 22 nt, Homo sapiens
ucuucucuguuuuggccaugug

SEQ ID NO: 2743 - RNA, 22 nt, Homo sapiens
cacauggccgaaacagagaagu

SEQ ID NO: 2744 - RNA, 22 nt, Homo sapiens
ucuucucuguuuuggccaugug

SEQ ID NO: 2745 - RNA, 21 nt, Homo sapiens
cugacuguugccguccuccag

SEQ ID NO: 2746 - RNA, 22 nt, Homo sapiens
aaauuauuguacaucggaugag

SEQ ID NO: 2747 - RNA, 22 nt, Homo sapiens
uucaacggguauuuauugagca

SEQ ID NO: 2748 - RNA, 22 nt, Homo sapiens
uucaacggguauuuauugagca

SEQ ID NO: 2749 - RNA, 21 nt, Homo sapiens
ucaauaaaugucuguugaauu

SEQ ID NO: 2750 - RNA, 19 nt, Homo sapiens
aagggaagauggugaccac

SEQ ID NO: 2751 - RNA, 22 nt, Homo sapiens
aaucaugugcagugccaauaug

SEQ ID NO: 2752 - RNA, 23 nt, Homo sapiens
uuuggcacuagcacauuuuugcu

SEQ ID NO: 2753 - RNA, 22 nt, Homo sapiens
cuauacaacuuacuacuuuccc

SEQ ID NO: 2754 - RNA, 22 nt, Homo sapiens
ugagguaguaaguuguauuguu

SEQ ID NO: 2755 - RNA, 22 nt, Homo sapiens
caagcucgcuucuaugggucug

SEQ ID NO: 2756 - RNA, 22 nt, Homo sapiens
aacccguagauccgaucuugug

SEQ ID NO: 2757 - RNA, 22 nt, Homo sapiens
caagcucgugucuguggguccg

SEQ ID NO: 2758 - RNA, 22 nt, Homo sapiens
cacccguagaaccgaccuugcg

(19) United States
(12) Patent Application Publication
(10) Pub. No.: US 2017/0114124 A1
(43) Pub. Date: Apr. 27, 2017

(54) SLIT-ROBO-Myo9-RHOA PATHWAY AND CANCER

(71) Applicant: Northwestern University, Evanston, IL (US)

(72) Inventors: Jane Ying Wu, Chicago, IL (US); Xiaoping Chen, Chicago, IL (US)

(21) Appl. No.: 15/331,357

(22) Filed: Oct. 21, 2016

Related U.S. Application Data
(60) Provisional application No. 62/244,973, filed on Oct. 22, 2015.

(51) Int. Cl. C07K 16/18; A61K 47/48; C12N 15/113; C12Q 1/68; G01N 33/574

(52) U.S. Cl. CPC C07K 16/18; C12Q 1/6886; G01N 33/57423; G01N 33/57438; C12N 15/113; A61K 47/48538; C12Q 2600/158; C07K 2317/24; C12N 2310/14

(57) ABSTRACT

Myosin 9 family members and Slit-Robo-Myo9-RhoA pathway genes in the treatment and/or diagnosis of cancer.

### CROSS REFERENCE TO RELATED APPLICATIONS

The present invention claims the priority benefit of U.S. Provisional Patent Application 62/244,973, filed Oct. 22, 2015, which is incorporated by reference in its entirety.

### FIELD

Provided herein are Myosin 9 (Myo9) family members and Slit-Robo-Myo9-RhoA pathway genes and gene products in the diagnosis and/or treatment of cancer.

### BACKGROUND

Lung cancer is a leading cause of death and a major health problem in both developed and developing countries (Spiro, 2005; herein incorporated by reference in its entirety). Significant efforts have been made in order to understand pathogenetic mechanisms underlying lung tumorigenesis (Peifer, 2012; Valastyan, 2011; Park, 2011; herein incorporated by reference in their entireties). A number of tumor promoting mutations have been found in EGFR and KRAS genes in lung cancer patients. Tumor suppressor genes for lung cancer have also been discovered that regulate cell cycle, cell proliferation and cell death, including TP53, p16, LKB1/STK11, NF1, RASSF1, APC, BRG1, PTEN, and RB (Sanchez-Cespedes, 2011; Vaahtomeri, 2011; herein incorporated by reference in their entireties). However, little is known about endogenous mechanisms that suppress lung cancer invasion and metastasis.

Pancreatic ductal adenocarcinoma (PDAC) is the most common form of pancreatic cancer and a leading cause of cancer-related deaths. Aggressive invasion and early metastasis make pancreatic cancer highly fatal, with a high 5-yr mortality rate (>95%) and a short median survival (Siegel, 2016; incorporated by reference in its entirety). An important feature of pancreatic cancer is its neural invasion (NI, also known as perineural invasion), a key risk factor for poor prognosis (Shimada, 2011; Bapat, 2011; Liang et al, 2016; incorporated by reference in their entireties). Gene profiling analyses have revealed gene networks and signal transduction pathways involved in PDAC (e.g. Whitsett, 2014; Qiao, 2010; Kozak et al, 2015; Neagu, 2015; incorporated by reference in their entireties), and a number of oncogenes and tumor suppressor genes have been associated with PDAC; however, an understanding of the molecular mechanisms suppressing NI, progression and metastasis of PDAC remains limited, making it difficult to develop effective treatments.

### SUMMARY

Disclosed are methods and compositions relating to the vertebrate members of the Myo9 gene family (e.g., Myo9a & Myo9b), polypeptides, nucleic acids, antibodies, and derivatives thereof in diagnosis of cancers (e.g., cancers that exhibit altered expression of or genetic mutations in the human Myo9 genes). Methods are provided for identifying altered expression or genetic and/or epigenetic changes in the Myo9 genes. Methods find specific applications in, for example, providing diagnostic and prognostic tools for a range of cancers, including cancers of the lung, breast, brain, prostate, pancreatic (e.g., PDAC), stomach, esophagus, liver, kidney, skin, head and neck, and ovaries. In some embodiments, methods are provided for the treatment of such cancers. In some embodiments, methods comprise the use of: (1) agents that specifically recognize Myo9 protein/polypeptides, including antibodies (e.g., monoclonal and polyclonal antibodies; e.g., humanized antibodies); (2) agents that interact with Myo9 protein/polypeptides, including proteins, polysaccharides, and nucleic acids (e.g., non-coding RNAs); (3) agents that specifically interact with Myo9 nucleic acids, including antisense nucleotides, small ribonucleic acids (e.g., siRNA, shRNA, and microRNA), and derivatives; and (4) dominant negative mutant forms of Myo9b proteins that block the activity of Myo9b in promoting tumorigenesis, invasion, and metastasis of cancer. In particular embodiments, the agents are anti-Myo9 antibodies and modulators of Myo9 expression or activities (such as anti-sense oligonucleotides, ribonucleic acids, or other forms of compounds).

In additional embodiments, the technology relates to modulating members of the Slit-Robo-Myo9-RhoA pathway that act upstream and/or downstream of Myo9. For example, in some embodiments the technology relates to increasing the expression and/or activity of Slit (e.g., Slit1, Slit2, Slit3) and/or increasing the expression and/or activity of Robo (Robo1, Robo2, Robo3, Robo4) and/or modulating the expression and/or activity of RhoA. For example, for treatment of subjects having decreased activity of Slit, Robo, and/or RhoA, the technology comprises methods and compositions for increasing the activity of Slit, Robo, and/or RhoA, e.g., by modulating genetic and/or epigenetic factors that decrease the activity of these genes and gene products. For treatment of subjects having a normal activity of Slit, Robo, and/or RhoA, the technology comprises methods and compositions for increasing the activity of Slit, Robo, and/or RhoA, e.g., to provide inhibition of Myo9.

### BRIEF DESCRIPTION OF THE DRAWINGS

FIG. 1. Slit2 inhibits cell migration by regulating RhoA activity in lung cancer cells. (A) The wound-healing assay was carried out using H1299 cells treated with control (Ctr), Slit2 (Sl) and Robo1N (RN) media. At 0 and 13 hr after wound formation, phase-contrast images of migrating cells were obtained using an inverted microscope. (Scale bar: 100 um; data represent 5 independent experiments). (B) Quantification of the cell migration distance over the 13-hr period. Data are presented as the mean±SEM (three independent experiments; p value by Mann-Whitney test). (C) H1299 cells were transfected with plasmids encoding either wild-type Cdc42-myc or Rac1-myc and treated 48 hrs post-transfection with mock control or Slit media for 5 min or 15 min. Cell lysates were then prepared, GST pull-down assay was carried out using GST-PBD, and immunoblotting with myc antibody to measure active or total Cdc42 or Rac1 levels in pull-down or total cell lysates respectively. The active RhoA or total RhoA levels were measured by GST pull-down with GST-RBD and analyzed by immunoblotting with specific anti-RhoA antibody. The levels of Slit protein in the culture media and the actin (as an internal loading control) were also shown. (D and E) Cell migration in H1299ctr or H1299Slit groups following transfection with a control vector or myc-tagged DN-RhoA plasmid as determined by the wound-healing assay. The images were taken at 0 and 12 hr following the wound formation (Scale bar: 100 um; n=5). Data are presented as the mean±SEM (3 independent experiments) and analyzed by Mann-Whitney test.

FIG. 2. Myo9b is a key player in Slit-Robo signaling in lung cancer cells. (A) Interaction of the endogenous Robo1 and Myo9b proteins in H1299 lung cancer cells as demonstrated by co-immunoprecipitation experiments. Immunoprecipitation was carried out using the control IgG (Ctr) or anti-Myo9b antibody followed by Western blotting analyses. The Robo1 protein was detected in proteins immnunoprecipitated by the specific anti-Myo9b, but not by the control antibody. (B) Interaction of purified Myo9b GAP domain (GST-GAP) and Robo1 intracellular domain (Robo-ICD) proteins as shown by the GST pull-down experiments. (C) H1299ctr and H1299Slit (stably expressing Slit) cells transfected with siRNA against Myo9b (siMyo9b-1) or control siRNA (siCtr) were examined for cell migration using the wound-healing assay. Representative images were shown at 0 and 12 hr after wound formation (Scale bar: 100 um). (D) The distance of cell migration (um) in different treatment groups shown in panel C was quantified and presented as the mean±SEM (***: p<0.0001; Mann-Whitney test). The data represent 5 independent experiments. (E) H1299 cells stably expressing Slit were transfected with siMyo9b-1 or siCtr. The GST pull-down and Western blotting assays were performed to measure the levels of active and total RhoA.

FIG. 3. The overall structure of Myo9b RhoGAP domain. (A) The domain organization of Myo9b protein. (B) A structure-based sequence alignment of Myo9b RhoGAP domains from different species, Homo sapiens (Hs) (NM_004145.3) (SEQ ID NO: 47), Mus musculus (Mm) (NM_001142323.1) (SEQ ID NO: 48), Rattus norvegicus (Rn) (NM_001271066.1) (SEQ ID NO: 49), and Danio rerio (Dr) (SEQ ID NO: 50) (XM_005171334.2). The residue numbers of Myo9b RhoGAP domain and the secondary structures are marked on the top. The residues involved in the formation of Patch I, II and III are highlighted with dots. The active arginine-finger of the RhoGAP domain is highlighted by the star. (C-D) Ribbon diagrams of the crystal structure of Myo9b RhoGAP domain from the side view (C) or top view (D). The alpha helical secondary structures (A0 to G) are labeled according to the canonical RhoGAP domain structure with both N- and C-termini marked. (E) A surface representation of Myo9b RhoGAP domain. In this diagram, the hydrophobic, positively charged, negatively charged residues and remaining residues are identified. Myo9b RhoGAP domain contains 3 patches (I to III) in the potential RhoA-binding site, similar to those in the p50RhoGAP protein. (F and G) A combined ribbon-stick model that illustrates detailed features of 3 patches. The side chains of the residues involved in the formation of Patch I, Patch III and Patch II are shown as sticks.

FIG. 4. Myo9b RhoGAP domain contains a unique region that specifically recognizes RhoA. (A) A ribbon diagram for the structural model of the Myo9b RhoGAP/RhoA complex. Myo9b RhoGAP domain and RhoA are identified. Myo9b RhoGAP domain interacts with RhoA through the three patches to form a stable complex. The Switch I, Switch II and A3 helix of RhoA are responsible for the binding to the RhoGAP domain and are labeled. The GDP and MgF3 are shown as sticks and spheres, respectively. (B) An “open-book” view of the interaction interfaces between Myo9b RhoGAP domain and RhoA by a surface representation. (C) A combined ribbon-stick model to illustrate the interaction interface between Patch II and the A3 helix in detail. The side chains of the residues involved in the interface packing between Patch II and the A3 helix are shown as sticks. (D) Mutations inside Patch II impair Myo9b GAP activity in inactivating RhoA. H1299 cells were transfected with the control vector (Ctr), or plasmids encoding either the wild-type Myo9b RhoGAP domain (Wt), or different mutants indicated. Cell extracts were subjected to GST pull-down assays to measure the activity of RhoA. (E) Mutations inside Patch II disrupted the binding between Myo9b RhoGAP domain and RhoA. GST pull-down experiments were performed using recombinant wild-type or mutant forms of GST-Myo9b RhoGAP domain and the cell lysates from HEK293 transfected with myc-RhoA plasmid.

FIG. 5. Slit-Robo signaling suppresses Myo9b RhoGAP activity. (A) Addition of the purified Robo-ICD suppresses Myo9b GAP activity in a dose-dependent manner. Extracts from HEK293 cells transfected with myc-RhoA plasmid were incubated with the different combinations of the purified Myo9b-GAP or control (MBP-only) or Robo-ICD at the molar ratio of 1:1, 1:2 or 1:3. The GTP-RhoA level was measured by GST pull-down coupled Western blotting assay. (B) Expression of Robo1 suppresses Myo9b GAP activity. H1299 cells were co-transfected with Flag-tagged Myo9b GAP and HA-tagged Robo1 at different doses. The GTP-RhoA level was measured as described in panel A. (C) Purified Robo-ICD blocks Myo9b GAP-RhoA interaction in a concentration-dependent manner. Lysates from HEK293 cells transfected with myc-RhoA plasmid were incubated with GST-GAP in the presence of different concentrations of Robo-ICD protein. The interaction of GAP and RhoA was examined using GST pull-down. (D) Slit overexpression led to increased GTP-RhoA by suppressing GAP activity of Myo9b. The parental H1299 cell line or H1299 cells that stably expressed Slit2 (marked as “Sl−” or “Sl+” respectively in corresponding lanes) were transfected with the control vector or the full-length Myo9b, or different deletion mutants of Myo9b (Myo9bGAP or Myo9bΔGAP). Extracts were subjected to GST pull-down experiment; and immunoblotting analyses of pull-down products were shown. (E) GTP-RhoA levels were quantified by the mean±SEM (three independent experiments; with p values determined by Mann-Whitney test). (F) Expression of a dominant negative mutant form of Robo1 lacking its intracellular domain (DNRobo) eliminated Slit-induced activation of RhoA in H1299 cells. GST pull-down coupled Western blotting experiments were performed to detect GTP-RhoA levels in H1299 cells following co-transfection with GFP-tagged DNRobo and Flag-tagged Myo9b-RhoGAP plasmids and treatment with the control or Slit media.

FIG. 6. Slit suppresses lung cancer invasion and metastasis. (A) The Slit2 mRNA level was measured by quantitative RT-PCR in human lung cancer samples and paired adjacent non-cancer control tissues (***: p<0.0001; Student's t test; n=25). (B) H1299ctr and H1299Slit cells were injected subcutaneously into nude mice. Tumor volume was monitored at different time points (**: p<0.001; Mann-Whitney test; n=10). (C) At day 38 following tumor cell injection, mice were sacrificed and tumor weight was measured. The difference of tumor sizes in two groups was quantified (***: p<0.0001; Mann-Whitney test). (D) Images of representative mice in each group, showing their subcutaneous tumors. (E) Images of subcutaneous tumors in the corresponding groups (scale bar: 10 mm). (F) Cross-sectional microscopic images of the subcutaneous tumors following H&E staining. The arrows mark local invasive tumors with irregular borders and microcapillaries adjacent to invading tumor cells (marked by arrowheads) in H1299ctr mice (Scale bar: 50 um). (G) Analyses of subcutaneous tumors in mice injected with the H1299ctr and H1299Slit cells. Y-axis: the average numbers of invasive micro-foci in the subcutaneous tumors (under 11.25× magnification views; ***: p<0.0001; Mann-Whitney test). (H) Lung metastases formed by H1299ctr and H1299Slit cells following subcutaneous implantation. Arrows mark metastatic foci detected on the pulmonary surface (Scale bar: 50 um). (I) Numbers of lung metastatic foci (Mets) per mouse in the H1299ctr and H1299Slit groups (***: p<0.0001; Mann-Whitney test). Data represent three independent experiments.

FIG. 7. Myo9b expression and clinicopathological characteristics in lung cancer samples. (A) Immunostaining was performed to detect Myo9b protein in the human lung cancer (LuCa; lower panels) and matched adjacent non-cancer control tissue (Ctr; upper panels) samples. Representative images of Myo9b staining on tissue microarray are shown at low (2×) or high (40×) magnifications (Scale bars: 200 um and 10 um respectively; n=60). (B) Myo9b expression was scored by immunostaining signals. Myo9b expression levels (−, +, ++, +++) correspond to immunostaining scores (0, 1, 2, 3) respectively (p<0.001; Pearson X2 test). (C) Myo9b mRNA levels were higher in lung cancer samples as compared with their paired adjacent control tissues. Y-axis: the Myo9b mRNA levels expressed as Log 10 median-centered intensity (*: p<0.05; Student's t test; n=25). (D) Myo9b mRNA levels in lung cancer samples of different stages (I-II and III-IV) (*: p<0.05; Student's t test; n=13 for I-II group and n=12 for III-IV group). (E) The Kaplan-Meier survival curves were shown for the overall survival (OS) in lung cancer patients with Myo9b expression levels detected by immmunostaining. Data from 31 samples were included in which survival information was available. P-value was analyzed by Log-rank test. (F) Kaplan-Meier plots of progression-free survival (PFS) of lung cancer patients stratified by lower quartile expression of Myo9b in the CaArray datasets, as indicated. Log-rank test p-value is displayed.

FIG. 8. A model depicting the Slit-Robo signal transduction pathway involving Myo9b and downstream RhoA. Slit interacts with the transmembrane receptor Robo to inhibit lung cancer cell migration in a Robo-dependent manner. The intracellular domain of Robo (Robo-ICD) binds to the RhoGAP domain of Myo9b and suppresses its GAP activity in converting GTP-RhoA to GDP-RhoA, leading to the increased level of GTP-RhoA and subsequent suppression of cell invasion and migration. GAP: GTPase activating protein; ICD: intracellular domain.

FIG. 9. Expression of genes in the Slit-Robo-Myo9b signaling pathway in lung cancer cells. (A) RT-PCR was performed with specific primers to assess the mRNA levels of corresponding genes (Table 5) in primary lung cancer samples (lower part: 3 pairs of lung tumor samples together with their adjacent non-tumor control tissue samples) or lung cancer cell lines (upper part) (H1299, A549, H719 and H841) and HEK293 cells. Lane 6 contains the negative control reaction (H1299) in which the reverse transcriptase was omitted. (B) Detection of Robo1 and Myo9b proteins in different lung cancer cell lines by Western blotting using corresponding specific antibodies. Actin was used as a loading control. (C) Wound-healing experiments were carried out using A549 cells treated by control, Slit and RoboN preparations. At 0 hr and 13 hr after wound formation, phase-contrast images were taken under an inverted microscope (Scale bar: 100 um). Data represent 5 independent experiments. (D) Quantification of the cell migration distance 13 hr after the wound formation. The forward cell migration distance was presented as the mean±SEM (3 independent experiments; ***: p<0.0001 by Mann-Whitney test). (E) GST pull-down experiments were carried out using cell lysates from stable H1299 cell lines overexpressing Slit2 (two groups) or control H1299 cells (Ctr) with GST-RBD/GST-PBD proteins. The active levels of RhoA, Cdc42 or Rac1 were examined by Western blotting using the corresponding antibodies. The actin was shown as an internal control. (F) Migration of H1299 cells transfected with myc-tagged CA-RhoA or control plasmids were examined by wound healing assay. The images were taken at 0 hr. and 12 hr (Scale bar: 100 um). Data represent 5 independent experiments. (G) Quantitative analysis was shown in a bar graph, representing the mean±SEM for triplicate measurements and p value was determined by Mann-Whitney test.

FIG. 10. Myo9b interacts with Robo1 and mediates Slit2-induced cell migration inhibition and RhoA activation. (A) Interaction of Robo1 with Myo9b was examined in HEK293 cells. Cells transfected with HA-Robo1 or control vector were lysed for immunoprecipitation (IP) using anti-HA antibody following immunoblotting with Myo9b antibody. (B) A diagram depicting the full-length (FL) Robo1 and its various deletion mutants. (C) Mapping Myo9b interaction domain in Robo1. Immunoprecipitation with anti-HA antibody from H1299 cell lysates transfected with various combinations of plasmids containing Myo9b and Robo deletion mutants, followed by immunoblotting with anti-Flag antibody. The Robo1 mutant lacking the intracellular domain (ΔICD) was served as the negative control. (D) A diagram illustrating the wild-type Myo9b and different Myo9b mutants. (E) Co-immunoprecipitation assay was performed to determine the requirement for Myo9b RhoGAP domain in association of Myo9b with Robo1. Antibodies used in immunoprecipitation and immunoblotting were as indicated. (F) The purified MBP-His6-Robo-ICD and MBP (maltose binding protein) proteins were shown in lanes 3 and 2 respectively with protein size markers in lane 1. (G) Yeast two-hybrid assay with Robo-ICD and Myo9b C1-GAP or GAP domain. (H) H1299ctr and H1299Slit (stably expressing Slit) cells transfected with siRNA against Myo9b (siMyo9b-2) or control siRNA (siCtr) were examined for cell migration using the wound-healing assay. Representative images were shown at 0 hr and 12 hr after wound formation (Scale bar: 100 um). Data represent 5 independent experiments. (I) The distance of cell migration in different treatment groups was quantified as the mean±SEM (***: p<0.0001; Mann-Whitney test). (J) H1299Slit cells were transfected with siMyo9b-2 or siCtr and GST pull-down assay and immunoblotting were performed to measure the RhoA activity.

FIG. 11. No significant difference was observed in cell proliferation between control and siMyo9bs-mediated H1299 cells treated with Slit (Sl) conditioned media. (A) H1299 cells transfected with siRNA control or siMyo9b were treated with the control or Sl conditioned media, followed by fixation in methanol and staining with crystal violet (0.1%). Representative images of stained cells in different groups are shown (Scale bar: 200 um). Data represent 5 independent experiments. (B) The cell numbers in corresponding groups were quantified using ImageJ software. Data were represented as the mean±SEM in 3 independent experiments and analyzed by one-way ANOVA.

FIG. 12. Myo9b inhibits RhoA activity. (A) H1299 cells were transfected with siCtr or siMyo9b-1/2 and the active levels of RhoA, Cdc42 or Rac1, were detected by Western blotting in cell lysates following the GST pull-down experiment (GST-RBD for GTP-RhoA, GST-PBD for GTP-bound Cdc42 or Rac1). The total input protein levels were shown in corresponding panels. Efficient downregulation of Myo9b expression by its specific siRNAs was shown by Western blotting using anti-myo9b antibody. Actin was used as an internal control. (B) H1299 cells were transfected with the control vector (Ctr), or plasmids encoding either the full-length Myo9b (FL), Myo9b lacking the RhoGAP domain (ΔGAP), the C1 motif and RhoGAP domain (C1-GAP) or the RhoGAP domain of Myo9b (GAP). Cell extracts were subjected to GST pull-down assays. (C) HEK293 cell lysates were prepared following transfection using myc-RhoA, Myc-Cdc42 or Myc-Rac1 plasmids. Cell lysates were incubated with purified GAP protein at different concentrations (1 ug, 5 ug, 10 ug, 25 ug and 50 ug) at 30° C. for 10 min. GST pull-down experiments were carried out to examine the active levels of corresponding small GTPases. BSA was used as a negative control.

FIG. 13. Sequence and structural comparison of Myo9b RhoGAP domain with RhoGAP domains in other proteins. (A) Structure-based sequence alignment of the RhoGAP domains from Myo9b (NM_004145.3) (SEQ ID NO: 51), GRAF (NM_205194.2) (SEQ ID NO: 52), p115 (NM_001666.4) (SEQ ID NO: 53), p50rhoGAP (NM_004308.3) (SEQ ID NO: 54), ArhGAP15 (NM_018460.3) (SEQ ID NO: 55), and cdGAP (NM_020754.3) (SEQ ID NO: 56). The identical residues and highly conserved residues are identified. The residue numbers of Myo9b RhoGAP domain and the secondary structures are marked on the top. The residues responsible for the formation of Patch II are highly variable and highlighted by a gray box and the region connecting αA1 and αB is variable and indicated by a black box. (B) Structural comparison of Myo9b and p50rhoGAP RhoGAP domains. A ribbon diagram of the superimposed RhoGAP domain structures of Myo9b and p50rhoGAP. The Myo9b and p50rhoGAP RhoGAP domains are highlited. The secondary structures and the N- and C-termini of the domains are marked. (C-E), A detailed comparison of the structure of Myo9b and p50rhoGAP RhoGAP domains. The central core four-helix bundles of two domains can be well superimposed, as shown in panel C. In contrast, the neighboring αA1 and the loop between αA1 and Ab show some differences between the two structures, as depicted in panel D. Moreover, the last helix αG of Myo9b is much longer than that of p50rhoGAP, although the functional significance of this feature is unclear at the current stage as shown in panel E.

FIG. 14. The structure of the p50RhoGAP RhoGAP/RhoA complex. (A) A ribbon diagram of the structure of the p50RhoGAP RhoGAP/RhoA complex (PDB code: 1OW3). p50RhoGAP RhoGAP domain and RhoA are identified. The GDP and MgF3 are shown as sticks and spheres, respectively. In this complex structure, the Switch I, Switch II and A3 helix of RhoA binds to the concave side of the RhoGAP domain formed by αA1, αB, αF, αG, and the αA/αA1 and αF/αG loops. The binding site of the RhoGAP domain can be further divided into three patches (Patch I, II and III). The active arginine-finger is located in the αA/αA1 loop. (B) An “open-book” view of the interaction interfaces between p50RhoGAP RhoGAP domain and RhoA. In this surface drawing, the hydrophobic, positively charged, negatively charged residues and remaining residues are highlighted. The diagram depicts the direct interaction between the Patch I, II and III in p50RhoGAP domain with the Switch II, Switch I and the A3 helix of RhoA, respectively.

FIG. 15. Comparison of RhoA, Cdc42 and Rac1. (A) Structure-based sequence alignment of the human RhoA (NM_001664.2) (SEQ ID NO: 57), Cdc42 (NM_001791.3) (SEQ ID NO: 58), and Rac1 (NM_006908.4) (SEQ ID NO: 59) proteins. The identical residues and highly conserved residues are identified. The residue numbers of RhoA and the secondary structures are marked above the peptide sequences. The Switch I, Switch II and P-Loop are also marked. The residues in RhoA, Cdc42 and Rac1 responsible for binding to Patch II of the RhoGAP domain are variable and highlighted by boxes. (B and C), A combined ribbon and stick model illustrates the potential interaction interfaces between Patch II of Myo9b RhoGAP domain and the A3 helix of Cdc42 (B) or Rac1 (C). In this structural comparison, RhoA in the Myo9b RhoGAP/RhoA complex was replaced by Cdc42 (PDB code: 1GRN) or Rac1 (PDB code: 1HE1) to show the potential contacts between Patch II and the A3 helix. In comparison to RhoA, D90 is replaced by S88 in Cdc42; and D90 and E97 are substituted with A88 and A95 respectively in Rac1. It is apparent that the A3 helix of Cdc42 or Rac1 would not be well recognized by the positively charged Path II of Myo9b RhoGAP domain.

FIG. 16. Myo9b RhoGAP was capable of inactivating the mutant forms of Cdc42 or Rac1. (A) Myo9b RhoGAP decreased the active level of the Cdc42(S88D/K94P) double mutant, in which serine and lysine at amino acid residue positions 88 and 94 respectively were changed to aspartic acid and proline. H1299 cells were transfected with the RhoGAP domain of Myo9b and plasmids encoding either the wild-type Cdc42 or the Cdc42(S88D/K94P) double mutant. Cell extracts were subjected to GST pull-down assays and Western blotting to measure the activity of Cdc42. (B) Myo9b RhoGAP inactivated the mutant form of Rac1. The alanine and arginine at 88, 94 and 95 amino acids of Rac1 were mutated to the corresponding amino acids of RhoA indicated to the Rac1(A88D/R94P/A95E) triple mutant. Extracts from H1299 cells transfected with the RhoGAP domain of Myo9b and plasmids encoding either the wildtype Rac1 or the Rac1(A88D/R94P/A95E) triple mutant were subjected to GST pull-down assays to examine the activity of Rac1. (C) Mutations of amino acid residues in helix A3 of Cdc42 increased the binding between Myo9b RhoGAP domain and Cdc42. GST pull-down experiments were carried out examine the interaction of recombinant GST-Myo9b RhoGAP domain and wild-type or mutant forms of myc-Cdc42 from the HEK293 cells. (D) Rac1 mutant increased the binding between Myo9b RhoGAP domain and Rac1. The interaction of GST-Myo9b RhoGAP domain with either the wild-type or the triple mutant forms of Rac1 from the HEK293 cells was measured using GST pull-down assay.

FIG. 17. Slit2 is down-regulated in lung cancer tissues, with the higher Slit2 expression associated with the longer survival of patients. (A) Slit2 gene expression in patients from Oncomine datasets. The maximum, 75th percentile, median, 25th percentile, minimum of corresponding datasets are indicated in the boxplots. Error bar: 10th-90th percentile. Y-axis: the Slit mRNA level is expressed as Log 2 median-centered intensity. P-values were determined by Student's t test (Okayama: n=20/226; Selamat: n=58/58; Landi: n=49/58 for Ctr/LuCa respectively). (B and D), Kaplan-Meier plots of overall survival (OS) or progression-free survival (PFS) of lung cancer patients stratified by median levels of Slit2 expression in the CaArray and GSE31210 datasets with Log-rank test p-values displayed. (C) Kaplan-Meier plots of OS of lung cancer patients at tumor grade III or with lymph node metastasis stratified by median Slit2 expression in the tumor samples, with Log-rank test p-value displayed. (E) The Oncoprint graph summarizes genetic alterations in Slit2, Slit3 or Robo1 genes across 178 lung cancer samples from Cancer cBioPortal dataset. The row represents individual genes and each small column represents a tumor sample. (F) Selected mutations of Slit2, Slit3 or Robo1 among lung cancer patients in TCGA dataset were shown above the diagram in the corresponding domains. (G) The plots demonstrated the relationship between expression of Slit2, Slit3 or Robo1 and CNA (DNA copy-number alterations) in lung tumors. Boxplots were divided by the putative CNA of genes (Homdel: homozygously deleted; Hetloss: heterozygously deleted; Diploid: two alleles present). mRNA levels of Slit2, Slit3 or Robo1 were measured by the number of RPKM (reads per kilobase per million reads sequenced) in the RNASeq data from lung carcinoma patients and expressed as Log 2 median-centered intensity.

FIG. 18. The specificity of Myo9b antibody as demonstrated by immunohistochemical staining and western blotting. (A) Tissue sections from the subcutaneous H1299Ctr tumors in nude mice described in FIG. 7 were stained with pre-immune IgG (Ctr), the purified Myo9b antibody preparation following antigen absorption (Abs) or the purified Myo9b antibody (Non-absorbed, Non-). The cytoplasmic staining was only observed in the Myo9b antibody group, indicating that the Myo9b antibody specifically detected the endogenous Myo9b protein (Scale bar: 50 um; n=10). (B) Myo9b immunostaining signals in panel A was quantified using ImageJ software. Data were presented as the mean±SEM (three independent experiments; p value by Student's t test). (C) Purified Myo9b-GAP domain fused to glutathione S-transferase (GST) protein was used to prepare Myo9b antibody. (D) Western blotting assay using H1299 cell lysates prepared from control siRNA (siCtr) or specific siMyo9b treated cells. The Myo9b band signals detected by the purified Myo9b antibody were reduced by specific siMyo9b. (E) Quantification of Myo9b Western blotting signals in panel C, presented as the mean±SEM (3 independent experiments; p value by Mann-Whitney test).

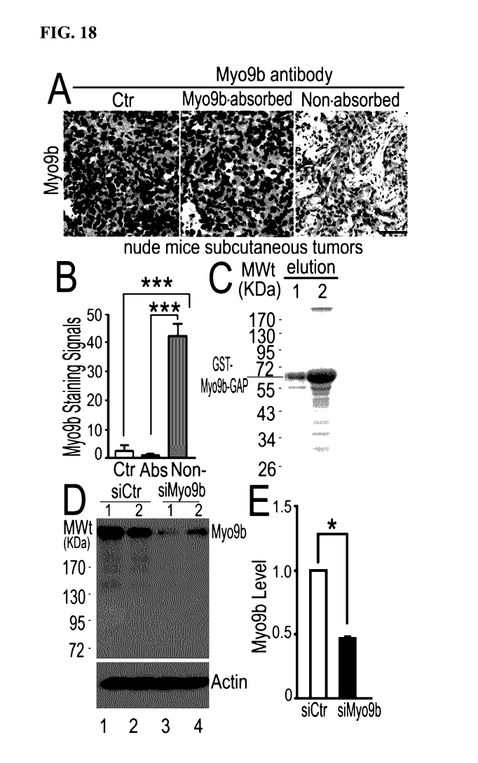

FIG. 19. Immunostaining of lung cancer samples using antibodies against Ecadherin, vimentin and Myo9b. Immunohistochemical staining was carried out using specific antibodies against E-cadherin, vimentin and Myo9b in the consecutive serial sections of a pathologically diagnosed human lung carcinoma sample. E-cadherin and vimentin proteins were utilized respectively as epithelial and mesenchymal markers respectively. Representative staining of these proteins in lung cancer is shown (Scale bars: 50 um, 100 um and 200 um).

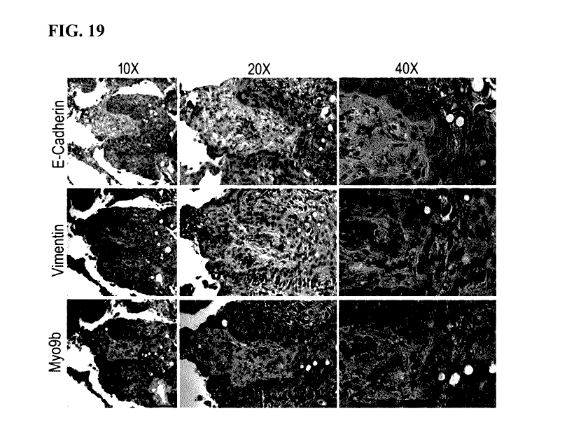

FIG. 20. Mechanism of Slit-Robo-Myo9b-RhoA pathway suppressing PDAC; however, embodiments herein are not limited to any particular mechanism of action and an understanding of the mechanism of action is not necessary to practice such embodiments. Slit interacts with the transmembrane receptor Robo that contains immunoglobulin domains (Ig) and fibronectin domains (FN) in its extracellular region. The intracellular domain (ICD) of Robo (Robo-ICD) binds to the RhoGAP domain of Myo9b and suppresses its GAP activity in converting GTP-RhoA to GDP-RhoA, leading to an increased level of GTP-RhoA. Slit activation of this pathway leads to suppression of NI and metastasis of PDAC. Myo9b contains a motor domain and a RhoGAP domain. The motor domain contains ATP- and actin-binding sites. GAP: GTPase activating protein. Although several Robo-interacting proteins have been identified, Myo9b expression is increased in PDAC samples, increased Myo9b expression predicts poor prognosis, this pathway suppresses NI and metastasis of PDAC.

FIG. 21. Reduced Slit2 expression is associated with worse prognosis in PDAC. Slit2 gene expression was examined using qPCR in our PDAC patient samples (A, B). Higher expression of Slit2 is correlated with reduced lymph node (LN) infiltration. (C) The lower Slit2 expression is associated with shorter patient survival. (D) Expression of Slit2 was examined in normal pancreas, immortal non-tumorigenic pancreatic cell line HPDE, and different PDAC cell lines. (E) Analyses of public datasets show that expression of Slit 1, 2, 3 was lower in PDAC samples than that in the control pancreatic tissue (Ctr).

FIG. 22. The Myo9b expression is increased in a significant fraction of PDAC patients. Specific anti-Myo9b was used in immunohistochemical staining of de-identified PDAC tissue samples. ˜95% of PDAC samples showed intense immunostaining signals (++ or +++).

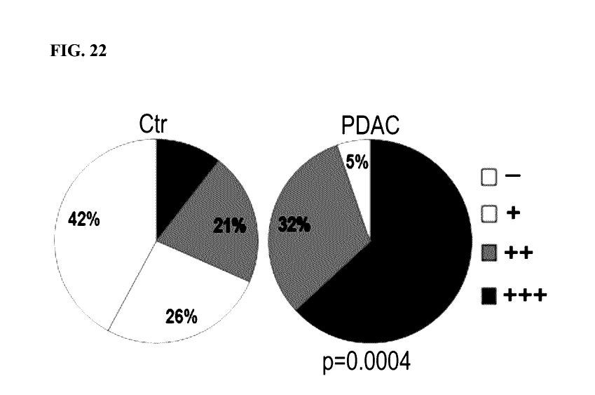

FIG. 23. Increased Myo9b expression is associated with poor prognosis of PDAC. Myo9b mRNA levels are increased in PDAC samples as compared to the control samples in multiple cohorts. (A) Myo9b mRNA levels were measured by qRT-PCR in a cohort of 12 PDAC patient tumor samples together with adjacent non-tumor control (Ctr) samples. (B) Analysis of control and PDAC samples form a TCGA dataset showing that Myo9b mRNA levels were increased in PDAC samples. (C) Kaplan-Meier survival curves showing that higher expression of Myo9b is associated with reduced probability of patient survival. Furthermore, genetic variants of Myo9b are associated with pancreatitis (Nijmeijer et al, 2013; incorporated by reference in its entirety), a risk factor for pancreatic cancer.

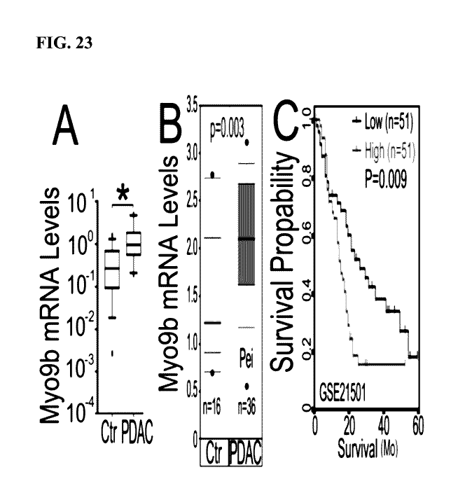

FIG. 24. Knockdown of Myo9b expression in PDAC cells reduces cancer neural invasion in Cancer-DRG co-culture in vitro. UACC462, a human PDAC cell line, was obtained from ATCC (ATCC# CRL2989) and used in the cancer cell co-cultures with dorsal root ganglion (DRG) cells as reported previously (Göhrig A et al, 2014). (A). Myo9b was downregulated by specific siRNA targeting the human Myo9b in UACC462 cells. Western blotting analysis was performed using the specific anti-Myo9b. (B). Neural invasion index was calculated by the percentage of cancer cells that were directly attached to the DRG axons in the total number of cancer cells imaged in the Cancer-DRG co-cultures.

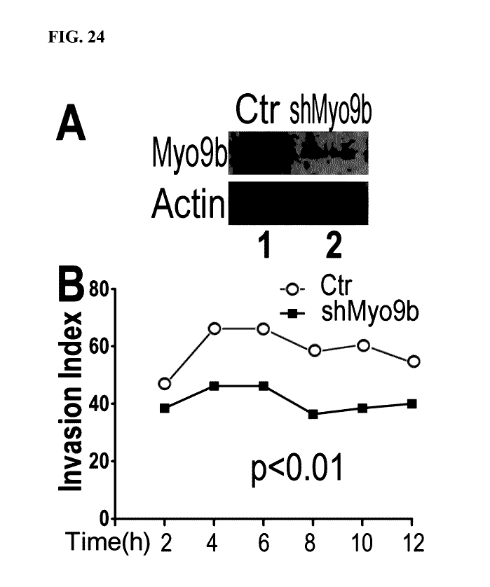

FIG. 25. Pilot data from SN injection experiments show that Slit suppressed NI of PDAC in vivo. (A, B) Representative images of nude mice and their hind legs following SN injection with either control MiaPaCa (Ctr) or MiaPaCa-Slit cells stably expressing Slit2 (Slit). (B) Images of hind leg that exhibited paralysis (left panel) or normal function (right panel). (C) Images of sciatic nerve receiving MiaPaCa-Ctr cells [left panel showing thickened nerve (a sign of NI)] or MiaPaCa-Slit cells (right panel showing the normal sciatic nerve) 6 weeks following injection. The black arrows mark the sciatic nerve; the green star marks the tumor. (D) Expression of Slit in MiaPaCa cells as detected by Western blotting analyses. (E). Tumor sizes in the control and Slit groups. F) Percentage of neural invasion in two groups of injected nude mice (n=12) with the Slit group showing reduced frequency of NI.

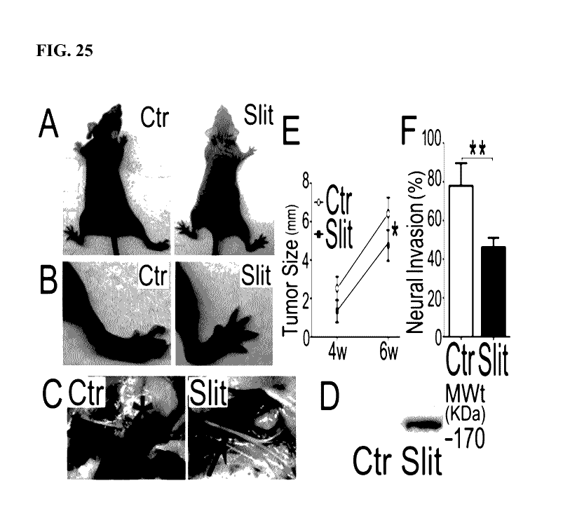

FIG. 26. Slit2 suppresses neural invasion: an EM analysis of cancer-nerve interaction in vivo. Our pilot TEM study using the sciatic nerve invasion model revealed exciting preliminary data: Slit2 expression suppressed cancer-nerve interaction. The control MiaPaCa cells (Ctr) or Slit2-expressing MiaPaCa cells (Slit) were injected into sciatic nerve in nude mice as described in FIG. 25. Sciatic nerves were dissected and analyzed using transmission electron microscopy (TEM). Left and right panels of corresponding groups are low and high magnification images of respective groups. The right panels show the corresponding areas inside black-line boxes within the regions outlined by white dotted lines. Clear signs of cancer-nerve interaction including cancer cells (marked by arrows) that engulfing axons (marked by “*”) were frequently detected in the Ctr group, whereas such PDAC cells were rarely found in the Slit group.

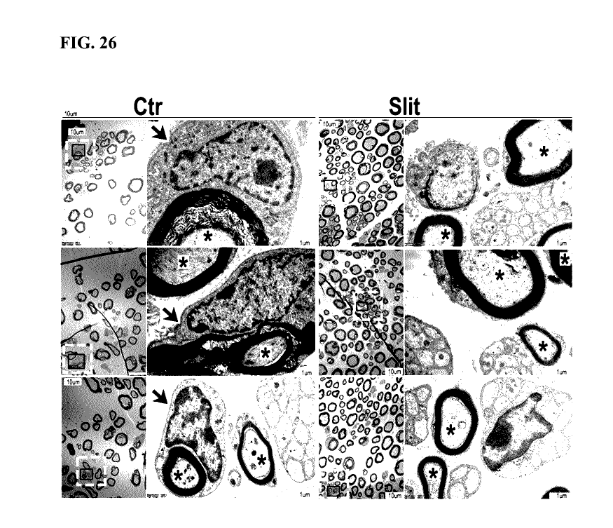

### DETAILED DESCRIPTION

Provided herein are Myosin 9 (Myo9) family members and Slit-Robo-Myo9-RhoA pathway genes and gene products in the treatment and/or diagnosis of cancer. In this detailed description of the various embodiments, for purposes of explanation, numerous specific details are set forth to provide a thorough understanding of the embodiments disclosed. One skilled in the art will appreciate, however, that these various embodiments may be practiced with or without these specific details. In other instances, structures and devices are shown in block diagram form. Furthermore, one skilled in the art can readily appreciate that the specific sequences in which methods are presented and performed are illustrative and it is contemplated that the sequences can be varied and still remain within the spirit and scope of the various embodiments disclosed herein.

All literature and similar materials cited in this application, including but not limited to, patents, patent applications, articles, books, treatises, and internet web pages are expressly incorporated by reference in their entirety for any purpose. Unless defined otherwise, all technical and scientific terms used herein have the same meaning as is commonly understood by one of ordinary skill in the art to which the various embodiments described herein belongs. When definitions of terms in incorporated references appear to differ from the definitions provided in the present teachings, the definition provided in the present teachings shall control. The section headings used herein are for organizational purposes only and are not to be construed as limiting the described subject matter in any way.

### DEFINITIONS

To facilitate an understanding of the present technology, a number of terms and phrases are defined below. Additional definitions are set forth throughout the detailed description. Throughout the specification and claims, the following terms take the meanings explicitly associated herein, unless the context clearly dictates otherwise. The phrase “in one embodiment” as used herein does not necessarily refer to the same embodiment, though it may. Furthermore, the phrase “in another embodiment” as used herein does not necessarily refer to a different embodiment, although it may. Thus, as described below, various embodiments of the invention may be readily combined, without departing from the scope or spirit of the invention. In addition, as used herein, the term “or” is an inclusive “or” operator and is equivalent to the term “and/or” unless the context clearly dictates otherwise. The term “based on” is not exclusive and allows for being based on additional factors not described, unless the context clearly dictates otherwise. In addition, throughout the specification, the meaning of “a”, “an”, and “the” include plural references. The meaning of “in” includes “in” and “on.”

The terms “protein” and “polypeptide” refer to compounds comprising amino acids joined via peptide bonds and are used interchangeably. A “protein” or “polypeptide” encoded by a gene is not limited to the amino acid sequence encoded by the gene, but includes post-translational modifications of the protein.

Where the term “amino acid sequence” is recited herein to refer to an amino acid sequence of a protein molecule, “amino acid sequence” and like terms, such as “polypeptide” or “protein” are not meant to limit the amino acid sequence to the complete, native amino acid sequence associated with the recited protein molecule. Furthermore, an “amino acid sequence” can be deduced from the nucleic acid sequence encoding the protein.

The term “portion” when used in reference to a protein (as in “a portion of a given protein”) refers to fragments of that protein. The fragments may range in size from four amino acid residues to the entire amino sequence minus one amino acid (for example, the range in size includes 4, 5, 6, 7, 8, 9, 10, or 11 . . . amino acids up to the entire amino acid sequence minus one amino acid).

The term “homolog” or “homologous” when used in reference to a polypeptide refers to a high degree of sequence identity between two polypeptides, or to a high degree of similarity between the three-dimensional structure or to a high degree of similarity between the active site and the mechanism of action. In a preferred embodiment, a homolog has a greater than 60% sequence identity, and more preferably greater than 75% sequence identity, and still more preferably greater than 90% sequence identity, with a reference sequence.

The terms “variant” and “mutant” when used in reference to a polypeptide refer to an amino acid sequence that differs by one or more amino acids from another, usually related polypeptide. The variant may have “conservative” changes, wherein a substituted amino acid has similar structural or chemical properties. One type of conservative amino acid substitutions refers to the interchangeability of residues having similar side chains. For example, a group of amino acids having aliphatic side chains is glycine, alanine, valine, leucine, and isoleucine; a group of amino acids having aliphatic-hydroxyl side chains is serine and threonine; a group of amino acids having amide-containing side chains is asparagine and glutamine; a group of amino acids having aromatic side chains is phenylalanine, tyrosine, and tryptophan; a group of amino acids having basic side chains is lysine, arginine, and histidine; and a group of amino acids having sulfur-containing side chains is cysteine and methionine. Preferred conservative amino acids substitution groups are: valine-leucine-isoleucine, phenylalanine-tyrosine, lysine-arginine, alanine-valine, and asparagine-glutamine. More rarely, a variant may have “non-conservative” changes (e.g., replacement of a glycine with a tryptophan). Similar minor variations may also include amino acid deletions or insertions (i.e., additions), or both. Guidance in determining which and how many amino acid residues may be substituted, inserted or deleted without abolishing biological activity may be found using computer programs well known in the art, for example, DNAStar software. Variants can be tested in functional assays. Preferred variants have less than 10%, and preferably less than 5%, and still more preferably less than 2% changes (whether substitutions, deletions, and so on).

The term “domain” when used in reference to a polypeptide refers to a subsection of the polypeptide which possesses a unique structural and/or functional characteristic; typically, this characteristic is similar across diverse polypeptides. The subsection typically comprises contiguous amino acids, although it may also comprise amino acids which act in concert or which are in close proximity due to folding or other configurations. Examples of a protein domain include the transmembrane domains, and the glycosylation sites.

The term “gene” refers to a nucleic acid (e.g., DNA or RNA) sequence that comprises coding sequences necessary for the production of an RNA, or a polypeptide or its precursor (e.g., proinsulin). A functional polypeptide can be encoded by a full length coding sequence or by any portion of the coding sequence as long as the desired activity or functional properties (e.g., enzymatic activity, ligand binding, signal transduction, etc.) of the polypeptide are retained. The term “portion” when used in reference to a gene refers to fragments of that gene. The fragments may range in size from a few nucleotides to the entire gene sequence minus one nucleotide. Thus, “a nucleotide comprising at least a portion of a gene” may comprise fragments of the gene or the entire gene.

The term “gene” also encompasses the coding regions of a structural gene and includes sequences located adjacent to the coding region on both the 5′ and 3′ ends for a distance of about 1 kb on either end such that the gene corresponds to the length of the full-length mRNA. The sequences which are located 5′ of the coding region and which are present on the mRNA are referred to as 5′ non-translated sequences. The sequences which are located 3′ or downstream of the coding region and which are present on the mRNA are referred to as 3′ non-translated sequences. The term “gene” encompasses both cDNA and genomic forms of a gene. A genomic form or clone of a gene contains the coding region interrupted with non-coding sequences termed “introns” or “intervening regions” or “intervening sequences.” Introns are segments of a gene which are transcribed into nuclear RNA (hnRNA); introns may contain regulatory elements such as enhancers. Introns are removed or “spliced out” from the nuclear or primary transcript; introns therefore are absent in the messenger RNA (mRNA) transcript. The mRNA functions during translation to specify the sequence or order of amino acids in a nascent polypeptide.

In addition to containing introns, genomic forms of a gene may also include sequences located on both the 5′ and 3′ end of the sequences which are present on the RNA transcript. These sequences are referred to as “flanking” sequences or regions (these flanking sequences are located 5′ or 3′ to the non-translated sequences present on the mRNA transcript). The 5′ flanking region may contain regulatory sequences such as promoters and enhancers which control or influence the transcription of the gene. The 3′ flanking region may contain sequences which direct the termination of transcription, posttranscriptional cleavage and polyadenylation.

As used herein, “modulation” or “to modulate” means either an increase (stimulation) or a decrease (inhibition) in the expression and/or activity of a gene and/or a gene product. For example, expression may be inhibited to potentially prevent tumor proliferation. “Modulation” may also be spatial or temporal modulation, e.g., a change in the time or location where expression or activity occurs.

The terms “oligonucleotide” or “polynucleotide” or “nucleotide” or “nucleic acid” refer to a molecule comprised of two or more deoxyribonucleotides or ribonucleotides, preferably more than three, and usually more than ten. The exact size will depend on many factors, which in turn depends on the ultimate function or use of the oligonucleotide. The oligonucleotide may be generated in any manner, including chemical synthesis, DNA replication, reverse transcription, or a combination thereof.

The terms “an oligonucleotide having a nucleotide sequence encoding a gene” or “a nucleic acid sequence encoding” a specified polypeptide refer to a nucleic acid sequence comprising the coding region of a gene or in other words the nucleic acid sequence which encodes a gene product. The coding region may be present in either a cDNA, genomic DNA or RNA form. When present in a DNA form, the oligonucleotide may be single-stranded (i.e., the sense strand) or double-stranded. Suitable control elements such as enhancers/promoters, splice junctions, polyadenylation signals, etc. may be placed in close proximity to the coding region of the gene if needed to permit proper initiation of transcription and/or correct processing of the primary RNA transcript. Alternatively, the coding region utilized in the expression vectors of the present invention may contain endogenous enhancers/promoters, splice junctions, intervening sequences, polyadenylation signals, etc. or a combination of both endogenous and exogenous control elements.

The term “recombinant” when made in reference to a nucleic acid molecule refers to a nucleic acid molecule which is comprised of segments of nucleic acid joined together by means of molecular biological techniques. The term “recombinant” when made in reference to a protein or a polypeptide refers to a protein molecule which is expressed using a recombinant nucleic acid molecule.

The terms “complementary” and “complementarity” refer to polynucleotides (i.e., a sequence of nucleotides) related by the base-pairing rules. For example, for the sequence “5′-A-G-T-3′,” is complementary to the sequence “3′-T-C-A-S′.” Complementarity may be “partial,” in which only some of the nucleic acids' bases are matched according to the base pairing rules. Or, there may be “complete” or “total” complementarity between the nucleic acids. The degree of complementarity between nucleic acid strands has significant effects on the efficiency and strength of hybridization between nucleic acid strands. This is of particular importance in amplification reactions, as well as detection methods which depend upon binding between nucleic acids.

The term “wild-type” when made in reference to a gene refers to a gene that has the characteristics of a gene isolated from a naturally occurring source. The term “wild-type” when made in reference to a gene product refers to a gene product that has the characteristics of a gene product isolated from a naturally occurring source. The term “naturally-occurring” as applied to an object refers to the fact that an object can be found in nature. For example, a polypeptide or polynucleotide sequence that is present in an organism (including viruses) that can be isolated from a source in nature and which has not been intentionally modified by man in the laboratory is naturally-occurring. A wild-type gene is frequently that gene which is most frequently observed in a population and is thus arbitrarily designated the “normal” or “wild-type” form of the gene. In contrast, the term “modified” or “mutant” when made in reference to a gene or to a gene product refers, respectively, to a gene or to a gene product which displays modifications in sequence and/or functional properties (i.e., altered characteristics) when compared to the wild-type gene or gene product. It is noted that naturally-occurring mutants can be isolated; these are identified by the fact that they have altered characteristics when compared to the wild-type gene or gene product.

The term “allele” refers to different variations in a gene; the variations include but are not limited to variants and mutants, polymorphic loci and single nucleotide polymorphic loci, frameshift and splice mutations. An allele may occur naturally in a population, or it might arise during the lifetime of any particular individual of the population.

Thus, the terms “variant” and “mutant” when used in reference to a nucleotide sequence refer to an nucleic acid sequence that differs by one or more nucleotides from another, usually related nucleotide acid sequence. A “variation” is a difference between two different nucleotide sequences; typically, one sequence is a reference sequence.

The term “antisense” refers to a deoxyribonucleotide sequence whose sequence of deoxyribonucleotide residues is in reverse 5′ to 3′ orientation in relation to the sequence of deoxyribonucleotide residues in a sense strand of a DNA duplex. A “sense strand” of a DNA duplex refers to a strand in a DNA duplex which is transcribed by a cell in its natural state into a “sense mRNA.” Thus an “antisense” sequence is a sequence having the same sequence as the non-coding strand in a DNA duplex. The term “antisense RNA” refers to a RNA transcript that is complementary to all or part of a target primary transcript or mRNA and that blocks the expression of a target gene by interfering with the processing, transport and/or translation of its primary transcript or mRNA. The complementarity of an antisense RNA may be with any part of the specific gene transcript, i.e., at the 5′ non-coding sequence, 3′ non-coding sequence, introns, or the coding sequence. In addition, as used herein, antisense RNA may contain regions of ribozyme sequences that increase the efficacy of antisense RNA to block gene expression. “Ribozyme” refers to a catalytic RNA and includes sequence-specific endoribonucleases. “Antisense inhibition” refers to the production of antisense RNA transcripts capable of preventing the expression of the target protein.

The term “primer” refers to an oligonucleotide, whether occurring naturally as in a purified restriction digest or produced synthetically, which is capable of acting as a point of initiation of synthesis when placed under conditions in which synthesis of a primer extension product which is complementary to a nucleic acid strand is induced, (e.g., in the presence of nucleotides and an inducing agent such as DNA polymerase and at a suitable temperature and pH). The primer is preferably single stranded for maximum efficiency in amplification, but may alternatively be double stranded. If double stranded, the primer is first treated to separate its strands before being used to prepare extension products. Preferably, the primer is an oligodeoxyribonucleotide. The primer must be sufficiently long to prime the synthesis of extension products in the presence of the inducing agent. The exact lengths of the primers will depend on many factors, including temperature, source of primer and the use of the method.

The term “probe” refers to an oligonucleotide (i.e., a sequence of nucleotides), whether occurring naturally as in a purified restriction digest or produced synthetically, recombinantly or by PCR amplification, that is capable of hybridizing to another oligonucleotide of interest. A probe may be single-stranded or double-stranded. Probes are useful in the detection, identification and isolation of particular gene sequences. It is contemplated that any probe used in the present invention will be labeled with any “reporter molecule,” so that is detectable in any detection system, including, but not limited to enzyme (e.g., ELISA, as well as enzyme-based histochemical assays), fluorescent, radioactive, and luminescent systems. It is not intended that the present invention be limited to any particular detection system or label.

The term “isolated” when used in relation to a nucleic acid, as in “an isolated oligonucleotide” refers to a nucleic acid sequence that is identified and separated from at least one contaminant nucleic acid with which it is ordinarily associated in its natural source. Isolated nucleic acid is present in a form or setting that is different from that in which it is found in nature. In contrast, non-isolated nucleic acids, such as DNA and RNA, are found in the state they exist in nature. Examples of non-isolated nucleic acids include: a given DNA sequence (e.g., a gene) found on the host cell chromosome in proximity to neighboring genes; RNA sequences, such as a specific mRNA sequence encoding a specific protein, found in the cell as a mixture with numerous other mRNAs which encode a multitude of proteins. However, isolated nucleic acid encoding a particular protein includes, by way of example, such nucleic acid in cells ordinarily expressing the protein, where the nucleic acid is in a chromosomal location different from that of natural cells, or is otherwise flanked by a different nucleic acid sequence than that found in nature. The isolated nucleic acid or oligonucleotide may be present in single-stranded or double-stranded form. When an isolated nucleic acid or oligonucleotide is to be utilized to express a protein, the oligonucleotide will contain at a minimum the sense or coding strand (i.e., the oligonucleotide may single-stranded), but may contain both the sense and anti-sense strands (i.e., the oligonucleotide may be double-stranded).

The term “purified” refers to molecules, either nucleic or amino acid sequences, that are removed from their natural environment, isolated or separated. An “isolated nucleic acid sequence” may therefore be a purified nucleic acid sequence. “Substantially purified” molecules are at least 60% free, preferably at least 75% free, and more preferably at least 90% free from other components with which they are naturally associated. As used herein, the term “purified” or “to purify” also refer to the removal of contaminants from a sample. The removal of contaminating proteins results in an increase in the percent of polypeptide of interest in the sample. In another example, recombinant polypeptides are expressed in plant, bacterial, yeast, or mammalian host cells and the polypeptides are purified by the removal of host cell proteins; the percent of recombinant polypeptides is thereby increased in the sample.

The term “composition comprising” a given polynucleotide sequence or polypeptide refers broadly to any composition containing the given polynucleotide sequence or polypeptide. The composition may comprise an aqueous solution. Compositions comprising polynucleotide sequences or fragments thereof may be employed as hybridization probes. In some embodiments, polynucleotide sequences are employed in an aqueous solution containing salts (e.g., NaCl), detergents (e.g., SDS), and other components (e.g., Denhardt's solution, dry milk, salmon sperm DNA, etc.).

The term “test compound” refers to any chemical entity, pharmaceutical, drug, and the like that can be used to treat or prevent a disease, illness, sickness, or disorder of bodily function, or otherwise alter the physiological or cellular status of a sample. Test compounds comprise both known and potential therapeutic compounds. A test compound can be determined to be therapeutic by screening using the screening methods of the present invention. A “known therapeutic compound” refers to a therapeutic compound that has been shown (e.g., through animal trials or prior experience with administration to humans) to be effective in such treatment or prevention.

As used herein, the term “antibody” is used in its broadest sense to refer to whole antibodies, monoclonal antibodies (including human, humanized, or chimeric antibodies), polyclonal antibodies, and antibody fragments that can bind antigen (e.g., Fab′, F′ (ab)2, Fv, single chain antibodies), comprising complementarity determining regions (CDRs) of the foregoing as long as they exhibit the desired biological activity.

As used herein, “antibody fragments” comprise a portion of an intact antibody, preferably the antigen binding or variable region of the intact antibody. Examples of antibody fragments include Fab, Fab′, F(ab′)2, and Fv fragments; diabodies; linear antibodies (Zapata et al., Protein Eng. 8(10): 1057-1062 (1995)); single-chain antibody molecules; and multispecific antibodies formed from antibody fragments.

An antibody that “specifically binds to” or is “specific for” a particular polypeptide or an epitope on a particular polypeptide is one that binds to that particular polypeptide or epitope on a particular polypeptide without substantially binding to any other polypeptide or polypeptide epitope.

As used herein, “active” or “activity” refers to native or naturally occurring biological and/or immunological activity.

As used herein the term, “in vitro” refers to an artificial environment and to processes or reactions that occur within an artificial environment. In vitro environments may include, but are not limited to, test tubes and cell cultures. The term “in vivo” refers to the natural environment (e.g., an animal or a cell) and to processes or reactions that occur within a natural environment.

As used herein, “inhibitor” refers to a molecule which eliminates, minimizes, or decreases the activity, e.g., the biological, enzymatic, chemical, or immunological activity, of a target.

As used herein the term “disease” refers to a deviation from the condition regarded as normal or average for members of a species, and which is detrimental to an affected individual under conditions that are not inimical to the majority of individuals of that species (e.g., diarrhea, nausea, fever, pain, inflammation, etc.).

As used herein, the term “administration” refers to the act of giving a drug, prodrug, antibody, or other agent, or therapeutic treatment to a physiological system (e.g., a subject or in vivo, in vitro, or ex vivo cells, tissues, and organs). Exemplary routes of administration to the human body can be through the eyes (ophthalmic), mouth (oral), skin (transdermal), nose (nasal), lungs (inhalant), oral mucosa (buccal), ear, by injection (e.g., intravenously, subcutaneously, intratumorally, intraperitoneally, etc.) and the like. “Coadministration” refers to administration of more than one chemical agent or therapeutic treatment (e.g., radiation therapy) to a physiological system (e.g., a subject or in vivo, in vitro, or ex vivo cells, tissues, and organs). As used herein, administration “in combination with” one or more further therapeutic agents includes simultaneous (concurrent) and consecutive administration in any order. “Coadministration” of therapeutic treatments may be concurrent, or in any temporal order or physical combination.

As used herein, the term “treating” includes reducing or alleviating at least one adverse effect or symptom of a disease or disorder through introducing in any way a therapeutic composition of the present technology into or onto the body of a subject. “Treatment” refers to both therapeutic treatment and prophylactic or preventative measures, wherein the object is to prevent or slow down (lessen) the targeted pathologic condition or disorder. Those in need of treatment include those already with the disorder as well as those prone to have the disorder or those in whom the disorder is to be prevented.

As used herein, “therapeutically effective dose” refers to an amount of a therapeutic agent sufficient to bring about a beneficial or desired clinical effect. Said dose can be administered in one or more administrations. However, the precise determination of what would be considered an effective dose may be based on factors individual to each patient, including, but not limited to, the patient's age, size, type or extent of disease, stage of the disease, route of administration, the type or extent of supplemental therapy used, ongoing disease process, and type of treatment desired (e.g., aggressive vs. conventional treatment).

As used herein, the term “effective amount” refers to the amount of a composition sufficient to effect beneficial or desired results. An effective amount can be administered in one or more administrations, applications, or dosages and is not intended to be limited to a particular formulation or administration route.

As used herein, the term “pharmaceutical composition” refers to the combination of an active agent with, as desired, a carrier, inert or active, making the composition especially suitable for diagnostic or therapeutic use in vitro, in vivo, or ex vivo.

As used herein, the terms “pharmaceutically acceptable” or “pharmacologically acceptable” refer to compositions that do not substantially produce adverse reactions, e.g., toxic, allergic, or immunological reactions, when administered to a subject.

As used herein, “carriers” include pharmaceutically acceptable carriers, excipients, or stabilizers which are nontoxic to the cell or mammal being exposed thereto at the dosages and concentrations employed. Often the physiologically acceptable carrier is an aqueous pH-buffered solution. Examples of physiologically acceptable carriers include buffers such as phosphate, citrate, and other organic acids; antioxidants including ascorbic acid; low molecular weight (less than about 10 residues) polypeptides; proteins, such as serum albumin, gelatin, or immunoglobulins; hydrophilic polymers such as polyvinylpyrrolidone; amino acids such as glycine, glutamine, asparagine, arginine, or lysine; monosaccharides, disaccharides, and other carbohydrates including glucose, mannose, or dextrins; chelating agents such as EDTA; sugar alcohols such as mannitol or sorbitol; salt-forming counterions such as sodium; and/or nonionic surfactants.

As used herein, the terms “patient” or “subject” refer to organisms to be treated by the compositions of the present technology or to be subject to various tests provided by the technology. The term “subject” includes animals, preferably mammals, including humans. In a preferred embodiment, the subject is a primate. In an even more preferred embodiment, the subject is a human.

As used herein, the term “sample” is used in its broadest sense. In one sense it can refer to animal cells or tissues. In another sense, it is meant to include a specimen or culture obtained from any source, such as biological and environmental samples. Biological samples may be obtained from plants or animals (including humans) and encompass fluids, solids, tissues, and gases. Environmental samples include environmental material such as surface matter, soil, water, and industrial samples. These examples are not to be construed as limiting the sample types applicable to the present technology.

As used herein, the term “transcriptional regulatory region” refers to the non-coding upstream regulatory sequence of a gene, also called the 5′ untranslated region (5′UTR).

As used herein, the terms “detect”, “detecting”, or “detection” may describe either the general act of discovering or discerning or the specific observation of a detectably labeled composition.

As used herein, the term “stage of cancer” refers to a qualitative or quantitative assessment of the level of advancement of a cancer. Criteria used to determine the stage of a cancer include, but are not limited to, the size of the tumor and the extent of metastases (e.g., localized or distant).

As used herein, the term “nucleic acid molecule” refers to any nucleic acid containing molecule, including but not limited to, DNA or RNA. The term encompasses sequences that include any of the known base analogs of DNA and RNA including, but not limited to, 4 acetylcytosine, 8-hydroxy-N6-methyladenosine, aziridinylcytosine, pseudoisocytosine, 5 (carboxyhydroxyl [symbol] methyl) uracil, 5-fluorouracil, 5 bromouracil, 5-carboxymethylaminomethyl 2 thiouracil, 5 carboxymethyl [symbol] aminomethyluracil, dihydrouracil, inosine, N6 isopentenyladenine, 1 methyladenine, 1-methylpseudo [symbol] uracil, 1 methylguanine, 1 methylinosine, 2,2-dimethyl [symbol] guanine, 2 methyladenine, 2 methylguanine, 3-methyl [symbol] cytosine, 5 methylcytosine, N6 methyladenine, 7 methylguanine, 5 methylaminomethyluracil, 5-methoxy [symbol] amino [symbol] methyl 2 thiouracil, beta D mannosylqueosine, 5′ methoxycarbonylmethyluracil, 5 methoxyuracil, 2 methylthio N6 isopentenyladenine, uracil 5 oxyacetic acid methylester, uracil 5 oxyacetic acid, oxybutoxosine, pseudouracil, queosine, 2 thiocytosine, 5-methyl-2 thiouracil, 2-thiouracil, 4 thiouracil, 5-methyluracil, N-uracil 5 oxyacetic acid methylester, uracil 5 oxyacetic acid, pseudouracil, queosine, 2-thiocytosine, and 2,6 diaminopurine.

As used herein, the term “tissue biopsy” refers to a biological material, which is isolated from a patient. The term “tissue”, as used herein, is an aggregate of cells that perform a particular function in an organism and encompasses cell lines and other sources of cellular material including, but not limited to, a biological fluid for example, blood, plasma, sputum, urine, cerebrospinal fluid, lavages, and leukophoresis samples.

As defined herein, “a tumor” is a neoplasm that may either be malignant or non-malignant. Tumors of the same tissue type originate in the same tissue, and may be divided into different subtypes based on their biological characteristics.

As used herein, the term “cancer” refers to a malignant disease caused or characterized by the proliferation of cells that have lost susceptibility to normal growth control. “Malignant disease” refers to a disease caused by cells that have gained the ability to invade either the tissue of origin or to travel to sites removed from the tissue of origin. Particular cancers related to the technology provided herein include, but are not limited to, lung cancer and pancreatic cancer.

Embodiments of the technology relate to Slit protein and nucleic acids encoding Slit protein. In some embodiments, the technology relates to the Slit proteins Slit1, Slit2, and/or Slit3. The neuronal guidance cue, Slit, is a family of secreted glycoproteins that were originally discovered to regulate axonal guidance and neuronal migration by binding to Roundabout (Robo) receptors (Brose, 1999; Li, 1999; Wu, 1999; Ypsilanti, 2010; herein incorporated by reference in their entireties). Subsequent studies demonstrate that Slit-Robo signaling also plays important roles outside of the nervous system, such as modulating chemokine activation and migration of cells from multiple lineages (Brantley-Sieders, 2011; Geutskens, 2010; Legg, 2008; Prasad, 2007; Wu, 2001; herein incorporated by reference in their entireties). Recent studies suggest that neuronal guidance molecule Slit plays important roles in cancer (Ballard, 2012; Mehlen, 2011; Nasarre, 2010; herein incorporated by reference in their entireties). For instance, the Slit2 gene is inactivated in multiple types of cancers, including lung cancer, often as a result of promoter hypermethylation or a loss of heterozygosity (LOH) (Dammann, 2005; Dallol, 2002; Kim, 2008; Tseng, 2010; Yu; 2010; herein incorporated by reference in their entireties). Nonetheless, the role of Slit signaling in lung cancer and underlying mechanisms are not clear.

To dissect the Slit-Robo signaling pathways, experiments were conducted during development of embodiments herein to identify proteins interacting with the Robo receptor and identified Myo9b (Myosin IXb) as a Robo-interacting protein. Myosin IX is an unconventional myosin family motor that moves along actin-filaments (Liao, 2010; van den Boom, 2007; herein incorporated by reference in their entireties). The vertebrate myosin IX family has two members, Myo9a and Myo9b. Accordingly, in some embodiments, the technology relates to Myo9 (e.g., Myo9a, Myo9b) proteins and nucleic acids encoding Myo9 (e.g., Myo9a, Myo9b) proteins. Myo9a is predominantly expressed in testis and brain (Abouhamed, 2009; herein incorporated by reference in its entirety), whereas Myo9b has been reported in the immune cells (Hanley, 2010; Xu, 2014; herein incorporated by reference in their entireties). Different from other unconventional myosins, Myo9b contains a unique RhoGAP domain in its tail region in addition to the head (motor) domain with ATP-binding and actin-binding sites and the neck domain with four isoleucine-glutamine (IQ) motifs (Post, 1998; herein incorporated by reference in its entirety). Using this RhoGAP domain, Myo9b negatively regulates the small G-protein RhoA, converting RhoA from the active GTP-bound form to the inactive GDP-bound form (Hanley, 2010; Saeki, 2005; Wirth, 1996; herein incorporated by reference in their entireties). The small G-protein RhoA plays an important role in modulating actin cytoskeleton during cell migration (Heasman, 2008; Parsons, 2010; herein incorporated by reference in their entireties). The structural basis for Myo9b function in regulating RhoA has been unknown. The mechanisms by which the extracellular signals from guidance cues are transmitted to RhoA or other GTPases thereby organizing coordinated changes in actin cytoskeleton to promote directional cell migration remain to be understood.

Experiments conducted during development of embodiments herein demonstrate that Myo9b is a previously unknown Robo-interacting protein that mediates the Slit inhibitory effect on lung cancer cell migration. Myo9b specifically suppresses RhoA activation through its RhoGAP domain. X-ray crystallography data reveal that the Myo9b RhoGAP domain contains a unique patch that specifically recognizes RhoA. In lung cancer cells, the intracellular domain of Robo directly interacts with Myo9b RhoGAP domain and inhibits its activity. Thus, the negative regulation of Myo9b by the Slit-Robo signaling in lung cancer cells activates RhoA and inhibits the cell migration. Experiments were conducted during development of embodiments herein demonstrating that Slit inhibits lung tumor invasion and metastasis in a xenograft mouse model. Myo9b is highly expressed in human lung cancer tissues as compared with the control samples. Increased Myo9b expression is associated with lymph node metastasis, advanced tumor stage and poor patient survival. These results uncover a previously unknown Slit-Robo-Myo9b-RhoA signaling pathway in inhibiting cell migration and suppressing lung cancer metastasis. See, e.g., Kong et al. (2015) “Myo9b is a key player in SLIT/ROBO-mediated lung tumor suppression” Journal of Clinical Investigation 125: 4407, incorporated herein by reference.

In vitro and in vivo experiments conducted during development of embodiments herein support an important role of Slit2 in suppressing lung cancer. Slit2 inhibits migration of lung cancer cells in a Robo-dependent manner. This is consistent with previous studies in breast cancer (Yuasa-Kawada, 2009; herein incorporated by reference in its entirety), and in other cancer studies such as cancer (Yiin, 2009; herein incorporated by reference in its entirety) and medulloblastoma (Werbowetski-Ogilvie, 2006; herein incorporated by reference in its entirety). Xenograft mouse model demonstrates that increased expression of Slit2 reduces tumor formation, local invasion and lung metastasis.

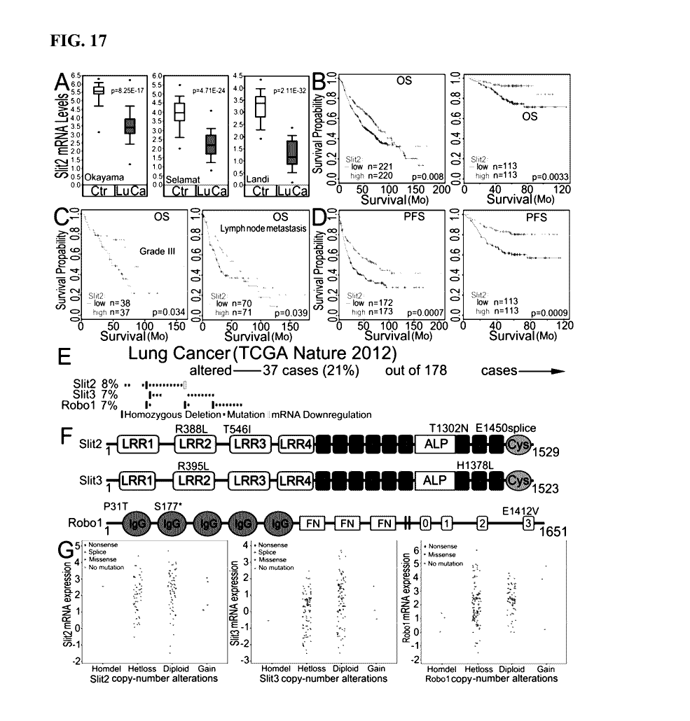

The expression of Slit1 is restricted to the brain whereas both Slit2 and Slit3 are highly expressed in the brain and lung tissues (Wu, 2001, Greenberg, 2004; herein incorporated by reference in their entireties). Experiments conducted during development of embodiments herein demonstrate that Slit2 is significantly down-regulated in human lung cancer and that low Slit2 expression is associated with poor survival of lung cancer patients. In developing or postnatal mouse lung tissues, Slit2 is expressed in the mesenchymal compartment and larger airway epithelium in the developing lung, whereas Slit3 expression is detected the endothelium of large vessels associated with conducting airways (Greenberg, 2004; herein incorporated by reference in its entirety). It is contemplated that the human Slit2/3 genes expressed in lung tissue plays a role in restricting lung cancer invasion and metastasis. Approximately 8% or 7% of lung cancer patients showed genetic alterations in the Slit2 or Slit3 genes respectively. In addition, mutations in the human Robo1 gene have been detected in ˜7% lung cancer cases (FIG. 17). Together, results indicate a role of Slit-Robo signaling in suppressing invasion and metastasis of lung cancer. Accordingly, in some embodiments the technology comprises increasing the expression and/or activity of Slit (e.g., Slit1, Slit2, and/or Slit3), e.g., using methods including, but not limited to, increasing Slit transcription, translation, and/or activity.

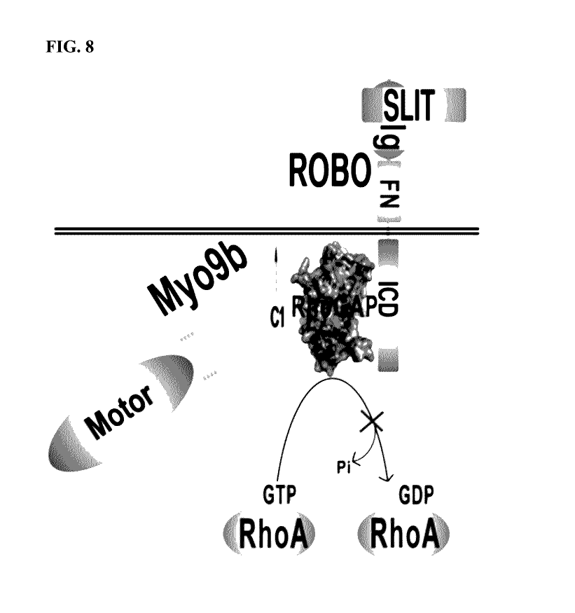

Myo9b is a RhoGAP protein that modulates lamellipodia protrusion and tail retraction by suppressing RhoA activation in migrating immune cells (Hanley, 2010; herein incorporated by reference in its entirety). The involvement of Myo9b in cancer has not been reported previously. Data herein indicate that Myo9b is a Robo-interacting protein that is highly expressed in human lung cancer. The intracellular domain of Robo1 interacts with Myo9b RhoGAP domain and suppresses the RhoGAP activity of Myo9b, as illustrated in FIG. 8. Thus, Slit2-Robo1 signaling suppresses the activity of Myo9b in converting GTP-RhoA to GDP-RhoA, leading to increased GTP-RhoA. Analyses of patient samples show that increased expression of Myo9b correlates with advanced stage of diseases, lymph node metastasis, poor overall survival and shortened progression-free survival. Experiments conducted during development of embodiments herein demonstrate a previously unknown pathway, Slit-Robo-Myo9b-RhoA, in mediating Slit-Robo signaling in lung cancer cells.

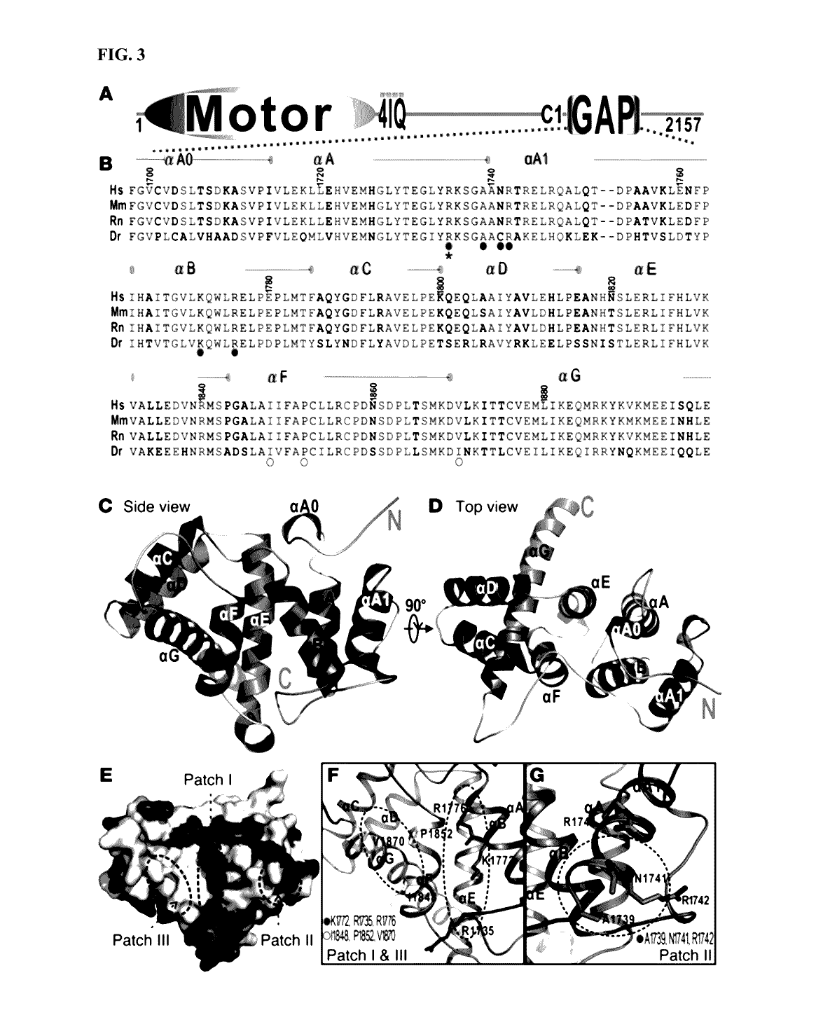

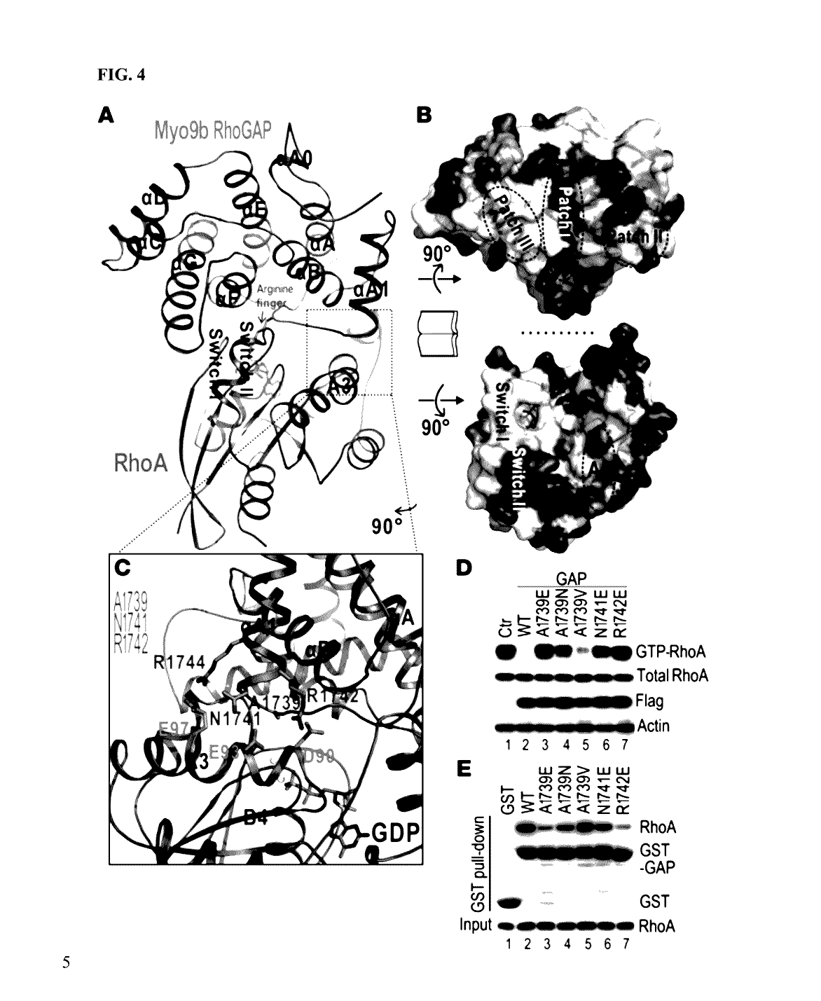

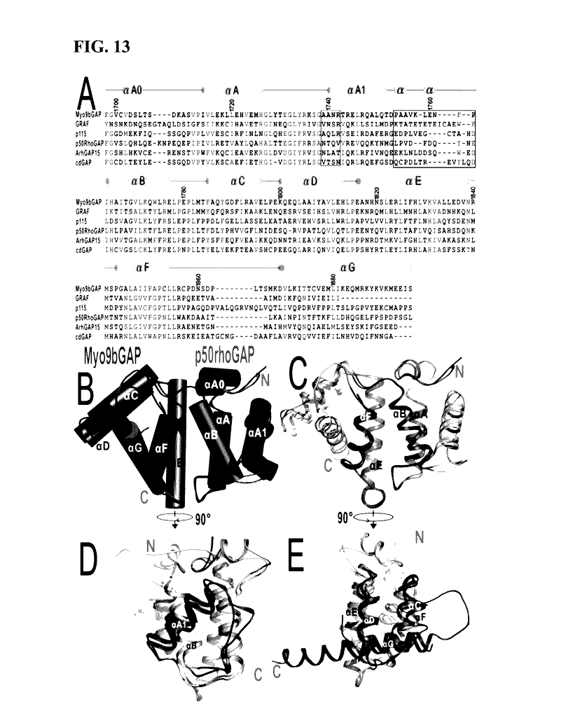

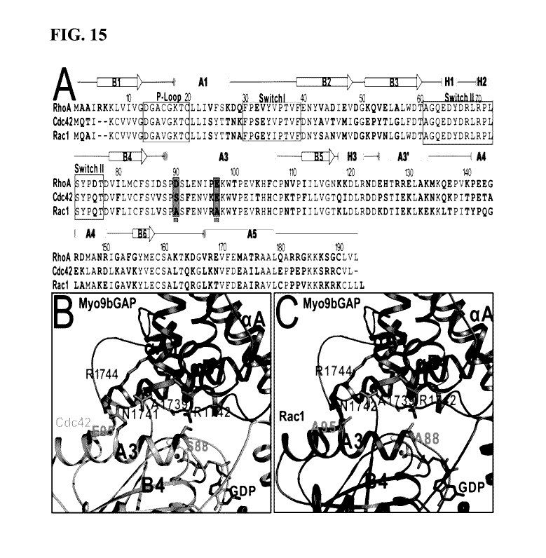

Myo9b contains a RhoGAP domain at its carboxyl terminus (FIG. 3A) that has been reported to inactivate RhoA but not Cdc42 or Rac1 (Muller, 1997; herein incorporated by reference in its entirety). However, the molecular mechanism underlying the specific GAP activity of this RhoGAP domain toward RhoA remained unclear. Experiments were conducted during development of embodiments herein to determine the structure of Myo9b RhoGAP domain (FIG. 3). In comparison with other RhoGAP domains, Myo9b RhoGAP domain contains a unique positively charged Patch II that can specifically recognize the negatively charged A3 helix of RhoA (FIGS. 4 and 13). Mutations in this Patch II region decrease the binding of Myo9b RhoGAP domain to RhoA and impaired the subsequent RhoA inactivation (FIG. 4). Moreover, the corresponding interaction site in Cd42 or Rac1 for Patch II is different from that in RhoA and would not be well recognized by Myo9b RhoGAP domain (FIG. 15). Consistent with previous data (Jelen, 2009; herein incorporated by reference in its entirety), the specificity of the RhoGAP domain for Rho GTPases is most likely determined by the interaction between Patch II and the A3 helix. Data reveal a structural basis for the RhoA-specific GAP activity of Myo9b GAP domain. The paradigm in which the unique positively charged Patch II identified in Myo9b RhoGAP domain specifically recognizes RhoA may also be extended to other proteins containing such RhoA-specific RhoGAP domains.

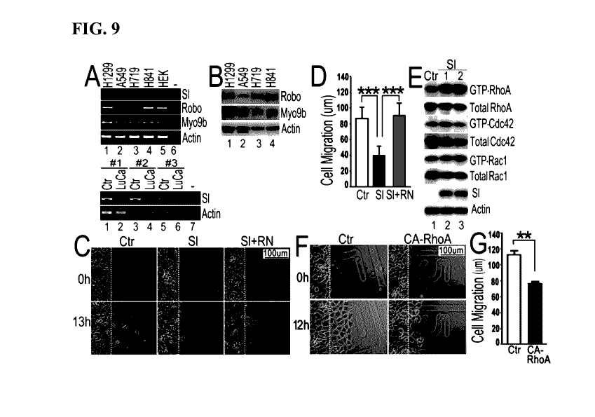

The Rho family of GTPases plays important roles in cell migration by modulating actin and microtubule dynamics, myosin activity, cell-extracellular matrix and cell-cell interactions (Heasman, 2008; Parsons, 2010; Ridley, 2011; herein incorporated by reference in their entireties). The roles of RhoA in cancer cell invasion and migration are highly complex. RhoA is capable of mediating stress fiber formation and generating contractile force needed for retraction of the trailing edge during cell migration (Ridley, 2011; Besson, 2004; herein incorporated by reference in their entireties). RhoA was also reported to function in membrane ruffling and lamellae formation (Kurokawa, 2005; herein incorporated by reference in its entirety). However, the expression and function of RhoA in lung cancer remain unclear. Data herein showes a constitutively active RhoA inhibits migration in lung cancer cells, indicating that activated RhoA suppresses lung cancer cell migration (FIGS. 9, F and G). Increased Slit2 expression or Slit2 treatment leads to RhoA activation in lung cancer cells. Lung cancer cells expressing Slit2 show significantly reduced cell migration in vitro and decreased cancer invasion and metastasis in vivo. These findings indicate that Slit2 activates RhoA signaling to inhibit lung cancer cell migration. miR-194 suppresses metastasis of non-small cell lung cancer through activation of RhoA pathway, producing enhanced development of actin stress fibers and impaired migration of cancer cells (Wu, 2014; herein incorporated by reference in its entirety). Ablation of p120-catenin enhances invasion and metastasis of human lung cancer cells by inactivating RhoA (Liu, 2009; herein incorporated by reference in its entirety). It is suggested that RhoA activity may have a tumor suppressive role in the diffuse gastric cancer and T cell lymphoma (Cools, 2014; Kakiuchi, 2014; Palomero, 2014; Sakata-Yanagimoto, 2014; Wang, 2014; Yoo, 2014; Zhou, 2014; herein incorporated by reference in their entireties).

Experiments conducted during development of embodiments herein indicate a model for the Slit-Robo-Myo9b-RhoA pathway in mediating Slit2 inhibitory effect on lung cancer cell migration (FIG. 8). Slit2 acts to suppress lung cancer cell migration in a Robo-dependent manner. The intracellular domain of Robo1 binds to the RhoGAP domain of Myo9b, resulting in the suppression of its GAP activity and leading to an increased level of GTP-RhoA. These data uncover a previously unknown role of Myo9b lung cancer.

Slit-Robo-srGAP-Cdc42 pathway plays a major role in neurons (Wong, 2001; herein incorporated by reference in its entirety); whereas USP33 is required for Slit-Robo signaling in commissural neurons and breast cancer cells (Yuasa-Kawada, Proc Natl Acad Sci, 2009; Yuasa-Kawada, Nat Neurosci, 2009; herein incorporated by reference in their entireties). Data herein demonstrate an important role of Slit-Robo-Myo9b-RhoA in lung cancer. The observation that Myo9b expression is increased in multiple cohorts of lung cancer samples also suggests Myo9b as a potential therapeutic target for lung cancer. It is conceivable that reducing/silencing Myo9b expression or blocking its activity in lung cancer cells may provide therapeutic benefits for patients suffering from metastatic lung cancer who show increased Myo9b expression. Provided herein is a previously unrecognized signal transduction pathway for Slit in suppressing lung cancer invasion and metastasis that involves Robo-Myo9b-RhoA.

In some embodiments, methods comprise comparing a biomarker (e.g., Myo9 (e.g., Myo9b, Myo9a); Slit (e.g., Slit1, Slit2, Slit3); Robo (e.g., Robo1, Robo2, Robo3, Robo4); RhoA; or another Slit-Robo-Myo9-RhoA pathway gene) level to a reference value/range or a threshold. In some embodiments, deviation of the biomarker(s) level from the reference value/range, or exceeding or failing to meet the threshold, is indicative of a diagnosis, prognosis, etc. for the subject.

In any of the embodiments described herein, each biomarker may be a protein biomarker. In any of the embodiments described herein, the method may comprise contacting biomarkers of the sample from the subject with a set of biomarker capture reagents, wherein each biomarker capture reagent of the set of biomarker capture reagents specifically binds to a biomarker being detected. In some embodiments, each biomarker capture reagent of the set of biomarker capture reagents specifically binds to a different biomarker being detected. In any of the embodiments described herein, each biomarker capture reagent may be an antibody or an aptamer. In some embodiments, the antibody that is used for diagnostic and/or prognostic technologies is the same or similar to an antibody described herein for use as a therapeutic (or a fragment, derivative, or modification of such an antibody).

In some embodiments, a biomarker is an RNA transcript. In any of the embodiments described herein, the method may comprise contacting biomarkers of the sample from the subject with a set of biomarker capture reagents, wherein each biomarker capture reagent of the set of biomarker capture reagents specifically binds to a biomarker being detected. In some embodiments, each biomarker capture reagent of the set of biomarker capture reagents specifically binds to a different biomarker being detected. In any of the embodiments described herein, each biomarker capture reagent may be a nucleic acid probe.

In any of the embodiments described herein, methods further comprise treating the subject for cancer. In some embodiments, treating the subject for cancer comprises a treatment regimen of administering one or more of a chemotherapeutic, radiation, surgery, etc. In some embodiments, biomarkers described herein are monitored before, during, and/or after treatment.

In some embodiments, Slit-Robo-Myo9-RhoA pathway genes provide biomarkers for the diagnosis and/or prognosis of cancer. In some embodiments, such biomarkers, and/or panels thereof (e.g., alone or with other biomarkers) have utility as cancer biomarkers. In some embodiments, such biomarkers are capable of discriminating cancer that is treatable and/or has a low to moderate likelihood of causing mortality from cancer that is unlikely to be responsive to one or more treatments or has a high likelihood of mortality.

In some embodiments, a biomarker detection/quantification assay is performed along with one or more additional assays in order to evaluate cancer in a subject (e.g., provide a prognosis). In some embodiments, a biomarker panel comprises 2, 3, 4, 5, 6, 7, 8, 9, 10, 11, 12, 13, 14, 15, 16, 17, 18, 19, 20 . . . 30 . . . 40, or more biomarkers. In some embodiments, a biomarker panel comprises fewer than 100 biomarkers (e.g., <100, <90, <80, <70, <60, <50, <40, <30, <20, <10, <5). In some embodiments, the number and identity of biomarkers in a panel are selected based on the sensitivity and specificity for the particular combination of biomarker values. The terms “sensitivity” and “specificity” are used herein with respect to the ability to correctly classify an individual, based on one or more biomarker levels detected in a biological sample. “Sensitivity” indicates the performance of the biomarker(s) with respect to correctly classifying individuals having cancer that is likely nonresponsive to treatment or has a high likelihood of causing mortality. “Specificity” indicates the performance of the biomarker(s) with respect to correctly classifying individuals who do not have cancer that is likely nonresponsive to treatment or has a high likelihood of causing mortality. For example, 85% specificity and 90% sensitivity for a panel of markers used to test a set of control samples and test samples indicates that 85% of the control samples were correctly classified as control samples by the panel, and 90% of the test samples were correctly classified as test samples by the panel.

In some embodiments, methods comprise contacting a sample or a portion of a sample from a subject with at least one detection/capture reagent, wherein each capture reagent specifically binds a biomarker (e.g., protein, nucleic acid, etc.) whose presence and/or level is being detected. In some embodiments, capture reagents are antibodies, aptamers, probes, etc. In some embodiments, a method comprises detecting the level of a first biomarker (or panel of biomarkers) by contacting a sample with detection and/or capture reagents specific for that biomarker and then detection one or more additional biomarkers.

In addition to testing biomarker levels as a stand-alone diagnostic/prognostic test, in some embodiments, biomarker levels are tested in conjunction with other markers or assays that are indicative of a particular cancer diagnosis/prognosis (e.g., imaging, biopsy, etc.). In addition to testing biomarker levels, information regarding the biomarkers may also be evaluated in conjunction with other types of data, particularly data that indicates an individual's risk for cancer (e.g., lifestyle, genetics, age, etc.). These various data can be assessed by automated methods, such as a computer program/software, which can be embodied in a computer or other apparatus/device.

The presence of a biomarker or a biomarker level for the biomarkers described herein can be detected using any of a variety of analytical methods. In one embodiment, a biomarker level is detected using a capture reagent. In various embodiments, the capture reagent is exposed to the biomarker in solution or is exposed to the biomarker while the capture reagent is immobilized on a solid support. In other embodiments, the capture reagent contains a feature that is reactive with a secondary feature on a solid support. In these embodiments, the capture reagent is exposed to the biomarker in solution, and then the feature on the capture reagent is used in conjunction with the secondary feature on the solid support to immobilize the biomarker on the solid support. The capture reagent is selected based on the type of analysis to be conducted. Capture reagents include but are not limited to aptamers, antibodies, adnectins, ankyrins, other antibody mimetics and other protein scaffolds, autoantibodies, chimeras, small molecules, F(ab′)2 fragments, single chain antibody fragments, Fv fragments, single chain Fv fragments, nucleic acids, lectins, ligand-binding receptors, affybodies, nanobodies, imprinted polymers, avimers, peptidomimetics, hormone receptors, cytokine receptors, and synthetic receptors, and modifications and fragments of these.

In some embodiments, biomarker presence or level is detected using a biomarker/capture reagent complex. In some embodiments, the biomarker presence or level is derived from the biomarker/capture reagent complex and is detected indirectly, such as, for example, as a result of a reaction that is subsequent to the biomarker/capture reagent interaction, but is dependent on the formation of the biomarker/capture reagent complex.

In some embodiments, biomarker presence or level is detected directly from the biomarker in a biological sample.

In some embodiments, biomarkers are detected using a multiplexed format that allows for the simultaneous detection of two or more biomarkers in a biological sample. In some embodiments of the multiplexed format, capture reagents are immobilized, directly or indirectly, covalently or non-covalently, in discrete locations on a solid support. In some embodiments, a multiplexed format uses discrete solid supports where each solid support has a unique capture reagent associated with that solid support, such as, for example quantum dots. In some embodiments, an individual device is used for the detection of each one of multiple biomarkers to be detected in a biological sample. Individual devices are configured to permit each biomarker in the biological sample to be processed simultaneously. For example, a microtiter plate can be used such that each well in the plate is used to analyze one or more of multiple biomarkers to be detected in a biological sample.

In one or more of the foregoing embodiments, a fluorescent tag is used to label a component of the biomarker/capture reagent complex to enable the detection of the biomarker level. In various embodiments, the fluorescent label is conjugated to a capture reagent specific to any of the biomarkers described herein using known techniques, and the fluorescent label is then used to detect the corresponding biomarker level. Suitable fluorescent labels include rare earth chelates, fluorescein and its derivatives, rhodamine and its derivatives, dansyl, allophycocyanin, PBXL-3, Qdot 605, Lissamine, phycoerythrin, Texas Red, and other such compounds.

In some embodiments, the detection method includes an enzyme/substrate combination that generates a detectable signal that corresponds to the biomarker level (e.g., using the techniques of ELISA, Western blotting, isoelectric focusing). Generally, the enzyme catalyzes a chemical alteration of the chromogenic substrate which can be measured using various techniques, including spectrophotometry, fluorescence, and chemiluminescence. Suitable enzymes include, for example, luciferases, luciferin, malate dehydrogenase, urease, horseradish peroxidase (HRPO), alkaline phosphatase, beta-galactosidase, glucoamylase, lysozyme, glucose oxidase, galactose oxidase, and glucose-6-phosphate dehydrogenase, uricase, xanthine oxidase, lactoperoxidase, microperoxidase, and the like.

In some embodiments, the biomarker levels for the biomarkers described herein are detected using any analytical methods including, singleplex aptamer assays, multiplexed aptamer assays, singleplex or multiplexed immunoassays, mRNA expression profiling, miRNA expression profiling, mass spectrometric analysis, histological/cytological methods, etc. as discussed below.

Immunoassay methods are based on the reaction of an antibody to its corresponding target or analyte and can detect the analyte in a sample depending on the specific assay format. To improve specificity and sensitivity of an assay method based on immuno-reactivity, monoclonal antibodies and fragments thereof are often used because of their specific epitope recognition. Polyclonal antibodies have also been successfully used in various immunoassays because of their increased affinity for the target as compared to monoclonal antibodies. Immunoassays have been designed for use with a wide range of biological sample matrices. Immunoassay formats have been designed to provide qualitative, semi-quantitative, and quantitative results.

Quantitative results are generated through the use of a standard curve created with known concentrations of the specific analyte to be detected. The response or signal from an unknown sample is plotted onto the standard curve, and a quantity or level corresponding to the target in the unknown sample is established.

Numerous immunoassay formats have been designed. ELISA or EIA can be quantitative for the detection of an analyte. This method relies on attachment of a label to either the analyte or the antibody and the label component includes, either directly or indirectly, an enzyme. ELISA tests may be formatted for direct, indirect, competitive, or sandwich detection of the analyte. Other methods rely on labels such as, for example, radioisotopes (I¹²⁵) or fluorescence. Additional techniques include, for example, agglutination, nephelometry, turbidimetry, Western blot, immunoprecipitation, immunocytochemistry, immunohistochemistry, flow cytometry, Luminex assay, and others (see ImmunoAssay: A Practical Guide, edited by Brian Law, published by Taylor & Francis, Ltd., 2005 edition; herein incorporated by reference in its entirety).

Exemplary assay formats include enzyme-linked immunosorbent assay (ELISA), radioimmunoassay, fluorescent, chemiluminescence, and fluorescence resonance energy transfer (FRET) or time resolved-FRET (TR-FRET) immunoassays. Examples of procedures for detecting biomarkers include biomarker immunoprecipitation followed by quantitative methods that allow size and peptide level discrimination, such as gel electrophoresis, capillary electrophoresis, planar electrochromatography, and the like.

Any of the methods for detection can be performed in any format that allows for any suitable preparation, processing, and analysis of the reactions. This can be, for example, in multi-well assay plates (e.g., 96 wells or 384 wells) or using any suitable array or microarray. Stock solutions for various agents can be made manually or robotically, and all subsequent pipetting, diluting, mixing, distribution, washing, incubating, sample readout, data collection and analysis can be done robotically using commercially available analysis software, robotics, and detection instrumentation capable of detecting a detectable label.

Measuring mRNA in a biological sample may, in some embodiments, be used as a surrogate for detection of the level of a corresponding protein in the biological sample. Thus, in some embodiments, a biomarker or biomarker panel described herein can be detected by detecting the appropriate RNA.

In some embodiments, mRNA expression levels are measured, e.g., to assess expression of a member of the Slit-Robo-Myo9-RhoA pathway (e.g., Myo9 (e.g., Myo9b, Myo9a); Slit (e.g., Slit1, Slit2, Slit3); Robo (e.g., Robo1, Robo2, Robo3, Robo4); RhoA; another Slit-Robo-Myo9-RhoA pathway gene; or another gene or gene product that modulates the Slit-Robo-Myo9-RhoA pathway). In some embodiments, mRNA expression (e.g., transcript absolute quantity and/or transcript relative quantity, e.g., relative to a reference and/or a control) is measured by reverse transcription quantitative polymerase chain reaction (RT-PCR followed with qPCR). RT-PCR is used to create a cDNA from the mRNA. The cDNA may be used in a qPCR assay to produce fluorescence as the DNA amplification process progresses. By comparison to a standard curve, qPCR can produce an absolute measurement such as number of copies of mRNA per cell. Embodiments provide that mRNA levels are quantified for isoforms and differently spliced forms of the RNA transcripts.

Myo9b has increased expression in a significant fraction of cancer (e.g., greater that 70% of lung cancers and greater than 63% of pancreatic cancer). Accordingly, qPCR assays find use, e.g., in measuring the levels of Myo9 (e.g., Myo9b) messenger RNAs, including differently spliced forms of Myo9, as a biomarker for pancreatic and/or lung cancer.

In additional embodiments, northern blots, microarrays, RNAseq, Invader assays, and/or RT-PCR combined with capillary electrophoresis is/are used to measure expression levels of mRNA in a sample. See Gene Expression Profiling: Methods and Protocols, Richard A. Shimkets, editor, Humana Press, 2004; herein incorporated by reference in its entirety.

In some embodiments, microRNA, pre-miRNA, and/or pri-miRNA expression levels are measured, e.g., to assess expression of a non-coding microRNA gene that modulates one or more members of the Slit-Robo-Myo9-RhoA pathway (e.g., Myo9 (e.g., Myo9b, Myo9a); Slit (e.g., Slit1, Slit2, Slit3); Robo (e.g., Robo1, Robo2, Robo3, Robo4); RhoA; another Slit-Robo-Myo9-RhoA pathway gene; or another gene or gene product that modulates the Slit-Robo-Myo9-RhoA pathway). In some embodiments, microRNA expression (e.g., transcript absolute quantity and/or transcript relative quantity, e.g., relative to a reference and/or a control) is measured by reverse transcription quantitative polymerase chain reaction (RT-PCR followed with qPCR). RT-PCR is used to create a cDNA from the microRNA. The cDNA may be used in a qPCR assay to produce fluorescence as the DNA amplification process progresses. By comparison to a standard curve, qPCR can produce an absolute measurement such as number of copies of microRNA per cell. Embodiments provide that microRNA levels are quantified for isoforms and differently spliced forms of a pre-microRNA, a pri-microRNA, and/or a microRNA.

In some embodiments, a biomarker described herein may be used in molecular imaging tests. For example, an imaging agent can be coupled to a capture reagent, which can be used to detect the biomarker in vivo.

In vivo imaging technologies provide non-invasive methods for determining the state of a particular disease in the body of an individual. For example, entire portions of the body, or even the entire body, may be viewed as a three dimensional image, thereby providing valuable information concerning morphology and structures in the body. Such technologies may be combined with the detection of the biomarkers described herein to provide information concerning the biomarker in vivo.

Advances in the use of in vivo molecular imaging technologies include the development of new contrast agents or labels, such as radiolabels and/or fluorescent labels, which can provide strong signals within the body; and the development of powerful new imaging technology, which can detect and analyze these signals from outside the body, with sufficient sensitivity and accuracy to provide useful information. The contrast agent can be visualized in an appropriate imaging system, thereby providing an image of the portion or portions of the body in which the contrast agent is located. The contrast agent may be bound to or associated with a capture reagent, with a peptide or protein, or an oligonucleotide (for example, for the detection of gene expression), or a complex containing any of these with one or more macromolecules and/or other particulate forms.

In some embodiments, the biomarkers described herein may be detected in a variety of tissue samples using histological or cytological methods. In some embodiments, one or more capture reagent/s specific to the corresponding biomarker/s are used in a cytological evaluation of a sample and may include one or more of the following: collecting a cell sample, fixing the cell sample, dehydrating, clearing, immobilizing the cell sample on a microscope slide, permeabilizing the cell sample, treating for analyte retrieval, staining, destaining, washing, blocking, and reacting with one or more capture reagent/s in a buffered solution. In another embodiment, the cell sample is produced from a cell block.

In some embodiments, one or more capture reagent/s specific to the corresponding biomarkers are used in a histological evaluation of a tissue sample and may include one or more of the following: collecting a tissue specimen, fixing the tissue sample, dehydrating, clearing, immobilizing the tissue sample on a microscope slide, permeabilizing the tissue sample, treating for analyte retrieval, staining, destaining, washing, blocking, rehydrating, and reacting with capture reagent/s in a buffered solution. In another embodiment, fixing and dehydrating are replaced with freezing.

In some embodiments, a biomarker “signature” for a given diagnostic or prognostic test contains one or more biomarkers (e.g., a set of markers), each marker having characteristic levels in the populations of interest. Characteristic levels, in some embodiments, may refer to the mean or average of the biomarker levels for the individuals in a particular group. In some embodiments, a diagnostic/prognostic method described herein can be used to assign an unknown sample from an individual into one of two or more groups: high risk cancer, lower risk cancer, treatment-responsive cancer, treatment-unresponsive cancer, healthy, etc. The assignment of a sample into one of two or more groups is known as classification, and the procedure used to accomplish this assignment is known as a classifier or a classification method. Classification methods may also be referred to as scoring methods. There are many classification methods that can be used to construct a diagnostic classifier from a set of biomarker levels. In some instances, classification methods are performed using supervised learning techniques in which a data set is collected using samples obtained from individuals within two (or more, for multiple classification states) distinct groups one wishes to distinguish. Since the class (group or population) to which each sample belongs is known in advance for each sample, the classification method can be trained to give the desired classification response. It is also possible to use unsupervised learning techniques to produce a diagnostic classifier.

In some embodiments, the results are analyzed and/or reported (e.g., to a patient, clinician, researcher, investigator, etc.). Results, analyses, and/or data (e.g., signature, disease score, diagnosis, recommended course, etc.) are identified and/or reported as an outcome/result of an analysis. A result may be produced by receiving or generating data (e.g., test results) and transforming the data to provide an outcome or result. An outcome or result may be determinative of an action to be taken. In some embodiments, results determined by methods described herein can be independently verified by further or repeat testing.

In some embodiments, analysis results are reported (e.g., to a health care professional (e.g., laboratory technician or manager; physician, nurse, or assistant, etc.), patient, researcher, investigator, etc.). In some embodiments, a result is provided on a peripheral, device, or component of an apparatus. For example, sometimes an outcome is provided by a printer or display. In some embodiments, an outcome is reported in the form of a report. Generally, an outcome can be displayed in a suitable format that facilitates downstream use of the reported information.

Generating and reporting results from the methods described herein comprises transformation of biological data (e.g., presence or level of biomarkers) into a representation of the characteristics of a subject (e.g., likelihood of mortality, likelihood corresponding to treatment, etc.). Such a representation reflects information not determinable in the absence of the method steps described herein. Converting biologic data into understandable characteristics of a subject allows actions to be taken in response such information.

In some embodiments, a downstream individual (e.g., clinician, patient, etc.), upon receiving or reviewing a report comprising one or more results determined from the analyses provided herein, will take specific steps or actions in response. For example, a decision about whether or not to treat the subject, and/or how to treat the subject is made.

The term “receiving a report” as used herein refers to obtaining, by a communication means, a written and/or graphical representation comprising results or outcomes of analysis. The report may be generated by a computer or by human data entry, and can be communicated using electronic means (e.g., over the internet, via computer, via fax, from one network location to another location at the same or different physical sites), or by another method of sending or receiving data (e.g., mail service, courier service and the like). In some embodiments the outcome is transmitted in a suitable medium, including, without limitation, in verbal, document, or file form. The file may be, for example, but not limited to, an auditory file, a computer readable file, a paper file, a laboratory file or a medical record file. A report may be encrypted to prevent unauthorized viewing.

As noted above, in some embodiments, systems and method described herein transform data from one form into another form (e.g., from biomarker levels to diagnostic/prognostic determination, etc.). In some embodiments, the terms “transformed”, “transformation”, and grammatical derivations or equivalents thereof, refer to an alteration of data from a physical starting material (e.g., biological sample, etc.) into a digital representation of the physical starting material (e.g., biomarker levels), a condensation/representation of that starting material (e.g., risk level), or a recommended action (e.g., treatment, no treatment, etc.).

Any combination of the biomarkers described herein (e.g., one or more members of the Slit-Robo-Myo9-RhoA pathway (e.g., Myo9 (e.g., Myo9b, Myo9a); Slit (e.g., Slit1, Slit2, Slit3); Robo (e.g., Robo1, Robo2, Robo3, Robo4); RhoA; another Slit-Robo-Myo9-RhoA pathway gene; or another gene or gene product that modulates the Slit-Robo-Myo9-RhoA pathway)) can be detected using a suitable kit, such as for use in performing the methods disclosed herein. The biomarkers described herein may be combined in any suitable combination, or may be combined with other markers not described herein. Furthermore, any kit can contain one or more detectable labels as described herein, such as a fluorescent moiety, etc.

In some embodiments, a kit includes (a) one or more capture reagents for detecting one or more biomarkers in a biological sample, and optionally (b) one or more software or computer program products for providing a diagnosis/prognosis for the individual from whom the biological sample was obtained. Alternatively, rather than one or more computer program products, one or more instructions for manually performing the above steps by a human can be provided.

In some embodiments, a kit comprises a solid support, a capture reagent, and a signal generating material. The kit can also include instructions for using the devices and reagents, handling the sample, and analyzing the data. Further the kit may be used with a computer system or software to analyze and report the result of the analysis of the biological sample.

The kits can also contain one or more reagents (e.g., solubilization buffers, detergents, washes, or buffers) for processing a biological sample. Any of the kits described herein can also include, e.g., buffers, blocking agents, mass spectrometry matrix materials, serum/plasma separators, antibody capture agents, positive control samples, negative control samples, software and information such as protocols, guidance and reference data.

In some embodiments, following a determination that a subject has suffers from cancer, the subject is appropriately treated. In some embodiments, therapy is administered to treat cancer. In some embodiments, therapy is administered to treat complications of cancer (e.g., surgery, radiation, chemotherapy). In some embodiments, treatment comprises palliative care.

Methods of treatment comprise, e.g., methods that inhibit and/or activate one or more members of the Slit-Robo-Myo9-RhoA pathway (e.g., Myo9 (e.g., Myo9b, Myo9a); Slit (e.g., Slit1, Slit2, Slit3); Robo (e.g., Robo1, Robo2, Robo3, Robo4); RhoA; another Slit-Robo-Myo9-RhoA pathway gene; or another gene or gene product that modulates the Slit-Robo-Myo9-RhoA pathway). In some embodiments, the technology relates to modulating (e.g., increasing) expression of Myo9b suppressor genes such as Slit (e.g., Slit1, Slit2, Slit3) and Robo (e.g., Robo1, Robo2, Robo3, Robo4) to suppress Myo9b activity. For example, EZH2 inhibitors may increase Slit2 expression in prostate cancer cells (Yu J et al, 2010 Oncogene), in colorectal cancer cells (Huang Z et al, 2015, Int. J. Ca), in lung cancer cells (Kong et al, 2015, JCI), pancreatic cancer cells (Göhrig A et a, 2014; Ca Reserach), breast cancer cells (Yuasa-Kawada et al, 2009, PNAS), medulloblastoma cancer cells (Werbowetski-Ogilvie et al, 2006, Oncogene), and glioblasoma cancer cells (Yiin et al, 2010).

In some embodiments the technology provides a method for inhibiting Myo9. In some embodiments, inhibiting Myo9 finds use to treat or study diseases involving aberrant activation of Myo9 or involving aberrant activity of other genes of gene products that is remedied by inhibiting the activity of Myo9.

In some embodiments, inhibiting the Myo9 activity is accomplished by means of an antibody that recognizes Myo9. In some embodiments, the anti-Myo9 antibody is a monoclonal antibody and in some embodiments the anti-Myo9 antibody is a polyclonal antibody. In some embodiments, the anti-Myo9 antibody is, for example, a human, humanized, or chimeric antibody. Monoclonal antibodies against target antigens are produced by a variety of techniques including conventional monoclonal antibody methodologies such as the somatic cell hybridization techniques of Köhler and Milstein (Nature, 256:495 (1975)). Although in some embodiments, somatic cell hybridization procedures are preferred, other techniques for producing monoclonal antibodies are contemplated as well (e.g., viral or oncogenic transformation of B lymphocytes). Embodiments are also provided in which an antibody modulates the activity of one or more members of the Slit-Robo-Myo9-RhoA pathway (e.g., Myo9 (e.g., Myo9b, Myo9a); Slit (e.g., Slit1, Slit2, Slit3); Robo (e.g., Robo1, Robo2, Robo3, Robo4); RhoA; another Slit-Robo-Myo9-RhoA pathway gene; or another gene or gene product that modulates the Slit-Robo-Myo9-RhoA pathway).

It is contemplated that antibodies specific for Myo9 find use in the experimental, diagnostic, and therapeutic methods described herein. In certain embodiments, the antibodies provided herein are used to detect the expression of Myo9 in biological samples. For example, a sample (e.g., comprising a tissue biopsy) can be sectioned and protein detected using, for example, immunofluorescence or immunohistochemistry. Alternatively, individual cells from a sample can be isolated, and protein expression detected on fixed or live cells by FACS analysis. Furthermore, the antibodies can be used on protein arrays to detect expression of Myo9. In other embodiments, the antibodies provided herein are used to increase the activity of RhoA by inhibiting Myo9 either in an in vitro cell-based assay or in an in vivo animal model. In some embodiments, antibodies are used to treat a human patient by administering a therapeutically effective amount of an antibody against Myo9 (e.g., an antibody that is specific for Myo9).

For the production of antibodies, various host animals can be immunized by injection with a Myo9 protein, a fragment of a Myo9 protein, and/or a Myo9 peptide (e.g., corresponding to a desired Myo9 epitope (e.g., a fragment of Myo9)). Appropriate host animals include, but are not limited to, rabbits, mice, rats, sheep, goats, etc. Antibodies to Myo9 can be raised by immunizing a host animal (e.g., by injection) with an antigen comprising a peptide, a portion, or the full protein of Myo9 (e.g., a protein or peptide fragment of Myo9) or a variant or modified version thereof. Antibodies can also be raised by immunization with a translation product of the gene encoding Myo9, e.g., MYO9A and/or MYO9B or variants or fragments thereof.

In some embodiments, the peptide is conjugated to an immunogenic carrier (e.g., diphtheria toxoid, bovine serum albumin (BSA), or keyhole limpet hemocyanin (KLH)). Various adjuvants are used to increase the immunological response, depending on the host species, including, but not limited to, Freund's (complete and incomplete), mineral gels such as aluminum hydroxide, surface active substances such as lysolecithin, pluronic polyols, polyanions, peptides, oil emulsions, keyhole limpet hemocyanins, dinitrophenol, and potentially useful human adjuvants such as BCG (Bacille Calmette-Guerin) and Corynebacterium parvum.

Some embodiments relate to polyclonal antibodies. Polyclonal antibodies can be prepared by any known method. Polyclonal antibodies can be raised by immunizing an animal (e.g., a rabbit, rat, mouse, donkey, etc) by multiple subcutaneous or intraperitoneal injections of the relevant antigen (a purified peptide fragment, full-length recombinant protein, fusion protein, etc.) optionally conjugated to KLH, serum albumin, etc., diluted in sterile saline, and combined with an adjuvant to form a stable emulsion. The polyclonal antibody is then recovered from blood, ascites, and the like, of an animal so immunized. Collected blood is clotted, and the serum decanted, clarified by centrifugation, and assayed for antibody titer. The polyclonal antibodies can be purified from serum or ascites according to standard methods in the art including affinity chromatography, ion-exchange chromatography, gel electrophoresis, dialysis, etc.

For preparation of monoclonal antibodies, any technique that provides for the production of antibody molecules by continuous cell lines in culture may be used (see e.g., Harlow and Lane, Antibodies: A Laboratory Manual, Cold Spring Harbor Laboratory Press, Cold Spring Harbor, N.Y.). Methods for production of monoclonal antibodies include, but are not limited to, the hybridoma technique originally developed by Köhler and Milstein and the trioma technique, the human B-cell hybridoma technique (See, e.g., Kozbor et al., Immunol. Today, 4:72 (1983)), and the EBV-hybridoma technique to produce human monoclonal antibodies (Cole et al., in Monoclonal Antibodies and Cancer Therapy, Alan R. Liss, Inc., pp. 77-96 (1985)).

In some embodiments provided herein, the antibodies are prepared from a hybridoma. Using the hybridoma method, a mouse, hamster, or other appropriate host animal, is immunized as described above to elicit the production by lymphocytes of antibodies that specifically bind to an immunizing antigen. Alternatively, lymphocytes can be immunized in vitro. Following immunization, the lymphocytes are isolated and fused with a suitable myeloma cell line using, for example, polyethylene glycol, to form hybridoma cells that can then be selected away from unfused lymphocytes and myeloma cells. Hybridomas that produce monoclonal antibodies directed specifically against a chosen antigen as determined by immunoprecipitation, immunoblotting, or by an in vitro binding assay such as radioimmunoassay (RIA) or enzyme-linked immunosorbent assay (ELISA) are then propagated in vitro (e.g., in culture) using standard methods (Goding, Monoclonal Antibodies: Principles and Practice, Academic Press, 1986) or in vivo as ascites tumors in an animal. The monoclonal antibodies can then be purified from the culture medium or ascites fluid as described for polyclonal antibodies above.

The preferred animal system for preparing hybridomas is the murine system. Hybridoma production in the mouse is a well-established procedure. Immunization protocols and techniques for isolation of immunized splenocytes for fusion are known in the art. Fusion partners (e.g., murine myeloma cells) and fusion procedures are also known. Embodiments of the technology herein provide antibodies (e.g., monoclonal antibodies) produced from a hybridoma prepared by immunizing mice with a peptide that is a portion or fragment of the Myo9 protein. For example, some embodiments provide an antibody or antigen-binding fragment than binds to Myo9 by immunizing with, e.g., a protein or peptide fragment of the Myo9 protein or a variant or modified version thereof. Some embodiments provide an antibody or antigen-binding fragment that binds to a protein or peptide, or variants or modified versions thereof, that is a translation product of the MYO9A gene or the MYO9B gene or variants or fragments thereof.

For example, embodiments of the technology herein provide monoclonal antibodies produced from a hybridoma prepared by immunizing mice with a peptide derived from Myo9. Also contemplated are methods and compositions related to antibodies prepared using a variant of a peptide derived from Myo9 and comprising one or more substitutions, deletions, insertions, or other changes, as long as said variant produces an antibody specific for Myo9. Producing polypeptides derived from Myo9 and similar sequences thereto can be accomplished according to various techniques well known in the art. For example, a polypeptide derived from Myo9 or a variant thereof can be produced using a bacterial expression system and a nucleic acid encoding a polypeptide derived from Myo9 or a variant thereof.

Moreover, human monoclonal antibodies directed against human proteins can be generated using transgenic mice carrying the complete human immune system rather than the mouse system. Splenocytes from the transgenic mice are immunized with the antigen of interest, which are used to produce hybridomas that secrete human monoclonal antibodies with specific affinities for epitopes from a human protein.

Monoclonal antibodies can also be generated by other methods known to those skilled in the art of recombinant DNA technology. For instance, combinatorial antibody display has can be utilized to produce monoclonal antibodies (see, e.g., Sastry et al., Proc. Nat. Acad. Sci. USA, 86: 5728 (1989); Huse et al., Science, 246: 1275 (1989); Orlandi et al., Proc. Nat. Acad. Sci. USA, 86:3833 (1989)). After immunizing an animal with an immunogen as described above, the antibody repertoire of the resulting B-cell pool is cloned. Methods are generally known for obtaining the DNA sequence of the variable regions of a diverse population of immunoglobulin molecules by using a mixture of oligomer primers and PCR. For instance, mixed oligonucleotide primers corresponding to the 5′ leader (signal peptide) sequences and/or framework 1 (FR1) sequences, as well as primers to a conserved 3′ region can be used to amplify and isolate the heavy and light chain variable regions from a number of murine antibodies (see. e.g., Larrick et al., Biotechniques, 11: 152 (1991)). A similar strategy can also been used to amplify human heavy and light chain variable regions from human antibodies (see, e.g., Larrick et al., Methods: Companion to Methods in Enzymology, 2: 106 (1991)).

Alternatively, monoclonal antibodies can also be made using recombinant DNA methods as described in U.S. Pat. No. 4,816,567, incorporated herein by reference. The polynucleotides encoding a monoclonal antibody are isolated (e.g., from mature B-cells or hybridoma cells), by, e.g., RT-PCR using oligonucleotide primers that specifically amplify the genes encoding the heavy and light chains of the antibody, and their sequences are determined using conventional procedures. The isolated polynucleotides encoding the heavy and light chains are then cloned into suitable expression vectors, which, when transfected into host cells such as E. coli cells, simian COS cells, Chinese hamster ovary (CHO) cells, or myeloma cells that do not otherwise produce immunoglobulin protein, cause monoclonal antibodies to be generated by the host cells. Also, recombinant monoclonal antibodies or fragments thereof of the desired species can be isolated from phage display libraries as described (McCafferty et al., 1990, Nature, 348:552-554; Clackson et al., 1991, Nature, 352:624-628; and Marks et al., 1991, J. Mol. Biol., 222:581-597).

The polynucleotide encoding a monoclonal antibody can further be modified in a number of different manners using recombinant DNA technology to generate alternative antibodies. In one embodiment, the constant domains of the light and heavy chains of, for example, a mouse monoclonal antibody can be substituted 1) for those regions of, for example, a human antibody to generate a chimeric antibody or 2) for a non-immunoglobulin polypeptide to generate a fusion antibody. In other embodiments, the constant regions are truncated or removed to generate the desired antibody fragment of a monoclonal antibody. Furthermore, site-directed or high-density mutagenesis of the variable region can be used to optimize specificity, affinity, etc. of a monoclonal antibody.

For example, also contemplated are chimeric mouse-human monoclonal antibodies, which can be produced by recombinant DNA techniques known in the art. For example, a gene encoding the constant region of a murine (or other species) monoclonal antibody molecule is digested with restriction enzymes to remove the region encoding the murine constant region, and the equivalent portion of a gene encoding a human constant region is substituted (see, e.g., Robinson et al., PCT/US86/02269; European Patent Application 184,187; European Patent Application 171,496; European Patent Application 173,494; WO 86/01533; U.S. Pat. No. 4,816,567; European Patent Application 125,023 (each of which is herein incorporated by reference in its entirety); Better et al., Science, 240:1041-1043 (1988); Liu et al., Proc. Nat. Acad. Sci. USA, 84:3439-3443 (1987); Liu et al., J. Immunol., 139:3521-3526 (1987); Sun et al., Proc. Nat. Acad. Sci. USA, 84:214-218 (1987); Nishimura et al., Canc. Res., 47:999-1005 (1987); Wood et al., Nature, 314:446-449 (1985); and Shaw et al., J. Natl. Cancer Inst., 80:1553-1559 (1988)).

The chimeric antibody can be further humanized by replacing sequences of the variable region that are not directly involved in antigen binding with equivalent sequences from human variable regions. General reviews of humanized chimeric antibodies are provided by S. L. Morrison, Science, 229:1202-1207 (1985) and by Oi et al., Bio Techniques, 4:214 (1986). Those methods include isolating, manipulating, and expressing the nucleic acid sequences that encode all or part of immunoglobulin variable regions from at least one of a heavy or light chain. Sources of such nucleic acid are well known to those skilled in the art. The recombinant DNA encoding the chimeric antibody, or fragment thereof, can then be cloned into an appropriate expression vector.

Suitable humanized antibodies can alternatively be produced by CDR substitution (see, e.g., U.S. Pat. No. 5,225,539; Jones et al., Nature, 321:552-525 (1986); Verhoeyan et al., Science, 239:1534 (1988); and Beidler et al., J. Immunol., 141:4053 (1988)). All of the CDRs of a particular human antibody may be replaced with at least a portion of a non-human CDR or only some of the CDRs may be replaced with non-human CDRs. It is only necessary to replace the number of CDRs important for binding of the humanized antibody to the Fc receptor.

An antibody can be humanized by any method that is capable of replacing at least a portion of a CDR of a human antibody with a CDR derived from a non-human antibody. The human CDRs may be replaced with non-human CDRs using oligonucleotide site-directed mutagenesis.

Also contemplated are chimeric and humanized antibodies in which specific amino acids have been substituted, deleted, or added. In particular, preferred humanized antibodies have amino acid substitutions in the framework region, such as to improve binding to the antigen. For example, in a humanized antibody having mouse CDRs, amino acids located in the human framework region can be replaced with the amino acids located at the corresponding positions in the mouse antibody. Such substitutions are known to improve binding of humanized antibodies to the antigen in some instances.

In certain embodiments provided herein, it is desirable to use an antibody fragment. Various techniques are known for the production of antibody fragments. Traditionally, these fragments are derived via proteolytic digestion of intact antibodies (for example Morimoto et al., 1993, Journal of Biochemical and Biophysical Methods 24:107-117 and Brennan et al., 1985, Science, 229:81). For example, papain digestion of antibodies produces two identical antigen-binding fragments, called Fab fragments, each with a single antigen-binding site, and a residual Fc fragment. Pepsin treatment yields an F(ab′)2 fragment that has two antigen-combining sites and is still capable of cross-linking antigen.

However, these fragments are now typically produced directly by recombinant host cells as described above. Thus Fab, Fv, and other antigen-binding antibody fragments can all be expressed in and secreted from E. coli or other host cells, thus allowing the production of large amounts of these fragments. Alternatively, such antibody fragments can be isolated from the antibody phage libraries discussed above. The antibody fragment can also be linear antibodies as described in U.S. Pat. No. 5,641,870, for example, and can be monospecific or bispecific. Other techniques for the production of antibody fragments will be apparent to the skilled practitioner.

Fv is the minimum antibody fragment which contains a complete antigen-recognition and antigen-binding site. This region consists of a dimer of one heavy-chain and one light-chain variable domain in tight, non-covalent association. It is in this configuration that the three CDRs of each variable domain interact to define an antigen-binding site on the surface of the VH-VL dimer. Collectively, the six CDRs confer antigen-binding specificity to the antibody. However, even a single variable domain (or half of an Fv comprising only three CDRs specific for an antigen) has the ability to recognize and bind antigen, although at a lower affinity than the entire binding site.

The Fab fragment also contains the constant domain of the light chain and the first constant domain (CH1) of the heavy chain. Fab fragments differ from Fab′ fragments by the addition of a few residues at the carboxy terminus of the heavy chain CH1 domain including one or more cysteines from the antibody hinge region. F(ab′)2 antibody fragments originally were produced as pairs of Fab′ fragments which have hinge cysteines between them. Other chemical couplings of antibody fragments are also known to the skilled artisan.

The technology herein provided also contemplates modifying an antibody to increase its serum half-life. This can be achieved, for example, by incorporating a salvage receptor binding epitope into the antibody fragment by mutation of the appropriate region in the antibody fragment or by incorporating the epitope into a peptide tag that is then fused to the antibody fragment at either end or in the middle (e.g., by DNA or peptide synthesis).

The technology embraces variants and equivalents which are substantially homologous to the chimeric, humanized, and human antibodies, or antibody fragments thereof, provided herein. These can contain, for example, conservative substitution mutations, e.g., the substitution of one or more amino acids by similar amino acids. For example, conservative substitution refers to the substitution of an amino acid with another within the same general class such as, for example, one acidic amino acid with another acidic amino acid, one basic amino acid with another basic amino acid, or one neutral amino acid by another neutral amino acid. What is intended by a conservative amino acid substitution is well known in the art.

An additional embodiment utilizes the techniques known in the art for the construction of Fab expression libraries (Huse et al., Science, 246:1275-1281 (1989)) to allow rapid and easy identification of monoclonal Fab fragments with the desired specificity.

Also, this technology encompasses bispecific antibodies that specifically recognize Myo9. Bispecific antibodies are antibodies that are capable of specifically recognizing and binding at least two different epitopes. Bispecific antibodies can be intact antibodies or antibody fragments. Techniques for making bispecific antibodies are common in the art (Millstein et al., 1983, Nature 305:537-539; Brennan et al., 1985, Science 229:81; Suresh et al, 1986, Methods in Enzymol. 121:120; Traunecker et al., 1991, EMBO J. 10:3655-3659; Shalaby et al., 1992, J. Exp. Med. 175:217-225; Kostelny et al., 1992, J. Immunol. 148:1547-1553; Gruber et al., 1994, J. Immunol. 152:5368; and U.S. Pat. No. 5,731,168).

Techniques described for the production of single chain antibodies (U.S. Pat. No. 4,946,778; herein incorporated by reference) can be adapted to produce specific single chain antibodies as desired. Single-chain Fv antibody fragments comprise the VH and VL domains of an antibody, wherein these domains are present in a single polypeptide chain. Preferably, the Fv polypeptide further comprises a polypeptide linker between the VH and VL domains that enables the single-chain Fv antibody fragments to form the desired structure for antigen binding. For a review of single-chain Fv antibody fragments, see Pluckthun in The Pharmacology of Monoclonal Antibodies, vol. 113, Rosenburg and Moore eds., Springer-Verlag, New York, pp. 269-315 (1994).

In some embodiments, the technology comprises use of an antibody conjugated to a second moiety, compound, etc. For example, embodiments provide an anti-Myo9 antibody conjugated to a therapeutic agent such as, e.g., a small molecule inhibitor of Mpo9, an anti-Myo9 antibody conjugated to a toxin of Mpo9, etc. Therapeutic agents include, but are not limited to, a radionuclide, a cytotoxin, a chemotherapeutic agent, a drug, a pro-drug, a toxin, an enzyme, an immunomodulator, an anti-angiogenic agent, a pro-apoptotic agent, a cytokine, a hormone, an oligonucleotide molecule (e.g., an antisense molecule or a gene), or a second antibody or fragment thereof. Methods of making immunoconjugates are provided by, e.g., U.S. Pat. Nos. 4,699,784; 4,824,659; 5,525,338; 5,677,427; 5,697,902; 5,716,595; 6,071,490; 6,187,284; 6,306,393; 6,548,275; 6,653,104; 6,962,702; 7,033,572; 7,147,856; and 7,259,240, each incorporated herein by reference. In some embodiments, the toxin is diphtheria toxin, Pseudomonas exotoxin, or Pseudomonas endotoxin. In some embodiments, the radionuclide is ^{103m}Rh, ¹⁰³Ru, ¹⁰⁵Rh, ¹⁰⁵Ru, ¹⁰⁷Hg, ¹⁰⁹Pd, ¹⁰⁹Pt, ¹¹¹Ag, ¹¹¹In, ^{113m}In, ¹¹⁹Sb, ¹¹C, ^{121m}Te, ^{122m}T, ¹²⁵I, ^{125m}Te, ¹²⁶I, ¹³¹I, ¹³³I, ¹³N, ¹⁴²Pr, ¹⁴³Pr, ¹⁴⁹Pm, ¹⁵²Dy, ¹⁵³Sm, ¹⁵O, ¹⁶¹Ho, ¹⁶¹Tb, ¹⁶⁵Tm, ¹⁶⁶Dy, ¹⁶⁶Ho, ¹⁶⁷Tm, ¹⁶⁸Tm, ¹⁶⁹Er, ¹⁶⁹Yb, ¹⁷⁷Lu, ¹⁸⁶Re, ¹⁸⁸Re, ^{189m}Os, ¹⁸⁹Re, ¹⁹²Ir, ¹⁹⁴Ir, ¹⁹⁷Pt, ¹⁹⁸Au, ¹⁹⁹Au, ²⁰¹Tl, ²⁰³Hg, ²¹¹At, ²¹¹Bi, ²¹¹Pb, ²¹²Bi, ²¹²Pb, ²¹³Bi, ²¹⁵Po, ²¹⁷At, ²¹⁹Rn, ²²¹Fr, ²²³Ra, ²²⁴Ac, ²²⁵Ac, ²²⁵Fm, ³²P, ³³P, ⁴⁷Sc, ⁵¹Cr, ⁵⁷Co, ⁵⁸Co, ⁵⁹Fe, ⁶²Cu, ⁶⁷Cu, ⁶⁷Ga, ⁷⁵Br, ⁷⁵Se, ⁷⁶Br, ⁷⁷As, ⁷⁷Br, ^{80m}Br, ⁸⁹Sr, ⁹⁰Y, ⁹⁵Ru, ⁹⁷Ru, ⁹⁹Mo, or ^{99m}Tc. In certain embodiments, the antibody or fragment comprises one or more chelating moieties, such as NOTA, DOTA, DTPA, TETA, Tscg-Cys, or Tsca-Cys. In certain embodiments, the chelating moiety forms a complex with a therapeutic or diagnostic cation, such as Group II, Group III, Group IV, Group V, transition, lanthanide, or actinide metal cations, Tc, Re, Bi, Cu, As, Ag, Au, At, or Pb.

In some embodiments, a nucleic acid is used to modulate one or more members of the Slit-Robo-Myo9-RhoA pathway (e.g., Myo9 (e.g., Myo9b, Myo9a); Slit (e.g., Slit1, Slit2, Slit3); Robo (e.g., Robo1, Robo2, Robo3, Robo4); RhoA; another Slit-Robo-Myo9-RhoA pathway gene; or another gene or gene product that modulates the Slit-Robo-Myo9-RhoA pathway). For example, in some embodiments a small interfering RNA (siRNA) is designed to target and degrade Myo9 mRNA to inhibit the activity of Myo9. siRNAs are double-stranded RNA molecules of 20-25 nucleotides in length. While not limited in their features, typically an siRNA is 21 nucleotides long and has 2-nt 3′ overhangs on both ends. Each strand has a 5′ phosphate group and a 3′ hydroxyl group. In vivo, this structure is the result of processing by Dicer, an enzyme that converts either long dsRNAs or small hairpin RNAs (shRNAs) into siRNAs. However, siRNAs can also be synthesized and exogenously introduced into cells to bring about the specific knockdown of a gene of interest. Essentially any gene of which the sequence is known can be targeted based on sequence complementarity with an appropriately tailored siRNA. For example, those of ordinary skill in the art can synthesize an siRNA (see, e.g., Elbashir, et al., Nature 411: 494 (2001); Elbashir, et al. Genes Dev 15:188 (2001); Tuschl T, et al., Genes Dev 13:3191 (1999)).

In some embodiments, RNAi is utilized to inhibit Myo9. In some embodiments, RNAi is used to modulate one or more members of the Slit-Robo-Myo9-RhoA pathway (e.g., Myo9 (e.g., Myo9b, Myo9a); Slit (e.g., Slit1, Slit2, Slit3); Robo (e.g., Robo1, Robo2, Robo3, Robo4); RhoA; another Slit-Robo-Myo9-RhoA pathway gene; or another gene or gene product that modulates the Slit-Robo-Myo9-RhoA pathway). RNAi represents an evolutionarily conserved cellular defense for controlling the expression of foreign genes in most eukaryotes, including humans. RNAi is typically triggered by double-stranded RNA (dsRNA) and causes sequence-specific degradation of single-stranded target RNAs (e.g., an mRNA). The mediators of mRNA degradation are small interfering RNAs (siRNAs), which are normally produced from long dsRNA by enzymatic cleavage in the cell. siRNAs are generally approximately twenty-one nucleotides in length (e.g. 21-23 nucleotides in length) and have a base-paired structure characterized by two-nucleotide 3′ overhangs. Following the introduction of a small RNA, or RNAi, into the cell, it is believed the sequence is delivered to an enzyme complex called RISC (RNA-induced silencing complex). RISC recognizes the target and cleaves it with an endonuclease. It is noted that if larger RNA sequences are delivered to a cell, an RNase III enzyme (e.g., Dicer) converts the longer dsRNA into 21-23 nt double-stranded siRNA fragments. In some embodiments, RNAi oligonucleotides are designed to target the junction region of fusion proteins. Chemically synthesized siRNAs have become powerful reagents for genome-wide analysis of mammalian gene function in cultured somatic cells. Beyond their value for validation of gene function, siRNAs also hold great potential as gene-specific therapeutic agents (see, e.g., Tuschl and Borkhardt, Molecular Intervent. 2002; 2(3): 158-67, herein incorporated by reference).

The transfection of siRNAs into animal cells results in the potent, long-lasting posttranscriptional silencing of specific genes (Caplen et al, Proc Natl Acad Sci U.S.A. 2001; 98: 9742-47; Elbashir et al., Nature. 2001; 411:4 94-98; Elbashir et al., Genes Dev. 2001; 15: 188-200; and Elbashir et al., EMBO J. 2001; 20: 6877-88, all of which are herein incorporated by reference). Methods and compositions for performing RNAi with siRNAs are described, for example, in U.S. Pat. No. 6,506,559, herein incorporated by reference.

siRNAs are extraordinarily effective at lowering the amounts of targeted RNA and their protein products, frequently to undetectable levels. The silencing effect can last several months, and is extraordinarily specific—a one-nucleotide mismatch between the target RNA and the central region of the siRNA is frequently sufficient to prevent silencing (Brummelkamp et al, Science 2002; 296: 550-53; and Holen et al, Nucleic Acids Res. 2002; 30: 1757-66, both of which are herein incorporated by reference).

An important factor in the design of siRNAs is the presence of accessible sites for siRNA binding. Bahoia et al., (J. Biol. Chem., 2003; 278: 15991-97; herein incorporated by reference) describe the use of a type of DNA array called a scanning array to find accessible sites in mRNAs for designing effective siRNAs. These arrays comprise oligonucleotides ranging in size from monomers to a certain maximum, usually Co-mers, synthesized using a physical barrier (mask) by stepwise addition of each base in the sequence. Thus, the arrays represent a full oligonucleotide complement of a region of the target gene. Hybridization of the target mRNA to these arrays provides an exhaustive accessibility profile of this region of the target mRNA. Such data are useful in the design of antisense oligonucleotides (e.g., ranging from 7mers to 25mers), where it is important to achieve a compromise between oligonucleotide length and binding affinity, e.g., to retain efficacy and target specificity (Sohail et al, Nucleic Acids Res., 2001; 29(10): 2041-45). Additional methods and concerns for selecting siRNAs are described, for example, in WO 05054270, WO05038054A1, WO03070966A2, J Mol Biol. 2005 May 13; 348(4):883-93, J Mol Biol. 2005 May 13; 348(4):871-81, and Nucleic Acids Res. 2003 Aug. 1; 31(15):4417-24, each of which is herein incorporated by reference in its entirety. In addition, software (e.g., the MWG online siMAX siRNA design tool) is commercially or publicly available for use in the selection and design of siRNAs and RNAi reagents.

In some embodiments, the present technology utilizes siRNA comprising blunt ends (See e.g., US20080200420, herein incorporated by reference in its entirety), overhangs (See e.g., US20080269147A1, herein incorporated by reference in its entirety), and/or locked nucleic acids (see e.g., WO2008/006369, WO2008/043753, and WO2008/051306, each of which is herein incorporated by reference in its entirety). In some embodiments, siRNAs are delivered via gene expression or using bacteria (See e.g., Xiang et al., Nature 24: 6 (2006) and WO06066048, each of which is herein incorporated by reference in its entirety).

In other embodiments, shRNA techniques (See e.g., 20080025958, herein incorporated by reference in its entirety) are utilized to modulate one or more members of the Slit-Robo-Myo9-RhoA pathway (e.g., Myo9 (e.g., Myo9b, Myo9a); Slit (e.g., Slit1, Slit2, Slit3); Robo (e.g., Robo1, Robo2, Robo3, Robo4); RhoA; another Slit-Robo-Myo9-RhoA pathway gene; or another gene or gene product that modulates the Slit-Robo-Myo9-RhoA pathway). A small hairpin RNA or short hairpin RNA (shRNA) is a sequence of RNA that makes a tight hairpin turn that can be used to silence gene expression via RNA interference. shRNA uses a vector introduced into cells and utilizes the U6 promoter to ensure that the shRNA is always expressed. This vector is usually passed on to daughter cells, allowing the gene silencing to be inherited. The shRNA hairpin structure is cleaved by the cellular machinery into siRNA, which is then bound to the RNA-induced silencing complex (RISC). This complex binds to and cleaves mRNAs that match the siRNA that is bound to it. shRNA is transcribed by RNA polymerase III.

The present technology also includes pharmaceutical compositions and formulations that include the RNAi compounds of the present invention as described below.

In some embodiments, the technology relates to use of (e.g., as a diagnostic biomarker) or targeting of a long non-coding RNA to modulate the activity of a member of the Slit-Robo-Myo9-RhoA pathway (e.g., Myo9 (e.g., Myo9b, Myo9a); Slit (e.g., Slit1, Slit2, Slit3); Robo (e.g., Robo1, Robo2, Robo3, Robo4); RhoA; another Slit-Robo-Myo9-RhoA pathway gene; or another gene or gene product that modulates the Slit-Robo-Myo9-RhoA pathway). In some embodiments, the technology described herein relates to use of (e.g., as a diagnostic biomarker) or targets that are long intervening (intronic and intergenic) non-coding RNAs, or “lncRNAs”, also known in the art as macroRNAs and efference RNAs (eRNAs). As used herein, the term “lncRNA”, or “long intervening non-coding RNA” refers broadly to targets of the present technology and include the “lncRNA gene” and the resultant “lncRNA transcript.” To the extent that the lncRNA transcript acts as an antisense molecule (whether in cis or trans to effect concordant or discordant regulation) to a second transcript (whether RNA or DNA), the family of lncRNA targets encompassed by the present technology also includes NATs (Natural Antisense Transcripts). Further, pseudogenes, whether arising from transposition or duplication, are also considered to fall within the broader family of lncRNA targets of the present technology.

“lncRNA genes” of the present technology are processed to produce “lncRNA transcripts” and these transcripts may be transcribed from either strand of the chromosomal DNA. Unless otherwise noted, the term “lncRNA” refers broadly to the lncRNA gene and the resultant lncRNA transcript. lncRNA genes may be as small as 1 kb (kilobase) or as large as 100 kb (kilobases) while lncRNA transcripts may range in size from 200 nucleotides to 20 kb.

lncRNA transcripts may be at least 200 nucleotides, at least 300 nucleotides, at least 400 nucleotides, at least 500 nucleotides, at least 600 nucleotides, at least 700 nucleotides, at least 800 nucleotides, at least 900 nucleotides, at least 1000 nucleotides, at least 5000 nucleotides, at least 10,000 nucleotides or at least 20,000 nucleotides, and range from 250-300 nucleotides, 300-400 nucleotides, 400-500 nucleotides, 500-600 nucleotides, 600-700 nucleotides, 700-800 nucleotides, 800-900 nucleotides, 900-1000 nucleotides, 1000-5000 nucleotides, 5000-10,000 nucleotides, or 10,000-20,000 nucleotides in length.

As used herein, the term “lncRNA gene” refers to the lncRNA that is encoded within the genome or in a genomic construct (whether natural or synthetic) and has at least one feature of a coding gene selected from the group including, but not limited to, (i) a promoter or promoter-like feature such as one or more proximal regulatory elements; (ii) one or more exons; (iii) a polyA signature; and (iv) which encodes a transcribed RNA, e.g., an lncRNA transcript. Endogenous lncRNA genes, e.g., those encoded within or engineered to be encoded by a host cell genome, are characterized by their intervening genomic location. This means that endogenous or wild-type lncRNAs may be intronic or intergenic.

“Intronic lncRNAs” are those found to be encoded substantially within an intron of a gene. “Intergenic lncRNAs” are those found to be encoded between two different genes. As used herein, the term “lncRNA transcript”, refers to an RNA transcript encoded by a lncRNA gene and which is (i) at least 200 nucleotides in length and (ii) does not encode a mature or complete protein product. lncRNA transcripts may encode peptides of 50 amino acids or less. It should be understood that lncRNA transcripts may be synthesized as lncRNA transcript variants, which may be engineered to encode peptides or proteins. “Intervening” when used in the context of lncRNAs means intronic or intergenic. lncRNAs find use in the present technology both as diagnostic biomarkers (e.g., for cancer) and as targets for therapy.

In some embodiments, the technology described herein uses antisense nucleic acid (e.g., an antisense DNA oligo, an antisense RNA oligo) to modulate the activity of a member of the Slit-Robo-Myo9-RhoA pathway (e.g., Myo9 (e.g., Myo9b, Myo9a); Slit (e.g., Slit1, Slit2, Slit3); Robo (e.g., Robo1, Robo2, Robo3, Robo4); RhoA; another Slit-Robo-Myo9-RhoA pathway gene; or another gene or gene product that modulates the Slit-Robo-Myo9-RhoA pathway). For example, in some embodiments, expression of a member of the Slit-Robo-Myo9-RhoA pathway is modulated using antisense compounds that specifically hybridize with one or more nucleic acids encoding a member of the Slit-Robo-Myo9-RhoA pathway. The specific hybridization of an oligomeric compound with its target nucleic acid interferes with the normal function of the nucleic acid. This modulation of function of a target nucleic acid by compounds that specifically hybridize to it is generally referred to as “antisense.” The functions of DNA to be interfered with include replication and transcription. The functions of RNA to be interfered with include all vital functions such as, for example, translocation of the RNA to the site of protein translation, translation of protein from the RNA, splicing of the RNA to yield one or more mRNA species, and catalytic activity that may be engaged in or facilitated by the RNA. The overall effect of such interference with target nucleic acid function is modulation of the expression of a member of the Slit-Robo-Myo9-RhoA pathway.

Antisense methods preferably target specific nucleic acids. “Targeting” an antisense compound to a particular nucleic acid usually refers to a multistep process that begins with identification of a nucleic acid sequence whose function is to be modulated. This may be, e.g., a cellular gene (or mRNA transcribed from the gene) whose expression is associated with a particular disorder or disease state, or a nucleic acid molecule from an infectious agent. Herein, the target is a nucleic acid molecule encoding a member of the Slit-Robo-Myo9-RhoA pathway. The targeting process also includes determination of a site or sites within this gene for the antisense interaction to occur such that the desired effect, e.g., detection or modulation of expression of the protein, will result. Herein, a preferred intragenic site is the region encompassing the translation initiation or termination codon of the open reading frame (ORF) of the gene. Since the translation initiation codon is typically 5′-AUG (in transcribed mRNA molecules; 5′-ATG in the corresponding DNA molecule), the translation initiation codon is also referred to as the “AUG codon,” the “start codon” or the “AUG start codon”. A minority of genes have a translation initiation codon having the RNA sequence 5′-GUG, 5′-UUG or 5′-CUG, and 5′-AUA, 5′-ACG and 5′-CUG have been shown to function in vivo. Thus, the terms “translation initiation codon” and “start codon” can encompass many codon sequences, even though the initiator amino acid in each instance is typically methionine (in eukaryotes) or formylmethionine (in prokaryotes). Eukaryotic and prokaryotic genes may have two or more alternative start codons, any one of which may be preferentially used for translation initiation in a particular cell type or tissue, or under a particular set of conditions. Herein, “start codon” and “translation initiation codon” refer to the codon or codons that are used in vivo to initiate translation of an mRNA molecule transcribed from a gene encoding a member of the Slit-Robo-Myo9-RhoA pathway, regardless of the sequence(s) of such codons.

Translation termination codon (or “stop codon”) of a gene may have one of three sequences (i.e., 5′-UAA, 5′-UAG and 5′-UGA; the corresponding DNA sequences are 5′-TAA, 5′-TAG and 5′-TGA, respectively). The terms “start codon region” and “translation initiation codon region” refer to a portion of such an mRNA or gene that encompasses from about 25 to about 50 contiguous nucleotides in either direction (i.e., 5′ or 3′) from a translation initiation codon. Similarly, the terms “stop codon region” and “translation termination codon region” refer to a portion of such an mRNA or gene that encompasses from about 25 to about 50 contiguous nucleotides in either direction (i.e., 5′ or 3′) from a translation termination codon.

The open reading frame (ORF) or “coding region,” which refers to the region between the translation initiation codon and the translation termination codon, is also a region that may be targeted effectively. Other target regions include the 5′ untranslated region (5′ UTR), referring to the portion of an mRNA in the 5′ direction from the translation initiation codon, and thus including nucleotides between the 5′ cap site and the translation initiation codon of an mRNA or corresponding nucleotides on the gene, and the 3′ untranslated region (3′ UTR), referring to the portion of an mRNA in the 3′ direction from the translation termination codon, and thus including nucleotides between the translation termination codon and 3′ end of an mRNA or corresponding nucleotides on the gene. The 5′ cap of an mRNA comprises an N7-methylated guanosine residue joined to the 5′-most residue of the mRNA via a 5′-5′ triphosphate linkage. The 5′ cap region of an mRNA is considered to include the 5′ cap structure itself as well as the first 50 nucleotides adjacent to the cap. The cap region may also be a preferred target region.

Although some eukaryotic mRNA transcripts are directly translated, many contain one or more regions, known as “introns,” that are excised from a transcript before it is translated. The remaining (and therefore translated) regions are known as “exons” and are spliced together to form a continuous mRNA sequence. mRNA splice sites (i.e., intron-exon junctions) may also be preferred target regions, and are particularly useful in situations where aberrant splicing is implicated in disease, or where an overproduction of a particular mRNA splice product is implicated in disease. Aberrant fusion junctions due to rearrangements or deletions are also preferred targets. It has also been found that introns can also be effective, and therefore preferred, target regions for antisense compounds targeted, for example, to DNA or pre-mRNA.

In some embodiments, target sites for antisense inhibition are identified using commercially available software programs (e.g., Biognostik, Gottingen, Germany; SysArris Software, Bangalore, India; Antisense Research Group, University of Liverpool, Liverpool, England; GeneTrove, Carlsbad, Calif.). In other embodiments, target sites for antisense inhibition are identified using the accessible site method described in PCT Pub. No. WO0198537A2, herein incorporated by reference.

Once one or more target sites have been identified, oligonucleotides are chosen that are sufficiently complementary to the target (i.e., hybridize sufficiently well and with sufficient specificity) to give the desired effect. For example, in preferred embodiments, antisense oligonucleotides are targeted to or near the start codon associated with a member of the Slit-Robo-Myo9-RhoA pathway.

In the context of this invention, “hybridization,” with respect to antisense compositions and methods, means hydrogen bonding, which may be Watson-Crick, Hoogsteen, or reversed Hoogsteen hydrogen bonding, between complementary nucleoside or nucleotide bases. For example, adenine and thymine are complementary nucleobases that pair through the formation of hydrogen bonds. It is understood that the sequence of an antisense compound need not be 100% complementary to that of its target nucleic acid to be specifically hybridizable. An antisense compound is specifically hybridizable when binding of the compound to the target DNA or RNA molecule interferes with the normal function of the target DNA or RNA to cause a loss of utility, and there is a sufficient degree of complementarity to avoid non-specific binding of the antisense compound to non-target sequences under conditions in which specific binding is desired (i.e., under physiological conditions in the case of in vivo assays or therapeutic treatment, and in the case of in vitro assays, under conditions in which the assays are performed).

Antisense compounds are commonly used as research reagents and diagnostics. For example, antisense oligonucleotides, which are able to inhibit gene expression with specificity, can be used to elucidate the function of particular genes. Antisense compounds are also used, for example, to distinguish between functions of various members of a biological pathway.

The specificity and sensitivity of antisense is also applied for therapeutic uses. For example, antisense oligonucleotides have been employed as therapeutic moieties in the treatment of disease states in animals and man. Antisense oligonucleotides have been safely and effectively administered to humans and numerous clinical trials are presently underway. It is thus established that oligonucleotides are useful therapeutic modalities that can be configured to be useful in treatment regimes for treatment of cells, tissues, and animals, especially humans.

While antisense oligonucleotides are preferred, other oligomeric antisense compounds, including but not limited to oligonucleotide mimetics may be used, such as are described below. Preferred antisense compounds comprise from about 8 to about 30 nucleobases (i.e., from about 8 to about 30 linked bases), although both longer and shorter sequences may be used. Particularly preferred antisense compounds are antisense oligonucleotides, even more preferably those comprising from about 12 to about 25 nucleobases.

Specific examples of preferred antisense compounds include oligonucleotides containing modified backbones or non-natural internucleoside linkages. As defined herein, oligonucleotides having modified backbones include those that retain a phosphorus atom in the backbone and those that do not have a phosphorus atom in the backbone. For the purposes of this specification, modified oligonucleotides that do not have a phosphorus atom in their internucleoside backbone can also be considered to be oligonucleosides.

Embodiments of the technology provide nucleic acids (e.g., DNA, RNA) that are modified in the nucleobases and/or in the connections between nucleobases. Embodiments provide for the use of such nucleic acids in the methods described herein, e.g., including but not limited to RNAi, siRNA, shRNA, antisense, and miRNA methods described.

Preferred modified oligonucleotide backbones include, for example, phosphorothioates, chiral phosphorothioates, phosphorodithioates, phosphotriesters, aminoalkylphosphotriesters, methyl and other alkyl phosphonates including 3′-alkylene phosphonates and chiral phosphonates, phosphinates, phosphoramidates including 3′-amino phosphoramidate and aminoalkylphosphoramidates, thionophosphoramidates, thionoalkylphosphonates, thionoalkylphosphotriesters, and boranophosphates having normal 3′-5′ linkages, 2′-5′ linked analogs of these, and those having inverted polarity wherein the adjacent pairs of nucleoside units are linked 3′-5′ to 5′-3′ or 2′-5′ to 5′-2′. Various salts, mixed salts and free acid forms are also included.

Preferred modified oligonucleotide backbones that do not include a phosphorus atom therein have backbones that are formed by short chain alkyl or cycloalkyl internucleoside linkages, mixed heteroatom and alkyl or cycloalkyl internucleoside linkages, or one or more short chain heteroatomic or heterocyclic internucleoside linkages. These include those having morpholino linkages (formed in part from the sugar portion of a nucleoside); siloxane backbones; sulfide, sulfoxide and sulfone backbones; formacetyl and thioformacetyl backbones; methylene formacetyl and thioformacetyl backbones; alkene containing backbones; sulfamate backbones; methyleneimino and methylenehydrazino backbones; sulfonate and sulfonamide backbones; amide backbones; and others having mixed N, O, S and CH₂ component parts.

In other preferred oligonucleotide mimetics, both the sugar and the internucleoside linkage (e.g., the backbone) of the nucleotide units are replaced with novel groups. The base units are maintained for hybridization with an appropriate nucleic acid target compound. One such oligomeric compound, an oligonucleotide mimetic that has been shown to have excellent hybridization properties, is referred to as a peptide nucleic acid (PNA). In PNA compounds, the sugar-backbone of an oligonucleotide is replaced with an amide containing backbone, in particular an aminoethylglycine backbone. The nucleobases are retained and are bound directly or indirectly to aza nitrogen atoms of the amide portion of the backbone. Methods for preparation of PNA compounds are well known (e.g., see U.S. Pat. Nos. 5,539,082; 5,714,331; and 5,719,262, and Nielsen et al., Science 254:1497 (1991), each of which is herein incorporated by reference).

Most preferred embodiments of the invention are oligonucleotides with phosphorothioate backbones and oligonucleosides with heteroatom backbones, and in particular —CH₂, —NH—O—CH₂—, —CH₂—N(CH₃)—O—CH₂— (known as a methylene (methylimino), or MMI backbone), —CH₂—O—N(CH₃)—CH₂—, —CH₂—N(CH₃)—N(CH₃)—CH₂—, and —O—N(CH₃)—CH₂—CH₂— (wherein the native phosphodiester backbone is represented as —O—P—O—CH₂—), amid backbone, and morpholino backbone structures, all of which are well known (e.g., see U.S. Pat. Nos. 5,489,677, 5,602,240, and 5,034,506).

Modified oligonucleotides may also contain one or more substituted sugar moieties. Preferred oligonucleotides comprise one of the following at the 2′ position: OH; F; O—, S—, or N-alkyl; O—, S—, or N-alkenyl; O—, S- or N-alkynyl; or O-alkyl-O-alkyl, wherein the alkyl, alkenyl and alkynyl may be substituted or unsubstituted C₁ to C₁₀ alkyl or C₂ to C₁₀ alkenyl and alkynyl. Particularly preferred are O[(CH₂)ₙO]ₘCH₃, O(CH₂)ₙ—OCH₃, O(CH₂)ₙ—NH₂, O(CH₂)ₙ—CH₃, O(CH₂)ₙ—ONH₂, and O(CH₂)ₙ—ON[(CH₂)nCH₃)]₂, where n and m are from 1 to about 10. Other preferred oligonucleotides comprise one of the following at the 2′ position: C₁ to C₁₀ lower alkyl, substituted lower alkyl, alkaryl, aralkyl, O-alkaryl or O-aralkyl, SH, SCH₃, OCN, Cl, Br, CN, CF₃, OCF₃, SOCH₃, SO₂CH₃, ONO₂, NO₂, N₃, NH₂, heterocycloalkyl, heterocycloalkaryl, aminoalkylamino, polyalkylamino, substituted silyl, an RNA cleaving group, a reporter group, an intercalator, a group for improving the pharmacokinetic properties of an oligonucleotide, or a group for improving the pharmacodynamic properties of an oligonucleotide, and other substituents having similar properties. A preferred modification includes 2′-methoxyethoxy (2′-O—CH₂CH₂OCH₃, also known as 2′-O-(2-methoxyethyl) or 2′-MOE) (Martin et al., Helv. Chim. Acta 78:486 [1995]) i.e., an alkoxyalkoxy group. A further preferred modification includes 2′-dimethylaminooxyethoxy (i.e., a O(CH₂)₂O N(CH₃)₂ group), also known as 2′-DMAOE, and 2′-dimethylaminoethoxyethoxy (also known in the art as 2′-O-dimethylaminoethoxyethyl or 2′-DMAEOE), i.e., 2′-O—CH₂—O—CH₂—N(CH₂)₂.

Other preferred modifications include 2′-methoxy(2′-O—CH₃), 2′-aminopropoxy(2′-OCH₂CH₂CH₂NH₂) and 2′-fluoro (2′-F). Similar modifications may also be made at other positions on the oligonucleotide, particularly the 3′ position of the sugar on the 3′ terminal nucleotide or in 2′-5′ linked oligonucleotides and the 5′ position of 5′ terminal nucleotide. Oligonucleotides may also have sugar mimetics such as cyclobutyl moieties in place of the pentofuranosyl sugar.

Oligonucleotides may also include nucleobase (often referred to in the art simply as “base”) modifications or substitutions. As used herein, “unmodified” or “natural” nucleobases include the purine bases adenine (A) and guanine (G), and the pyrimidine bases thymine (T), cytosine (C) and uracil (U). Modified nucleobases are well known (e.g., see U.S. Pat. No. 3,687,808) and include other synthetic and natural nucleobases (for which the A, G, T, C and U abbreviations for the bases are used in the following examples), such as 5-methylcytosine (5-me-C), 5-hydroxymethyl C, xanthine, hypoxanthine, 2-amino-A, 6-methyl or 2-propyl and other alkyl derivatives of A and G, 2-thio-U, 2-thio-T and 2-thio-C, 5-halo-U and -C, 5-propynyl U and C, 6-azo U, C and T, 5-uracil (pseudouracil), 4-thio-U, 8-halo, 8-amino, 8-thiol, 8-thioalkyl, 8-hydroxyl and other 8-substituted A and G, 5-halo substituted U and C, particularly 5-bromo, 5-trifluoromethyl and other 5-substituted U and C, 7-methyl-G and 7-methyl-A, 8-aza-G and 8-aza-A, 7-deaza-G and 7-deaza-A and 3-deaza-G and 3-deaza-A. Certain of these nucleobases are particularly useful for increasing the binding affinity of the oligomeric compounds of the invention. These include 5-substituted pyrimidines, 6-azapyrimidines and N-2, N-6 and O-6 substituted purines, including 2-aminopropyladenine, 5-propynyl-U and 5-propynyl-C. 5-methyl-C substitutions are known to increase nucleic acid duplex stability and are preferred base substitutions in some embodiments, even more particularly when combined with 2′-O-methoxyethyl sugar modifications.

Another modification of the oligonucleotides involves chemically linking to the oligonucleotide one or more moieties or conjugates that enhance the activity, cellular distribution or cellular uptake of the oligonucleotide. Such moieties include but are not limited to lipid moieties such as a cholesterol moiety, cholic acid, a thioether, (e.g., hexyl-S-tritylthiol), a thiocholesterol, an aliphatic chain, (e.g., dodecandiol or undecyl residues), a phospholipid, (e.g., di-hexadecyl-rac-glycerol or triethylammonium 1,2-di-O-hexadecyl-rac-glycero-3-H-phosphonate), a polyamine or a polyethylene glycol chain or adamantane acetic acid, a palmityl moiety, or an octadecylamine or hexylamino-carbonyl-oxycholesterol moiety.

One skilled in the relevant art knows well how to generate oligonucleotides containing the above-described modifications. The present invention is not limited to the oligonucleotides described above. Any suitable modification or substitution may be used.

It is not necessary for all positions in a given compound to be uniformly modified, and in fact more than one of the aforementioned modifications may be incorporated in a single compound or even at a single nucleoside within an oligonucleotide. Antisense compounds may be chimeric compounds. “Chimeric antisense compounds” as used herein, are antisense compounds, particularly oligonucleotides, which contain two or more chemically distinct regions, each made up of at least one monomer unit, i.e., a nucleotide in the case of an oligonucleotide compound. These oligonucleotides typically contain at least one region wherein the oligonucleotide is modified so as to confer upon the oligonucleotide increased resistance to nuclease degradation, increased cellular uptake, and/or increased binding affinity for the target nucleic acid. An additional region of the oligonucleotide may serve as a substrate for enzymes capable of cleaving RNA:DNA or RNA:RNA hybrids. By way of example, RNaseH is a cellular endonuclease that cleaves the RNA strand of an RNA:DNA duplex. Activation of RNase H, therefore, results in cleavage of the RNA target, thereby greatly enhancing the efficiency of oligonucleotide inhibition of gene expression. Consequently, comparable results can often be obtained with shorter oligonucleotides when chimeric oligonucleotides are used, compared to phosphorothioate deoxyoligonucleotides hybridizing to the same target region. Cleavage of the RNA target can be routinely detected by gel electrophoresis and, if necessary, associated nucleic acid hybridization techniques known in the art.

Chimeric antisense compounds may be formed as composite structures of two or more oligonucleotides, modified oligonucleotides, oligonucleosides and/or oligonucleotide mimetics as described above.

Other embodiments include pharmaceutical compositions and formulations that include the nucleic acid compounds as described herein.

In addition, it is contemplated that a member of the Slit-Robo-Myo9-RhoA pathway (e.g., Myo9 (e.g., Myo9b, Myo9a); Slit (e.g., Slit1, Slit2, Slit3); Robo (e.g., Robo1, Robo2, Robo3, Robo4); RhoA; another Slit-Robo-Myo9-RhoA pathway gene; or another gene or gene product that modulates the Slit-Robo-Myo9-RhoA pathway) can be modulated (e.g., activated, inhibited) by chemicals (e.g., a small molecule, e.g., a pharmacological agent) or other biological agents that bind to and/or modulate the activity of a member of the Slit-Robo-Myo9-RhoA pathway (e.g., Myo9 (e.g., Myo9b, Myo9a); Slit (e.g., Slit1, Slit2, Slit3); Robo (e.g., Robo1, Robo2, Robo3, Robo4); RhoA; another Slit-Robo-Myo9-RhoA pathway gene; or another gene or gene product that modulates the Slit-Robo-Myo9-RhoA pathway).

For example, one of ordinary skill in the art can design and produce RNA aptamers or other nucleic acids that specifically recognize and bind to Myo9, for instance by using SELEX or other in vitro evolution methods known in the art. Furthermore, Myo9 activity can be inhibited by specifically degrading Myo9 or inducing an altered conformation of Myo9 such that it is less effective in inhibiting RhoA. In some embodiments, the Myo9 inhibitor is a “designed ankyrin repeat protein” (DARPin) (see, e.g., Stumpp M T & Amstutz P, “DARPins: a true alternative to antibodies”, Curr Opin Drug Discov Devel 2007, 10(2): 153-59, incorporated herein in its entirety for all purposes). In some embodiments, Myo9 is inhibited by a small molecule, e.g., a small molecule that binds to Myo9 and blocks its function (e.g., inhibits its binding and/or other interaction (e.g., an inhibitory interaction) with RhoA).

In some embodiments, chemical compounds promote the degradation and/or decrease the stability of Myo9 messenger RNA. In some embodiments, chemical compounds inhibit Myo9 (e.g., Myo9b) activity by interacting with the myosin head domain, the actin-binding domain, and/or the GTPase activating protein (GAP) domain of the Myo9 (e.g., Myo9b) protein.

It is contemplated that altering Myo9 activity can be effected by inhibiting the expression of Myo9, for instance, by inhibiting the transcription of Myo9, by inhibiting the translation of Myo9, by inhibiting the processing of the Myo9 mRNA, by inhibiting the processing of the Myo9 polypeptide, by inhibiting the folding of the Myo9 polypeptide, or by inhibiting trafficking of Myo9 within a cell. Myo9 activity can be altered by changes in chromatin structure or other means of epigenetic regulation of Myo9 (e.g., changes in DNA methylation). Also, Myo9 activity may be altered by specifically sequestering Myo9 in a vesicle or other cellular compartment that hinders its action upon RhoA.

Modulating a member of the Slit-Robo-Myo9-RhoA pathway (e.g., Myo9 (e.g., Myo9b, Myo9a); Slit (e.g., Slit1, Slit2, Slit3); Robo (e.g., Robo1, Robo2, Robo3, Robo4); RhoA; another Slit-Robo-Myo9-RhoA pathway gene; or another gene or gene product that modulates the Slit-Robo-Myo9-RhoA pathway) finds use in therapies to treat disease (e.g., cancer). For example, in some embodiments, inhibiting Myo9 finds use in therapies to treat disease (e.g., cancer). Accordingly, provided herein are therapies comprising inhibiting Myo9 to benefit individuals suffering from disease (e.g., cancer). In particular, as discussed herein, cancerous disease states demonstrate aberrant Myo9 activity. While the therapies are not limited in their route of administration, embodiments of the technology provided herein deliver the Myo9 inhibitor via intratumor injection, intravenous systemic administration, using an adenoviral delivery vehicle, and/or by intranasal administration (e.g., using an intranasal spray or mist).

In certain embodiments, a physiologically appropriate solution containing an effective concentration of an antibody specific for a member of the Slit-Robo-Myo9-RhoA pathway (e.g., Myo9 (e.g., Myo9b, Myo9a); Slit (e.g., Slit1, Slit2, Slit3); Robo (e.g., Robo1, Robo2, Robo3, Robo4); RhoA; another Slit-Robo-Myo9-RhoA pathway gene; or another gene or gene product that modulates the Slit-Robo-Myo9-RhoA pathway) is administered topically, intraocularly, parenterally, orally, intranasally, intravenously, intramuscularly, subcutaneously, or by any other effective means. In certain embodiments, a physiologically appropriate solution containing an effective concentration of an antibody specific for Myo9 is administered topically, intraocularly, parenterally, orally, intranasally, intravenously, intramuscularly, subcutaneously, or by any other effective means. Alternatively, a tissue can receive a physiologically appropriate composition (e.g., a solution such as a saline or phosphate buffer, a suspension, or an emulsion, which is sterile) containing an effective concentration of an antibody specific for Myo9 via direct injection with a needle or via a catheter or other delivery tube. Any effective imaging device such as X-ray, sonogram, or fiber-optic visualization system may be used to locate the target tissue and guide the administration. In another alternative, a physiologically appropriate solution containing an effective concentration of an antibody specific for Myo9 can be administered systemically into the blood circulation to treat tissue that cannot be directly reached or anatomically isolated. Such manipulations have in common the goal of placing an effective concentration of an antibody specific for Myo9 in sufficient contact with the target tissue to permit the antibody specific for Myo9 to contact the tissue, e.g., a tumorous tissue.

With respect to administration of a Myo9 inhibitor (e.g., an antibody specific for Myo9) to a subject, it is contemplated that the Myo9 inhibitor be administered in a pharmaceutically effective amount. One of ordinary skill recognizes that a pharmaceutically effective amount varies depending on the therapeutic agent used, the subject's age, condition, and sex, and on the extent of the disease in the subject. Generally, the dosage should not be so large as to cause adverse side effects, such as hyperviscosity syndromes, pulmonary edema, congestive heart failure, and the like. The dosage can also be adjusted by the individual physician or veterinarian to achieve the desired therapeutic goal.

As used herein, the actual amount encompassed by the term “pharmaceutically effective amount” will depend on the route of administration, the type of subject being treated, and the physical characteristics of the specific subject under consideration. These factors and their relationship to determining this amount are well known to skilled practitioners in the medical, veterinary, and other related arts. This amount and the method of administration can be tailored to achieve optimal efficacy but will depend on such factors as weight, diet, concurrent medication, and other factors that those skilled in the art will recognize.

In some embodiments, a Myo9 inhibitor (e.g., an antibody specific for Myo9) according to the technology provided herein is administered in a pharmaceutically effective amount. In some embodiments, a Myo9 inhibitor (e.g., an antibody specific for Myo9) is administered in a therapeutically effective dose. The dosage amount and frequency are selected to create an effective level of the Myo9 inhibitor without substantially harmful effects. When administered, the dosage of a Myo9 inhibitor (e.g., an antibody specific for Myo9) will generally range from 0.001 to 10,000 mg/kg/day or dose (e.g., 0.01 to 1000 mg/kg/day or dose; 0.1 to 100 mg/kg/day or dose).

Pharmaceutical compositions preferably comprise one or more compounds of the present invention associated with one or more pharmaceutically acceptable carriers, diluents, or excipients. Pharmaceutically acceptable carriers are known in the art such as those described in, for example, Remingtons Pharmaceutical Sciences, Mack Publishing Co. (A. R. Gennaro ed., 1985).

In some embodiments, a single dose of a Myo9 inhibitor (e.g., an antibody specific for Myo9) according to the technology provided herein is administered to a subject. In other embodiments, multiple doses are administered over two or more time points, separated by hours, days, weeks, etc. In some embodiments, compounds are administered over a long period of time (e.g., chronically), for example, for a period of months or years (e.g., 1, 2, 3, 4, 5, 6, 7, 8, 9, 10, 11, 12, or more months or years; e.g., for the lifetime of the subject). In such embodiments, compounds may be taken on a regular scheduled basis (e.g., daily, weekly, etc.) for the duration of the extended period.

In some embodiments, a Myo9 inhibitor (e.g., an antibody specific for Myo9) according to the technology provided herein is co-administered with another compound or more than one other compound (e.g., 2 or 3 or more other compounds).

In some embodiments, methods of monitoring treatment of cancer are provided. In some embodiments, the present methods of detecting biomarkers are carried out at a time 0. In some embodiments, the method is carried out again at a time 1, and optionally, a time 2, and optionally, a time 3, etc., in order to monitor the progression of cancer or to monitor the effectiveness of one or more treatments of cancer. Time points for detection may be separated by, for example at least 4 hours, at least 8 hours, at least 12 hours, at least 1 day, at least 2 days, at least 4 days, at least 1 week, at least 2 weeks, at least 1 month, at least 2 months, at least 3 months, at least 4 months, at least 6 months, or by 1 year or more. In some embodiments, a treatment regimen is altered based upon the results of monitoring (e.g., upon determining that a first treatment is ineffective). In some embodiments, the level of intervention may be altered.

In some embodiments, the technology provides methods for in vivo imaging. For example, the technology includes use of an antibody (e.g., a labeled antibody) or a nucleic acid (e.g., a probe). In vivo imaging technologies include but are not limited to radionuclide imaging; positron emission tomography (PET); computerized axial tomography, X-ray, or magnetic resonance imaging method; fluorescence detection; and chemiluminescent detection. In some embodiments, in vivo imaging techniques are used to visualize the presence of or expression of a biomarker in an animal (e.g., a human or non-human mammal). For example, in some embodiments, cancer marker mRNA or protein is labeled using a labeled antibody or nucleic acid specific for the cancer marker. A specifically bound and labeled antibody or nucleic acid can be detected in an individual using an in vivo imaging method, including, but not limited to, radionuclide imaging, positron emission tomography, computerized axial tomography, X-ray, or magnetic resonance imaging method, fluorescence detection, and chemiluminescent detection. Methods for generating antibodies to the disclosed biomarkers are described herein. In some embodiments, in vivo imaging finds use to identify or locate a tumor. In some embodiments, in vivo imaging finds use in identifying the boundaries of a tumor.

The in vivo imaging methods that use the compositions disclosed herein that detect the biomarkers discussed herein or products derived from them are useful in the diagnosis of disease, such as cancers that express the cancer markers disclosed herein. In vivo imaging visualizes the presence of a marker indicative of the cancer, allowing diagnosis and/or prognosis without the use of an unpleasant biopsy. For example, the presence of a marker indicative of cancers likely to metastasize can be detected. The in vivo imaging methods can further be used to detect metastatic cancers in other parts of the body.

In some embodiments, reagents (e.g., antibodies, nucleic acids, etc.) specific for one or more biomarkers described herein are fluorescently labeled. The labeled antibodies or nucleic acids are introduced into a subject (e.g., orally or parenterally). Fluorescently labeled antibodies are detected using any suitable method or system (e.g., see U.S. Pat. No. 6,198,107, herein incorporated by reference).

In other embodiments, antibodies are radioactively labeled. The use of antibodies for in vivo diagnosis is well known in the art, e.g., by using an antibody-based labeling system to image tumors (see Sumerdon et al., Nucl. Med. Biol 17:247-254 [1990], Griffin et al., J. Clin. Onc. 9:631-640 [1991], and Lauffer, Magnetic Resonance in Medicine 22:339-342 [1991]). The label used with an antibody-based system will depend on the imaging modality chosen, for example, radioactive labels such as indium-111, technetium-99m, or iodine-131 for use with planar scans or single photon emission computed tomography (SPECT), positron emitting labels such as fluorine-19 for use with positron emission tomography (PET), and paramagnetic ions such as gadolinium (III) or manganese (II) for use with MM.

Radioactive metals with half-lives ranging from 1 hour to 3.5 days are available for conjugation to antibodies, such as scandium-47 (3.5 days), gallium-67 (2.8 days), gallium-68 (68 minutes), technetiium-99m (6 hours), and indium-111 (3.2 days), of which gallium-67, technetium-99m, and indium-111 are preferable for gamma camera imaging and gallium-68 is preferable for positron emission tomography.

A useful method of labeling antibodies with such radiometals is by means of a bifunctional chelating agent, such as diethylenetriaminepentaacetic acid (DTPA), as described, for example, by Khaw et al. (Science 209:295 [1980]) for In-111 and Tc-99m, and by Scheinberg et al. (Science 215:1511 [1982]). Other chelating agents may also be used, but the 1-(p-carboxymethoxybenzyl)EDTA and the carboxycarbonic anhydride of DTPA are advantageous because their use permits conjugation without affecting the antibody immunoreactivity substantially.

Another method for coupling DPTA to proteins is by use of the cyclic anhydride of DTPA, as described by Hnatowich et al. (Int. J. Appl. Radiat. Isot. 33:327 [1982]) for labeling of albumin with In-111, but which can be adapted for labeling of antibodies. A suitable method of labeling antibodies with Tc-99m is known (e.g., see Crockford et al., U.S. Pat. No. 4,323,546, herein incorporated by reference).

A preferred method of labeling immunoglobulins with Tc-99m is that described by Wong et al. (Int. J. Appl. Radiat. Isot., 29:251 [1978]) for plasma protein, and recently applied successfully by Wong et al. (J. Nucl. Med., 23:229 [1981]) for labeling antibodies.

In the case of the radiometals conjugated to the specific antibody, it is likewise desirable to introduce as high a proportion of the radiolabel as possible into the antibody molecule without destroying its immunospecificity. A further improvement may be achieved by effecting radiolabeling in the presence of the specific biomarker to insure that the antigen binding site on the antibody is protected. The antigen is separated after labeling.

In still further embodiments, in vivo biophotonic imaging (Xenogen, Almeda, Calif.) is used for in vivo imaging. This real-time in vivo imaging utilizes luciferase, an enzyme that catalyzes a light-emitting reaction. The luciferase gene is incorporated into cells, microorganisms, and animals, so that when the biomarker is active a light emission occurs which is captured as an image and analyzed by using a CCD camera and appropriate software.

In some embodiments, the embodiments of the technology provided herein are used in drug screening assays (e.g., to screen for anticancer drugs). These screening methods use cancer biomarkers that include those described herein that are part of or are associated with the Slit-Robo-Myo9-RhoA pathway, but are not limited only to those biomarkers. For example, an embodiment may screen for compounds that modulate (e.g., decrease) the expression of Myo9. Compounds or agents to be screened for may interfere with transcription (e.g., by interacting with a promoter region), may interfere with mRNA produced from the rearrangement (e.g., by RNA interference, antisense technologies, etc.), or may interfere with pathways that are upstream or downstream of the biological activity of the gene rearrangement. In some embodiments, candidate compounds are antisense or interfering RNA agents (e.g., oligonucleotides) directed against cancer biomarkers. In other embodiments, candidate compounds are antibodies or small molecules that specifically bind to a cancer marker regulator or expression product associated with the Slit-Robo-Myo9, RhoA pathway and/or inhibit aberrant biological function of members of this pathway.

In some embodiments, candidate compounds are evaluated for their ability to alter cancer marker expression by contacting a compound with a cell expressing a cancer marker and then assaying for the effect of the candidate compounds on expression. In some embodiments, the effect of candidate compounds on expression of a cancer marker gene is assayed for by detecting the level of cancer marker mRNA expressed by the cell. mRNA expression can be detected by any suitable method.

In other embodiments, the effect of candidate compounds on expression of a cancer marker genes is assayed by measuring the level of polypeptide encoded by the cancer markers. The level of polypeptide expressed can be measured using any suitable method, including but not limited to, those disclosed herein.

The test compounds can be obtained using any of the numerous approaches in combinatorial library methods known in the art, including biological libraries; peptoid libraries (libraries of molecules having the functionalities of peptides, but with a novel, non-peptide backbone, which are resistant to enzymatic degradation but which nevertheless remain bioactive; see, e.g., Zuckennann et al., J. Med. Chem. 37: 2678-85 [1994]); spatially addressable parallel solid phase or solution phase libraries; synthetic library methods requiring deconvolution; the ‘one-bead one-compound’ library method; and synthetic library methods using affinity chromatography selection. The biological library and peptoid library approaches are preferred for use with peptide libraries, while the other four approaches are applicable to peptide, non-peptide oligomer or small molecule libraries of compounds (Lam (1997) Anticancer Drug Des. 12:145).

Examples of methods for the synthesis of molecular libraries can be found in the art, for example in: DeWitt et al., Proc. Natl. Acad. Sci. U.S.A. 90:6909 [1993]; Erb et al., Proc. Nad. Acad. Sci. USA 91:11422 [1994]; Zuckermann et al., J. Med. Chem. 37:2678 [1994]; Cho et al., Science 261:1303 [1993]; Carrell et al., Angew. Chem. Int. Ed. Engl. 33.2059 [1994]; Carell et al., Angew. Chem. Int. Ed. Engl. 33:2061 [1994]; and Gallop et al., J. Med. Chem. 37:1233 [1994].

Libraries of compounds may be presented in solution (e.g., Houghten, Biotechniques 13:412-421 [1992]), or on beads (Lam, Nature 354:82-84 [1991]), chips (Fodor, Nature 364:555-556 [1993]), bacteria or spores (U.S. Pat. No. 5,223,409; herein incorporated by reference), plasmids (Cull et al., Proc. Nad. Acad. Sci. USA 89:18651869 [1992]) or on phage (Scott and Smith, Science 249:386-390 [1990]; Devlin Science 249:404-406 [1990]; Cwirla et al., Proc. Natl. Acad. Sci. 87:6378-6382 [1990]; Felici, J. Mol. Biol. 222:301 [1991]).

In some embodiments, the technology provided herein relates to gene therapy. For example, some embodiments use genetic manipulation to modulate the expression of biomarkers such as those described herein that are part of or are associated with the Slit-Robo-Myo9-RhoA pathway. Examples of genetic manipulation include, but are not limited to, gene knockout (such as by removing the genetic rearrangement from the chromosome using, e.g., by recombination), gene knock-down, expression of antisense constructs with or without inducible promoters, and the like. Delivery of nucleic acid construct to cells in vitro or in vivo may be conducted using any suitable method. A suitable method is one that introduces the nucleic acid construct into the cell such that the desired event occurs (e.g., expression of an antisense construct). Genetic therapy may also be used to deliver siRNA or other interfering molecules that are expressed in vivo (e.g., upon stimulation by an inducible promoter).

Introduction of molecules carrying genetic information into cells is achieved by any of various methods including, but not limited to, directed injection of naked DNA constructs, bombardment with gold particles loaded with said constructs, and macromolecule-mediated gene transfer using, for example, liposomes, biopolymers, and the like. Preferred methods use gene delivery vehicles derived from viruses, including, but not limited to, adenoviruses, retroviruses, vaccinia viruses, and adeno-associated viruses. Because of the higher efficiency as compared to retroviruses, vectors derived from adenoviruses are the preferred gene delivery vehicles for transferring nucleic acid molecules into host cells in vivo. Adenoviral vectors and their use in gene transfer are well known (e.g., see PCT publications WO 00/12738 and WO 00/09675 and U.S. Pat. Nos. 6,033,908, 6,019,978, 6,001,557, 5,994,132, 5,994,128, 5,994,106, 5,981,225, 5,885,808, 5,872,154, 5,830,730, and 5,824,544, each of which is herein incorporated by reference in its entirety). Such vectors and methods have been shown to provide very efficient in vivo gene transfer into a variety of solid tumors in animal models and into human solid tumor xenografts in immune-deficient mice.

Vectors may be administered to subject in a variety of well known ways, e.g., administered into tumors or tissue associated with tumors by using direct injection or administration via the blood or lymphatic circulation (See e.g., PCT publication 99/02685 herein incorporated by reference in its entirety). Exemplary dose levels of adenoviral vector are preferably 108 to 1011 vector particles added to the perfusate.

In some embodiments, the technology comprises use of gene editing or genome editing. Gene editing, or genome editing, is a type of genetic engineering in which DNA is inserted, replaced, or removed from a genome using nucleases. The nucleases may be artificially engineered. Alternately, the nucleases may be found in nature. The nucleases create specific double-stranded breaks (DSBs) at desired locations in the genome. The cell's endogenous repair mechanisms subsequently repairs the induced break(s) by natural processes, such as homologous recombination (HR) and non-homologous end-joining (NHEJ). Nucleases include, for example, Zinc Finger Nucleases (ZFNs), Transcription Activator-Like Effector Nucleases (TALENs), CRISPR, (e.g., the CRISPR/Cas system), and engineered meganuclease re-engineered homing endonucleases. CRISPR nucleases include for example a Cas nuclease, a Cpf1 nuclease, a C2c1 nuclease, a C2c3 nuclease, and a C2c3 nuclease. In some embodiments, gene editing or genome editing comprises use of CRISPR.

In some embodiments, the nuclease comprises a CRISPR/Cas system. The CRISPR (clustered regularly interspaced short palindromic repeats) locus, which encodes RNA components of the system, and the Cas (CRISPR-associated) locus, which encodes proteins (Jansen et al., 2002. Mol. Microbiol. 43: 1565-1575; Makarova et al., 2002. Nucleic Acids Res. 30: 482-496; Makarova et al., 2006. Biol. Direct 1: 7; Haft et al., 2005. PLoS Comput. Biol. 1: e60) make up the gene sequences of the CRISPR/Cas nuclease system. CRISPR loci in microbial hosts contain a combination of CRISPR-associated (Cas) genes as well as non-coding RNA elements capable of programming the specificity of the CRISPR-mediated nucleic acid cleavage.

The Type II CRISPR is one of the most well characterized systems and carries out targeted DNA double-strand breaks in four sequential steps. First, two non-coding RNAs, the pre-crRNA array and tracrRNA, are transcribed from the CRISPR locus. Second, tracrRNA hybridizes to the repeat regions of the pre-crRNA and mediates the processing of pre-crRNA into mature crRNAs containing individual spacer sequences. Third, the mature crRNA:tracrRNA complex directs Cas9 to the target DNA via Watson-Crick base-pairing between the spacer on the crRNA and the protospacer on the target DNA next to the protospacer adjacent motif (PAM), an additional requirement for target recognition. Finally, Cas9 mediates cleavage of target DNA to create a double-stranded break within the protospacer. Activity of the CRISPR/Cas system comprises of three steps: (i) insertion of alien DNA sequences into the CRISPR array to prevent future attacks, in a process called “adaptation”, (ii) expression of the relevant proteins, as well as expression and processing of the array, followed by (iii) RNA-mediated interference with the alien nucleic acid. Thus, in the bacterial cell, several of the so-called “Cas” proteins are involved with the natural function of the CRISPR/Cas system and serve roles in functions such as insertion of the alien DNA.

In certain embodiments, Cas protein may be a “functional derivative” of a naturally occurring Cas protein. A “functional derivative” of a native sequence polypeptide is a compound having a qualitative biological property in common with a native sequence polypeptide. “Functional derivatives” include, but are not limited to, fragments of a native sequence and derivatives of a native sequence polypeptide and its fragments, provided that they have a biological activity in common with a corresponding native sequence polypeptide. A biological activity contemplated herein is the ability of the functional derivative to hydrolyze a DNA substrate into fragments. The term “derivative” encompasses both amino acid sequence variants of polypeptide, covalent modifications, and fusions thereof. Suitable derivatives of a Cas polypeptide or a fragment thereof include but are not limited to mutants, fusions, covalent modifications of Cas protein or a fragment thereof. Cas protein, which includes Cas protein or a fragment thereof, as well as derivatives of Cas protein or a fragment thereof, may be obtainable from a cell or produced in vitro or by a combination of these two procedures. The cell may be a cell that naturally produces Cas protein or a cell that naturally produces Cas protein and is genetically engineered to produce the endogenous Cas protein at a higher expression level or to produce a Cas protein from an exogenously introduced nucleic acid, which encodes a Cas that is the same as or different from the endogenous Cas. In some cases, the cell does not naturally produce Cas protein and is genetically engineered to produce a Cas protein.

The method also includes introducing single-guide RNAs (sgRNAs) into the cell or the organism. The guide RNAs (sgRNAs) include nucleotide sequences that are complementary to the target chromosomal DNA. The sgRNAs can be, for example, engineered single chain guide RNAs that comprise a crRNA sequence (complementary to the target DNA sequence) and a common tracrRNA sequence, or as crRNA-tracrRNA hybrids. The sgRNAs can be introduced into the cell or the organism as a DNA (with an appropriate promoter), as an in vitro transcribed RNA, or as a synthesized RNA.

In addition, ZFPs and/or TALEs have been fused to nuclease domains to create ZFNs and TALENs, a functional entity that is able to recognize its intended nucleic acid target through its engineered (ZFP or TALE) DNA-binding domain and cause the DNA to be cut near the DNA-binding site via the nuclease activity. See, e.g., Kim et al. (1996) Proc Nat'l Acad Sci USA 93(3):1156-1160. More recently, such nucleases have been used for genome modification in a variety of organisms. See, for example, United States Patent Publications 20030232410; 20050208489; 20050026157; 20050064474; 20060188987; 20060063231; and International Publication WO 07/014,275.

Thus, the methods and compositions described herein are broadly applicable and may involve any nuclease of interest. Non-limiting examples of nucleases include meganucleases, TALENs, and zinc finger nucleases. The nuclease may comprise heterologous DNA-binding and cleavage domains (e.g., zinc finger nucleases; meganuclease DNA-binding domains with heterologous cleavage domains) or, alternatively, the DNA-binding domain of a naturally occurring nuclease may be altered to bind to a selected target site (e.g., a meganuclease that has been engineered to bind to site different than the cognate binding site).

In any of the nucleases described herein, the nuclease can comprise an engineered TALE DNA-binding domain and a nuclease domain (e.g., endonuclease and/or meganuclease domain), also referred to as TALENs. Methods and compositions for engineering these TALEN proteins for robust, site-specific interaction with the target sequence of the user's choosing have been published (see U.S. Pat. No. 8,586,526). In some embodiments, the TALEN comprises an endonuclease (e.g., Fold) cleavage domain or cleavage half-domain. In other embodiments, the TALE-nuclease is a mega TAL. These mega TAL nucleases are fusion proteins comprising a TALE DNA-binding domain and a meganuclease cleavage domain. The meganuclease cleavage domain is active as a monomer and does not require dimerization for activity. (See Boissel et al., (2013) Nucl Acid Res: 1-13). In addition, the nuclease domain may also exhibit DNA-binding functionality.

In still further embodiments, the nuclease comprises a compact TALEN (cTALEN). These are single chain fusion proteins linking a TALE DNA-binding domain to a TevI nuclease domain. The fusion protein can act as either a nickase localized by the TALE region, or can create a double-strand break, depending upon where the TALE DNA-binding domain is located with respect to the TevI nuclease domain (see Beurdeley et al. (2013) Nat Comm: 1-8). Any TALENs may be used in combination with additional TALENs (e.g., one or more TALENs (cTALENs or FokI-TALENs) with one or more mega-TALs) or other DNA cleavage enzymes.

In certain embodiments, the nuclease comprises a meganuclease (homing endonuclease) or a portion thereof that exhibits cleavage activity. Naturally occurring meganucleases recognize 15-40 base-pair cleavage sites. Exemplary homing endonucleases include I-SceI, I-CeuI, PI-PspI, PI-Sce, I-SceIV, I-CsmI, I-PanI, I-SceII, I-PpoI, I-SceIII, I-CreI, I-TevI, I-TevII and I-TevIII. Their recognition sequences are known. See also U.S. Pat. No. 5,420,032; U.S. Pat. No. 6,833,252; Belfort et al. (1997) Nucleic Acids Res. 25:3379-3388; Dujon et al. (1989) Gene 82:115-118; Perler et al. (1994) Nucleic Acids Res. 22, 1125-1127; Jasin (1996) Trends Genet. 12:224-228; Gimble et al. (1996) J. Mol. Biol. 263:163-180; Argast et al. (1998) J. Mol. Biol. 280:345-353 and the New England Biolabs catalogue.

DNA-binding domains from naturally occurring meganucleases have been used to promote site-specific genome modification in plants, yeast, Drosophila, mammalian cells and mice, but this approach has been limited to the modification of either homologous genes that conserve the meganuclease recognition sequence (Monet et al. (1999), Biochem. Biophysics. Res. Common. 255: 88-93) or to pre-engineered genomes into which a recognition sequence has been introduced (Route et al. (1994), Mol. Cell. Biol. 14: 8096-106; Chilton et al. (2003), Plant Physiology. 133: 956-65; Puchta et al. (1996), Proc. Natl. Acad. Sci. USA 93: 5055-60; Rong et al. (2002), Genes Dev. 16: 1568-81; Gouble et al. (2006), J. Gene Med. 8(5):616-622). Accordingly, attempts have been made to engineer meganucleases to exhibit novel binding specificity at medically or biotechnologically relevant sites (Porteus et al. (2005), Nat. Biotechnol. 23: 967-73; Sussman et al. (2004), J. Mol. Biol. 342: 31-41; Epinat et al. (2003), Nucleic Acids Res. 31: 2952-62; Chevalier et al. (2002) Molec. Cell 10:895-905; Epinat et al. (2003) Nucleic Acids Res. 31:2952-2962; Ashworth et al. (2006) Nature 441:656-659; Paques et al. (2007) Current Gene Therapy 7:49-66; U.S. Patent Publication Nos. 20070117128; 20060206949; 20060153826; 20060078552; and 20040002092). In addition, naturally occurring or engineered DNA-binding domains from meganucleases can be operably linked with a cleavage domain from a heterologous nuclease (e.g., FokI), and/or cleavage domains from meganucleases can be operably linked with a heterologous DNA-binding domain (e.g., ZFP or TALE).

In other embodiments, the nuclease is a zinc finger nuclease (ZFN) or TALE DNA-binding domain-nuclease fusion (TALEN). ZFNs and TALENs comprise a DNA-binding domain (zinc finger protein or TALE DNA-binding domain) that has been engineered to bind to a target site of choice and cleavage domain or a cleavage half-domain (e.g., from a restriction and/or meganuclease as described herein).

As described in detail above, zinc finger binding domains and TALE DNA-binding domains can be engineered to bind to a sequence of choice. See, for example, Beerli et al. (2002) Nature Biotechnol. 20:135-141; Pabo et al. (2001) Ann. Rev. Biochem. 70:313-340; Isalan et al. (2001) Nature Biotechnol. 19:656-660; Segal et al. (2001) Curr. Opin. Biotechnol. 12:632-637; Choo et al. (2000) Curr. Opin. Struct. Biol. 10:411-416. An engineered zinc finger binding domain or TALE protein can have a novel binding specificity compared to a naturally occurring protein. Engineering methods include, but are not limited to, rational design and various types of selection. Rational design includes, for example, using databases comprising triplet (or quadruplet) nucleotide sequences and individual zinc finger or TALE amino acid sequences, in which each triplet or quadruplet nucleotide sequence is associated with one or more amino acid sequences of zinc fingers or TALE repeat units which bind the particular triplet or quadruplet sequence. See, for example, U.S. Pat. Nos. 6,453,242 and 6,534,261, incorporated by reference herein in their entireties.

Selection of target sites and methods for design and construction of fusion proteins (and polynucleotides encoding same) are known to those of skill in the art and described in detail in U.S. Pat. Nos. 7,888,121 and 8,409,861, incorporated by reference in their entireties herein.

In addition, as disclosed in these and other references, zinc finger domains, TALEs, and/or multi-fingered zinc finger proteins may be linked together using any suitable linker sequences, including for example, linkers of 5 or more amino acids in length. See, e.g., U.S. Pat. Nos. 6,479,626; 6,903,185; and 7,153,949 for exemplary linker sequences of 6 or more amino acids in length. The proteins described herein may include any combination of suitable linkers between the individual zinc fingers of the protein. See, also, U.S. Provisional Patent Application No. 61/343,729.

Thus, nucleases such as ZFNs, TALENs and/or meganucleases can comprise any DNA-binding domain and any nuclease (cleavage) domain (cleavage domain, cleavage half-domain). As noted above, the cleavage domain may be heterologous to the DNA-binding domain, for example a zinc finger or TAL-effector DNA-binding domain and a cleavage domain from a nuclease or a meganuclease DNA-binding domain and cleavage domain from a different nuclease. Heterologous cleavage domains can be obtained from any endonuclease or exonuclease. Exemplary endonucleases from which a cleavage domain can be derived include, but are not limited to, restriction endonucleases and homing endonucleases. See, for example, 2002-2003 Catalogue, New England Biolabs, Beverly, Mass.; and Belfort et al. (1997) Nucleic Acids Res. 25:3379-3388. Additional enzymes which cleave DNA are known (e.g., 51 Nuclease; mung bean nuclease; pancreatic DNase I; micrococcal nuclease; yeast HO endonuclease; see also Linn et al. (eds.) Nucleases, Cold Spring Harbor Laboratory Press, 1993). One or more of these enzymes (or functional fragments thereof) can be used as a source of cleavage domains and cleavage half-domains.

Similarly, a cleavage half-domain can be derived from any nuclease or portion thereof, as set forth above, that requires dimerization for cleavage activity. In general, two fusion proteins are required for cleavage if the fusion proteins comprise cleavage half-domains. Alternatively, a single protein comprising two cleavage half-domains can be used. The two cleavage half-domains can be derived from the same endonuclease (or functional fragments thereof), or each cleavage half-domain can be derived from a different endonuclease (or functional fragments thereof). In addition, the target sites for the two fusion proteins are preferably disposed, with respect to each other, such that binding of the two fusion proteins to their respective target sites places the cleavage half-domains in a spatial orientation to each other that allows the cleavage half-domains to form a functional cleavage domain, e.g., by dimerizing. Thus, in certain embodiments, the near edges of the target sites are separated by 5-8 nucleotides or by 15-18 nucleotides. However any integral number of nucleotides or nucleotide pairs can intervene between two target sites (e.g., from 2 to 50 nucleotide pairs or more). In general, the site of cleavage lies between the target sites.

Restriction endonucleases (restriction enzymes) are present in many species and are capable of sequence-specific binding to DNA (at a recognition site) and cleaving DNA at or near the site of binding. Certain restriction enzymes (e.g., Type IIS) cleave DNA at sites removed from the recognition site and have separable binding and cleavage domains. For example, the Type IIS enzyme Fok I catalyzes double-stranded cleavage of DNA at 9 nucleotides from its recognition site on one strand and 13 nucleotides from its recognition site on the other. See, for example, U.S. Pat. Nos. 5,356,802; 5,436,150 and 5,487,994; as well as Li et al. (1992) Proc. Natl. Acad. Sci. USA 89:4275-4279; Li et al. (1993) Proc. Natl. Acad. Sci. USA 90:2764-2768; Kim et al. (1994a) Proc. Natl. Acad. Sci. USA 91:883-887; Kim et al. (1994b) J. Biol. Chem. 269:31,978-31,982. Thus, in one embodiment, fusion proteins comprise the cleavage domain (or cleavage half-domain) from at least one Type IIS restriction enzyme and one or more zinc finger binding domains, which may or may not be engineered.

An exemplary Type IIS restriction enzyme, whose cleavage domain is separable from the binding domain, is FokI. This particular enzyme is active as a dimer, as described by Bitinaite et al. (1998) Proc. Natl. Acad. Sci. USA 95: 10,570-10,575. Accordingly, for the purposes of the present disclosure, the portion of the FokI enzyme used in the disclosed fusion proteins is considered a cleavage half-domain. Thus, for targeted double-stranded cleavage and/or targeted replacement of cellular sequences using zinc finger-FokI fusions, two fusion proteins, each comprising a FokI cleavage half-domain, can be used to reconstitute a catalytically active cleavage domain. Alternatively, a single polypeptide molecule containing a zinc finger binding domain and two FokI cleavage half-domains can also be used. Parameters for targeted cleavage and targeted sequence alteration using zinc finger-FokI fusions are provided elsewhere in this disclosure.

A cleavage domain or cleavage half-domain can be any portion of a protein that retains cleavage activity, or that retains the ability to multimerize (e.g., dimerize) to create a functional cleavage domain.

Exemplary Type IIS restriction enzymes are described in International Publication WO 07/014,275, incorporated herein in its entirety. Additional restriction enzymes also contain separable binding and cleavage domains, and these are contemplated by the present disclosure. See, for example, Roberts et al. (2003) Nucleic Acids Res. 31:418-420.

In certain embodiments, the cleavage domain comprises one or more engineered cleavage half-domain (also referred to as dimerization domain mutants) that minimize or prevent homodimerization, as described, for example, in U.S. Pat. Nos. 7,914,796; 8,034,598 and 8,623,618; and U.S. Patent Publication No. 20110201055, the disclosures of all of which are incorporated by reference in their entireties herein Amino acid residues at positions 446, 447, 479, 483, 484, 486, 487, 490, 491, 496, 498, 499, 500, 531, 534, 537, and 538 of FokI are all targets for influencing dimerization of the FokI cleavage half-domains.

Engineered cleavage half-domains described herein can be prepared using any suitable method, for example, by site-directed mutagenesis of wild-type cleavage half-domains (FokI) as described in U.S. Pat. Nos. 7,914,796; 8,034,598 and 8,623,618; and U.S. Patent Publication No. 20110201055.

Alternatively, nucleases may be assembled in vivo at the nucleic acid target site using so-called “split-enzyme” technology (see e.g., U.S. Patent Publication No. 20090068164). Components of such split enzymes may be expressed either on separate expression constructs or can be linked in one open reading frame where the individual components are separated, for example, by a self-cleaving 2A peptide or IRES sequence. Components may be individual zinc finger binding domains or domains of a meganuclease nucleic acid binding domain.

Nucleases can be screened for activity prior to use, for example in a yeast-based chromosomal system as described in WO 2009/042163 and 20090068164. Nuclease expression constructs can be readily designed using methods known in the art. See, e.g., United States Patent Publications 20030232410; 20050208489; 20050026157; 20050064474; 20060188987; 20060063231; and International Publication WO 07/014,275. Expression of the nuclease may be under the control of a constitutive promoter or an inducible promoter, for example the galactokinase promoter which is activated (de-repressed) in the presence of raffinose and/or galactose and repressed in presence of glucose.

Accordingly, embodiments of the technology described herein relate to the use of gene editing or genome editing to modulate the expression and/or activity of a member of the Slit-Robo-Myo9-RhoA pathway (e.g., Myo9 (e.g., Myo9b, Myo9a); Slit (e.g., Slit1, Slit2, Slit3); Robo (e.g., Robo1, Robo2, Robo3, Robo4); RhoA; another Slit-Robo-Myo9-RhoA pathway gene; or another gene or gene product that modulates the Slit-Robo-Myo9-RhoA pathway). For example, some embodiments comprise increasing the activity or expression of one or more of Slit (e.g., Slit1, Slit2, Slit3); Robo (e.g., Robo1, Robo2, Robo3, Robo4); and/or RhoA. Some embodiments comprise use of gene editing or genome editing to overexpress Slit (e.g., Slit1, Slit2, Slit3). Some embodiments comprise use of gene editing or genome editing to overexpress Robo (e.g., Robo1, Robo2, Robo3, Robo4). Some embodiments comprise use of gene editing or genome editing to overexpress RhoA. Further, some embodiments comprise use of gene editing or genome editing to decrease the expression or activity of Myo9 (e.g., Myo9a, Myo9b).

### EXPERIMENTAL

### Example 1

### Slit-Robo-Myo9b-RhoA Pathway Implicated in Cell Migration and Lung Cancer Metastasis

### Results

### Slit2 Inhibits Cell Migration by Regulating RhoA Activity in Lung Cancer Cells.

To investigate the involvement of Slit-Robo signaling in lung cancer pathogenesis, we first examined the expression of Slit2 and its receptor Robo1 by RT-PCR in various cell lines derived from human lung cancer, including H1299 cells. In most lung cancer cell lines surveyed and a significant fraction of primary lung cancer samples examined, Slit2 expression was low or non-detectable (FIG. 9A). Robo1 protein, however, was detected in these lung cancer cell lines by Western blotting using a specific anti-Robo1 antibody (FIG. 9B).

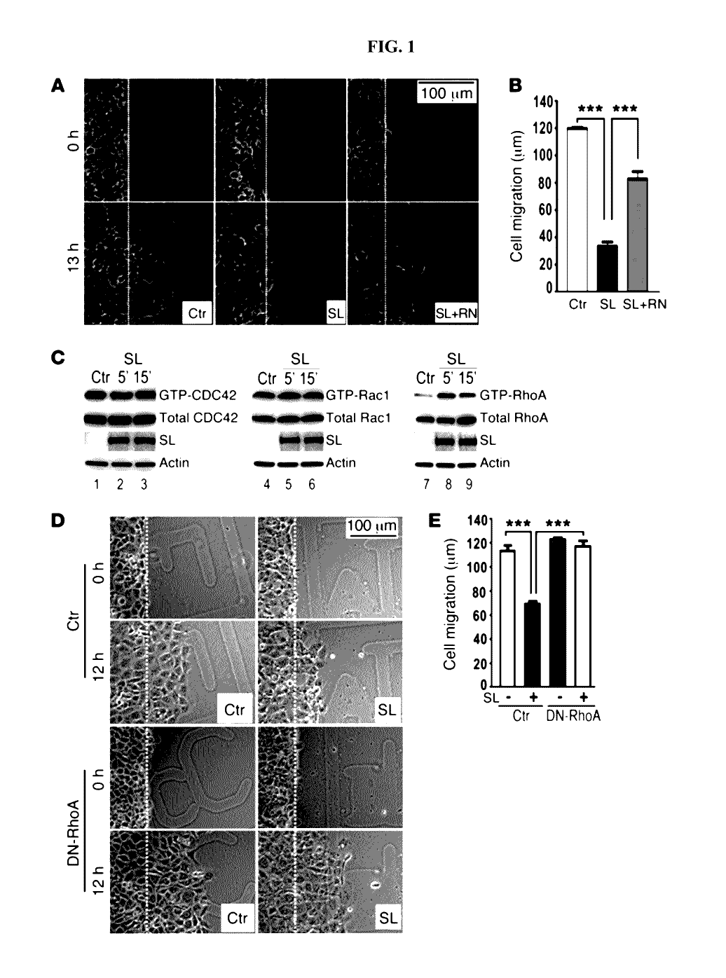

To examine the effect of Slit2 on lung cancer cell migration, we set up a wound-healing assay using H1299 lung cancer cells. The cells were treated with the mock control (Ctr) or Slit2 (Sl)-containing media after the wound formation. Slit2 treatment significantly inhibited the migration of H1299 cells (FIGS. 1, A and B). To test the role of Robo1 in mediating Slit2 activity in lung cancer cells, we used the Robo1N (RN), the soluble extracellular domain of Robo1 capable of blocking Slit signaling (Li, 1999; Wu, 2001; Yuasa-Kawada, Proc Natl Acad Sci USA, 2009; herein incorporated by reference in their entireties). Addition of Robo1N to the wound-healing assay effectively reversed the inhibitory effect of Slit2 on lung cancer cell migration (FIGS. 1, A and B), indicating that Slit2 inhibits H1299 cell migration in a Robo1-dependent manner. We also tested Slit2 activity in another human lung cancer cell line A549. Similarly, Slit2 suppressed the migration of A549 cells, and Robo1N abolished the Slit2 inhibitory effect (FIGS. 9, C and D).

To investigate the molecular mechanism underlying Slit2 function in lung cancer cells, we examined the signal transduction pathways involved. Slit reduces the level of active Cdc42 in neurons and that Slit-Robo GAP1 (srGAP1) is required for Slit-induced suppression of Cdc42 activity in neuronal migration (Wong, 2001; herein incorporated by reference in its entirety). Experiments were conducted during development of embodiments herein to determine if the srGAP1-Cdc42 pathway, observed in neurons, mediated the Slit inhibitory effect on lung cancer cell migration. We systematically examined the effect of Slit2 on small GTPases including Cdc42, Rac1 and RhoA. H1299 cells expressing myc-tagged Cdc42 or Rac1 were treated with control or Slit2 for 5 min or 15 min. Cell lysates were then incubated with GST-Pak Binding Domain (GST-PBD), a protein domain specifically interacting with active (GTP-bound) Cdc42 or Rac1 (Heasman, 2008; herein incorporated by reference in its entirety). Extracts from H1299 cells treated with control or Slit2 for 5 min or 15 min were incubated with a GST-Rhotekin Binding Domain (GST-RBD) selectively binding to the active form of RhoA (Jaffe, 2005; herein incorporated by reference in its entirety). The GTPases were detected in H1299 cell lysates by immunoblotting with corresponding antibodies following the GST pull-down experiment. Slit2 treatment did not affect the active levels of either Cdc42 or Rac1 (FIG. 1C, lanes 1-3 and lanes 4-6, respectively). In contrast, Slit2 treatment significantly increased the active RhoA level (FIG. 1C, lanes 7-9), indicating that Slit2 specifically modulates RhoA activity in lung cancer cells. Another method was then used to examine Slit2 activity in H1299 cells. Instead of adding Slit2 to the culture media, we made two stable H1299 cell lines that overexpressed the human Slit2 gene. The similar activity in activating RhoA, but not Cdc42 or Rac1, was detected when Slit was expressed in H1299 cells (FIG. 9E). The observation that Slit2 activates RhoA in lung cancer cells, but not Cdc42 or Rac1 suggests that the mechanism by which Slit2 regulates small GTPase activity in lung cancer cells is distinct from that in neurons.

To examine whether changes in the RhoA activity were required for Slit2 inhibition of lung cancer cell migration, we transfected H1299ctr (Ctr) and H1299Slit (Sl) cells with a dominant negative form (DN) of RhoA that was described previously (Wong, 2001; herein incorporated by reference in its entirety). Wound-healing experiments were carried out to determine the effect of RhoA on cell migration. Expression of the DN-RhoA mutant significantly reduced the Slit2 inhibitory effect on migration of H1299 cells (FIGS. 1, D and E), indicating that Slit2 inhibits lung cancer cell migration in a RhoA-dependent manner. The above data show that Slit2 inhibits cell migration by regulating RhoA activity in lung cancer cells.

Myo9b Interacts with Robo1 and Mediates Slit2-Induced Inhibition of Cell Migration and Activation of RhoA.

To dissect the Slit-Robo signaling pathways, experiments were conducted during development of embodiments herein to search for proteins interacting with the intracellular domain of the Robo1 protein (Yuasa-Kawada, Proc Natl Acad Sci USA. 2009; Wong, 2001; herein incorporated by reference in their entireties). From yeast two-hybrid screens, one group of cDNA clones was identified to encode the Myo9b protein, in addition to previously reported genes, including srGAPs (Wong, 2001; herein incorporated by reference in its entirety) and USP33 (Yuasa-Kawada, 2009; herein incorporated by reference in its entirety). RT-PCR and Western blotting experiments show that Myo9b was expressed in lung cancer cell lines (FIGS. 9, A and B), in contrast to a previous report that Myo9b expression was restricted to the immune system (Hanley, 2010; herein incorporated by reference in its entirety).

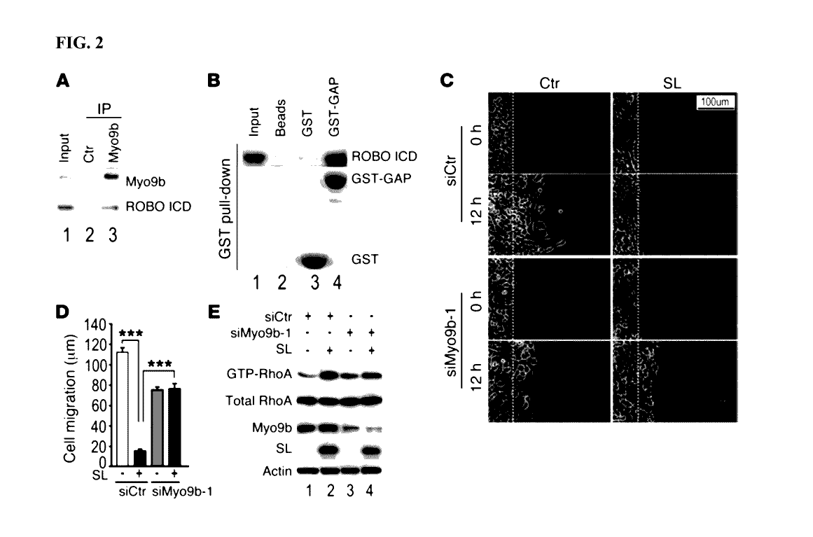

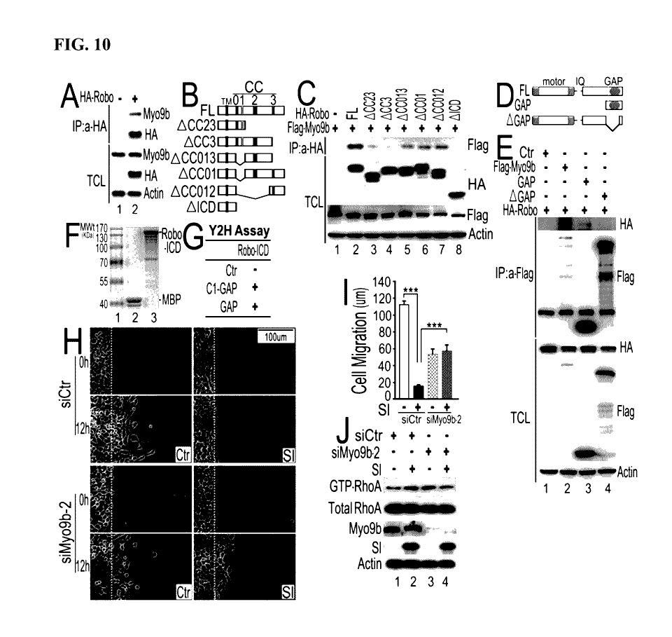

To confirm the interaction between Robo1 and Myo9b in mammalian cells, we carried out co-immunoprecipitation (co-IP) experiments using HEK293 cells transfected with plasmids encoding Robo1 containing a HA-tag (HA-Robo) or a vector control. Myo9b was detected in the immunoprecipitates formed with a specific anti-HA antibody from the HA-Robo expressing cells but not from the control cells (FIG. 10A). To examine whether the endogenously expressed Robo1 and Myo9b proteins were associated with each other in lung cancer cells, H1299 cell lysates were immunoprecipitated using anti-Myo9b or control IgG followed by immunoblotting with anti-Robo1. Robo1 was detected in the proteins immunoprecipitated by anti-Myo9b antibody, but not by control IgG (Ctr) (FIG. 2A). These results indicate that the endogenous Robo1 interacts with Myo9b in H1299 lung cancer cells.

Robo1 is a transmembrane receptor containing five immunoglobulin (Ig) domains, three fibronectin (Fn) III repeats in the extracellular region and four cytoplasmic conserved (CC) motifs in the intracellular region (Li, 1999; herein incorporated by reference in its entirety). Myo9b contains a motor domain in the head region, four IQ motifs in the neck region and a RhoGAP domain in the tail region (Post, 1998; herein incorporated by reference in its entirety). To characterize the domain(s) of Robo1 and Myo9b involved in Robo1-Myo9b interaction, we utilized a panel of Robo1 deletion mutants (FIG. 10B) (Yuasa-Kawada, 1999; herein incorporated by reference in its entirety) and constructed a series of Myo9b mutants (FIG. 10D). A co-IP assay was carried out using lysates of H1299 cells that had been co-transfected with Flag-tagged Myo9b (Flag-Myo9b) and HA-tagged Robo1 (the full-length or deletion mutants). All four CC motifs in the intracellular domain (ICD) of Robo1 contributed to the Robo1-Myo9b interaction, whereas deletion of the intracellular domain completely eliminated the interaction between Robo1 and Myo9b (FIG. 10C). On the other hand, the RhoGAP domain in Myo9b is required for its association with Robo1 (FIG. 10E). Therefore, Myo9b interacts with the intracellular domain of Robo1 via the RhoGAP domain.

Experiments were conducted during development of embodiments herein to determine if Myo9b could directly interact with Robo1. GST pull-down experiments were performed using purified proteins: GST-tagged Myo9b RhoGAP domain (GST-GAP) and MBP-His6-tagged Robo-ICD domain (MBP-His6-RoboICD; (MBP: maltose-binding protein)) (see FIG. 10F for individual purified proteins). Following GST-pull down, the GST-GAP protein associated with MBP-His6-RoboICD was detected by Western blotting using anti-His antibody (FIG. 2B), demonstrating that purified Myo9b RhoGAP domain directly interacted with Robo-ICD domain. Consistently, the interaction between Robo-ICD and Myo9b (either GAP alone or C1-GAP (Protein kinase C conserved region 1 and RhoGAP domain)) was detected in the yeast two-hybrid assay (FIG. 10G). These experiments indicate that Myo9b RhoGAP domain can directly interact with the intracellular domain of Robo1.

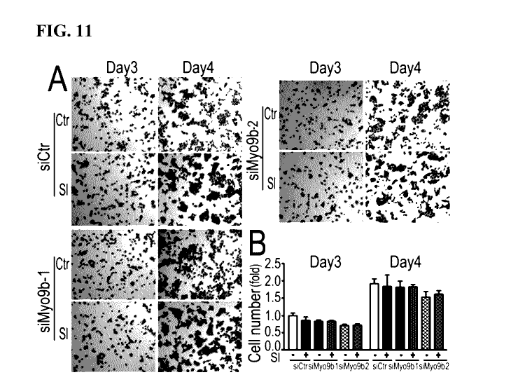

Because Myo9b interacts with Robo1 and displays the RhoA-specific GAP activity, we next tested if Myo9b played a role in mediating Slit-Robo signaling in suppressing cell migration and activating RhoA in lung cancer cells. The wound-healing experiments were carried out following knocking down Myo9b expression using two independent Myo9b-specific siRNAs (siMyo9b) in H1299 cells. Immunoblotting experiments confirmed that siMyo9b efficiently reduced Myo9b expression in H1299 cells (FIGS. 2E and 10J). Specific siRNAs against Myo9b, but not control siRNAs (siNC), abolished Slit activity in inhibiting cell migration, indicating that the inhibitory effect of Slit on lung cancer cell migration depends on Myo9b (FIGS. 2, C and D and FIGS. 10, H and I). No difference was observed in cell proliferation between control and siMyo9bs-mediated H1299 cells treated with Slit-containing media (FIGS. 11, A and B). In addition, the GST pull-down experiments were performed using the GST-RBD to examine RhoA activation after H1299 cells were transfected with the control or two Myo9b-specific siRNAs. When Myo9b was down-regulated in H1299 cells, Slit-induced RhoA activation was significantly reduced (FIGS. 2E and 10J), demonstrating that Myo9b is required for Slit-induced RhoA activation in H1299 cells. Together, these data show that Myo9b is indeed important for Slit-Robo signaling in lung cancer cells.

### Myo9b Inactivates RhoA Through its RhoGAP Domain in Lung Cancer Cells.

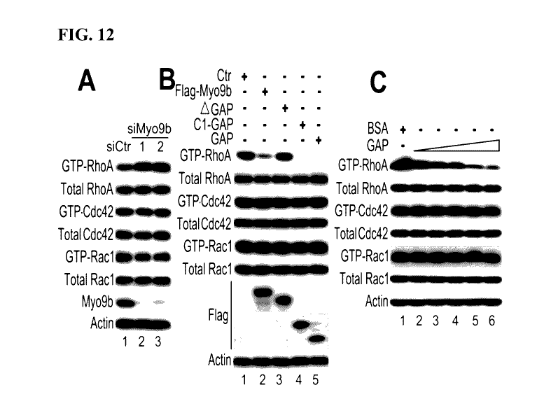

Since Myo9b is critical for Slit-Robo signaling in regulating RhoA activity, we next test whether Myo9b can specifically inactivate RhoA in lung cancer cells. We examined the effect of Myo9b on small GTPases, including RhoA, Cdc42 and Rac1 in H1299 cells. Following transfection with the control or two Myo9b-specific siRNAs, H1299 cell lysates were prepared and incubated with GST-RBD or GST-PBD respectively. The specific GTPase activity was determined by immunoblotting with corresponding antibodies following the GST pull-down experiment. When Myo9b was down-regulated, the level of active RhoA, GTP-RhoA, was significantly increased, whereas the levels of either active Cdc42 or active Rac1 were not affected (FIG. 12A). These data support that Myo9b specifically inactivates RhoA in lung cancer cells. To examine whether the GAP activity of Myo9b depends on its RhoGAP domain, the GST pull-down experiment was carried out using H1299 cells following transfection with plasmids expressing either the wild-type or deletion mutant Myo9b as Flag-tagged proteins, or the vector control. As expected, the full-length Myo9b, the C1-GAP or the RhoGAP domain of Myo9b reduced the level of active RhoA, but not those of Cdc42 or Rac1 (FIG. 12B). To further examine effects of Myo9b RhoGAP domain on small GTPase activity, we prepared highly purified Myo9b RhoGAP domain protein for the GST pull-down experiments. Extracts from HEK293T cells respectively transfected with myc-RhoA, myc-Cdc42 or myc-Rac1 incubated with the purified RhoGAP domain at different concentrations. Then, the GST pull-down experiments were carried out with active levels of small GTPases detected by Western blotting. Consistent with our observation presented above and previous studies of Myo9b, the purified Myo9b RhoGAP domain protein inhibits RhoA in a dose-dependent manner without affecting Cdc42 or Rac1 (FIG. 12C), indicating that Myo9b RhoGAP domain specifically modulates RhoA activity.

### Structural Analyses of the RhoGAP Domain in Myo9b

To examine the mechanism underlying the specific GAP activity of Myo9b for RhoA, we crystallized Myo9b RhoGAP domain and determined its structure at 2.2A resolution (FIG. 3 and Table 4). From the overall structure, Myo9b RhoGAP domain adopts a canonical RhoGAP fold with nine α-helices (namely, αA0 to αG according to the canonical RhoGAP domain structure) (FIG. 3, B-D). Among these helices, αA, αB, αE and αF are arranged as a four-helix bundle, forming the core of the structure (FIGS. 3, C and D). This four-helix bundle is capped by the shortest helix αA0 that is likely to complete the core structure. One side of the central four-helix bundle (the αA/αB side) immediately packs with αA1 (FIGS. 3, C and D), whereas at its opposite side (the αE/αF side), αC and αD forms a helical hairpin that protrudes from the four-helix bundle core. Interestingly, the last helix αG exhibits a perpendicular orientation to the other helices (except for αA0) and crosses the deep cave formed between the αC/αD helical hairpin and central four-helix bundle (FIGS. 3, C and D).

TABLE 4
Data collection and refinement statistics
of Myo9b RhoGAP domain

| Data collection | | |
|---|---|---|
| Space group | P2₁2₁2₁ | |
| Unit cell parameters(Å) | a = 77.1, b = 85.0, c = 134.1 | |
| Resolution range(Å) | 50.0-2.20 | (2.32-2.20)^{a} |
| No. of unique reflections | 45424 | (6528) |
| Redundancy | 6.9 | (7.1) |
| I/σ(I) | 10.5 | (3.0) |
| CC(1/2) | 0.996 | (0.896) |
| Rpim | 0.047 | (0.247) |
| Rmerge (%)^{b} | 11.3 | (61.5) |
| Completeness (%) | 99.9 | (100.0) |
| Structure refinement | | |
| Resolution (Å) | 50.0-2.20 | (2.25-2.20) |
| Rwork^{c}/Rfree (%)^{d} | 20.7(25.5)/23.7(32.6) | |
| R.M.S.D bonds (Å)/angles (°) | 0.003/0.70 | |
| Average B factor | 55.40 | |
| No. of atoms | | |
| Protein atoms | 6503 | |
| Water molecules | 338 | |
| No. of reflections | | |
| Working set | 43349 | |
| Test set | 1995 | |
| Ramachandran plot | | |

| | |
|---|---|
| Most favored regions (%) | 92.1 |
| Additionally allowed (%) | 6.4 |
| Generously allowed (%) | 0.8 |
| Disallowed (%) | 0.7 |

^{a}The values in parentheses refer to the highest resolution shell.
^{b}R_{merge} = ΣₕΣᵢ|Iᵢ(h) − <I(h)>|/ΣₕΣᵢIᵢ(h), where I is the observed intensity and <I> is the average intensity of multiple observations of symmetry-related reflection h.
^{c}R_{work} is the R_{factor} for the working dataset. R_{factor} = Σ∥Fo| − |Fc∥/Σ|Fo| where |Fo| and |Fc| are observed and calculated structure factor amplitudes respectively.
^{d}R_{free} is the cross-validation R_{factor} computed for a randomly chosen subset of 5% of the total number of reflections, which were not used during refinement.

The overall structure of Myo9b RhoGAP domain is similar to that of the canonical RhoGAP domain of p50rhoGAP, especially in the central four-helix bundle (FIG. 13). Consistent with this feature, the structure-based sequence alignment showed that the residues responsible for the four-helix bundle formation are highly conserved among different members of the RhoGAP family (FIG. 13A). In contrast to the central four-helix bundle, the neighboring αA1, αG and αC/αD helical hairpin show some differences between the Myo9b RhoGAP and p50rhoGAP, e.g., the loop between αA1 and αB of Myo9b RhoGAP is shorter than that of p50rhoGAP, whereas the last helix αG is relatively longer (FIGS. 13, B-E). These structural differences may distinguish Myo9b RhoGAP domain from RhoGAP domains in other proteins.
Myo9b RhoGAP Domain Contains a Unique Patch that Specifically Recognizes RhoA.

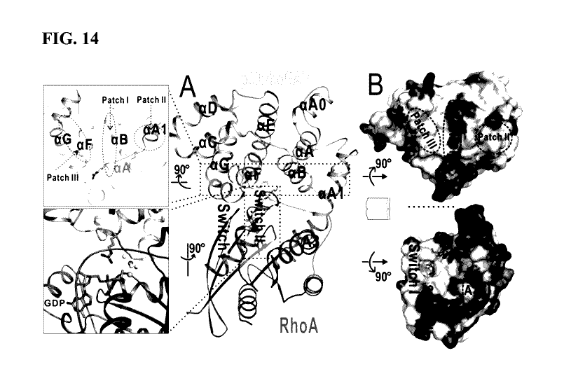

Based on the structure of the p50rhoGAP/RhoA complex (FIG. 14), the RhoA-binding site within the RhoGAP domain is formed by αA1, αB, αF, αG, and the αA/αA1 and αF/αG loops and can be divided into three patches (Patch I, II and III) with the active arginine-finger located in the αA/αA1 loop. Consistent with its structural similarity to p50rhoGAP domain (FIG. 14), Myo9b RhoGAP domain contains a similar site with three patches potentially forming a binding surface to interact with RhoA (FIGS. 3, E-G). More specifically, Patch I consists of R1735 from the αA1/αB loop and K1772 and R1776 from αB (FIGS. 3, B and F); Patch II is composed of A1739, N1741, R1742 and R1744 from the N-terminal half of αA1 (FIGS. 3, B and G); and Patch III is formed by 11848 and P1852 from αF and V1870 from αG (FIGS. 3, B and F). With these patches forming a potential binding site (FIG. 3E and FIG. 14), we confirmed that Myo9b RhoGAP domain indeed directly interacted with RhoA using the GST pull-down assay (FIG. 4E).

The above described similarity between Myo9b RhoGAP and p50rhoGAP domains prompted us to build a structural model of the Myo9b RhoGAP/RhoA complex by replacing the RhoGAP domain in the p50rhoGAP RhoGAP/RhoA complex structure by Myo9b RhoGAP domain. This model was further refined by molecular dynamics simulation in solution. As expected, the final structural model of the Myo9b RhoGAP/RhoA complex resembles that of the p50rhoGAP RhoGAP/RhoA complex, adopting a similar interaction mode with three patches binding to RhoA (Figure, 4, A and B and FIG. 14). Specifically, the positively charged Patch I interacts with negatively charged Switch II, Patch II packs with the A3 helix, and Patch III forms extensive hydrophobic contacts with Switch I. Because Patch I and III of the RhoGAP domain are highly conserved but Patch II is diverse in different RhoGAP family proteins (FIG. 13A and FIG. 14), the specificity of Myo9b RhoGAP domain toward RhoA may likely be determined by its Patch II. Consistent with this feature, the structure-based sequence alignment of different Rho family proteins (RhoA, Cdc42 and Rac1) shows that Switch I and Switch II responsible for interacting with Patch III and I of the RhoGAP domain are also highly conserved (FIG. 15), whereas the sequences for the A3 helix, the corresponding site for binding to Patch II, are diverse.

To further understand the specific recognition of RhoA by Myo9b RhoGAP domain, we analyzed the interaction interface between Patch II and the A3 helix. Patch II of Myo9b RhoGAP domain is enriched in positively charged residues (R1742 and R1744) that form electrostatic interactions with negatively charged residues (D90 and E97) in the A3 helix of RhoA (FIG. 4C). Moreover, N1741 in Patch II is likely to interact with E93 in the A3 helix; and A1739 in Patch II also forms hydrophobic interactions with the A3 helix (FIG. 4C). In contrast, D90 is replaced by S88 in Cdc42; and D90 and E97 are substituted with A88 and A95, respectively, in Rac1. These amino acid residue substitutions make Cdc42 and Rac1 poor candidates to interact with Myo9b RhoGAP domain because of the positively charged residues within Patch II of Myo9b RhoGAP domain (FIG. 15). Consistent with these structure-based analyses, point mutations in Patch II of Myo9b RhoGAP domain to reverse its charged property, including A1739E, N1741E and R1742E, each significantly decreased the binding of Myo9b RhoGAP domain to RhoA, thereby impairing subsequent inactivation of RhoA (FIGS. 4, D and E). In contrast, the A1739V mutation did not affect Myo9bRhoGAP-RhoA interaction, whereas the A1739N mutation did (FIGS. 4, D and E), again, supporting an essential role of hydrophobic interaction between A1739 of Myo9bGAP and the A3 helix of RhoA. Taken together, Myo9b RhoGAP domain contains a unique positively charged patch II that specifically recognizes and inactivates RhoA.

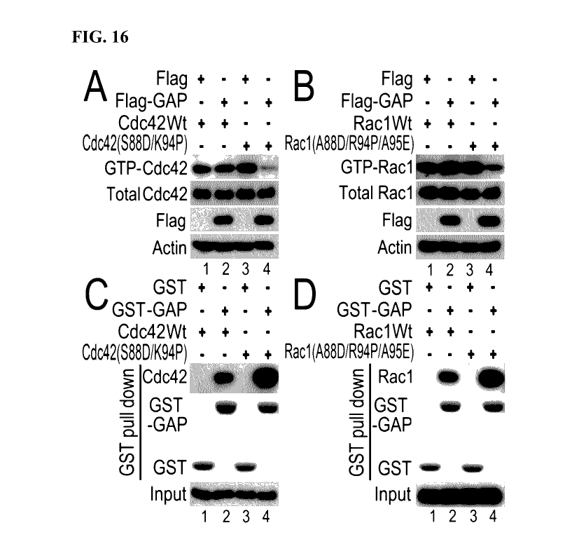

Additionally, we substituted corresponding amino acid residues in the A3 helix of Cdc42 or Rac1 to those of RhoA for binding to Patch II of RhoGAP, generating mutant forms of Cdc42S88D/K94P or Rac1A88D/R94P/A95E. In GST pull-down experiment, we demonstrated that Myo9b RhoGAP domain inactivated these mutant forms of Cdc42 or Rac1, but not wild-type form of Cdc42 or Rac1 (FIGS. 16, A and B). Consistent with this, the binding of Myo9b RhoGAP to mutant forms of Cdc42 or Rac1 dramatically increased as compared with the wild-type form (FIGS. 16,C and D), supporting that the specific electrostatic interaction between Patch II and the A3 helix may determine the specific recognition of RhoA by Myo9b RhoGAP domain.

### Slit-Robo Signaling Inactivates Myo9b RhoGAP Domain

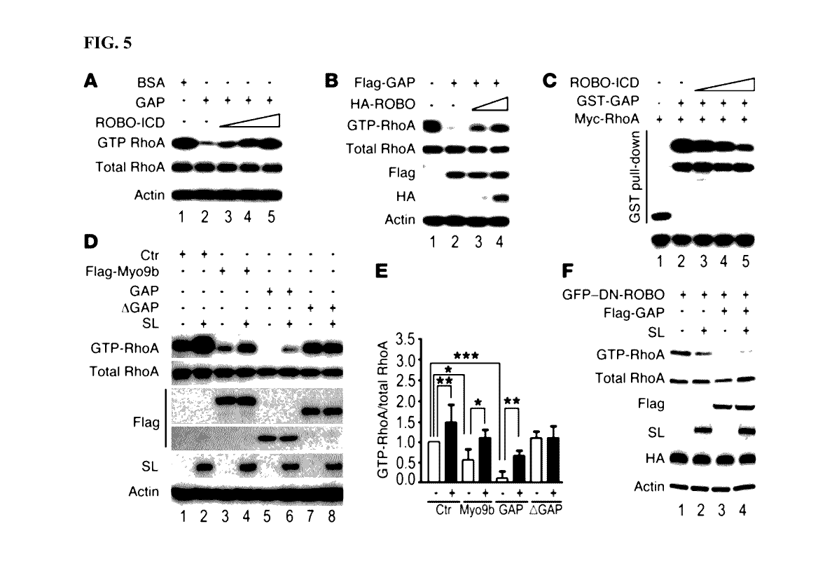

Detection of the interaction between the intracellular domain (ICD) of Robo1 and Myo9b RhoGAP domain prompted us to investigate whether Robo1 affected activity of Myo9b RhoGAP domain. The GST pull-down assay was performed using GST-RBD and HEK293T cell lysates transfected with myc-RhoA in the presence of various combinations of purified proteins: Myo9b RhoGAP and Robo-ICD (as MBP-tagged protein; (MBP: maltose-binding protein)). The effect of Robo-ICD on Myo9b RhoGAP activity was determined by Western blotting analyses of the pull-down products. Interestingly, Myo9b inhibitory activity on RhoA was suppressed by the addition of Robo-ICD in a dose-dependent manner (FIG. 5A, lanes 3-5), whereas addition of the control protein, MBP-tag alone, did not show any effect (FIG. 5A lane 2). Similarly, GST pull-down experiments using H1299 lung cancer cells that co-expressed a Flag-tagged Myo9b RhoGAP protein together with different levels of the full-length HA-tagged Robo1 (HA-Robo) demonstrated that Robo1 suppressed Myo9b GAP activity in inactivating RhoA (FIG. 5B). Taken together, these results indicate the intracellular domain (ICD) of Robo suppresses its GAP activity in converting GTP-RhoA to GDP-RhoA.

We next tested whether Robo-ICD interfered with the RhoGAP-RhoA interaction by performing the GST pull-down experiments using purified Robo-ICD protein and HEK293T cell lysates transfected with plasmids encoding myc-tagged RhoA. These experiments show that Robo-ICD protein blocks the interaction of RhoGAP with RhoA in a concentration-dependent manner (FIG. 5C), providing one mechanistic explanation for the inhibition of Myo9b RhoGAP activity by Robo-ICD.

To further characterize the effect of Slit2 on Myo9b-RhoGAP activity in lung cancer cells, the GST-RBD pull-down experiments were carried out in a stable H1299 cell line that overexpressed Slit2 following transfection with plasmids expressing either the wild-type or mutant Myo9b as Flag-tagged proteins, or the vector control. The activity of Myo9b in reducing the active RhoA level was suppressed by Slit2, leading to the increased levels of GTP-RhoA (FIG. 5D, compare lane 4 with lane 3; and lane 6 with lane 5, respectively). Interestingly, in the presence of a Myo9b mutant lacking its GAP domain (ΔGAP), Slit activity in increasing GTP-RhoA levels was blocked (FIG. 5D, compare lane 8 with lane 7), supporting that Slit activates RhoA by suppressing the activity of Myo9b in converting active GTP-RhoA to inactive GDP-RhoA and that the RhoGAP domain of Myo9b is required for Slit-induced RhoA activation.

We further tested if Slit inhibited Myo9b RhoGAP activity through the Robo1 receptor. We co-expressed a Robo1 mutant lacking its intracellular domain as a GFP-tagged protein (GFP-DNRobo) and Flag-tagged Myo9b-GAP in H1299 cells. In these cells, Slit2 treatment failed to induce RhoA activation, demonstrating that the inhibitory effect of Slit2 on Myo9b GAP activity is Robo1-dependent (FIG. 5F).

### Slit2 Suppresses Lung Cancer Invasion and Metastasis

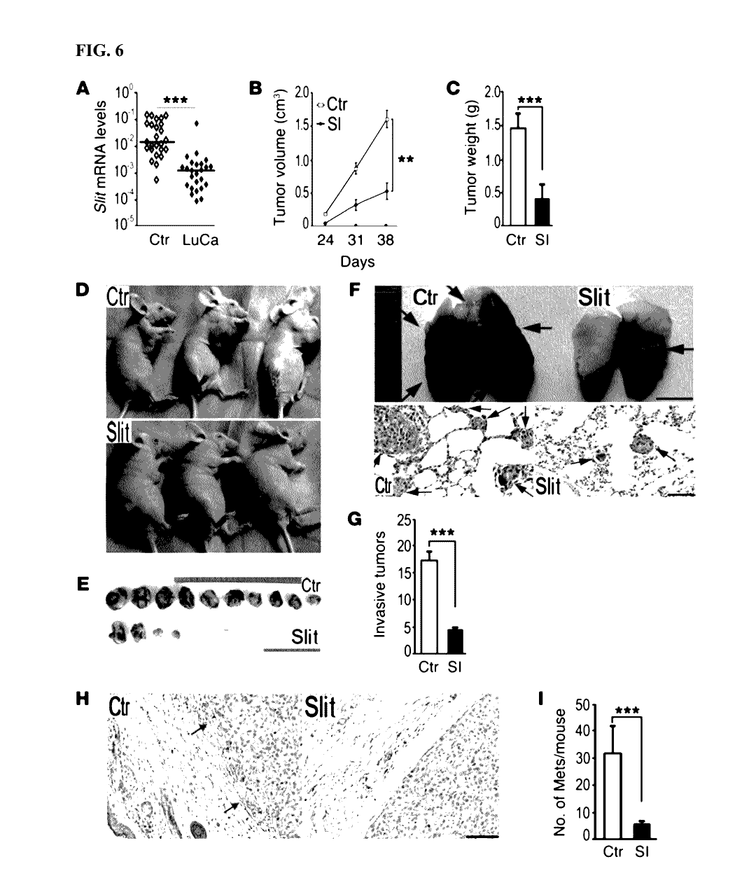

The data presented above define a Slit-Robo-Myo9b-RhoA signaling pathway that inhibits the lung cancer cell migration in vitro. These data also indicate that Slit2 is a suppressor for lung cancer. To investigate the role of Slit2 in lung cancer patients, 25 pairs of lung tumor samples were collected with the adjacent non-tumor tissues. The expression of Slit2 mRNA was analyzed by real-time RT-PCR with GAPDH as an internal control. Slit2 expression was significantly decreased in lung tumors as compared with the paired adjacent control tissues (FIG. 6A). To survey more patients, we analyzed published datasets for Slit2 expression in lung cancer using Oncomine database (www.oncomine.org) and gene microarray data analysis tools (34). Data from multiple datasets show that Slit2 gene expression is significantly down-regulated in human lung cancer samples as compared with controls (FIG. 17A), consistent with a previous report with a relatively small cohort (18). Kaplan-Meier analyses of different human lung cancer microarray datasets, including CaArray and GSE31210 (35) show that higher levels of Slit2 expression are associated with longer overall survival (OS) and progression-free survival (PFS) of patients (FIGS. 17, B and D). Even among late-stage lung cancer patients with tumor grade III, lower Slit expression is associated with shorter overall survival time (FIG. 17C). Importantly, in lung cancer patients with lymph node metastasis, higher Slit expression still correlates with better prognosis of overall survival (FIG. 17C). Our analysis of 178 lung cancer samples from the published TCGA (The Cancer Genome Atlas) dataset (Gao, 2013; herein incorporated by reference in its entirety) demonstrates that approximately 8%, 7% and 7% of the human lung cancer cases showed genetic alterations (including homozygous deletion and mutations) in the Slit2, Slit3 or Robo1 genes respectively (FIGS. 17, E-G), suggesting that Slit or Robo1 gene plays an important role in lung cancer pathogenesis. Taken together, these data strongly support a role of Slit in suppressing lung cancer in humans.

To determine whether Slit2 could suppress lung cancer invasion/metastasis in vivo, a xenograft animal model using H1299 cells was established in which the endogenous Slit2 expression was low. Stable H1299 cell lines were prepared expressing human Slit2 or the vector control (Sl or Ctr, respectively) and examined in the animal model. Following subcutaneous inoculation of either H1299ctr or H1299Slit cells into nude mice, tumor formation was monitored. By 24 days after tumor cell injection, palpable tumors were detected. Animals were euthanized and examined for local tumor invasion and lung metastasis. In the H1299ctr group, all 10 mice injected developed subcutaneous tumors, whereas in the H1299Slit group only 7 out of 10 mice showed detectable subcutaneous tumors (FIG. 6E). The average volume and weight of primary tumors in the H1299ctr group were significantly greater than those of Slit-expressing tumors in the H1299Slit group (FIG. 6, B-D), demonstrating that Slit expression in H1299 cells suppresses tumor growth or invasion in vivo. Histological examination of these primary tumors revealed that the majority of H1299ctr tumors exhibited local invasion with irregular borders and numerous microcapillaries adjacent to invading tumor cells; whereas most tumors derived from H1299Slit cells were surrounded by fibrous capsules with smooth/clear borders (FIGS. 6, F and G, and Table 1). These findings indicate that Slit expression significantly reduced invasion by H1299 lung cancer cells in vivo. Additional histological examination of mouse lung tissues was carried out to evaluate lung metastasis. In the H1299ctr group, all mice showed lung metastasis, whereas only 3 out of 10 mice in the H1299Slit group exhibited lung metastasis (FIG. 6, H and Table 2). In these mice, H1299Slit cells induced much reduced lung metastasis as compared with the H1299ctr cells, both in the sizes and numbers of lung metastatic tumors (FIGS. 6, H and I and Table 2). These results demonstrate that Slit inhibits lung cancer invasion and metastasis in the mouse model, supporting that Slit2 is a lung cancer suppressor gene.

TABLE 1
The number of mice with non-invasive
tumors in the corresponding groups

| | Noninvasive Tumors | |
|---|---|---|
| Groups | with Smooth Borders | p (X² test) |
| Ctr | 1/10 | 0.0345 |
| Slit | 5/7 | |

TABLE 2
Fractions of mice with lung metastasis in two groups

| Groups | Lung Metastasis | p (X² test) |
|---|---|---|
| Ctr | 10/10 | 0.0031 |
| Slit | 3/10 | |

Myo9b is Highly Expressed in Lung Cancer; and the High Myo9b Expression is Correlated with Lung Cancer Progression

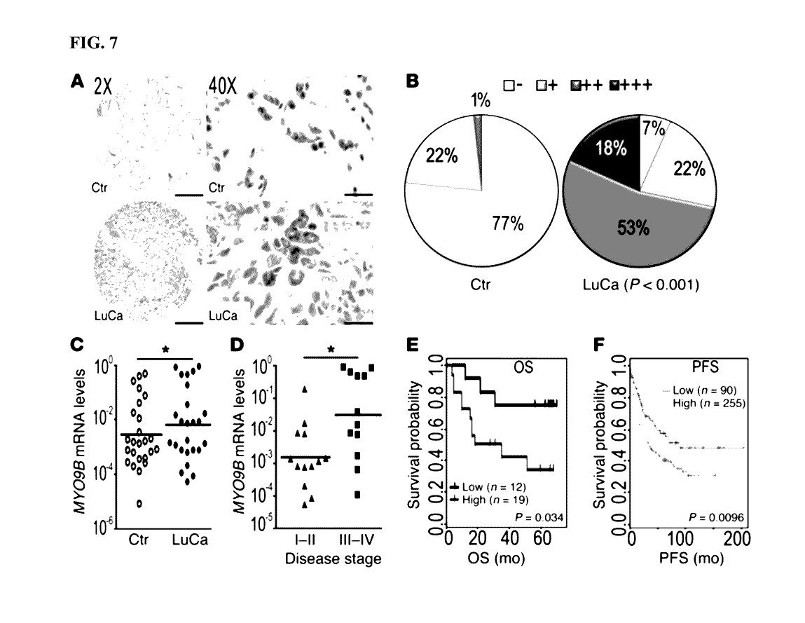

We investigated the potential involvement of Myo9b in human lung cancer. First, we examined Myo9b expression in a tissue-array panel containing 60 human lung cancer samples with corresponding matched adjacent non-tumor tissues. Fifty-six out of 60 cases (93%) of the lung cancer tissues showed positive Myo9b immunostaining signals, whereas only 14 out of 60 cases (23%) of the para-tumor control tissue samples were Myo9b-positive (FIG. 7A for a representative tissue pair and FIG. 7B). Consistent with our immunostaining data, real-time RT-PCR analyses of a separate cohort of 25 pairs of human lung cancer samples with controls showed that the mRNA level of Myo9b was significantly increased in lung cancer samples as compared with the adjacent non-tumor tissue controls (FIG. 7C), further supporting the finding that Myo9b expression is up-regulated in human lung cancer.

We further analyzed the correlation between Myo9b expression and clinicopathological features of lung cancer patients in our lung cancer samples. There was no significant correlation between Myo9b expression and patient age, genders or tumor sizes. However, the majority of patients who have lymph node metastasis showed increased Myo9b expression (15 out of 16 cases, Table 3). Thus, Myo9b expression was correlated with lymph node metastasis, suggesting that Myo9b may promote lung cancer metastasis. In addition, 35 out of 43 lung cancer patients with high Myo9b expression had advanced pathological stages (Table 3). Consistently, the mRNA levels of Myo9b are higher in lung tumors with higher grades (FIG. 7D). Therefore, increased Myo9b expression is associated with lung cancer progression in patients.

TABLE 3
Correlation between Myo9b expression and clinicopathological
features of lung cancer patients

| | Myo9b expression | | | |
|---|---|---|---|---|
| Groups | Low | High | n | p (X2 test) |
| Age (years) | | | | |
| <60 | 8 | 18 | 26 | 0.7772 |
| ≧60 | 9 | 25 | 34 | |
| Gender | | | | |
| Male | 13 | 32 | 45 | 1.0000 |
| Female | 4 | 11 | 15 | |
| Tumor Size | | | | |
| <3 cm | 12 | 23 | 35 | 0.2599 |
| ≧3 cm | 5 | 20 | 25 | |
| Lymph Node Metastasis | | | | |
| N0 | 16 | 28 | 44 | 0.0251 |
| N1 + N2 | 1 | 15 | 16 | |
| Tumor Stage | | | | |
| I | 10 | 8 | 18 | 0.0042 |
| II-III | 7 | 35 | 42 | |
| Total | 17 | 43 | 60 | |

The correlation between the overall survival of the patients and the Myo9b expression levels was examined using the Kaplan-Meier method. Higher Myo9b expression was associated with lower probability of overall survival (FIG. 7E). Analyses of the published lung cancer datasets (35) consistently showed that patients with higher levels of Myo9b expression had significantly shorter progression-free survival than those expressing lower levels of Myo9b (FIG. 7F). These results support the notion that Myo9b functions as a promoting factor in lung cancer progression.

### Materials and Methods

### Crystallization, Data Collection and Structure Determination

The crystal of Myo9b RhoGAP domain (15 mg/ml in 50 mM Tris-HCl, pH 8.0, 100 mM NaCl, 1 mM EDTA, 1 mM DTT) was obtained at 16° C. using the vapor diffusion method (sitting drop) in 0.2 M NH4Ac, 0.1 M Bis-Tris pH 6.0 and 20% PEG 3350. Before being flash-frozen in liquid nitrogen, the crystal was cryo-protected with the mother liquor supplemented with 1 M LiAc. Diffraction data were collected at the beamline BL17U of the Shanghai Synchrotron Radiation Facility with a wavelength of 0.979 Å at 100K. The dataset was processed and scaled using iMOSFLM (Battye, 2011; herein incorporated by reference in its entirety) and SCALA module in the CCP4 suite (Dodson, 1997; herein incorporated by reference in its entirety). The structure of Myo9b RhoGAP domain was solved by the molecular replacement method using p50rhoGAP RhoGAP domain (PDB code: 1OW3) as a research model with PHASER (McCoy, 2007; herein incorporated by reference in its entirety). The structure model was further manually built with COOT (Emsley, 2004; herein incorporated by reference in its entirety) and refined with Phenix (Adams, 2010; herein incorporated by reference in its entirety). The overall quality of the final structural model of Myo9b RhoGAP domain was assessed by PROCHECK (Laskowski, 1993; herein incorporated by reference in its entirety). The protein structure figures were prepared using the program PyMOL (http://www.pymol.org). The statistics for the data collection and structural refinement are summarized in Table 4. The coordinate of the crystal structure of Myo9b RhoGAP domain is deposited in the Protein Data Bank (PDB) with the accession number 5C5S.

### Molecular Dynamics Simulations

The initial structural model of the Myo9b RhoGAP/RhoA complex was obtained by replacing the RhoGAP domain in the p50rhoGAP RhoGAP/RhoA complex structure (PDB code: 1OW3) by Myo9b RhoGAP domain. The model structure was then soaked in a 96×96×96 Å3 water box, which included 26 Mg2+ and 48 Cl− to neutralize the system. The NAMD package (Phillips, 2005; herein incorporated by reference in its entirety) and CHARMM22 all-atom force field (MacKerell, 1998; herein incorporated by reference in its entirety) were used for energy minimization and molecular dynamics simulations. Under periodic boundary condition, a 12 Å cut-off was used for van der Waals interactions; and Particle Mesh Ewald summation was used to calculate the electrostatic interactions. Four independent simulations were performed. For each simulation, energy was first minimized in multi-steps to avoid any possible clashes. The energy-minimized system was then equilibrated for 10 ns with temperature controlled at 310 K by Langevin dynamics and pressure controlled at 1 atm by Lagevin piston method. With the equilibrated structures, 50 ns free dynamics simulation was performed for each system. The simulation trajectories were analyzed with VIVID (Humphrey, 1996; herein incorporated by reference in its entirety).

### Animal Experiments

H1299ctr and H1299Slit cells were inoculated subcutaneously into the right flank (6×106 cells per mouse; n=10) of 6 week-old female BALB/c nude mice as described previously (Tong, 2009; herein incorporated by reference in its entirety). Tumor volumes (V) were measured every week and calculated using the equation V(mm³)=a×b2/2, in which a was the largest dimension and b was the perpendicular diameter. Animals were euthanized when the largest primary tumor grew to approximately 1000 mm³ or animal condition deteriorated. The tumors were removed with lung tissues dissected for histological examination. Tissues were fixed in 4% paraformaldehyde and embedded in paraffin. H&E staining and immunohistochemical analyses were performed on tissue sections as previously reported (Yiin, 2009; herein incorporated by reference in its entirety).

### Tissue Microarray and Immunohistochemical Staining

De-identified lung tumor tissue samples were collected following institutional and national guidelines from 60 consented patients in Xijing Hospital, Xi'an, China. The study cohort consisted of tumors and corresponding adjacent non-tumor lung tissues from the same patients. Array blocks were sectioned to produce serial 4 um sections for H&E and immunohistochemical staining. Tissue microarray sections were immunostained with the Myo9b antibody.

### Immunohistochemical Analysis of Human Lung Cancer Tissues

Immunohistochemistry was performed on formalin-fixed, paraffin-embedded human lung cancer tissue sections. The polyclonal anti-Myo9b (1:350) was used with HRP-conjugated goat anti-rabbit second antibody and DAB for cover development. As a control, the samples were incubated with the pre-immune rabbit IgG instead of primary antibody. The Myo9b immunostaining was scored according to the signal intensity and distribution as 0, 1, 2, 3, in which 0, <5%; 1, 5%-25%; 2, 25%-50% and 3, >50%. Cytoplasmic yellow granule-like staining of tumor cells and the staining accounted for >50% in tissue sections were considered as strong Myo9b staining and scored as 3. Tissues with score ≦1 were considered as low expression, whereas scores ≧2 were considered as high expression.

### Microarray Data Analysis

The Oncomine database and gene microarray analysis tool (34), a repository for published cDNA microarray data (www.oncomine.org), were explored for Slit2 mRNA expression in lung cancer and control samples. Oncomine algorithms were used to conduct statistical analyses on the differences in Slit2 expression. For patient survival analyses, association between Slit2 or Myo9b expression and overall survival or progression-free survival was assessed by Kaplan-Meier plotter followed by significance evaluation with log-rank test (Gyorffy, 2013; herein incorporated by reference in its entirety). Studies reporting analytical results with a p value less than 0.05 were considered significant. Cbioportal software (www.cbioportal.org) was performed to analyze correlation of gene mutation and gene expression in 178 human lung cancers (Gao, 2013; herein incorporated by reference in its entirety).

### Statistical Analyses

Comparisons were performed using a 2-tailed Student's t test, Mann-Whitney test, or one-way ANOVA, as indicated. Pearson X2 test was used to evaluate the relationship between Myo9b expression and clinicopathological features. Survival curves were calculated by the Kaplan-Meier method, and comparison was carried out using the log-rank test. P<0.05 was considered as statistically significant. All statistical calculations were carried out using GraphPad Prism 5 software or SPSS 13.0.

TABLE 5
Primer pairs

| RT-PCR primers | | | SEQ ID NO: |
|---|---|---|---|
| Slit2 | Forward primer: | 5′-GGTGTCCTCTGTGATGAAGAG-3′ | 1 |
| | Reverse primer: | 5′-GTGTTTAGGAGACACACCTCG-3′ | 2 |
| Robo1 | Forward primer: | 5′-GCATCGCTGGAAGTAGCCATAC-3′ | 3 |
| | Reverse primer: | 5′-GTGTATGAACCCATGTCACCAGC-3′ | 4 |
| Myo9b | Forward primer: | 5′-ATGAGTGTGAAAGAGGCAG-3′ | 5 |
| | Reverse primer: | 5′-CCACCAGCTGCATATGCA-3′; | 6 |
| Actin | Forward primer: | 5′-TCCCCCAACTTGAGATGTATGAAG-3′ | 7 |
| | Reverse primer: | 5′-AACTGGTCTCAAGTCAGTGTACAGG-3′ | 8 |
| Real time PCR primers | | | |
| Slit2 | Forward primer: | 5′-GCATCCTAACTCCAAAGGGA-3′ | 9 |
| | Reverse primer: | 5′-CTTGTTGTCTTGGCAGTCGT-3′ | 10 |
| Myo9b | Forward primer: | 5′-CGAGAAGTTCAGGAGCAACA-3′ | 11 |
| | Reverse primer: | 5′-GACCAGGTTGGTGTCCTTCT-3′ | 12 |
| GAPDH | Forward primer: | 5′-GGAGCGAGATCCCTCCAAAAT-3′ | 13 |
| | Reverse primer: | 5′-GGCTGTTGTCATACTTCTCATGG-3′ | 14 |
| Myo9b deletion mutants primers | | | |
| Myo9b-FL | Forward primer: | 5′-AGTGAATTCATGAGTGTGAAAGAGGCAG-3′ | 15 |
| | Reverse primer: | 5′-AGTGCGGCCGCTCAGCCATTGGTCTGGC-3′ | 16 |
| Myo9b-C1-GAP | Forward primer- | 5′-AGTGCGGCCGCGCACGTGTTCGCCAGCTACCAGG | 17 |
| | | TTAGCATCCCGCAGTCGTGCGAGCA-3′, | |
| | Forward primer- | 5′-GCAGTCGTGCGAGCAGTGCCTCTCCTATATCTGGC | 18 |
| | | TCATGGACAAGGCCCTGCTCTGCAGCGTGTG-3′ | |
| | Reverse primer: | 5′-AGTGAATTCTTACCGGTAGGTGATGTCC-3′ | 19 |
| Myo9b-GAP | Forward primer: | 5′-AGTGAATTCATGAGTGTGAAAGAGGCAG-3′ | 20 |
| | Reverse primer: | 5′-ATGTCTAGACCCGTTGTGCTCCTGGACA-3′ | 21 |
| | Forward primer: | 5′-ATGGTCGACCTGCCGGAGCTGGA000AA-3′ | 22 |
| | Reverse primer: | 5′-AGTGCGGCCGCTCAGCCATTGGTCTGGC-3′ | 23 |
| GAP point mutants primers | | | |
| GAP Wt | Forward primer: | 5′-CCGGAATTCCCAGGCGTTGAGCCTGGCC-3′ | 24 |
| | Reverse primer: | 5′-ATAAGAATGCGGCCGCTCAAGCATTTTGTCGCAGGAGC-3′ | 25 |
| GAP A1739E | Forward primer: | 5′-CCGCAAGTCGGGTGAGGCCAACCGCACTCG-3′ | 26 |
| | Reverse primer: | 5′-CGAGTGCGGTTGGCCTCACCCGACTTGCGG-3′ | 27 |
| GAP A1739N | Forward primer: | 5′-CTCTACCGCAAGTCGGGTAATGCCAACCGCACT-3 | 28 |
| | Reverse primer: | 5′-AGTGCGGTTGGCATTACGACTTGCGGTAGAG-3 | 29 |
| GAP A1739V | Forward primer: | 5′-CGCAAGTCGGGTGTTGCCAACCGCACT-3′ | 30 |
| | Reverse primer: | 5′-AGTGCGGTTGGCAACACCCGACTTGCG-3′ | 31 |
| GAP N1741E | Forward primer: | 5′-GTCGGGTGCTGCCGAGCGCACTCGGGAGC-3′ | 32 |
| | Reverse primer: | 5′-GCTCCCGAGTGCGCTCGGCAGCACCCGAC-3′ | 33 |
| GAP R1742E | Forward primer: | 5′-CGGGTGCTGCCAACGAGACTCGGGAGCTCCG-3′ | 34 |
| | Reverse primer: | 5′-CGGAGCTCCCGAGTCTCGTTGGCAGCACCCG-3′ | 35 |
| Cdc42 point mutants primers | | | |
| Cdc42S88D/K94P | Forward primer1 | 5′-CCATCTTCATTTGAAAACGTGC | 36 |
| | | CAGAAAAGTGGGTGCCTGAGAT-3′ | |
| | Reverse primer1 | 5′-ATCTCAGGCACCCACTTTTCTG | 37 |
| | | GCACGTTTTCAAATGAAGATGG-3′ | |
| | Forward primer2 | 5′-CTGTTTTTCAGTGGTCTCTCCAG | 38 |
| | | ATTCATTTGAAAACGTGCCAGAA-3′ | |
| | Reverse primer2 | 5′-TTCTGGCACGTTTTCAAATGAAT | 39 |
| | | CTGGAGAGACCACTGAAAAACAG-3′ | |
| Rac1 point mutants primers | | | |
| Rac1A88D/R94P/A9E | Forward primer1 | 5′-GCATCATTTGAAAATGTCCCT | 40 |
| | | GAAAAGTGGTATCCTGAGGTG-3′ | |
| | Reverse primer1 | 5′-CACCTCAGGATACCACTTTTC | 41 |
| | | AGGGACATTTTCAAATGATGC-3′ | |
| | Forward primer2 | 5′-GCTTTTCCCTTGTGAGTCCTGA | 42 |
| | | TTCATTTGAAAATGTCCCTGAA-3′ | |
| | Reverse primer2 | 5′-TTCAGGGACATTTTCAAATGAA | 43 |
| | | TCAGGACTCACAAGGGAAAAGC-3′ | |
| Myo9b siRNAs | | | |
| siMyo9b-1 | Forward primer: | 5′-CGAGAUCACGAUGCACUGGTT-3′ | 44 |
| siMyo9b-2 | Forward primer: | 5′-AGCUGUUCCUCUCGAAGUCTT-3′ | 45 |
| siCtr | Forward primer: | 5′-UUCUCCGAACGUGUCACGUTT-3′ | 46 |

### Example 2

### Slit-Robo-Myo9b-RhoA Pathway in Pancreatic Ductal Adenocarcinoma (PDAC)

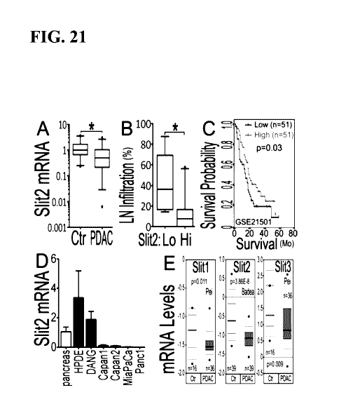

Experiments conducted during development of embodiments herein demonstrate that Cdc42, Rac1, PI3K, AKT and beta-catenin are not changed in PDAC cells upon Slit treatment, and that Myo9b interacts with Robo in PDAC cells and down-regulating Myo9b suppresses cancer-nerve interaction. Reduced expression of Slit2 and increased Myo9b expression have been detected in multiple PDAC cohorts (see FIG. 21). Increased expression of Slit2 or decreased expression of Myo9b is correlated with longer survival of PDAC patients (Göhrig et al, 2014: incorporated by reference in its entirety) (e.g., FIG. 21).

### Examining Expression Profiles of Slit-Robo Pathway Genes in PDAC Samples

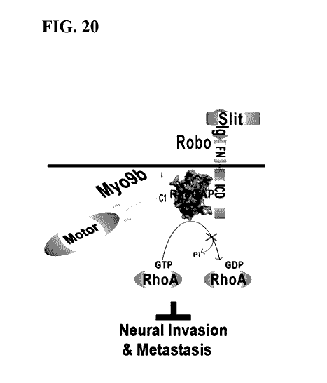

Using qPCR, Western blotting, and immunohistochemistry, expression of Slit, Robo and known downstream signal transduction genes was examined in a collection of PDAC samples and cell lines. Data show that in a significant fraction of PDAC samples, expression of Slit2/3 (FIGS. 20 and 21; Göhrig et al, 2014; incorporated by reference in its entirety) and of Robot/2 genes is reduced, whereas Myo9b expression is increased. Consistently, analyses of PDAC datasets show that low expression of Slit2/3, or high Myo9b expression is associated poor prognosis (e.g., FIG. 21).

Validation at the protein level is by immunohistochemical staining and Western blotting analyses using ˜80 pairs of PDAC samples (tumor vs adjacent non-tumor samples). p53 expression is examined, because p53 is a critical tumor suppressor gene frequently inactivated in PDAC. Other genes associated with PDAC are also examined. Both male and female samples are included because data suggest that some of Slit-Robo pathway genes show gender-specific expression changes in PDAC samples.

### Identifying Genetic and Epigenetic Alterations in Slit-Robo Pathway Genes in PDAC

An RNA-sequencing study have commenced on cohorts of PDAC patient samples. Analyses of RNA-seq data together with published datasets has begun by focusing on the Slit-Robo-Myo9b pathway. A number of PDAC-associated mutations lead to truncation (*) and frame-shift (fs) that are predicted to cause losses of function of Slit and Robo genes. Exepriments focus on selected mutations to test the effects of these mutations on Slit-Robo signaling. In particular, experiments test missense mutations in Slit2/3 and Robo1/2 genes within domains required for Slit-Robo interaction, or interaction between Robo1/2 genes and their interaction partners (such as Myo9b) because such mutations may likely alter Slit-Robo signaling (Wu et al, 1999; Yuasa-Kawada et al, 2009; Wen et al, 2014; incorporated by reference in their entireties). Two mutations, W908* in Robo1 and Q867* in Robo2 lead to the formation of Robo truncation products that lack the intracellular signaling domain, mimicking the dominant-negative mutant (DN-Robo1; Wong et al, 2001; Wen et al 2014; incorporated by reference in their entireties). In addition, mutations in the functional domains (e.g. domains involved in Slit-Robo interaction) have been identified. Constructs for selected PDAC-associated mutant Slit-Robo genes are used to carry out biochemical assays to test if these mutations affect Slit-Robo interaction or protein localization or modulate Slit signaling in PDAC-nerve interaction. Systematic analysis of RNA-seq data and validation of RNA-seq data using quantitative RT-PCR allows detection of different splicing isoforms. This not only identifies new mutations but also help understand whether defective Slit RNA species are produced, or downstream genes are affected among the PDAC samples with elevated Slit1 gene expression. DNA methylation mediated gene silencing of Slit2/3 and Robo1/2 found that these genes are silenced in a sizable fraction of PDAC patients. Experiments systematically investigate DNA methylation of Slit2/3 and Robo1/2 genes in PDAC samples.

### Testing the Role of Slit Signaling in Cancer Cell Death, Proliferation and Anchorage Independent Growth

To exclude possible indirect effects on NI or metastasis via cell death or cell proliferation, Slit-Robo signaling affects PDAC cell death and cell proliferation are analyzed. Experiments were conducted during development of embodiments herein to establish assays to examine effects on pancreatic cancer cell death and proliferation. By measuring BrdU incorporation, LDH release, fluorimetric caspase assays, TUNEL and nuclear staining, Slit effects on cell proliferation and cell death have been examined in PDAC cells (Göhrig et al, 2014; incorporated by reference in its entirety). For example, in pancreatic (e.g, MiaPaCa, CaPan2, AsPc1, or DANG) cancer cells treated with control or Slit for 24 to 96 hrs, no effects have been detected on cell proliferation or cell death. Stable MiaPaCa cell lines have been prepared (MiaPaCa-Slit) that express Slit2 as a myc-tagged protein at the level similar to the endogenous Slit2 level of the normal pancreatic tissue (as detected by qPCR). To rule out that the data obtained on cell proliferation assay were biased results from monolayer cultures, 3-D cultures are used, including organoid culture and microfluidic chamber cultures, to examine cell proliferation, cell death and anchorage-independent cell growth. Slit-expressing and Slit-non-expressing pancreatic cancer cells are compared to test effects of Slit on cell death and proliferation these PDAC cells. Similarly, effects of Robo and Myo9b genes on cancer cell death and proliferation are examined using these established assays.

### Examining Slit Effects on PDAC Cell Invasion and Migration Using Chemo-Invasion, Transwell Migration, Wound-Closure and Time-Lapse Microscopy Assays

To examine ability of PDAC cells to invade into matrigel-reconstituted basement membrane, an in vitro chemo-invasion assay was established by modifying published protocols for cancer cell invasion using 48-well chambers (Yuasa-Kawada et al, 2009; incorporated by reference in its entirety). Experiments show that Slit treatment significantly reduced cancer cell invasion by either MiaPaCa (derived from a male patient) or AsPc1 (derived from a female patient). MiaPaca, DANG and AsPc1 cell lines are used because they recapitulate the PDAC cell features in invasion and metastasis in animal models with MiaPaca and DANG showing high propensity for NI and metastasis, whereas AsPc1 showed a low frequency of NI (Koide et al, 2006; incorporated by reference in its entirety). Transwell migration experiments show that Slit significantly inhibits migration of PDAC cells (Göhrig et al, 2014; incorporated by reference in its entirety). Two PDAC-associated mutations in Robo1 (W908*, P939Q) are engineered and compared with the wild-type Robo1 in cell invasion and migration assays using PDAC cell lines. These two mutations are selected because they are recurrent mutations identified in multiple PDAC patients. The W908* mutation is predicted to result in the formation of truncated Robo protein that only contains extracellular domain of Robo receptor that lacks the intracellular domain mediating signaling, thus, a loss of function mutation or dominant negative mutant that blocks Robo signaling. Alternatively, The W908* mutation could lead to non-sense mediated decay of the RNA, again behaving as a loss of function mutation. The P939Q mutation resides in the region critical for interacting with downstream Robo-interacting proteins and is predicted to interfere with Robo intracellular signaling.

### Slit-Robo Signaling in Suppressing NI and Metastasis in a Xenograft Model

An orthotopic mouse model has been established that demonstrates that Slit expression inhibits PDAC invasion and metastasis. The role of Myo9b in Slit signaling is tested in this model using MiaPaCa-Slit cells in which Myo9b has been knocked out using the Cas9-CRISPR method (Heidenreich and Zhang, 2016; incorporated by reference in its entirety). SCID mice are used in each group so that statistically powerful data can be obtained. The number of mice needed is determined using statistical modeling using parametric mixture modeling analysis with parametric bootstrapping (Page et al, 2006; incorporated by reference in its entirety). The expected discovery rate (EDR) equals D/(B+D) when true positives (TP) equal D/(C+D) and true negatives (TN) equal to A/(A+B). Enough mice are used to observe a change of 20% in metastasis, with a standard deviation of 5, 90% power and alpha level of 0.05 in the treatment groups. A 10% loss rate is included in the sample size calculation. To facilitate real-time monitoring of PDAC metastasis and quantitative analyses in vivo, bioluminescence imaging assays have been developed. Luciferase-expressing MiaPaCa cells including corresponding parental control cells (in which Slit2 gene expression is not detectable), MiaPaCa-Slit (overexpressing Slit) (2 groups, 60 mice/group, 1×10⁶ cells per mouse) are injected into pancreas with ultra-sound guidance of 6 wk old female mice (Gohrig et al, 2014). Mice are monitored twice a week for cancer progression using bioluminescence imaging. With MiaPaCa cells, ˜8 weeks following tumor implantation or if mouse conditions deteriorate, animals are euthanized for histology. Tumors, mesentery and surrounding tissues are dissected with NI and metastasis examined. Immunostaining for NI markers (Artemin, TrkA; GDNFRa1) and quantification of NI is performed (Gohrig et al, 2014; He 2014; incorporated by reference in their entireties). These 3 markers are selected because they are associated with NI and up-regulated in both cancer and nerve cells (Bapat 2011; He 2014; incorporated by reference in its entirety).

Lentivirus-mediated short-hairpin RNAs (shRNAs) are used to test the role of Robo1/2, and Myo9b in cancer NI and metastasis using the orthotopic tumor model. Robo1 and 2 are expressed in the pancreatic cancer cells; and their deletion and focal copy number losses have been found in human PDAC samples. Expression of Slit2 was reduced, whereas Myo9b expression was increased in PDAC.

To test the role of Robo1/2 in pancreatic cancer, experiments conducted during development of embodiments herein have demonstrated that knocking down Robo1 in DANG cells enhanced cancer invasion and metastasis without affecting angiogenesis, supporting that Slit-Robo signaling is critical for limiting pancreatic cancer NI and metastasis.

### Sciatic Nerve NI Model

A sciatic nerve (SN) injection model (Gil et al, 2007; incorporated by reference in its entirety) was improved to recapitulate NI of PDAC in vivo. PDAC cells (3×10⁵ cells) were injected into the perineurium of the sciatic nerve (distal to the bifurcation of the tibial and common peroneal nerves) of SCID mice and monitored for neural invasion. Injection MiaPaCa-Slit cells led to reduced NI than that of MiaPaCa-Ctr cells not expressing Slit, indicating that Slit expression suppresses NI.

Pdx1-Cre; LSL-Kras^{G12D} (KC) and Pdx1-Cre-GFP; LSL-Kras^{G12D}; LSL-p53R172H/+ (KPC) Mouse Models to Test the Role Slit Pathway Genes in NI, Progression and Metastasis in Immune-Competent Mice.

KRAS mutations have been associated with >90% PDAC patients with activation mutations most frequently at KRAS-G12 residue (Almoguera et al. 1988; incorporated by reference in its entirety). KC mice develop pancreatic intraepithelial neoplasia (PanIN) lesions that recapitulate human precancerous lesions (Hingorani et al, 2003; Morton et al, 2010; incorporated by reference in their entireties); whereas KPC mice develop metastatic pancreatic cancer with median survival of ˜4 months (Morton et al, 2010; Tseng et al, 2010; Qiu et al, 2011; incorporated by reference in their entireties). Therefore, experiments are conducted during development of embodiments herein to test if deleting Slit2/3 in KC mice promotes cancer progression, and to examine if Myo9b deletion in PKC mice delays cancer invasion and metastasis. Experiments focus on Slit2/3 and Myo9b genes to examine their role in NI. Among the 3 mammalian Slit genes, only Slit2 and Slit3 are expressed in the pancreas (Wu et al, 2001; incorporated by reference in its entirety). Higher expression of Slit2/3 is associated with better prognosis in PDAC patients. Multiple mutations have been identified in Slit2/3 genes in PDAC patients (FIG. 21).

Because Slit2KO mice do not survive postnatally, conditional knock-out (cKO) mice are used. Slit2 and Slit3 cKO mice have been obtained and Myo9b cKO mice have been generated using Cas9/CRISPR technology. KC mice are crossed with Slit2-cKO or Slit2/3-cKO mice to test whether Slit2/3 deficiency in the pancreas promote cancer invasion and metastasis. KPC mice will be crossed with Myo9b cKO to test whether Myo9b deletion delays or reduces neural invasion and metastasis. As shown in Table 6, 4 groups of mice are used, including wild type (WT) and cKO mice, 60 mice (30 males, 30 females) per group. Mice are euthanized at 5-8 months of age or when tumor burdens become difficult to tolerate, with their tumors and surrounding tissues collected for molecular and histological analyses by experienced pathologists. Tumor invasion, especially neural invasion, and mesentery metastasis is carefully evaluated and quantified.

TABLE 6
Using conditional KO mice to determine the
role of Slit2/3, Myo9b in pancreatic cancer

| | | | | | Termination |
|---|---|---|---|---|---|
| | Mouse | # | Pdx1-cre; | LSL- | point |
| Groups | Genotype | mice | LSL-Kras^{G12D} | p53R172H | (mo) |
| 1 | WT | 60 | + | − | 8 |
| 2 | SLIT2 cKO | 60 | + | − | 8 |
| 3 | SLIT2/3 | 60 | + | − | 5 |
| | double cKO | | | | |
| 4 | Myo9b-cKO | 60 | + | − | 8 |

### Example 3

### miRNAs

Experiments were conducted during development of embodiments herein to identify ncRNAs (non-coding RNAs), in particular microRNAs (miRNAs), that regulate Myo9b expression and/or function, either by modulating translation or altering mRNA stability of human Myo9b. Using a variety of available bioinformatic tools, such as MirWalk (zmfumm.uni-heidelberg.de/apps/zmf/mirwalk2; Dweep, et al. J Biomed Inform 44, 839-47 (2011); incorporated by reference in its entirety), MirMap (mirmap.ezlab.org; Vejnar & Zdobnov. Nucleic Acids Research 2012 Dec. 1; 40(22):11673-83; incorporated by reference in its entirety), TargetScan (targetscan.org/; Agarwal et al. eLife 2015; 4:e05005; incorporated by reference in its entirety), MirAnda (microrna.org/microrna/home.do; Enright et al. Genome Biology (2003) 5; R1; incorporated by reference in its entirety), and RNA22 (cm.jefferson.edu/rna22/; Miranda et al (2006) Cell, 126, 1203-1217; incorporated by reference in its entirety), prospective miRNAs were identified in the 3′ UTR (SEQ ID NO: 60), 5′ UTR (SEQ ID NO: 61), and promoter (SEQ ID NO: 61) regions of human Myo9b. miRNA corresponding to SEQ ID NOS: 63-2758 were identified in the Myo9b 3′ UTR. Tables 7a and 7b demonstrate miRNA sequences (as indicated by ID Nos., see, e.g., mirbase.org; mirgate.bioinfo.cnio.es; microrna.gr/mirpub) from the Myo9b 5′ UTR. Tables 8a and 8b demonstrate miRNA sequences (as indicated by ID Nos., see, e.g., mirbase.org; mirgate.bioinfo.cnio.es; microrna.gr/mirpub) from the Myo9b promoter.

TABLE 7a
Myo9b 5′ UTR miRNAs (all sequences correspond to Gene: MYBO9B; EntrezID:
4650; RefseqID NM_00130065).

| | | miRWalk | | | | | | |
|---|---|---|---|---|---|---|---|---|
| | | UTR | miRWalk | | | | | |
| miRNA | MIMATid | start | UTR Seq | miRWalk | miRanda | RNA22 | Targetscan | SUM |
| hsa-miR-4254 | MIMAT0016884 | 93 | AGCUCCAGG | 1 | 1 | 1 | 0 | 3 |
| hsa-miR-3922- | MIMAT0018197 | 88 | AGGCCAG | 1 | 1 | 1 | 0 | 3 |
| 3p | | | | | | | | |
| hsa-miR-3176 | MIMAT0015053 | 88 | AGGCCAG | 1 | 1 | 1 | 0 | 3 |
| hsa-miR-3178 | MIMAT0015055 | 107 | CGCGCCCC | 1 | 1 | 1 | 0 | 3 |
| hsa-miR-1202 | MIMAT0005865 | 29 | GCUGGCA | 1 | 1 | 1 | 0 | 3 |
| hsa-miR-4743- | MIMAT0019874 | 44 | UCCGGCC | 1 | 1 | 0 | 0 | 2 |
| 5p | | | | | | | | |
| hsa-miR-6778- | MIMAT0027457 | 121 | GGGAGGCA | 1 | 1 | 0 | 0 | 2 |
| 3p | | | | | | | | |
| hsa-miR-1908- | MIMAT0026916 | 79 | CGGCGGCCG | 1 | 1 | 0 | 0 | 2 |
| 3p | | | | | | | | |
| hsa-miR-3943 | MIMAT0018359 | n/a | n/a | 1 | 0 | 1 | 0 | 2 |
| hsa-miR-4300 | MIMAT0016853 | n/a | n/a | 1 | 0 | 1 | 0 | 2 |
| hsa-miR-1914- | MIMAT0007890 | n/a | n/a | 1 | 0 | 1 | 0 | 2 |
| 3p | | | | | | | | |
| hsa-miR-4746- | MIMAT0019880 | 64 | GGGACCGG | 1 | 1 | 0 | 0 | 2 |
| 5p | | | | | | | | |
| hsa-miR-6781- | MIMAT0027462 | 57 | CCGGCCCG | 1 | 1 | 0 | 0 | 2 |
| 5p | | | | | | | | |
| hsa-miR-3944- | MIMAT0018360 | n/a | n/a | 1 | 0 | 1 | 0 | 2 |
| 3p | | | | | | | | |
| hsa-miR-1909- | MIMAT0007883 | n/a | n/a | 1 | 0 | 1 | 0 | 2 |
| 3p | | | | | | | | |
| hsa-miR-3972 | MIMAT0019357 | 27 | GGGCUGGC | 1 | 1 | 0 | 0 | 2 |
| | | | AG | | | | | |
| hsa-miR-718 | MIMAT0012735 | n/a | n/a | 1 | 0 | 1 | 0 | 2 |
| hsa-miR-6836- | MIMAT0027575 | 121 | GGGAGGCA | 1 | 1 | 0 | 0 | 2 |
| 3p | | | U | | | | | |
| hsa-miR-1470 | MIMAT0007348 | n/a | n/a | 1 | 0 | 1 | 0 | 2 |
| hsa-miR-3194- | MIMAT0015078 | n/a | n/a | 0 | 1 | 1 | 0 | 2 |
| 5p | | | | | | | | |
| hsa-miR-133a- | MIMAT0000427 | n/a | n/a | 1 | 0 | 1 | 0 | 2 |
| 3p | | | | | | | | |
| hsa-miR-2116- | MIMAT0011161 | 120 | UGGGAGG | 1 | 0 | 1 | 0 | 2 |
| 3p | | | | | | | | |
| hsa-miR-6850- | MIMAT0027601 | 44 | UCCGGCCGG | 1 | 1 | 0 | 0 | 2 |
| 3p | | | | | | | | |
| hsa-miR-4487 | MIMAT0019021 | 90 | GCCAGCUC | 1 | 1 | 0 | 0 | 2 |
| hsa-miR-1471 | MIMAT0007349 | n/a | n/a | 1 | 0 | 1 | 0 | 2 |
| hsa-miR-519d- | MIMAT0002853 | n/a | n/a | 1 | 0 | 1 | 0 | 2 |
| 3p | | | | | | | | |
| hsa-miR-484 | MIMAT0002174 | n/a | n/a | 0 | 1 | 1 | 0 | 2 |
| hsa-miR-6763- | MIMAT0027427 | 47 | GGCCGGGG | 1 | 1 | 0 | 0 | 2 |
| 3p | | | A | | | | | |
| hsa-miR-133b | MIMAT0000770 | n/a | n/a | 1 | 0 | 1 | 0 | 2 |
| hsa-miR-4665- | MIMAT0019740 | 81 | GCGGCCGAG | 1 | 1 | 0 | 0 | 2 |
| 3p | | | | | | | | |
| hsa-miR-3196 | MIMAT0015080 | 109 | CGCCCCG | 1 | 0 | 1 | 0 | 2 |
| hsa-miR-6869- | MIMAT0027639 | 74 | GCGCGCGGC | 1 | 1 | 0 | 0 | 2 |
| 3p | | | G | | | | | |
| hsa-miR-4707- | MIMAT0019808 | 24 | GGCGGGCU | 1 | 1 | 0 | 0 | 2 |
| 3p | | | | | | | | |
| hsa-miR-663a | MIMAT0003326 | n/a | n/a | 1 | 0 | 1 | 0 | 2 |
| hsa-miR-4707- | MIMAT0019807 | 48 | GCCGGGG | 1 | 1 | 0 | 0 | 2 |
| 5p | | | | | | | | |
| hsa-miR-6874- | MIMAT0027648 | 91 | CCAGCUCCA | 1 | 1 | 0 | 0 | 2 |
| 5p | | | | | | | | |
| hsa-miR-663b | MIMAT0005867 | n/a | n/a | 1 | 0 | 1 | 0 | 2 |
| hsa-miR-6727- | MIMAT0027355 | 110 | GCCCCGAG | 1 | 1 | 0 | 0 | 2 |
| 5p | | | | | | | | |
| hsa-miR-4722- | MIMAT0019837 | 29 | GCUGGCAG | 1 | 1 | 0 | 0 | 2 |
| 3p | | G | | | | | | |
| hsa-miR-615- | MIMAT0004804 | n/a | n/a | 1 | 0 | 1 | 0 | 2 |
| 5p | | | | | | | | |
| hsa-miR-6878- | MIMAT0027657 | 87 | GAGGCCAG | 1 | 1 | 0 | 0 | 2 |
| 3p | | | | | | | | |
| hsa-miR-3155a | MIMAT0015029 | n/a | n/a | 0 | 1 | 1 | 0 | 2 |
| hsa-miR-4281 | MIMAT0016907 | 61 | CCCGGGACC | 1 | 1 | 0 | 0 | 2 |

TABLE 7b
Myo9b 5′ UTR miRNAs (all sequences correspond to Gene: MYBO9B; EntrezID:
4650; RefseqID NM_00130065).

| miRNA | MIMATid | SPMS | SL | SeedS | SeedE | pvalue | Target Seq |
|---|---|---|---|---|---|---|---|
| hsa-miR-1202 | MIMAT0005865 | 2 | 7 | 35 | 29 | 0.0092 | GCUGGCA |
| hsa-miR-1908-3p | MIMAT0026916 | 1 | 9 | 148 | 140 | 0.0006 | GGCGGCCGG |
| hsa-miR-1908-3p | MIMAT0026916 | 2 | 9 | 87 | 79 | 0.0006 | CGGCGGCCG |
| hsa-miR-2116-3p | MIMAT0011161 | 1 | 7 | 126 | 120 | 0.0092 | UGGGAGG |
| hsa-miR-3176 | MIMAT0015053 | 2 | 7 | 94 | 88 | 0.0092 | AGGCCAG |
| hsa-miR-3178 | MIMAT0015055 | 1 | 8 | 114 | 107 | 0.0023 | CGCGCCCC |
| hsa-miR-3196 | MIMAT0015080 | 1 | 7 | 115 | 109 | 0.0092 | CGCCCCG |
| hsa-miR-3922-3p | MIMAT0018197 | 2 | 7 | 94 | 88 | 0.0092 | AGGCCAG |
| hsa-miR-3972 | MIMAT0019357 | 1 | 10 | 36 | 27 | 0.0001 | GGGCUGGCAG |
| hsa-miR-4254 | MIMAT0016884 | 2 | 9 | 101 | 93 | 0.0006 | AGCUCCAGG |
| hsa-miR-4281 | MIMAT0016907 | 2 | 9 | 69 | 61 | 0.0006 | CCCGGGACC |
| hsa-miR-4442 | MIMAT0018960 | 1 | 7 | 49 | 43 | 0.0092 | GUCCGGC |
| hsa-miR-4487 | MIMAT0019021 | 2 | 8 | 97 | 90 | 0.0023 | GCCAGCUC |
| hsa-miR-4665-3p | MIMAT0019740 | 1 | 9 | 89 | 81 | 0.0006 | GCGGCCGAG |
| hsa-miR-4707-3p | MIMAT0019808 | 1 | 8 | 31 | 24 | 0.0023 | GGCGGGCU |
| hsa-miR-4707-5p | MIMAT0019807 | 2 | 7 | 54 | 48 | 0.0092 | GCCGGGG |
| hsa-miR-4722-3p | MIMAT0019837 | 2 | 9 | 37 | 29 | 0.0006 | GCUGGCAGG |
| hsa-miR-4743-5p | MIMAT0019874 | 2 | 7 | 50 | 44 | 0.0092 | UCCGGCC |
| hsa-miR-4746-5p | MIMAT0019880 | 1 | 8 | 71 | 64 | 0.0023 | GGGACCGG |
| hsa-miR-532-3p | MIMAT0004780 | 1 | 7 | 126 | 120 | 0.0092 | UGGGAGG |
| hsa-miR-532-5p | MIMAT0002888 | 1 | 7 | 130 | 124 | 0.0092 | AGGCAUG |
| hsa-miR-6727-5p | MIMAT0027355 | 1 | 8 | 117 | 110 | 0.0023 | GCCCCGAG |
| hsa-miR-6763-3p | MIMAT0027427 | 2 | 9 | 55 | 47 | 0.0006 | GGCCGGGGA |
| hsa-miR-6764-5p | MIMAT0027428 | 1 | 7 | 124 | 118 | 0.0092 | CCUGGGA |
| hsa-miR-6775-5p | MIMAT0027450 | 1 | 7 | 116 | 110 | 0.0092 | GCCCCGA |
| hsa-miR-6778-3p | MIMAT0027457 | 1 | 8 | 128 | 121 | 0.0023 | GGGAGGCA |
| hsa-miR-6781-5p | MIMAT0027462 | 1 | 8 | 64 | 57 | 0.0023 | CCGGCCCG |
| hsa-miR-6836-3p | MIMAT0027575 | 1 | 9 | 129 | 121 | 0.0006 | GGGAGGCAU |
| hsa-miR-6850-3p | MIMAT0027601 | 1 | 10 | 53 | 44 | 0.0001 | UCCGGCCGGG |
| hsa-miR-6850-3p | MIMAT0027601 | 2 | 9 | 52 | 44 | 0.0006 | UCCGGCCGG |
| hsa-miR-6869-3p | MIMAT0027639 | 1 | 10 | 83 | 74 | 0.0001 | GCGCGCGGCG |
| hsa-miR-6874-5p | MIMAT0027648 | 2 | 9 | 99 | 91 | 0.0006 | CCAGCUCCA |
| hsa-miR-6878-3p | MIMAT0027657 | 1 | 8 | 94 | 87 | 0.0023 | GAGGCCAG |

TABLE 8a
Myo9b promoter miRNAs (all sequences correspond to Gene: MYBO9B; EntrezID:
4650; RefseqID NM_00130065).

| | | miRWal | | | | | | |
|---|---|---|---|---|---|---|---|---|
| | | k UTR | miRWalk | miRW | miRan | Targets | SU | Range |
| miRNA | MIMATid | Start | UTR Seq | alk | da | can | M | (kb) |
| hsa-miR-106a- | MIMAT0004 | n/a | n/a | 1 | 1 | 1 | 3 | 2 |
| 3p | 517 | | | | | | | |
| hsa-miR-10b- | MIMAT0004 | n/a | n/a | 1 | 1 | 1 | 3 | 2 |
| 3p | 556 | | | | | | | |
| hsa-miR-1193 | MIMAT0015 | n/a | n/a | 1 | 1 | 1 | 3 | 2 |
| | 049 | | | | | | | |
| hsa-miR-1200 | MIMAT0005 | n/a | n/a | 1 | 1 | 1 | 3 | 2 |
| | 863 | | | | | | | |
| hsa-miR-1203 | MIMAT0005 | n/a | n/a | 1 | 1 | 1 | 3 | 2 |
| | 866 | | | | | | | |
| hsa-miR-122- | MIMAT0004 | n/a | n/a | 1 | 1 | 1 | 3 | 2 |
| 3p | 590 | | | | | | | |
| hsa-miR-1226- | MIMAT0005 | n/a | n/a | 1 | 1 | 1 | 3 | 2 |
| 3p | 577 | | | | | | | |
| hsa-miR-1229- | MIMAT0005 | n/a | n/a | 1 | 1 | 1 | 3 | 2 |
| 3p | 584 | | | | | | | |
| hsa-miR-1231 | MIMAT0005 | n/a | n/a | 1 | 1 | 1 | 3 | 2 |
| | 586 | | | | | | | |
| hsa-miR-1234- | MIMAT0005 | 624 | GTTCGCCAG | 1 | 1 | 1 | 3 | 2 |
| 3p | 589 | | G | | | | | |
| hsa-miR-1237- | MIMAT0022 | 299 | ATAGGAATC | 1 | 1 | 1 | 3 | 2 |
| 5p | 946 | | | | | | | |
| hsa-miR-1238- | MIMAT0005 | n/a | n/a | 1 | 1 | 1 | 3 | 2 |
| 3p | 593 | | | | | | | |
| hsa-miR-1254 | MIMAT0005 | 1405 | CGAGGACG | 1 | 1 | 1 | 3 | 2 |
| | 905 | | C | | | | | |
| hsa-miR-1258 | MIMAT0005 | n/a | n/a | 1 | 1 | 1 | 3 | 2 |
| | 909 | | | | | | | |
| hsa-miR-125a- | MIMAT0000 | n/a | n/a | 1 | 1 | 1 | 3 | 2 |
| 5p | 443 | | | | | | | |
| hsa-miR-125b- | MIMAT0004 | 1327 | TCGGCCCCA | 1 | 1 | 1 | 3 | 2 |
| 1-3p | 592 | | | | | | | |
| hsa-miR-125b- | MIMAT0000 | n/a | n/a | 1 | 1 | 1 | 3 | 2 |
| 5p | 423 | | | | | | | |
| hsa-miR-1260b | MIMAT0015 | n/a | n/a | 1 | 1 | 1 | 3 | 2 |
| | 041 | | | | | | | |
| hsa-miR-1262 | MIMAT0005 | n/a | n/a | 1 | 1 | 1 | 3 | 2 |
| | 914 | | | | | | | |
| hsa-miR-1266- | MIMAT0005 | n/a | n/a | 1 | 1 | 1 | 3 | 2 |
| 5p | 920 | | | | | | | |
| hsa-miR-1273f | MIMAT0020 | n/a | n/a | 1 | 1 | 1 | 3 | 2 |
| | 601 | | | | | | | |
| hsa-miR-128- | MIMAT0026 | n/a | n/a | 1 | 1 | 1 | 3 | 2 |
| 1-5p | 477 | | | | | | | |
| hsa-miR-128- | MIMAT0031 | 666 | CTAACCCGA | 1 | 1 | 1 | 3 | 2 |
| 2-5p | 095 | | | | | | | |
| hsa-miR-1288- | MIMAT0026 | 120 | ACTGACACA | 1 | 1 | 1 | 3 | 2 |
| 5p | 743 | | | | | | | |
| hsa-miR-1291 | MIMAT0005 | n/a | n/a | 1 | 1 | 1 | 3 | 2 |
| | 881 | | | | | | | |
| hsa-miR-1292- | MIMAT0022 | n/a | n/a | 1 | 1 | 1 | 3 | 2 |
| 3p | 948 | | | | | | | |
| hsa-miR-1304- | MIMAT0022 | 1790 | CCAGACGCG | 1 | 1 | 1 | 3 | 2 |
| 3p | 720 | | | | | | | |
| hsa-miR-1304- | MIMAT0005 | n/a | n/a | 1 | 1 | 1 | 3 | 2 |
| 5p | 892 | | | | | | | |
| hsa-miR-1306- | MIMAT0022 | n/a | n/a | 1 | 1 | 1 | 3 | 2 |
| 5p | 726 | | | | | | | |
| hsa-miR-1307- | MIMAT0022 | n/a | n/a | 1 | 1 | 1 | 3 | 2 |
| 5p | 727 | | | | | | | |
| hsa-miR-130a- | MIMAT0000 | 1811 | CCTCCCCTC | 1 | 1 | 1 | 3 | 2 |
| 3p | 425 | | | | | | | |
| hsa-miR-130b- | MIMAT0000 | 1811 | CCTCCCCTC | 1 | 1 | 1 | 3 | 2 |
| 3p | 691 | | | | | | | |
| hsa-miR-130b- | MIMAT0004 | n/a | n/a | 1 | 1 | 1 | 3 | 2 |
| 5p | 680 | | | | | | | |
| hsa-miR-1321 | MIMAT0005 | n/a | n/a | 1 | 1 | 1 | 3 | 2 |
| | 952 | | | | | | | |
| hsa-miR-1343- | MIMAT0019 | n/a | n/a | 1 | 1 | 1 | 3 | 2 |
| 3p | 776 | | | | | | | |
| hsa-miR-135b- | MIMAT0004 | n/a | n/a | 1 | 1 | 1 | 3 | 2 |
| 3p | 698 | | | | | | | |
| hsa-miR-138- | MIMAT0000 | n/a | n/a | 1 | 1 | 1 | 3 | 2 |
| 5p | 430 | | | | | | | |
| hsa-miR-143- | MIMAT0000 | n/a | n/a | 1 | 1 | 1 | 3 | 2 |
| 3p | 435 | | | | | | | |
| hsa-miR-143- | MIMAT0004 | n/a | n/a | 1 | 1 | 1 | 3 | 2 |
| 5p | 599 | | | | | | | |
| hsa-miR-145- | MIMAT0004 | n/a | n/a | 1 | 1 | 1 | 3 | 2 |
| 3p | 601 | | | | | | | |
| hsa-miR-1469 | MIMAT0007 | n/a | n/a | 1 | 1 | 1 | 3 | 2 |
| | 347 | | | | | | | |
| hsa-miR-149- | MIMAT0004 | n/a | n/a | 1 | 1 | 1 | 3 | 2 |
| 3p | 609 | | | | | | | |
| hsa-miR-153- | MIMAT0026 | 1925 | AGCGCTGGC | 1 | 1 | 1 | 3 | 2 |
| 5p | 480 | | | | | | | |
| hsa-miR-1825 | MIMAT0006 | n/a | n/a | 1 | 1 | 1 | 3 | 2 |
| | 765 | | | | | | | |
| hsa-miR-188- | MIMAT0000 | n/a | n/a | 1 | 1 | 1 | 3 | 2 |
| 5p | 457 | | | | | | | |
| hsa-miR-1908- | MIMAT0007 | 47 | ACCCCATCT | 1 | 1 | 1 | 3 | 2 |
| 5p | 881 | | CT | | | | | |
| hsa-miR-1913 | MIMAT0007 | n/a | n/a | 1 | 1 | 1 | 3 | 2 |
| | 888 | | | | | | | |
| hsa-miR-1914- | MIMAT0007 | n/a | n/a | 1 | 1 | 1 | 3 | 2 |
| 5p | 889 | | | | | | | |
| hsa-miR-1915- | MIMAT0007 | n/a | n/a | 1 | 1 | 1 | 3 | 2 |
| 3p | 892 | | | | | | | |
| hsa-miR-192- | MIMAT0000 | 1255 | ACAGTAAAG | 1 | 1 | 1 | 3 | 2 |
| 5p | 222 | | | | | | | |
| hsa-miR-193a- | MIMAT0004 | 732 | TTTTACATA | 1 | 1 | 1 | 3 | 2 |
| 5p | 614 | | | | | | | |
| hsa-miR-193b- | MIMAT0004 | n/a | n/a | 1 | 1 | 1 | 3 | 2 |
| 5p | 767 | | | | | | | |
| hsa-miR-1976 | MIMAT0009 | n/a | n/a | 1 | 1 | 1 | 3 | 2 |
| | 451 | | | | | | | |
| hsa-miR-199a- | MIMAT0000 | n/a | n/a | 1 | 1 | 1 | 3 | 2 |
| 5p | 231 | | | | | | | |
| hsa-miR-199b- | MIMAT0000 | n/a | n/a | 1 | 1 | 1 | 3 | 2 |
| 5p | 263 | | | | | | | |
| hsa-miR-19a- | MIMAT0000 | n/a | n/a | 1 | 1 | 1 | 3 | 2 |
| 3p | 073 | | | | | | | |
| hsa-miR-19a- | MIMAT0004 | n/a | n/a | 1 | 1 | 1 | 3 | 2 |
| 5p | 490 | | | | | | | |
| hsa-miR-19b- | MIMAT0004 | 1282 | GCGCGTTTC | 1 | 1 | 1 | 3 | 2 |
| 1-5p | 491 | | | | | | | |
| hsa-miR-19b- | MIMAT0004 | 1282 | GCGCGTTTC | 1 | 1 | 1 | 3 | 2 |
| 2-5p | 492 | | | | | | | |
| hsa-miR-19b- | MIMAT0000 | n/a | n/a | 1 | 1 | 1 | 3 | 2 |
| 3p | 074 | | | | | | | |
| hsa-miR-200b- | MIMAT0000 | n/a | n/a | 1 | 1 | 1 | 3 | 2 |
| 3p | 318 | | | | | | | |
| hsa-miR-200c- | MIMAT0000 | n/a | n/a | 1 | 1 | 1 | 3 | 2 |
| 3p | 617 | | | | | | | |
| hsa-miR-2053 | MIMAT0009 | n/a | n/a | 1 | 1 | 1 | 3 | 2 |
| | 978 | | | | | | | |
| hsa-miR-210- | MIMAT0000 | n/a | n/a | 1 | 1 | 1 | 3 | 2 |
| 3p | 267 | | | | | | | |
| hsa-miR-210- | MIMAT0026 | n/a | n/a | 1 | 1 | 1 | 3 | 2 |
| 5p | 475 | | | | | | | |
| hsa-miR-211- | MIMAT0022 | n/a | n/a | 1 | 1 | 1 | 3 | 2 |
| 3p | 694 | | | | | | | |
| hsa-miR-212- | MIMAT0022 | n/a | n/a | 1 | 1 | 1 | 3 | 2 |
| 5p | 695 | | | | | | | |
| hsa-miR-214- | MIMAT0000 | n/a | n/a | 1 | 1 | 1 | 3 | 2 |
| 3p | 271 | | | | | | | |
| hsa-miR-215- | MIMAT0000 | 1255 | ACAGTAAAG | 1 | 1 | 1 | 3 | 2 |
| 5p | 272 | | | | | | | |
| hsa-miR-216b- | MIMAT0026 | n/a | n/a | 1 | 1 | 1 | 3 | 2 |
| 3p | 721 | | | | | | | |
| hsa-miR-224- | MIMAT0009 | n/a | n/a | 1 | 1 | 1 | 3 | 2 |
| 3p | 198 | | | | | | | |
| hsa-miR-2277- | MIMAT0017 | n/a | n/a | 1 | 1 | 1 | 3 | 2 |
| 5p | 352 | | | | | | | |
| hsa-miR-2355- | MIMAT0016 | n/a | n/a | 1 | 1 | 1 | 3 | 2 |
| 5p | 895 | | | | | | | |
| hsa-miR-2392 | MIMAT0019 | n/a | n/a | 1 | 1 | 1 | 3 | 2 |
| | 043 | | | | | | | |
| hsa-miR-23a- | MIMAT0004 | n/a | n/a | 1 | 1 | 1 | 3 | 2 |
| 5p | 496 | | | | | | | |
| hsa-miR-23b- | MIMAT0004 | n/a | n/a | 1 | 1 | 1 | 3 | 2 |
| 5p | 587 | | | | | | | |
| hsa-miR-2467- | MIMAT0019 | n/a | n/a | 1 | 1 | 1 | 3 | 2 |
| 5p | 952 | | | | | | | |
| hsa-miR-26b- | MIMAT0004 | n/a | n/a | 1 | 1 | 1 | 3 | 2 |
| 3p | 500 | | | | | | | |
| hsa-miR-296- | MIMAT0000 | n/a | n/a | 1 | 1 | 1 | 3 | 2 |
| 5p | 690 | | | | | | | |
| hsa-miR-301a- | MIMAT0000 | n/a | n/a | 1 | 1 | 1 | 3 | 2 |
| 3p | 688 | | | | | | | |
| hsa-miR-301b | MIMAT0004 | 1811 | CCTCCCCTC | 1 | 1 | 1 | 3 | 2 |
| | 958 | | | | | | | |
| hsa-miR-3064- | MIMAT0019 | 1230 | TAACGATAA | 1 | 1 | 1 | 3 | 2 |
| 5p | 864 | | | | | | | |
| hsa-miR-3116 | MIMAT0014 | n/a | n/a | 1 | 1 | 1 | 3 | 2 |
| | 978 | | | | | | | |
| hsa-miR-3124- | MIMAT0019 | n/a | n/a | 1 | 1 | 1 | 3 | 2 |
| 3p | 200 | | | | | | | |
| hsa-miR-3125 | MIMAT0014 | n/a | n/a | 1 | 1 | 1 | 3 | 2 |
| | 988 | | | | | | | |
| hsa-miR-3126- | MIMAT0014 | n/a | n/a | 1 | 1 | 1 | 3 | 2 |
| 5p | 989 | | | | | | | |
| hsa-miR-3130- | MIMAT0014 | n/a | n/a | 1 | 1 | 1 | 3 | 2 |
| 3p | 994 | | | | | | | |
| hsa-miR-3135a | MIMAT0015 | n/a | n/a | 1 | 1 | 1 | 3 | 2 |
| | 001 | | | | | | | |
| hsa-miR-3135b | MIMAT0018 | n/a | n/a | 1 | 1 | 1 | 3 | 2 |
| | 985 | | | | | | | |
| hsa-miR-3147 | MIMAT0015 | n/a | n/a | 1 | 1 | 1 | 3 | 2 |
| | 019 | | | | | | | |
| hsa-miR- | MIMAT0015 | n/a | n/a | 1 | 1 | 1 | 3 | 2 |
| 3150a-3p | 023 | | | | | | | |
| hsa-miR- | MIMAT0019 | n/a | n/a | 1 | 1 | 1 | 3 | 2 |
| 3150a-5p | 206 | | | | | | | |
| hsa-miR- | MIMAT0018 | 848 | CTGCTGCGA | 1 | 1 | 1 | 3 | 2 |
| 3150b-3p | 194 | | | | | | | |
| hsa-miR- | MIMAT0019 | n/a | n/a | 1 | 1 | 1 | 3 | 2 |
| 3150b-5p | 226 | | | | | | | |
| hsa-miR-3152- | MIMAT0019 | n/a | n/a | 1 | 1 | 1 | 3 | 2 |
| 5p | 207 | | | | | | | |
| hsa-miR-3162- | MIMAT0019 | n/a | n/a | 1 | 1 | 1 | 3 | 2 |
| 3p | 213 | | | | | | | |
| hsa-miR-3164 | MIMAT0015 | n/a | n/a | 1 | 1 | 1 | 3 | 2 |
| | 038 | | | | | | | |
| hsa-miR-3170 | MIMAT0015 | n/a | n/a | 1 | 1 | 1 | 3 | 2 |
| | 045 | | | | | | | |
| hsa-miR-3173- | MIMAT0019 | n/a | n/a | 1 | 1 | 1 | 3 | 2 |
| 5p | 214 | | | | | | | |
| hsa-miR-3176 | MIMAT0015 | n/a | n/a | 1 | 1 | 1 | 3 | 2 |
| | 053 | | | | | | | |
| hsa-miR-3180 | MIMAT0018 | n/a | n/a | 1 | 1 | 1 | 3 | 2 |
| | 178 | | | | | | | |
| hsa-miR-3180- | MIMAT0015 | n/a | n/a | 1 | 1 | 1 | 3 | 2 |
| 3p | 058 | | | | | | | |
| hsa-miR-3181 | MIMAT0015 | n/a | n/a | 1 | 1 | 1 | 3 | 2 |
| | 061 | | | | | | | |
| hsa-miR-3186- | MIMAT0015 | n/a | n/a | 1 | 1 | 1 | 3 | 2 |
| 5p | 067 | | | | | | | |
| hsa-miR-3190- | MIMAT0022 | n/a | n/a | 1 | 1 | 1 | 3 | 2 |
| 3p | 839 | | | | | | | |
| hsa-miR-3191- | MIMAT0015 | n/a | n/a | 1 | 1 | 1 | 3 | 2 |
| 3p | 075 | | | | | | | |
| hsa-miR-3193 | MIMAT0015 | n/a | n/a | 1 | 1 | 1 | 3 | 2 |
| | 077 | | | | | | | |
| hsa-miR-3196 | MIMAT0015 | n/a | n/a | 1 | 1 | 1 | 3 | 2 |
| | 080 | | | | | | | |
| hsa-miR-3197 | MIMAT0015 | n/a | n/a | 1 | 1 | 1 | 3 | 2 |
| | 082 | | | | | | | |
| hsa-miR-324- | MIMAT0000 | n/a | n/a | 1 | 1 | 1 | 3 | 2 |
| 3p | 762 | | | | | | | |
| hsa-miR-328- | MIMAT0000 | n/a | n/a | 1 | 1 | 1 | 3 | 2 |
| 3p | 752 | | | | | | | |
| hsa-miR-329- | MIMAT0001 | n/a | n/a | 1 | 1 | 1 | 3 | 2 |
| 3p | 629 | | | | | | | |
| hsa-miR-33b- | MIMAT0004 | 638 | AGCAGCTGC | 1 | 1 | 1 | 3 | 2 |
| 3p | 811 | | | | | | | |
| hsa-miR-345- | MIMAT0022 | n/a | n/a | 1 | 1 | 1 | 3 | 2 |
| 3p | 698 | | | | | | | |
| hsa-miR-34b- | MIMAT0004 | n/a | n/a | 1 | 1 | 1 | 3 | 2 |
| 3p | 676 | | | | | | | |
| hsa-miR-3605- | MIMAT0017 | n/a | n/a | 1 | 1 | 1 | 3 | 2 |
| 5p | 981 | | | | | | | |
| hsa-miR-3616- | MIMAT0017 | n/a | n/a | 1 | 1 | 1 | 3 | 2 |
| 5p | 995 | | | | | | | |
| hsa-miR-3619- | MIMAT0017 | n/a | n/a | 1 | 1 | 1 | 3 | 2 |
| 5p | 999 | | | | | | | |
| hsa-miR-3620- | MIMAT0018 | n/a | n/a | 1 | 1 | 1 | 3 | 2 |
| 3p | 001 | | | | | | | |
| hsa-miR-3621 | MIMAT0018 | 326 | GGGGTGCA | 1 | 1 | 1 | 3 | 2 |
| | 002 | | GG | | | | | |
| hsa-miR-362- | MIMAT0004 | n/a | n/a | 1 | 1 | 1 | 3 | 2 |
| 3p | 683 | | | | | | | |
| hsa-miR-3652 | MIMAT0018 | n/a | n/a | 1 | 1 | 1 | 3 | 2 |
| | 072 | | | | | | | |
| hsa-miR-3657 | MIMAT0018 | n/a | n/a | 1 | 1 | 1 | 3 | 2 |
| | 077 | | | | | | | |
| hsa-miR-3665 | MIMAT0018 | n/a | n/a | 1 | 1 | 1 | 3 | 2 |
| | 087 | | | | | | | |
| hsa-miR-3666 | MIMAT0018 | n/a | n/a | 1 | 1 | 1 | 3 | 2 |
| | 088 | | | | | | | |
| hsa-miR-3667- | MIMAT0018 | n/a | n/a | 1 | 1 | 1 | 3 | 2 |
| 5p | 089 | | | | | | | |
| hsa-miR-3674 | MIMAT0018 | n/a | n/a | 1 | 1 | 1 | 3 | 2 |
| | 097 | | | | | | | |
| hsa-miR-3675- | MIMAT0018 | n/a | n/a | 1 | 1 | 1 | 3 | 2 |
| 5p | 098 | | | | | | | |
| hsa-miR-3689d | MIMAT0019 | n/a | n/a | 1 | 1 | 1 | 3 | 2 |
| | 008 | | | | | | | |
| hsa-miR-3692- | MIMAT0018 | n/a | n/a | 1 | 1 | 1 | 3 | 2 |
| 5p | 121 | | | | | | | |
| hsa-miR-371b- | MIMAT0019 | n/a | n/a | 1 | 1 | 1 | 3 | 2 |
| 3p | 893 | | | | | | | |
| hsa-miR-374c- | MIMAT0022 | n/a | n/a | 1 | 1 | 1 | 3 | 2 |
| 3p | 735 | | | | | | | |
| hsa-miR-378a- | MIMAT0000 | n/a | n/a | 1 | 1 | 1 | 3 | 2 |
| 5p | 731 | | | | | | | |
| hsa-miR-378j | MIMAT0024 | 409 | ACAGACACC | 1 | 1 | 1 | 3 | 2 |
| | 612 | | | | | | | |
| hsa-miR-3916 | MIMAT0018 | n/a | n/a | 1 | 1 | 1 | 3 | 2 |
| | 190 | | | | | | | |
| hsa-miR-3922- | MIMAT0018 | 1528 | GAGGGTCCT | 1 | 1 | 1 | 3 | 2 |
| 3p | 197 | | | | | | | |
| hsa-miR-3929 | MIMAT0018 | n/a | n/a | 1 | 1 | 1 | 3 | 2 |
| | 206 | | | | | | | |
| hsa-miR-3934- | MIMAT0022 | n/a | n/a | 1 | 1 | 1 | 3 | 2 |
| 3p | 975 | | | | | | | |
| hsa-miR-3934- | MIMAT0018 | n/a | n/a | 1 | 1 | 1 | 3 | 2 |
| 5p | 349 | | | | | | | |
| hsa-miR-3975 | MIMAT0019 | n/a | n/a | 1 | 1 | 1 | 3 | 2 |
| | 360 | | | | | | | |
| hsa-miR-3977 | MIMAT0019 | n/a | n/a | 1 | 1 | 1 | 3 | 2 |
| | 362 | | | | | | | |
| hsa-miR-4258 | MIMAT0016 | n/a | n/a | 1 | 1 | 1 | 3 | 2 |
| | 879 | | | | | | | |
| hsa-miR-4259 | MIMAT0016 | n/a | n/a | 1 | 1 | 1 | 3 | 2 |
| | 880 | | | | | | | |
| hsa-miR-4260 | MIMAT0016 | n/a | n/a | 1 | 1 | 1 | 3 | 2 |
| | 881 | | | | | | | |
| hsa-miR-4266 | MIMAT0016 | n/a | n/a | 1 | 1 | 1 | 3 | 2 |
| | 892 | | | | | | | |
| hsa-miR-4267 | MIMAT0016 | n/a | n/a | 1 | 1 | 1 | 3 | 2 |
| | 893 | | | | | | | |
| hsa-miR-4270 | MIMAT0016 | n/a | n/a | 1 | 1 | 1 | 3 | 2 |
| | 900 | | | | | | | |
| hsa-miR-4271 | MIMAT0016 | n/a | n/a | 1 | 1 | 1 | 3 | 2 |
| | 901 | | | | | | | |
| hsa-miR-4284 | MIMAT0016 | n/a | n/a | 1 | 1 | 1 | 3 | 2 |
| | 915 | | | | | | | |
| hsa-miR-429 | MIMAT0001 | n/a | n/a | 1 | 1 | 1 | 3 | 2 |
| | 536 | | | | | | | |
| hsa-miR-4294 | MIMAT0016 | n/a | n/a | 1 | 1 | 1 | 3 | 2 |
| | 849 | | | | | | | |
| hsa-miR-4295 | MIMAT0016 | 1811 | CCTCCCCTC | 1 | 1 | 1 | 3 | 2 |
| | 844 | | | | | | | |
| hsa-miR-4301 | MIMAT0016 | n/a | n/a | 1 | 1 | 1 | 3 | 2 |
| | 850 | | | | | | | |
| hsa-miR-4316 | MIMAT0016 | n/a | n/a | 1 | 1 | 1 | 3 | 2 |
| | 867 | | | | | | | |
| hsa-miR-4319 | MIMAT0016 | n/a | n/a | 1 | 1 | 1 | 3 | 2 |
| | 870 | | | | | | | |
| hsa-miR-4419a | MIMAT0018 | n/a | n/a | 1 | 1 | 1 | 3 | 2 |
| | 931 | | | | | | | |
| hsa-miR-4419b | MIMAT0019 | 704 | TCGTCGCTG | 1 | 1 | 1 | 3 | 2 |
| | 034 | | | | | | | |
| hsa-miR-4423- | MIMAT0019 | n/a | n/a | 1 | 1 | 1 | 3 | 2 |
| 5p | 232 | | | | | | | |
| hsa-miR-4430 | MIMAT0018 | 1805 | GCCTGTCCT | 1 | 1 | 1 | 3 | 2 |
| | 945 | | C | | | | | |
| hsa-miR-4433- | MIMAT0020 | 128 | AGGAGAAC | 1 | 1 | 1 | 3 | 2 |
| 5p | 956 | | T | | | | | |
| hsa-miR-4435 | MIMAT0018 | n/a | n/a | 1 | 1 | 1 | 3 | 2 |
| | 951 | | | | | | | |
| hsa-miR- | MIMAT0019 | n/a | n/a | 1 | 1 | 1 | 3 | 2 |
| 4436b-5p | 940 | | | | | | | |
| hsa-miR-4441 | MIMAT0018 | n/a | n/a | 1 | 1 | 1 | 3 | 2 |
| | 959 | | | | | | | |
| hsa-miR-4446- | MIMAT0018 | n/a | n/a | 1 | 1 | 1 | 3 | 2 |
| 3p | 965 | | | | | | | |
| hsa-miR-4461 | MIMAT0018 | n/a | n/a | 1 | 1 | 1 | 3 | 2 |
| | 983 | | | | | | | |
| hsa-miR-4469 | MIMAT0018 | n/a | n/a | 1 | 1 | 1 | 3 | 2 |
| | 996 | | | | | | | |
| hsa-miR-4482- | MIMAT0020 | n/a | n/a | 1 | 1 | 1 | 3 | 2 |
| 3p | 958 | | | | | | | |
| hsa-miR-4486 | MIMAT0019 | 604 | GGAGAGGC | 1 | 1 | 1 | 3 | 2 |
| | 020 | | A | | | | | |
| hsa-miR-4488 | MIMAT0019 | 299 | ATAGGAATC | 1 | 1 | 1 | 3 | 2 |
| | 022 | | | | | | | |
| hsa-miR-4492 | MIMAT0019 | n/a | n/a | 1 | 1 | 1 | 3 | 2 |
| | 027 | | | | | | | |
| hsa-miR-4495 | MIMAT0019 | n/a | n/a | 1 | 1 | 1 | 3 | 2 |
| | 030 | | | | | | | |
| hsa-miR-4498 | MIMAT0019 | n/a | n/a | 1 | 1 | 1 | 3 | 2 |
| | 033 | | | | | | | |
| hsa-miR-4502 | MIMAT0019 | n/a | n/a | 1 | 1 | 1 | 3 | 2 |
| | 038 | | | | | | | |
| hsa-miR-4510 | MIMAT0019 | n/a | n/a | 1 | 1 | 1 | 3 | 2 |
| | 047 | | | | | | | |
| hsa-miR-4518 | MIMAT0019 | n/a | n/a | 1 | 1 | 1 | 3 | 2 |
| | 055 | | | | | | | |
| hsa-miR-451b | MIMAT0019 | n/a | n/a | 1 | 1 | 1 | 3 | 2 |
| | 840 | | | | | | | |
| hsa-miR-4523 | MIMAT0019 | 928 | TCTATCGGT | 1 | 1 | 1 | 3 | 2 |
| | 061 | | | | | | | |
| hsa-miR-4532 | MIMAT0019 | n/a | n/a | 1 | 1 | 1 | 3 | 2 |
| | 071 | | | | | | | |
| hsa-miR-4534 | MIMAT0019 | n/a | n/a | 1 | 1 | 1 | 3 | 2 |
| | 073 | | | | | | | |
| hsa-miR-454- | MIMAT0003 | n/a | n/a | 1 | 1 | 1 | 3 | 2 |
| 3p | 885 | | | | | | | |
| hsa-miR-4638- | MIMAT0019 | n/a | n/a | 1 | 1 | 1 | 3 | 2 |
| 5p | 695 | | | | | | | |
| hsa-miR-4639- | MIMAT0019 | 1379 | TGCCGGAG | 1 | 1 | 1 | 3 | 2 |
| 5p | 697 | | G | | | | | |
| hsa-miR-4645- | MIMAT0019 | n/a | n/a | 1 | 1 | 1 | 3 | 2 |
| 3p | 706 | | | | | | | |
| hsa-miR-4649- | MIMAT0019 | n/a | n/a | 1 | 1 | 1 | 3 | 2 |
| 3p | 712 | | | | | | | |
| hsa-miR-4649- | MIMAT0019 | n/a | n/a | 1 | 1 | 1 | 3 | 2 |
| 5p | 711 | | | | | | | |
| hsa-miR-4653- | MIMAT0019 | n/a | n/a | 1 | 1 | 1 | 3 | 2 |
| 3p | 719 | | | | | | | |
| hsa-miR-4660 | MIMAT0019 | n/a | n/a | 1 | 1 | 1 | 3 | 2 |
| | 728 | | | | | | | |
| hsa-miR-4667- | MIMAT0019 | n/a | n/a | 1 | 1 | 1 | 3 | 2 |
| 3p | 744 | | | | | | | |
| hsa-miR-4677- | MIMAT0019 | 1235 | ATAATTCCA | 1 | 1 | 1 | 3 | 2 |
| 3p | 761 | | | | | | | |
| hsa-miR-4677- | MIMAT0019 | n/a | n/a | 1 | 1 | 1 | 3 | 2 |
| 5p | 760 | | | | | | | |
| hsa-miR-4679 | MIMAT0019 | n/a | n/a | 1 | 1 | 1 | 3 | 2 |
| | 763 | | | | | | | |
| hsa-miR-4682 | MIMAT0019 | 578 | TTGGGGACA | 1 | 1 | 1 | 3 | 2 |
| | 767 | | | | | | | |
| hsa-miR-4689 | MIMAT0019 | n/a | n/a | 1 | 1 | 1 | 3 | 2 |
| | 778 | | | | | | | |
| hsa-miR-4691- | MIMAT0019 | n/a | n/a | 1 | 1 | 1 | 3 | 2 |
| 3p | 782 | | | | | | | |
| hsa-miR-4693- | MIMAT0019 | n/a | n/a | 1 | 1 | 1 | 3 | 2 |
| 5p | 784 | | | | | | | |
| hsa-miR-4697- | MIMAT0019 | n/a | n/a | 1 | 1 | 1 | 3 | 2 |
| 5p | 791 | | | | | | | |
| hsa-miR-4701- | MIMAT0019 | n/a | n/a | 1 | 1 | 1 | 3 | 2 |
| 3p | 799 | | | | | | | |
| hsa-miR-4706 | MIMAT0019 | n/a | n/a | 1 | 1 | 1 | 3 | 2 |
| | 806 | | | | | | | |
| hsa-miR-4707- | MIMAT0019 | n/a | n/a | 1 | 1 | 1 | 3 | 2 |
| 3p | 808 | | | | | | | |
| hsa-miR-4715- | MIMAT0019 | n/a | n/a | 1 | 1 | 1 | 3 | 2 |
| 3p | 825 | | | | | | | |
| hsa-miR-4716- | MIMAT0019 | n/a | n/a | 1 | 1 | 1 | 3 | 2 |
| 5p | 826 | | | | | | | |
| hsa-miR-4717- | MIMAT0019 | 1179 | CGTGGGAG | 1 | 1 | 1 | 3 | 2 |
| 5p | 829 | | GAT | | | | | |
| hsa-miR-4719 | MIMAT0019 | n/a | n/a | 1 | 1 | 1 | 3 | 2 |
| | 832 | | | | | | | |
| hsa-miR-4722- | MIMAT0019 | n/a | n/a | 1 | 1 | 1 | 3 | 2 |
| 5p | 836 | | | | | | | |
| hsa-miR-4725- | MIMAT0019 | n/a | n/a | 1 | 1 | 1 | 3 | 2 |
| 3p | 844 | | | | | | | |
| hsa-miR-4726- | MIMAT0019 | n/a | n/a | 1 | 1 | 1 | 3 | 2 |
| 3p | 846 | | | | | | | |
| hsa-miR-4727- | MIMAT0019 | n/a | n/a | 1 | 1 | 1 | 3 | 2 |
| 5p | 847 | | | | | | | |
| hsa-miR-4728- | MIMAT0019 | 869 | CCCTACAAC | 1 | 1 | 1 | 3 | 2 |
| 5p | 849 | | | | | | | |
| hsa-miR-4730 | MIMAT0019 | n/a | n/a | 1 | 1 | 1 | 3 | 2 |
| | 852 | | | | | | | |
| hsa-miR-4737 | MIMAT0019 | n/a | n/a | 1 | 1 | 1 | 3 | 2 |
| | 863 | | | | | | | |
| hsa-miR-4738- | MIMAT0019 | n/a | n/a | 1 | 1 | 1 | 3 | 2 |
| 5p | 866 | | | | | | | |
| hsa-miR-4739 | MIMAT0019 | n/a | n/a | 1 | 1 | 1 | 3 | 2 |
| | 868 | | | | | | | |
| hsa-miR-4749- | MIMAT0019 | 1566 | CAGCCCTAT | 1 | 1 | 1 | 3 | 2 |
| 3p | 886 | | G | | | | | |
| hsa-miR-4749- | MIMAT0019 | n/a | n/a | 1 | 1 | 1 | 3 | 2 |
| 5p | 885 | | | | | | | |
| hsa-miR-4753- | MIMAT0019 | 1224 | ACTGCATAA | 1 | 1 | 1 | 3 | 2 |
| 3p | 891 | | | | | | | |
| hsa-miR-4756- | MIMAT0019 | n/a | n/a | 1 | 1 | 1 | 3 | 2 |
| 5p | 899 | | | | | | | |
| hsa-miR-4770 | MIMAT0019 | n/a | n/a | 1 | 1 | 1 | 3 | 2 |
| | 924 | | | | | | | |
| hsa-miR-4772- | MIMAT0019 | n/a | n/a | 1 | 1 | 1 | 3 | 2 |
| 3p | 927 | | | | | | | |
| hsa-miR-4772- | MIMAT0019 | n/a | n/a | 1 | 1 | 1 | 3 | 2 |
| 5p | 926 | | | | | | | |
| hsa-miR-4774- | MIMAT0019 | n/a | n/a | 1 | 1 | 1 | 3 | 2 |
| 3p | 930 | | | | | | | |
| hsa-miR-4778- | MIMAT0019 | n/a | n/a | 1 | 1 | 1 | 3 | 2 |
| 5p | 936 | | | | | | | |
| hsa-miR-4779 | MIMAT0019 | 1889 | TAGGGGCG | 1 | 1 | 1 | 3 | 2 |
| | 938 | | G | | | | | |
| hsa-miR-4782- | MIMAT0019 | n/a | n/a | 1 | 1 | 1 | 3 | 2 |
| 5p | 944 | | | | | | | |
| hsa-miR-4784 | MIMAT0019 | 849 | TGCTGCGAC | 1 | 1 | 1 | 3 | 2 |
| | 948 | | | | | | | |
| hsa-miR-4787- | MIMAT0019 | n/a | n/a | 1 | 1 | 1 | 3 | 2 |
| 5p | 956 | | | | | | | |
| hsa-miR-4790- | MIMAT0019 | n/a | n/a | 1 | 1 | 1 | 3 | 2 |
| 5p | 961 | | | | | | | |
| hsa-miR-491- | MIMAT0004 | n/a | n/a | 1 | 1 | 1 | 3 | 2 |
| 3p | 765 | | | | | | | |
| hsa-miR-491- | MIMAT0002 | n/a | n/a | 1 | 1 | 1 | 3 | 2 |
| 5p | 807 | | | | | | | |
| hsa-miR-495- | MIMAT0022 | n/a | n/a | 1 | 1 | 1 | 3 | 2 |
| 5p | 924 | | | | | | | |
| hsa-miR-498 | MIMAT0002 | n/a | n/a | 1 | 1 | 1 | 3 | 2 |
| | 824 | | | | | | | |
| hsa-miR-5001- | MIMAT0021 | 428 | GAGGGGCA | 1 | 1 | 1 | 3 | 2 |
| 5p | 021 | | A | | | | | |
| hsa-miR-5002- | MIMAT0021 | n/a | n/a | 1 | 1 | 1 | 3 | 2 |
| 3p | 024 | | | | | | | |
| hsa-miR-5004- | MIMAT0021 | 186 | CACTCCAGC | 1 | 1 | 1 | 3 | 2 |
| 5p | 027 | | | | | | | |
| hsa-miR-5006- | MIMAT0021 | n/a | n/a | 1 | 1 | 1 | 3 | 2 |
| 5p | 033 | | | | | | | |
| hsa-miR-5008- | MIMAT0021 | 831 | TGGGCTCCT | 1 | 1 | 1 | 3 | 2 |
| 3p | 040 | | GGG | | | | | |
| hsa-miR-5088- | MIMAT0027 | 1599 | GAAGCCCCA | 1 | 1 | 1 | 3 | 2 |
| 3p | 041 | | | | | | | |
| hsa-miR-5090 | MIMAT0021 | n/a | n/a | 1 | 1 | 1 | 3 | 2 |
| | 082 | | | | | | | |
| hsa-miR-5095 | MIMAT0020 | n/a | n/a | 1 | 1 | 1 | 3 | 2 |
| | 600 | | | | | | | |
| hsa-miR-515- | MIMAT0002 | n/a | n/a | 1 | 1 | 1 | 3 | 2 |
| 3p | 827 | | | | | | | |
| hsa-miR-5195- | MIMAT0021 | n/a | n/a | 1 | 1 | 1 | 3 | 2 |
| 5p | 126 | | | | | | | |
| hsa-miR-519e- | MIMAT0002 | n/a | n/a | 1 | 1 | 1 | 3 | 2 |
| 3p | 829 | | | | | | | |
| hsa-miR-522- | MIMAT0002 | n/a | n/a | 1 | 1 | 1 | 3 | 2 |
| 3p | 868 | | | | | | | |
| hsa-miR-523- | MIMAT0002 | 712 | GCAAAACAA | 1 | 1 | 1 | 3 | 2 |
| 3p | 840 | | | | | | | |
| hsa-miR-532- | MIMAT0004 | n/a | n/a | 1 | 1 | 1 | 3 | 2 |
| 3p | 780 | | | | | | | |
| hsa-miR-541- | MIMAT0004 | n/a | n/a | 1 | 1 | 1 | 3 | 2 |
| 3p | 920 | | | | | | | |
| hsa-miR-548e- | MIMAT0026 | n/a | n/a | 1 | 1 | 1 | 3 | 2 |
| 5p | 736 | | | | | | | |
| hsa-miR-552- | MIMAT0003 | n/a | n/a | 1 | 1 | 1 | 3 | 2 |
| 3p | 215 | | | | | | | |
| hsa-miR-5571- | MIMAT0022 | n/a | n/a | 1 | 1 | 1 | 3 | 2 |
| 3p | 258 | | | | | | | |
| hsa-miR-5572 | MIMAT0022 | n/a | n/a | 1 | 1 | 1 | 3 | 2 |
| | 260 | | | | | | | |
| hsa-miR-5587- | MIMAT0022 | n/a | n/a | 1 | 1 | 1 | 3 | 2 |
| 3p | 290 | | | | | | | |
| hsa-miR-5589- | MIMAT0022 | n/a | n/a | 1 | 1 | 1 | 3 | 2 |
| 5p | 297 | | | | | | | |
| hsa-miR-566 | MIMAT0003 | n/a | n/a | 1 | 1 | 1 | 3 | 2 |
| | 230 | | | | | | | |
| hsa-miR-5682 | MIMAT0022 | 1079 | CAACTTCTT | 1 | 1 | 1 | 3 | 2 |
| | 470 | | | | | | | |
| hsa-miR-5702 | MIMAT0022 | 1684 | AAAGCCGG | 1 | 1 | 1 | 3 | 2 |
| | 495 | | A | | | | | |
| hsa-miR-5706 | MIMAT0022 | n/a | n/a | 1 | 1 | 1 | 3 | 2 |
| | 500 | | | | | | | |
| hsa-miR-571 | MIMAT0003 | n/a | n/a | 1 | 1 | 1 | 3 | 2 |
| | 236 | | | | | | | |
| hsa-miR-573 | MIMAT0003 | 1013 | GCAGAATTA | 1 | 1 | 1 | 3 | 2 |
| | 238 | | | | | | | |
| hsa-miR-5739 | MIMAT0023 | n/a | n/a | 1 | 1 | 1 | 3 | 2 |
| | 116 | | | | | | | |
| hsa-miR-5787 | MIMAT0023 | n/a | n/a | 1 | 1 | 1 | 3 | 2 |
| | 252 | | | | | | | |
| hsa-miR-585- | MIMAT0026 | n/a | n/a | 1 | 1 | 1 | 3 | 2 |
| 5p | 618 | | | | | | | |
| hsa-miR-592 | MIMAT0003 | n/a | n/a | 1 | 1 | 1 | 3 | 2 |
| | 260 | | | | | | | |
| hsa-miR-596 | MIMAT0003 | n/a | n/a | 1 | 1 | 1 | 3 | 2 |
| | 264 | | | | | | | |
| hsa-miR-6081 | MIMAT0023 | n/a | n/a | 1 | 1 | 1 | 3 | 2 |
| | 706 | | | | | | | |
| hsa-miR-6087 | MIMAT0023 | 45 | AAACCCCAT | 1 | 1 | 1 | 3 | 2 |
| | 712 | | | | | | | |
| hsa-miR-6088 | MIMAT0023 | n/a | n/a | 1 | 1 | 1 | 3 | 2 |
| | 713 | | | | | | | |
| hsa-miR-6127 | MIMAT0024 | n/a | n/a | 1 | 1 | 1 | 3 | 2 |
| | 610 | | | | | | | |
| hsa-miR-6129 | MIMAT0024 | n/a | n/a | 1 | 1 | 1 | 3 | 2 |
| | 613 | | | | | | | |
| hsa-miR-6130 | MIMAT0024 | n/a | n/a | 1 | 1 | 1 | 3 | 2 |
| | 614 | | | | | | | |
| hsa-miR-6133 | MIMAT0024 | n/a | n/a | 1 | 1 | 1 | 3 | 2 |
| | 617 | | | | | | | |
| hsa-miR-6134 | MIMAT0024 | n/a | n/a | 1 | 1 | 1 | 3 | 2 |
| | 618 | | | | | | | |
| hsa-miR-615- | MIMAT0004 | n/a | n/a | 1 | 1 | 1 | 3 | 2 |
| 5p | 804 | | | | | | | |
| hsa-miR-6165 | MIMAT0024 | n/a | n/a | 1 | 1 | 1 | 3 | 2 |
| | 782 | | | | | | | |
| hsa-miR-627- | MIMAT0026 | n/a | n/a | 1 | 1 | 1 | 3 | 2 |
| 3p | 623 | | | | | | | |
| hsa-miR-629- | MIMAT0003 | n/a | n/a | 1 | 1 | 1 | 3 | 2 |
| 3p | 298 | | | | | | | |
| hsa-miR-642a- | MIMAT0003 | 1658 | CTCAAGGCC | 1 | 1 | 1 | 3 | 2 |
| 5p | 312 | | | | | | | |
| hsa-miR-649 | MIMAT0003 | 1266 | CCAGGCGCC | 1 | 1 | 1 | 3 | 2 |
| | 319 | | | | | | | |
| hsa-miR-6504- | MIMAT0025 | 1231 | AACGATAAT | 1 | 1 | 1 | 3 | 2 |
| 5p | 464 | | | | | | | |
| hsa-miR- | MIMAT0025 | n/a | n/a | 1 | 1 | 1 | 3 | 2 |
| 6511a-3p | 479 | | | | | | | |
| hsa-miR- | MIMAT0025 | n/a | n/a | 1 | 1 | 1 | 3 | 2 |
| 6511b-3p | 848 | | | | | | | |
| hsa-miR-6513- | MIMAT0025 | n/a | n/a | 1 | 1 | 1 | 3 | 2 |
| 3p | 483 | | | | | | | |
| hsa-miR-654- | MIMAT0003 | n/a | n/a | 1 | 1 | 1 | 3 | 2 |
| 5p | 330 | | | | | | | |
| hsa-miR-657 | MIMAT0003 | n/a | n/a | 1 | 1 | 1 | 3 | 2 |
| | 335 | | | | | | | |
| hsa-miR-658 | MIMAT0003 | n/a | n/a | 1 | 1 | 1 | 3 | 2 |
| | 336 | | | | | | | |
| hsa-miR-660- | MIMAT0022 | 1569 | CCCTATGGC | 1 | 1 | 1 | 3 | 2 |
| 3p | 711 | | G | | | | | |
| hsa-miR-661 | MIMAT0003 | 729 | AGGTTTTAC | 1 | 1 | 1 | 3 | 2 |
| | 324 | | A | | | | | |
| hsa-miR-663a | MIMAT0003 | n/a | n/a | 1 | 1 | 1 | 3 | 2 |
| | 326 | | | | | | | |
| hsa-miR-6720- | MIMAT0025 | n/a | n/a | 1 | 1 | 1 | 3 | 2 |
| 3p | 851 | | | | | | | |
| hsa-miR-6727- | MIMAT0027 | n/a | n/a | 1 | 1 | 1 | 3 | 2 |
| 5p | 355 | | | | | | | |
| hsa-miR-6729- | MIMAT0027 | n/a | n/a | 1 | 1 | 1 | 3 | 2 |
| 5p | 359 | | | | | | | |
| hsa-miR-6730- | MIMAT0027 | 561 | GTGAGACCC | 1 | 1 | 1 | 3 | 2 |
| 3p | 362 | | | | | | | |
| hsa-miR-6732- | MIMAT0027 | n/a | n/a | 1 | 1 | 1 | 3 | 2 |
| 3p | 366 | | | | | | | |
| hsa-miR-6736- | MIMAT0027 | 383 | CTGGTGAGA | 1 | 1 | 1 | 3 | 2 |
| 5p | 373 | | | | | | | |
| hsa-miR-6737- | MIMAT0027 | n/a | n/a | 1 | 1 | 1 | 3 | 2 |
| 3p | 376 | | | | | | | |
| hsa-miR-6742- | MIMAT0027 | n/a | n/a | 1 | 1 | 1 | 3 | 2 |
| 3p | 386 | | | | | | | |
| hsa-miR-6743- | MIMAT0027 | n/a | n/a | 1 | 1 | 1 | 3 | 2 |
| 3p | 388 | | | | | | | |
| hsa-miR-6746- | MIMAT0027 | n/a | n/a | 1 | 1 | 1 | 3 | 2 |
| 3p | 393 | | | | | | | |
| hsa-miR-6749- | MIMAT0027 | n/a | n/a | 1 | 1 | 1 | 3 | 2 |
| 3p | 399 | | | | | | | |
| hsa-miR-6749- | MIMAT0027 | n/a | n/a | 1 | 1 | 1 | 3 | 2 |
| 5p | 398 | | | | | | | |
| hsa-miR-6752- | MIMAT0027 | 1538 | ACTCCCCAC | 1 | 1 | 1 | 3 | 2 |
| 3p | 405 | | | | | | | |
| hsa-miR-6752- | MIMAT0027 | n/a | n/a | 1 | 1 | 1 | 3 | 2 |
| 5p | 404 | | | | | | | |
| hsa-miR-6754- | MIMAT0027 | 866 | GGTCCCTAC | 1 | 1 | 1 | 3 | 2 |
| 5p | 408 | | | | | | | |
| hsa-miR-6760- | MIMAT0027 | n/a | n/a | 1 | 1 | 1 | 3 | 2 |
| 3p | 421 | | | | | | | |
| hsa-miR-6763- | MIMAT0027 | n/a | n/a | 1 | 1 | 1 | 3 | 2 |
| 5p | 426 | | | | | | | |
| hsa-miR-6764- | MIMAT0027 | n/a | n/a | 1 | 1 | 1 | 3 | 2 |
| 5p | 428 | | | | | | | |
| hsa-miR-6765- | MIMAT0027 | n/a | n/a | 1 | 1 | 1 | 3 | 2 |
| 3p | 431 | | | | | | | |
| hsa-miR-6771- | MIMAT0027 | n/a | n/a | 1 | 1 | 1 | 3 | 2 |
| 3p | 443 | | | | | | | |
| hsa-miR-6773- | MIMAT0027 | n/a | n/a | 1 | 1 | 1 | 3 | 2 |
| 3p | 447 | | | | | | | |
| hsa-miR-6775- | MIMAT0027 | n/a | n/a | 1 | 1 | 1 | 3 | 2 |
| 3p | 451 | | | | | | | |
| hsa-miR-6775- | MIMAT0027 | n/a | n/a | 1 | 1 | 1 | 3 | 2 |
| 5p | 450 | | | | | | | |
| hsa-miR-6776- | MIMAT0027 | n/a | n/a | 1 | 1 | 1 | 3 | 2 |
| 5p | 452 | | | | | | | |
| hsa-miR-6778- | MIMAT0027 | n/a | n/a | 1 | 1 | 1 | 3 | 2 |
| 3p | 457 | | | | | | | |
| hsa-miR- | MIMAT0027 | n/a | n/a | 1 | 1 | 1 | 3 | 2 |
| 6780b-5p | 572 | | | | | | | |
| hsa-miR-6783- | MIMAT0027 | n/a | n/a | 1 | 1 | 1 | 3 | 2 |
| 3p | 467 | | | | | | | |
| hsa-miR-6783- | MIMAT0027 | n/a | n/a | 1 | 1 | 1 | 3 | 2 |
| 5p | 466 | | | | | | | |
| hsa-miR-6785- | MIMAT0027 | n/a | n/a | 1 | 1 | 1 | 3 | 2 |
| 5p | 470 | | | | | | | |
| hsa-miR-6787- | MIMAT0027 | n/a | n/a | 1 | 1 | 1 | 3 | 2 |
| 5p | 474 | | | | | | | |
| hsa-miR-6789- | MIMAT0027 | n/a | n/a | 1 | 1 | 1 | 3 | 2 |
| 5p | 478 | | | | | | | |
| hsa-miR-6790- | MIMAT0027 | n/a | n/a | 1 | 1 | 1 | 3 | 2 |
| 3p | 481 | | | | | | | |
| hsa-miR-6793- | MIMAT0027 | n/a | n/a | 1 | 1 | 1 | 3 | 2 |
| 3p | 487 | | | | | | | |
| hsa-miR-6797- | MIMAT0027 | n/a | n/a | 1 | 1 | 1 | 3 | 2 |
| 5p | 494 | | | | | | | |
| hsa-miR-6799- | MIMAT0027 | n/a | n/a | 1 | 1 | 1 | 3 | 2 |
| 3p | 499 | | | | | | | |
| hsa-miR-6804- | MIMAT0027 | n/a | n/a | 1 | 1 | 1 | 3 | 2 |
| 3p | 509 | | | | | | | |
| hsa-miR-6805- | MIMAT0027 | 298 | GATAGGAAT | 1 | 1 | 1 | 3 | 2 |
| 5p | 510 | | | | | | | |
| hsa-miR-1208 | MIMAT0005 | n/a | n/a | 1 | 0 | 1 | 2 | 2 |
| | 873 | | | | | | | |
| hsa-miR-1224- | MIMAT0005 | n/a | n/a | 1 | 0 | 1 | 2 | 2 |
| 3p | 459 | | | | | | | |
| hsa-miR-122- | MIMAT0000 | n/a | n/a | 1 | 0 | 1 | 2 | 2 |
| 5p | 421 | | | | | | | |
| hsa-miR-1227- | MIMAT0005 | n/a | n/a | 1 | 0 | 1 | 2 | 2 |
| 3p | 580 | | | | | | | |
| hsa-miR-1227- | MIMAT0022 | n/a | n/a | 0 | 1 | 1 | 2 | 2 |
| 5p | 941 | | | | | | | |
| hsa-miR-1228- | MIMAT0005 | n/a | n/a | 1 | 0 | 1 | 2 | 2 |
| 5p | 582 | | | | | | | |
| hsa-miR-1247- | MIMAT0022 | n/a | n/a | 1 | 0 | 1 | 2 | 2 |
| 3p | 721 | | | | | | | |
| hsa-miR-1248 | MIMAT0005 | n/a | n/a | 0 | 1 | 1 | 2 | 2 |
| | 900 | | | | | | | |
| hsa-miR-1260a | MIMAT0005 | n/a | n/a | 0 | 1 | 1 | 2 | 2 |
| | 911 | | | | | | | |
| hsa-miR-1268a | MIMAT0005 | n/a | n/a | 0 | 1 | 1 | 2 | 2 |
| | 922 | | | | | | | |
| hsa-miR-1268b | MIMAT0018 | n/a | n/a | 0 | 1 | 1 | 2 | 2 |
| | 925 | | | | | | | |
| hsa-miR-1271- | MIMAT0022 | n/a | n/a | 1 | 0 | 1 | 2 | 2 |
| 3p | 712 | | | | | | | |
| hsa-miR-1285- | MIMAT0022 | n/a | n/a | 1 | 0 | 1 | 2 | 2 |
| 5p | 719 | | | | | | | |
| hsa-miR-1290 | MIMAT0005 | n/a | n/a | 1 | 0 | 1 | 2 | 2 |
| | 880 | | | | | | | |
| hsa-miR-1298- | MIMAT0005 | n/a | n/a | 1 | 0 | 1 | 2 | 2 |
| 5p | 800 | | | | | | | |
| hsa-miR-1301- | MIMAT0005 | n/a | n/a | 1 | 0 | 1 | 2 | 2 |
| 3p | 797 | | | | | | | |
| hsa-miR-1343- | MIMAT0027 | n/a | n/a | 1 | 0 | 1 | 2 | 2 |
| 5p | 038 | | | | | | | |
| hsa-miR-139- | MIMAT0004 | n/a | n/a | 1 | 0 | 1 | 2 | 2 |
| 3p | 552 | | | | | | | |
| hsa-miR-146b- | MIMAT0004 | n/a | n/a | 1 | 0 | 1 | 2 | 2 |
| 3p | 766 | | | | | | | |
| hsa-miR-149- | MIMAT0000 | n/a | n/a | 1 | 0 | 1 | 2 | 2 |
| 5p | 450 | | | | | | | |
| hsa-miR-1539 | MIMAT0007 | n/a | n/a | 1 | 0 | 1 | 2 | 2 |
| | 401 | | | | | | | |
| hsa-miR-1915- | MIMAT0007 | n/a | n/a | 0 | 1 | 1 | 2 | 2 |
| 5p | 891 | | | | | | | |
| hsa-miR-197- | MIMAT0000 | n/a | n/a | 1 | 0 | 1 | 2 | 2 |
| 3p | 227 | | | | | | | |
| hsa-miR-202- | MIMAT0002 | n/a | n/a | 1 | 0 | 1 | 2 | 2 |
| 5p | 810 | | | | | | | |
| hsa-miR-219a- | MIMAT0000 | n/a | n/a | 1 | 0 | 1 | 2 | 2 |
| 5p | 276 | | | | | | | |
| hsa-miR-223- | MIMAT0000 | n/a | n/a | 1 | 0 | 1 | 2 | 2 |
| 3p | 280 | | | | | | | |
| hsa-miR-2276- | MIMAT0026 | n/a | n/a | 1 | 0 | 1 | 2 | 2 |
| 5p | 921 | | | | | | | |
| hsa-miR-24-3p | MIMAT0000 | n/a | n/a | 1 | 0 | 1 | 2 | 2 |
| | 080 | | | | | | | |
| hsa-miR-29a- | MIMAT0000 | n/a | n/a | 1 | 0 | 1 | 2 | 2 |
| 3p | 086 | | | | | | | |
| hsa-miR-29b- | MIMAT0000 | n/a | n/a | 1 | 0 | 1 | 2 | 2 |
| 3p | 100 | | | | | | | |
| hsa-miR-29c- | MIMAT0000 | n/a | n/a | 1 | 0 | 1 | 2 | 2 |
| 3p | 681 | | | | | | | |
| hsa-miR-29c- | MIMAT0004 | n/a | n/a | 1 | 0 | 1 | 2 | 2 |
| 5p | 673 | | | | | | | |
| hsa-miR-3127- | MIMAT0019 | n/a | n/a | 1 | 0 | 1 | 2 | 2 |
| 3p | 201 | | | | | | | |
| hsa-miR-3134 | MIMAT0015 | n/a | n/a | 1 | 0 | 1 | 2 | 2 |
| | 000 | | | | | | | |
| hsa-miR-3158- | MIMAT0015 | n/a | n/a | 1 | 0 | 1 | 2 | 2 |
| 3p | 032 | | | | | | | |
| hsa-miR-3187- | MIMAT0015 | n/a | n/a | 1 | 0 | 1 | 2 | 2 |
| 3p | 069 | | | | | | | |
| hsa-miR-3188 | MIMAT0015 | n/a | n/a | 1 | 0 | 1 | 2 | 2 |
| | 070 | | | | | | | |
| hsa-miR-3192- | MIMAT0015 | n/a | n/a | 0 | 1 | 1 | 2 | 2 |
| 5p | 076 | | | | | | | |
| hsa-miR-3655 | MIMAT0018 | n/a | n/a | 1 | 0 | 1 | 2 | 2 |
| | 075 | | | | | | | |
| hsa-miR-3664- | MIMAT0019 | n/a | n/a | 1 | 0 | 1 | 2 | 2 |
| 3p | 220 | | | | | | | |
| hsa-miR-3672 | MIMAT0018 | n/a | n/a | 0 | 1 | 1 | 2 | 2 |
| | 095 | | | | | | | |
| hsa-miR-3680- | MIMAT0018 | n/a | n/a | 1 | 0 | 1 | 2 | 2 |
| 3p | 107 | | | | | | | |
| hsa-miR-3685 | MIMAT0018 | n/a | n/a | 1 | 0 | 1 | 2 | 2 |
| | 113 | | | | | | | |
| hsa-miR-379- | MIMAT0004 | n/a | n/a | 1 | 0 | 1 | 2 | 2 |
| 3p | 690 | | | | | | | |
| hsa-miR-380- | MIMAT0000 | n/a | n/a | 1 | 0 | 1 | 2 | 2 |
| 3p | 735 | | | | | | | |
| hsa-miR-382- | MIMAT0000 | n/a | n/a | 1 | 0 | 1 | 2 | 2 |
| 5p | 737 | | | | | | | |
| hsa-miR-384 | MIMAT0001 | n/a | n/a | 1 | 0 | 1 | 2 | 2 |
| | 075 | | | | | | | |
| hsa-miR-411- | MIMAT0004 | n/a | n/a | 1 | 0 | 1 | 2 | 2 |
| 3p | 813 | | | | | | | |
| hsa-miR-411- | MIMAT0003 | n/a | n/a | 1 | 0 | 1 | 2 | 2 |
| 5p | 329 | | | | | | | |
| hsa-miR-4257 | MIMAT0016 | n/a | n/a | 1 | 0 | 1 | 2 | 2 |
| | 878 | | | | | | | |
| hsa-miR-4277 | MIMAT0016 | n/a | n/a | 0 | 1 | 1 | 2 | 2 |
| | 908 | | | | | | | |
| hsa-miR-4279 | MIMAT0016 | n/a | n/a | 1 | 0 | 1 | 2 | 2 |
| | 909 | | | | | | | |
| hsa-miR-4287 | MIMAT0016 | n/a | n/a | 1 | 0 | 1 | 2 | 2 |
| | 917 | | | | | | | |
| hsa-miR-4288 | MIMAT0016 | n/a | n/a | 1 | 0 | 1 | 2 | 2 |
| | 918 | | | | | | | |
| hsa-miR-4315 | MIMAT0016 | n/a | n/a | 0 | 1 | 1 | 2 | 2 |
| | 866 | | | | | | | |
| hsa-miR-4324 | MIMAT0016 | n/a | n/a | 1 | 0 | 1 | 2 | 2 |
| | 876 | | | | | | | |
| hsa-miR-432- | MIMAT0002 | n/a | n/a | 1 | 0 | 1 | 2 | 2 |
| 5p | 814 | | | | | | | |
| hsa-miR-4433- | MIMAT0018 | n/a | n/a | 0 | 1 | 1 | 2 | 2 |
| 3p | 949 | | | | | | | |
| hsa-miR-4438 | MIMAT0018 | n/a | n/a | 1 | 0 | 1 | 2 | 2 |
| | 956 | | | | | | | |
| hsa-miR-4478 | MIMAT0019 | n/a | n/a | 0 | 1 | 1 | 2 | 2 |
| | 006 | | | | | | | |
| hsa-miR-4489 | MIMAT0019 | n/a | n/a | 1 | 0 | 1 | 2 | 2 |
| | 023 | | | | | | | |
| hsa-miR-4497 | MIMAT0019 | n/a | n/a | 1 | 0 | 1 | 2 | 2 |
| | 032 | | | | | | | |
| hsa-miR-4505 | MIMAT0019 | n/a | n/a | 0 | 1 | 1 | 2 | 2 |
| | 041 | | | | | | | |
| hsa-miR-4508 | MIMAT0019 | n/a | n/a | 1 | 0 | 1 | 2 | 2 |
| | 045 | | | | | | | |
| hsa-miR-450b- | MIMAT0004 | n/a | n/a | 1 | 0 | 1 | 2 | 2 |
| 5p | 909 | | | | | | | |
| hsa-miR-4533 | MIMAT0019 | n/a | n/a | 1 | 0 | 1 | 2 | 2 |
| | 072 | | | | | | | |
| hsa-miR-4641 | MIMAT0019 | n/a | n/a | 1 | 0 | 1 | 2 | 2 |
| | 701 | | | | | | | |
| hsa-miR-4667- | MIMAT0019 | n/a | n/a | 1 | 0 | 1 | 2 | 2 |
| 5p | 743 | | | | | | | |
| hsa-miR-4668- | MIMAT0019 | n/a | n/a | 0 | 1 | 1 | 2 | 2 |
| 5p | 745 | | | | | | | |
| hsa-miR-4676- | MIMAT0019 | n/a | n/a | 1 | 0 | 1 | 2 | 2 |
| 5p | 758 | | | | | | | |
| hsa-miR-4685- | MIMAT0019 | n/a | n/a | 1 | 0 | 1 | 2 | 2 |
| 3p | 772 | | | | | | | |
| hsa-miR-4687- | MIMAT0019 | n/a | n/a | 1 | 0 | 1 | 2 | 2 |
| 3p | 775 | | | | | | | |
| hsa-miR-4688 | MIMAT0019 | n/a | n/a | 1 | 0 | 1 | 2 | 2 |
| | 777 | | | | | | | |
| hsa-miR-4697- | MIMAT0019 | n/a | n/a | 1 | 0 | 1 | 2 | 2 |
| 3p | 792 | | | | | | | |
| hsa-miR-4700- | MIMAT0019 | n/a | n/a | 1 | 0 | 1 | 2 | 2 |
| 5p | 796 | | | | | | | |
| hsa-miR-4701- | MIMAT0019 | n/a | n/a | 1 | 0 | 1 | 2 | 2 |
| 5p | 798 | | | | | | | |
| hsa-miR-4713- | MIMAT0019 | n/a | n/a | 1 | 0 | 1 | 2 | 2 |
| 5p | 820 | | | | | | | |
| hsa-miR-4733- | MIMAT0019 | n/a | n/a | 1 | 0 | 1 | 2 | 2 |
| 3p | 858 | | | | | | | |
| hsa-miR-4769- | MIMAT0019 | n/a | n/a | 1 | 0 | 1 | 2 | 2 |
| 3p | 923 | | | | | | | |
| hsa-miR-4782- | MIMAT0019 | n/a | n/a | 1 | 0 | 1 | 2 | 2 |
| 3p | 945 | | | | | | | |
| hsa-miR-4786- | MIMAT0019 | n/a | n/a | 0 | 1 | 1 | 2 | 2 |
| 5p | 954 | | | | | | | |
| hsa-miR-4794 | MIMAT0019 | n/a | n/a | 1 | 0 | 1 | 2 | 2 |
| | 967 | | | | | | | |
| hsa-miR-483- | MIMAT0002 | n/a | n/a | 1 | 0 | 1 | 2 | 2 |
| 3p | 173 | | | | | | | |
| hsa-miR-484 | MIMAT0002 | n/a | n/a | 1 | 0 | 1 | 2 | 2 |
| | 174 | | | | | | | |
| hsa-miR-485- | MIMAT0002 | n/a | n/a | 0 | 1 | 1 | 2 | 2 |
| 5p | 175 | | | | | | | |
| hsa-miR-4999- | MIMAT0021 | n/a | n/a | 0 | 1 | 1 | 2 | 2 |
| 3p | 018 | | | | | | | |
| hsa-miR-499b- | MIMAT0019 | n/a | n/a | 1 | 0 | 1 | 2 | 2 |
| 3p | 898 | | | | | | | |
| hsa-miR-5001- | MIMAT0021 | n/a | n/a | 1 | 0 | 1 | 2 | 2 |
| 3p | 022 | | | | | | | |
| hsa-miR-5008- | MIMAT0021 | n/a | n/a | 1 | 0 | 1 | 2 | 2 |
| 5p | 039 | | | | | | | |
| hsa-miR-500a- | MIMAT0004 | n/a | n/a | 1 | 0 | 1 | 2 | 2 |
| 5p | 773 | | | | | | | |
| hsa-miR-500b- | MIMAT0027 | n/a | n/a | 0 | 1 | 1 | 2 | 2 |
| 3p | 032 | | | | | | | |
| hsa-miR-504- | MIMAT0026 | n/a | n/a | 0 | 1 | 1 | 2 | 2 |
| 3p | 612 | | | | | | | |
| hsa-miR-5047 | MIMAT0020 | n/a | n/a | 1 | 0 | 1 | 2 | 2 |
| | 541 | | | | | | | |
| hsa-miR-505- | MIMAT0004 | n/a | n/a | 1 | 0 | 1 | 2 | 2 |
| 5p | 776 | | | | | | | |
| hsa-miR-507 | MIMAT0002 | n/a | n/a | 1 | 0 | 1 | 2 | 2 |
| | 879 | | | | | | | |
| hsa-miR-508- | MIMAT0002 | n/a | n/a | 1 | 0 | 1 | 2 | 2 |
| 3p | 880 | | | | | | | |
| hsa-miR-5088- | MIMAT0021 | n/a | n/a | 1 | 0 | 1 | 2 | 2 |
| 5p | 080 | | | | | | | |
| hsa-miR-5096 | MIMAT0020 | n/a | n/a | 0 | 1 | 1 | 2 | 2 |
| | 603 | | | | | | | |
| hsa-miR-520g- | MIMAT0002 | n/a | n/a | 0 | 1 | 1 | 2 | 2 |
| 3p | 858 | | | | | | | |
| hsa-miR-520h | MIMAT0002 | n/a | n/a | 0 | 1 | 1 | 2 | 2 |
| | 867 | | | | | | | |
| hsa-miR-544b | MIMAT0015 | n/a | n/a | 1 | 0 | 1 | 2 | 2 |
| | 004 | | | | | | | |
| hsa-miR- | MIMAT0022 | n/a | n/a | 1 | 0 | 1 | 2 | 2 |
| 548as-3p | 268 | | | | | | | |
| hsa-miR-550a- | MIMAT0020 | n/a | n/a | 1 | 0 | 1 | 2 | 2 |
| 3-5p | 925 | | | | | | | |
| hsa-miR-550a- | MIMAT0004 | n/a | n/a | 1 | 0 | 1 | 2 | 2 |
| 5p | 800 | | | | | | | |
| hsa-miR-550b- | MIMAT0022 | n/a | n/a | 0 | 1 | 1 | 2 | 2 |
| 2-5p | 737 | | | | | | | |
| hsa-miR-551a | MIMAT0003 | n/a | n/a | 1 | 0 | 1 | 2 | 2 |
| | 214 | | | | | | | |
| hsa-miR-5580- | MIMAT0022 | n/a | n/a | 1 | 0 | 1 | 2 | 2 |
| 5p | 273 | | | | | | | |
| hsa-miR-582- | MIMAT0003 | n/a | n/a | 1 | 0 | 1 | 2 | 2 |
| 5p | 247 | | | | | | | |
| hsa-miR-588 | MIMAT0003 | n/a | n/a | 1 | 0 | 1 | 2 | 2 |
| | 255 | | | | | | | |
| hsa-miR-593- | MIMAT0004 | n/a | n/a | 1 | 0 | 1 | 2 | 2 |
| 3p | 802 | | | | | | | |
| hsa-miR-593- | MIMAT0003 | n/a | n/a | 1 | 0 | 1 | 2 | 2 |
| 5p | 261 | | | | | | | |
| hsa-miR-597- | MIMAT0003 | n/a | n/a | 1 | 0 | 1 | 2 | 2 |
| 5p | 265 | | | | | | | |
| hsa-miR-6084 | MIMAT0023 | n/a | n/a | 1 | 0 | 1 | 2 | 2 |
| | 709 | | | | | | | |
| hsa-miR-6090 | MIMAT0023 | n/a | n/a | 1 | 0 | 1 | 2 | 2 |
| | 715 | | | | | | | |
| hsa-miR-632 | MIMAT0003 | n/a | n/a | 1 | 0 | 1 | 2 | 2 |
| | 302 | | | | | | | |
| hsa-miR-637 | MIMAT0003 | n/a | n/a | 1 | 0 | 1 | 2 | 2 |
| | 307 | | | | | | | |
| hsa-miR-6499- | MIMAT0025 | n/a | n/a | 1 | 0 | 1 | 2 | 2 |
| 3p | 451 | | | | | | | |
| hsa-miR-6505- | MIMAT0025 | n/a | n/a | 1 | 0 | 1 | 2 | 2 |
| 5p | 466 | | | | | | | |
| hsa-miR-6516- | MIMAT0030 | n/a | n/a | 1 | 0 | 1 | 2 | 2 |
| 5p | 417 | | | | | | | |
| hsa-miR-662 | MIMAT0003 | n/a | n/a | 1 | 0 | 1 | 2 | 2 |
| | 325 | | | | | | | |
| hsa-miR-665 | MIMAT0004 | n/a | n/a | 0 | 1 | 1 | 2 | 2 |
| | 952 | | | | | | | |
| hsa-miR-670- | MIMAT0026 | n/a | n/a | 1 | 0 | 1 | 2 | 2 |
| 3p | 640 | | | | | | | |
| hsa-miR-6721- | MIMAT0025 | n/a | n/a | 1 | 0 | 1 | 2 | 2 |
| 5p | 852 | | | | | | | |
| hsa-miR-6729- | MIMAT0027 | n/a | n/a | 1 | 0 | 1 | 2 | 2 |
| 3p | 360 | | | | | | | |
| hsa-miR-6738- | MIMAT0027 | n/a | n/a | 0 | 1 | 1 | 2 | 2 |
| 3p | 378 | | | | | | | |
| hsa-miR-6743- | MIMAT0027 | n/a | n/a | 1 | 0 | 1 | 2 | 2 |
| 5p | 387 | | | | | | | |
| hsa-miR-6755- | MIMAT0027 | n/a | n/a | 1 | 0 | 1 | 2 | 2 |
| 5p | 410 | | | | | | | |
| hsa-miR-6756- | MIMAT0027 | n/a | n/a | 1 | 0 | 1 | 2 | 2 |
| 3p | 413 | | | | | | | |
| hsa-miR-6765- | MIMAT0027 | n/a | n/a | 1 | 0 | 1 | 2 | 2 |
| 5p | 430 | | | | | | | |
| hsa-miR-6766- | MIMAT0027 | n/a | n/a | 1 | 0 | 1 | 2 | 2 |
| 3p | 433 | | | | | | | |
| hsa-miR- | MIMAT0027 | n/a | n/a | 1 | 0 | 1 | 2 | 2 |
| 6769b-5p | 620 | | | | | | | |
| hsa-miR-6781- | MIMAT0027 | n/a | n/a | 1 | 0 | 1 | 2 | 2 |
| 3p | 463 | | | | | | | |
| hsa-miR-6784- | MIMAT0027 | n/a | n/a | 1 | 0 | 1 | 2 | 2 |
| 3p | 469 | | | | | | | |
| hsa-miR-6786- | MIMAT0027 | n/a | n/a | 1 | 0 | 1 | 2 | 2 |
| 3p | 473 | | | | | | | |
| hsa-miR-6792- | MIMAT0027 | n/a | n/a | 1 | 0 | 1 | 2 | 2 |
| 3p | 485 | | | | | | | |
| hsa-miR-6802- | MIMAT0027 | n/a | n/a | 1 | 0 | 1 | 2 | 2 |
| 3p | 505 | | | | | | | |
| hsa-miR-6812- | MIMAT0027 | n/a | n/a | 1 | 0 | 1 | 2 | 2 |
| 3p | 525 | | | | | | | |
| hsa-miR-6812- | MIMAT0027 | n/a | n/a | 1 | 0 | 1 | 2 | 2 |
| 5p | 524 | | | | | | | |
| hsa-miR-6815- | MIMAT0027 | n/a | n/a | 1 | 0 | 1 | 2 | 2 |
| 5p | 530 | | | | | | | |
| hsa-miR-6816- | MIMAT0027 | 80 | GGGGTGGT | 1 | 0 | 1 | 2 | 2 |
| 5p | 532 | | GGC | | | | | |
| hsa-miR-6817- | MIMAT0027 | n/a | n/a | 1 | 0 | 1 | 2 | 2 |
| 5p | 534 | | | | | | | |
| hsa-miR-6818- | MIMAT0027 | 1221 | TTCACTGCA | 1 | 0 | 1 | 2 | 2 |
| 3p | 537 | | | | | | | |
| hsa-miR-6820- | MIMAT0027 | n/a | n/a | 1 | 0 | 1 | 2 | 2 |
| 3p | 541 | | | | | | | |
| hsa-miR-6821- | MIMAT0027 | n/a | n/a | 1 | 0 | 1 | 2 | 2 |
| 3p | 543 | | | | | | | |
| hsa-miR-6823- | MIMAT0027 | n/a | n/a | 1 | 0 | 1 | 2 | 2 |
| 5p | 546 | | | | | | | |
| hsa-miR-6825- | MIMAT0027 | n/a | n/a | 1 | 0 | 1 | 2 | 2 |
| 3p | 551 | | | | | | | |
| hsa-miR-6825- | MIMAT0027 | n/a | n/a | 1 | 0 | 1 | 2 | 2 |
| 5p | 550 | | | | | | | |
| hsa-miR-6827- | MIMAT0027 | n/a | n/a | 1 | 0 | 1 | 2 | 2 |
| 5p | 554 | | | | | | | |
| hsa-miR-6829- | MIMAT0027 | n/a | n/a | 1 | 0 | 1 | 2 | 2 |
| 3p | 559 | | | | | | | |
| hsa-miR-6832- | MIMAT0027 | n/a | n/a | 1 | 0 | 1 | 2 | 2 |
| 5p | 564 | | | | | | | |
| hsa-miR-6836- | MIMAT0027 | n/a | n/a | 1 | 0 | 1 | 2 | 2 |
| 3p | 575 | | | | | | | |
| hsa-miR-6839- | MIMAT0027 | n/a | n/a | 1 | 0 | 1 | 2 | 2 |
| 5p | 580 | | | | | | | |
| hsa-miR-6840- | MIMAT0027 | n/a | n/a | 1 | 0 | 1 | 2 | 2 |
| 3p | 583 | | | | | | | |
| hsa-miR-6841- | MIMAT0027 | n/a | n/a | 1 | 0 | 1 | 2 | 2 |
| 5p | 584 | | | | | | | |
| hsa-miR-6842- | MIMAT0027 | n/a | n/a | 1 | 0 | 1 | 2 | 2 |
| 3p | 587 | | | | | | | |
| hsa-miR-6842- | MIMAT0027 | n/a | n/a | 1 | 0 | 1 | 2 | 2 |
| 5p | 586 | | | | | | | |
| hsa-miR-6843- | MIMAT0027 | n/a | n/a | 1 | 0 | 1 | 2 | 2 |
| 3p | 588 | | | | | | | |
| hsa-miR-6844 | MIMAT0027 | 1277 | TCTAAGCGC | 1 | 0 | 1 | 2 | 2 |
| | 589 | | G | | | | | |
| hsa-miR-6845- | MIMAT0027 | n/a | n/a | 1 | 0 | 1 | 2 | 2 |
| 3p | 591 | | | | | | | |
| hsa-miR-6847- | MIMAT0027 | n/a | n/a | 1 | 0 | 1 | 2 | 2 |
| 5p | 594 | | | | | | | |
| hsa-miR-6848- | MIMAT0027 | n/a | n/a | 1 | 0 | 1 | 2 | 2 |
| 3p | 597 | | | | | | | |
| hsa-miR-6851- | MIMAT0027 | n/a | n/a | 1 | 0 | 1 | 2 | 2 |
| 3p | 603 | | | | | | | |
| hsa-miR-6851- | MIMAT0027 | 1466 | ACGCCATCG | 1 | 0 | 1 | 2 | 2 |
| 5p | 602 | | | | | | | |
| hsa-miR-6852- | MIMAT0027 | n/a | n/a | 1 | 0 | 1 | 2 | 2 |
| 5p | 604 | | | | | | | |
| hsa-miR-6854- | MIMAT0027 | n/a | n/a | 1 | 0 | 1 | 2 | 2 |
| 5p | 608 | | | | | | | |
| hsa-miR-6855- | MIMAT0027 | n/a | n/a | 1 | 0 | 1 | 2 | 2 |
| 5p | 610 | | | | | | | |
| hsa-miR-6856- | MIMAT0027 | n/a | n/a | 1 | 0 | 1 | 2 | 2 |
| 3p | 613 | | | | | | | |
| hsa-miR-6858- | MIMAT0027 | n/a | n/a | 1 | 0 | 1 | 2 | 2 |
| 5p | 616 | | | | | | | |
| hsa-miR-6859- | MIMAT0027 | n/a | n/a | 1 | 0 | 1 | 2 | 2 |
| 3p | 619 | | | | | | | |
| hsa-miR-6859- | MIMAT0027 | n/a | n/a | 1 | 0 | 1 | 2 | 2 |
| 5p | 618 | | | | | | | |
| hsa-miR-6862- | MIMAT0027 | n/a | n/a | 1 | 0 | 1 | 2 | 2 |
| 3p | 626 | | | | | | | |
| hsa-miR-6864- | MIMAT0027 | n/a | n/a | 1 | 0 | 1 | 2 | 2 |
| 3p | 629 | | | | | | | |
| hsa-miR-6865- | MIMAT0027 | n/a | n/a | 1 | 0 | 1 | 2 | 2 |
| 3p | 631 | | | | | | | |
| hsa-miR-6866- | MIMAT0027 | n/a | n/a | 1 | 0 | 1 | 2 | 2 |
| 3p | 633 | | | | | | | |
| hsa-miR-6867- | MIMAT0027 | 1656 | ACCTCAAGG | 1 | 0 | 1 | 2 | 2 |
| 3p | 635 | | | | | | | |
| hsa-miR-6869- | MIMAT0027 | n/a | n/a | 1 | 0 | 1 | 2 | 2 |
| 3p | 639 | | | | | | | |
| hsa-miR-6869- | MIMAT0027 | n/a | n/a | 1 | 0 | 1 | 2 | 2 |
| 5p | 638 | | | | | | | |
| hsa-miR-6871- | MIMAT0027 | n/a | n/a | 1 | 0 | 1 | 2 | 2 |
| 3p | 643 | | | | | | | |
| hsa-miR-6871- | MIMAT0027 | n/a | n/a | 1 | 0 | 1 | 2 | 2 |
| 5p | 642 | | | | | | | |
| hsa-miR-6873- | MIMAT0027 | n/a | n/a | 1 | 0 | 1 | 2 | 2 |
| 5p | 646 | | | | | | | |
| hsa-miR-6875- | MIMAT0027 | n/a | n/a | 1 | 0 | 1 | 2 | 2 |
| 5p | 650 | | | | | | | |
| hsa-miR-6877- | MIMAT0027 | n/a | n/a | 1 | 0 | 1 | 2 | 2 |
| 5p | 654 | | | | | | | |
| hsa-miR-6879- | MIMAT0027 | n/a | n/a | 1 | 0 | 1 | 2 | 2 |
| 3p | 659 | | | | | | | |
| hsa-miR-6882- | MIMAT0027 | 535 | CTGAAGCAG | 1 | 0 | 1 | 2 | 2 |
| 3p | 665 | | | | | | | |
| hsa-miR-6883- | MIMAT0027 | n/a | n/a | 1 | 0 | 1 | 2 | 2 |
| 5p | 666 | | | | | | | |
| hsa-miR-6886- | MIMAT0027 | n/a | n/a | 1 | 0 | 1 | 2 | 2 |
| 5p | 672 | | | | | | | |
| hsa-miR-6887- | MIMAT0027 | 1543 | CCACCTCCG | 1 | 0 | 1 | 2 | 2 |
| 3p | 675 | | CT | | | | | |
| hsa-miR-6888- | MIMAT0027 | n/a | n/a | 1 | 0 | 1 | 2 | 2 |
| 5p | 676 | | | | | | | |
| hsa-miR-6889- | MIMAT0027 | n/a | n/a | 1 | 0 | 1 | 2 | 2 |
| 3p | 679 | | | | | | | |
| hsa-miR-6890- | MIMAT0027 | n/a | n/a | 1 | 0 | 1 | 2 | 2 |
| 5p | 680 | | | | | | | |
| hsa-miR-6892- | MIMAT0027 | 1658 | CTCAAGGCC | 1 | 0 | 1 | 2 | 2 |
| 3p | 685 | | | | | | | |
| hsa-miR-6895- | MIMAT0027 | n/a | n/a | 1 | 0 | 1 | 2 | 2 |
| 3p | 691 | | | | | | | |
| hsa-miR-7106- | MIMAT0028 | n/a | n/a | 1 | 0 | 1 | 2 | 2 |
| 3p | 110 | | | | | | | |
| hsa-miR-7106- | MIMAT0028 | n/a | n/a | 1 | 0 | 1 | 2 | 2 |
| 5p | 109 | | | | | | | |
| hsa-miR-7107- | MIMAT0028 | n/a | n/a | 1 | 0 | 1 | 2 | 2 |
| 5p | 111 | | | | | | | |
| hsa-miR-7108- | MIMAT0028 | n/a | n/a | 1 | 0 | 1 | 2 | 2 |
| 3p | 114 | | | | | | | |
| hsa-miR-7110- | MIMAT0028 | n/a | n/a | 1 | 0 | 1 | 2 | 2 |
| 5p | 117 | | | | | | | |
| hsa-miR-7113- | MIMAT0028 | n/a | n/a | 1 | 0 | 1 | 2 | 2 |
| 3p | 124 | | | | | | | |
| hsa-miR-7113- | MIMAT0028 | n/a | n/a | 1 | 0 | 1 | 2 | 2 |
| 5p | 123 | | | | | | | |
| hsa-miR-7151- | MIMAT0028 | n/a | n/a | 1 | 0 | 1 | 2 | 2 |
| 3p | 213 | | | | | | | |
| hsa-miR-7154- | MIMAT0028 | n/a | n/a | 1 | 0 | 1 | 2 | 2 |
| 3p | 219 | | | | | | | |
| hsa-miR-7155- | MIMAT0028 | n/a | n/a | 1 | 0 | 1 | 2 | 2 |
| 5p | 220 | | | | | | | |
| hsa-miR-7156- | MIMAT0028 | n/a | n/a | 1 | 0 | 1 | 2 | 2 |
| 3p | 223 | | | | | | | |
| hsa-miR-7157- | MIMAT0028 | n/a | n/a | 1 | 0 | 1 | 2 | 2 |
| 3p | 225 | | | | | | | |
| hsa-miR-7160- | MIMAT0028 | n/a | n/a | 1 | 0 | 1 | 2 | 2 |
| 5p | 230 | | | | | | | |
| hsa-miR-744- | MIMAT0004 | n/a | n/a | 1 | 0 | 1 | 2 | 2 |
| 5p | 945 | | | | | | | |
| hsa-miR-761 | MIMAT0010 | n/a | n/a | 1 | 0 | 1 | 2 | 2 |
| | 364 | | | | | | | |
| hsa-miR-762 | MIMAT0010 | n/a | n/a | 1 | 0 | 1 | 2 | 2 |
| | 313 | | | | | | | |
| hsa-miR-764 | MIMAT0010 | n/a | n/a | 1 | 0 | 1 | 2 | 2 |
| | 367 | | | | | | | |
| hsa-miR-765 | MIMAT0003 | n/a | n/a | 1 | 0 | 1 | 2 | 2 |
| | 945 | | | | | | | |
| hsa-miR-7704 | MIMAT0030 | n/a | n/a | 1 | 0 | 1 | 2 | 2 |
| | 019 | | | | | | | |
| hsa-miR-7706 | MIMAT0030 | n/a | n/a | 1 | 0 | 1 | 2 | 2 |
| | 021 | | | | | | | |
| hsa-miR-7845- | MIMAT0030 | n/a | n/a | 1 | 0 | 1 | 2 | 2 |
| 5p | 420 | | | | | | | |
| hsa-miR-7846- | MIMAT0030 | n/a | n/a | 1 | 0 | 1 | 2 | 2 |
| 3p | 421 | | | | | | | |
| hsa-miR-7854- | MIMAT0030 | n/a | n/a | 1 | 0 | 1 | 2 | 2 |
| 3p | 429 | | | | | | | |
| hsa-miR-7855- | MIMAT0030 | n/a | n/a | 1 | 0 | 1 | 2 | 2 |
| 5p | 430 | | | | | | | |
| hsa-miR-7974 | MIMAT0031 | n/a | n/a | 1 | 0 | 1 | 2 | 2 |
| | 177 | | | | | | | |
| hsa-miR-7976 | MIMAT0031 | n/a | n/a | 1 | 0 | 1 | 2 | 2 |
| | 179 | | | | | | | |
| hsa-miR-7978 | MIMAT0031 | n/a | n/a | 1 | 0 | 1 | 2 | 2 |
| | 181 | | | | | | | |
| hsa-miR-8082 | MIMAT0031 | n/a | n/a | 1 | 0 | 1 | 2 | 2 |
| | 009 | | | | | | | |
| hsa-miR-8089 | MIMAT0031 | n/a | n/a | 1 | 0 | 1 | 2 | 2 |
| | 016 | | | | | | | |
| hsa-miR-873- | MIMAT0022 | n/a | n/a | 1 | 0 | 1 | 2 | 2 |
| 3p | 717 | | | | | | | |
| hsa-miR-873- | MIMAT0004 | n/a | n/a | 1 | 0 | 1 | 2 | 2 |
| 5p | 953 | | | | | | | |
| hsa-miR-874- | MIMAT0004 | 1524 | CCCCGAGG | 1 | 0 | 1 | 2 | 2 |
| 3p | 911 | | GT | | | | | |
| hsa-miR-876- | MIMAT0004 | n/a | n/a | 1 | 0 | 1 | 2 | 2 |
| 5p | 924 | | | | | | | |
| hsa-miR-885- | MIMAT0004 | n/a | n/a | 1 | 0 | 1 | 2 | 2 |
| 3p | 948 | | | | | | | |
| hsa-miR-888- | MIMAT0004 | n/a | n/a | 1 | 0 | 1 | 2 | 2 |
| 3p | 917 | | | | | | | |
| hsa-miR-92a- | MIMAT0004 | n/a | n/a | 1 | 0 | 1 | 2 | 2 |
| 2-5p | 508 | | | | | | | |
| hsa-miR-92b- | MIMAT0004 | n/a | n/a | 1 | 0 | 1 | 2 | 2 |
| 5p | 792 | | | | | | | |
| hsa-miR-937- | MIMAT0004 | n/a | n/a | 1 | 0 | 1 | 2 | 2 |
| 3p | 980 | | | | | | | |
| hsa-miR-937- | MIMAT0022 | n/a | n/a | 1 | 0 | 1 | 2 | 2 |
| 5p | 938 | | | | | | | |
| hsa-miR-939- | MIMAT0004 | n/a | n/a | 1 | 0 | 1 | 2 | 2 |
| 5p | 982 | | | | | | | |
| hsa-miR-9-5p | MIMAT0000 | n/a | n/a | 1 | 0 | 1 | 2 | 2 |
| | 441 | | | | | | | |

TABLE 8b
Myo9b promoter miRNAs (all sequences correspond to Gene: MYBO9B; EntrezID:
4650; RefseqID NM_00130065).

| | | SPM | | Seed | Seed | pvalu | Range | |
|---|---|---|---|---|---|---|---|---|
| miRNA | MIMATid | S | SL | S | E | e | (kb) | Target Seq |
| hsa-miR-1234-3p | MIMAT000558 | 2 | 1 | 624 | 633 | 0.009 | 2kb | GTTCGCCAGG |
| | 9 | | 0 | | | 5 | | |
| hsa-miR-1237-5p | MIMAT002294 | 2 | 9 | 299 | 307 | 0.037 | 2kb | ATAGGAATC |
| | 6 | | | | | 4 | | |
| hsa-miR-1254 | MIMAT000590 | 2 | 9 | 1405 | 1413 | 0.037 | 2kb | CGAGGACGC |
| | 5 | | | | | 4 | | |
| hsa-miR-125b-1- | MIMAT000459 | 2 | 9 | 1327 | 1335 | 0.037 | 2kb | TCGGCCCCA |
| 3p | 2 | | | | | 4 | | |
| hsa-miR-128-2-5p | MIMAT003109 | 2 | 9 | 666 | 674 | 0.037 | 2kb | CTAACCCGA |
| | 5 | | | | | 4 | | |
| hsa-miR-1288-5p | MIMAT002674 | 2 | 9 | 120 | 128 | 0.037 | 2kb | ACTGACACA |
| | 3 | | | | | 4 | | |
| hsa-miR-1304-3p | MIMAT002272 | 1 | 9 | 1790 | 1798 | 0.037 | 2kb | CCAGACGCG |
| | 0 | | | | | 4 | | |
| hsa-miR-130a-3p | MIMAT000042 | 2 | 9 | 1811 | 1819 | 0.037 | 2kb | CCTCCCCTC |
| | 5 | | | | | 4 | | |
| hsa-miR-130b-3p | MIMAT000069 | 2 | 9 | 1811 | 1819 | 0.037 | 2kb | CCTCCCCTC |
| | 1 | | | | | 4 | | |
| hsa-miR-153-5p | MIMAT002648 | 1 | 1 | 1924 | 1933 | 0.009 | 2kb | GAGCGCTGGC |
| | 0 | | 0 | | | 5 | | |
| hsa-miR-153-5p | MIMAT002648 | 2 | 9 | 1925 | 1933 | 0.037 | 2kb | AGCGCTGGC |
| | 0 | | | | | 4 | | |
| hsa-miR-1908-5p | MIMAT000788 | 2 | 1 | 47 | 57 | 0.002 | 2kb | ACCCCATCTCT |
| | 1 | | 1 | | | 4 | | |
| hsa-miR-192-5p | MIMAT000022 | 2 | 9 | 1255 | 1263 | 0.037 | 2kb | ACAGTAAAG |
| | 2 | | | | | 4 | | |
| hsa-miR-193a-5p | MIMAT000461 | 1 | 9 | 732 | 740 | 0.037 | 2kb | TTTTACATA |
| | 4 | | | | | 4 | | |
| hsa-miR-19b-1-5p | MIMAT000449 | 2 | 9 | 1282 | 1290 | 0.037 | 2kb | GCGCGTTTC |
| | 1 | | | | | 4 | | |
| hsa-miR-19b-2-5p | MIMAT000449 | 2 | 9 | 1282 | 1290 | 0.037 | 2kb | GCGCGTTTC |
| | 2 | | | | | 4 | | |
| hsa-miR-215-5p | MIMAT000027 | 2 | 9 | 1255 | 1263 | 0.037 | 2kb | ACAGTAAAG |
| | 2 | | | | | 4 | | |
| hsa-miR-301b | MIMAT000495 | 2 | 9 | 1811 | 1819 | 0.037 | 2kb | CCTCCCCTC |
| | 8 | | | | | 4 | | |
| hsa-miR-3064-5p | MIMAT001986 | 1 | 9 | 1230 | 1238 | 0.037 | 2kb | TAACGATAA |
| | 4 | | | | | 4 | | |
| hsa-miR-3150b-3p | MIMAT001819 | 1 | 9 | 848 | 856 | 0.037 | 2kb | CTGCTGCGA |
| | 4 | | | | | 4 | | |
| hsa-miR-33b-3p | MIMAT000481 | 1 | 1 | 637 | 646 | 0.009 | 2kb | CAGCAGCTGC |
| | 1 | | 0 | | | 5 | | |
| hsa-miR-33b-3p | MIMAT000481 | 2 | 9 | 638 | 646 | 0.037 | 2kb | AGCAGCTGC |
| | 1 | | | | | 4 | | |
| hsa-miR-3621 | MIMAT001800 | 2 | 1 | 326 | 335 | 0.009 | 2kb | GGGGTGCAGG |
| | 2 | | 0 | | | 5 | | |
| hsa-miR-378j | MIMAT002461 | 2 | 9 | 409 | 417 | 0.037 | 2kb | ACAGACACC |
| | 2 | | | | | 4 | | |
| hsa-miR-3922-3p | MIMAT001819 | 2 | 9 | 1528 | 1536 | 0.037 | 2kb | GAGGGTCCT |
| | 7 | | | | | 4 | | |
| hsa-miR-4295 | MIMAT001684 | 2 | 9 | 1811 | 1819 | 0.037 | 2kb | CCTCCCCTC |
| | 4 | | | | | 4 | | |
| hsa-miR-4419b | MIMAT001903 | 1 | 9 | 704 | 712 | 0.037 | 2kb | TCGTCGCTG |
| | 4 | | | | | 4 | | |
| hsa-miR-4430 | MIMAT001894 | 2 | 1 | 1805 | 1814 | 0.009 | 2kb | GCCTGTCCTC |
| | 5 | | 0 | | | 5 | | |
| hsa-miR-4433-5p | MIMAT002095 | 2 | 9 | 128 | 136 | 0.037 | 2kb | AGGAGAACT |
| | 6 | | | | | 4 | | |
| hsa-miR-4486 | MIMAT001902 | 1 | 9 | 604 | 612 | 0.037 | 2kb | GGAGAGGCA |
| | 0 | | | | | 4 | | |
| hsa-miR-4488 | MIMAT001902 | 1 | 1 | 298 | 307 | 0.009 | 2kb | GATAGGAATC |
| | 2 | | 0 | | | 5 | | |
| hsa-miR-4488 | MIMAT001902 | 2 | 9 | 299 | 307 | 0.037 | 2kb | ATAGGAATC |
| | 2 | | | | | 4 | | |
| hsa-miR-4523 | MIMAT001906 | 1 | 9 | 928 | 936 | 0.037 | 2kb | TCTATCGGT |
| | 1 | | | | | 4 | | |
| hsa-miR-4639-5p | MIMAT001969 | 1 | 9 | 1379 | 1387 | 0.037 | 2kb | TGCCGGAGG |
| | 7 | | | | | 4 | | |
| hsa-miR-4677-3p | MIMAT001976 | 2 | 9 | 1235 | 1243 | 0.037 | 2kb | ATAATTCCA |
| | 1 | | | | | 4 | | |
| hsa-miR-4682 | MIMAT001976 | 2 | 9 | 578 | 586 | 0.037 | 2kb | TTGGGGACA |
| | 7 | | | | | 4 | | |
| hsa-miR-4717-5p | MIMAT001982 | 2 | 1 | 1179 | 1189 | 0.002 | 2kb | CGTGGGAGGAT |
| | 9 | | 1 | | | 4 | | |
| hsa-miR-4728-5p | MIMAT001984 | 2 | 9 | 869 | 877 | 0.037 | 2kb | CCCTACAAC |
| | 9 | | | | | 4 | | |
| hsa-miR-4749-3p | MIMAT001988 | 2 | 1 | 1566 | 1575 | 0.009 | 2kb | CAGCCCTATG |
| | 6 | | 0 | | | 5 | | |
| hsa-miR-4753-3p | MIMAT001989 | 2 | 9 | 1224 | 1232 | 0.037 | 2kb | ACTGCATAA |
| | 1 | | | | | 4 | | |
| hsa-miR-4779 | MIMAT001993 | 1 | 9 | 1889 | 1897 | 0.037 | 2kb | TAGGGGCGG |
| | 8 | | | | | 4 | | |
| hsa-miR-4784 | MIMAT001994 | 1 | 1 | 848 | 857 | 0.009 | 2kb | CTGCTGCGAC |
| | 8 | | 0 | | | 5 | | |
| hsa-miR-4784 | MIMAT001994 | 2 | 9 | 849 | 857 | 0.037 | 2kb | TGCTGCGAC |
| | 8 | | | | | 4 | | |
| hsa-miR-5001-5p | MIMAT002102 | 1 | 9 | 428 | 436 | 0.037 | 2kb | GAGGGGCAA |
| | 1 | | | | | 4 | | |
| hsa-miR-5004-5p | MIMAT002102 | 2 | 9 | 186 | 194 | 0.037 | 2kb | CACTCCAGC |
| | 7 | | | | | 4 | | |
| hsa-miR-5008-3p | MIMAT002104 | 2 | 1 | 831 | 842 | 0.000 | 2kb | TGGGCTCCTGG |
| | 0 | | 2 | | | 6 | | G |
| hsa-miR-5088-3p | MIMAT002704 | 1 | 9 | 1599 | 1607 | 0.037 | 2kb | GAAGCCCCA |
| | 1 | | | | | 4 | | |
| hsa-miR-523-3p | MIMAT000284 | 2 | 9 | 712 | 720 | 0.037 | 2kb | GCAAAACAA |
| | 0 | | | | | 4 | | |
| hsa-miR-5682 | MIMAT002247 | 1 | 9 | 1079 | 1087 | 0.037 | 2kb | CAACTTCTT |
| | 0 | | | | | 4 | | |
| hsa-miR-5702 | MIMAT002249 | 1 | 9 | 1684 | 1692 | 0.037 | 2kb | AAAGCCGGA |
| | 5 | | | | | 4 | | |
| hsa-miR-573 | MIMAT000323 | 1 | 9 | 1013 | 1021 | 0.037 | 2kb | GCAGAATTA |
| | 8 | | | | | 4 | | |
| hsa-miR-6087 | MIMAT002371 | 2 | 9 | 45 | 53 | 0.037 | 2kb | AAACCCCAT |
| | 2 | | | | | 4 | | |
| hsa-miR-642a-5p | MIMAT000331 | 2 | 9 | 1658 | 1666 | 0.037 | 2kb | CTCAAGGCC |
| | 2 | | | | | 4 | | |
| hsa-miR-649 | MIMAT000331 | 1 | 9 | 1266 | 1274 | 0.037 | 2kb | CCAGGCGCC |
| | 9 | | | | | 4 | | |
| hsa-miR-6504-5p | MIMAT002546 | 1 | 1 | 1230 | 1239 | 0.009 | 2kb | TAACGATAAT |
| | 4 | | 0 | | | 5 | | |
| hsa-miR-6504-5p | MIMAT002546 | 2 | 9 | 1231 | 1239 | 0.037 | 2kb | AACGATAAT |
| | 4 | | | | | 4 | | |
| hsa-miR-660-3p | MIMAT002271 | 2 | 1 | 1569 | 1578 | 0.009 | 2kb | CCCTATGGCG |
| | 1 | | 0 | | | 5 | | |
| hsa-miR-661 | MIMAT000332 | 2 | 1 | 729 | 738 | 0.009 | 2kb | AGGTTTTACA |
| | 4 | | 0 | | | 5 | | |
| hsa-miR-6730-3p | MIMAT002736 | 2 | 9 | 561 | 569 | 0.037 | 2kb | GTGAGACCC |
| | 2 | | | | | 4 | | |
| hsa-miR-6736-5p | MIMAT002737 | 1 | 9 | 383 | 391 | 0.037 | 2kb | CTGGTGAGA |
| | 3 | | | | | 4 | | |
| hsa-miR-6752-3p | MIMAT002740 | 2 | 9 | 1538 | 1546 | 0.037 | 2kb | ACTCCCCAC |
| | 5 | | | | | 4 | | |
| hsa-miR-6754-5p | MIMAT002740 | 1 | 9 | 866 | 874 | 0.037 | 2kb | GGTCCCTAC |
| | 8 | | | | | 4 | | |
| hsa-miR-6805-5p | MIMAT002751 | 2 | 9 | 298 | 306 | 0.037 | 2kb | GATAGGAAT |
| | 0 | | | | | 4 | | |
| hsa-miR-6816-5p | MIMAT002753 | 2 | 1 | 80 | 90 | 0.002 | 2kb | GGGGTGGTGGC |
| | 2 | | 1 | | | 4 | | |
| hsa-miR-6818-3p | MIMAT002753 | 1 | 9 | 1221 | 1229 | 0.037 | 2kb | TTCACTGCA |
| | 7 | | | | | 4 | | |
| hsa-miR-6844 | MIMAT002758 | 2 | 1 | 1277 | 1286 | 0.009 | 2kb | TCTAAGCGCG |
| | 9 | | 0 | | | 5 | | |
| hsa-miR-6851-5p | MIMAT002760 | 1 | 9 | 1466 | 1474 | 0.037 | 2kb | ACGCCATCG |
| | 2 | | | | | 4 | | |
| hsa-miR-6867-3p | MIMAT002763 | 2 | 9 | 1656 | 1664 | 0.037 | 2kb | ACCTCAAGG |
| | 5 | | | | | 4 | | |
| hsa-miR-6882-3p | MIMAT002766 | 1 | 1 | 534 | 543 | 0.009 | 2kb | TCTGAAGCAG |
| | 5 | | 0 | | | 5 | | |
| hsa-miR-6882-3p | MIMAT002766 | 2 | 9 | 535 | 543 | 0.037 | 2kb | CTGAAGCAG |
| | 5 | | | | | 4 | | |
| hsa-miR-6887-3p | MIMAT002767 | 2 | 1 | 1543 | 1553 | 0.002 | 2kb | CCACCTCCGCT |
| | 5 | | 1 | | | 4 | | |
| hsa-miR-6892-3p | MIMAT002768 | 1 | 9 | 1658 | 1666 | 0.037 | 2kb | CTCAAGGCC |
| | 5 | | | | | 4 | | |
| hsa-miR-874-3p | MIMAT000491 | 2 | 1 | 1524 | 1533 | 0.009 | 2kb | CCCCGAGGGT |
| | 1 | | 0 | | | 5 | | |

### REFERENCES

The following references, some of which are cited above by ‘Author, year’, are herein incorporated by reference in their entireties.
Cooper W A, Lam D C, O'Toole S A, and Minna J D. Molecular biology of lung cancer. J Thorac Dis. 2013; 5 (Suppl 5):S479-90.
Peifer M, Fernández-Cuesta L, Sos M L, et al. Integrative genome analyses identify key somatic driver mutations of small-cell lung cancer. Nat Genet. 2012; 44(10):1104-10
Valastyan S, and Weinberg R A. Tumor metastasis: molecular insights and evolving paradigms. Cell. 2011; 147(2):275-92.
Park K S, Martelotto L G, Peifer M, Sos M L, Karnezis A N, Mahjoub M R, Bernard K, Conklin J F, Szczepny A, Yuan J, Guo R, Ospina B, Falzon J, Bennett S, Brown T J, Markovic A, Devereux W L, Ocasio C A, Chen J K, Stearns T, Thomas R K, Dorsch M, Buonamici S, Watkins D N, Peacock C D, Sage J. A crucial requirement for Hedgehog signaling in small cell lung cancer. Nat Med. 2011; 17(11):1504-8
Brose K, Bland K S, Wang K H, Arnott D, Henzel W, Goodman C S, Tessier-Lavigne M, and Kidd T. Slit proteins bind Robo receptors and have an evolutionarily conserved role in repulsive axon guidance. Cell. 1999; 96(6):795-806.
Li H S, Chen J H, Wu W, Fagaly T, Zhou L, Yuan W, Dupuis S, Jiang Z H, Nash W, Gick C, et al. Vertebrate slit, a secreted ligand for the transmembrane protein roundabout, is a repellent for olfactory bulb axons. Cell. 1999; 96(6):807-18.
Wu W, Wong K, Chen J, Jiang Z, Dupuis S, Wu J Y, and Rao Y. Directional guidance of neuronal migration in the olfactory system by the protein Slit. Nature. 1999; 400(6742):331-6.
Ypsilanti A R, Zagar Y, and Chedotal A. Moving away from the midline: new developments for Slit and Robo. Development. 2010; 137(12):1939-52.
Brantley-Sieders D M, Dunaway C M, Rao M, Short S, Hwang Y, Gao Y, Li D, Jiang A, Shyr Y, Wu J Y, et al. Angiocrine factors modulate tumor proliferation and motility through EphA2 repression of Slit2 tumor suppressor function in endothelium. Cancer Res. 2011; 71(3):976-87.
Geutskens S B, Hordijk P L, and van Hennik P B. The chemorepellent Slit3 promotes monocyte migration. J Immunol. 2010; 185(12):7691-8.
Legg J A, Herbert J M, Clissold P, and Bicknell R. Slits and Roundabouts in cancer, tumour angiogenesis and endothelial cell migration. Angiogenesis. 2008; 11(1):13-21.
Prasad A, Qamri Z, Wu J, and Ganju R K. Slit-2/Robo-1 modulates the CXCL12/CXCR4-induced chemotaxis of T cells. J Leukoc Biol. 2007; 82(3):465-76.
Wu J Y, Feng L, Park H T, Havlioglu N, Wen L, Tang H, Bacon K B, Jiang Z, Zhang X, and Rao Y. The neuronal repellent Slit inhibits leukocyte chemotaxis induced by chemotactic factors. Nature. 2001; 410(6831):948-52.
Ballard M S, and Hinck L. A roundabout way to cancer. Adv Cancer Res. 2012; 114:187-235.
Mehlen P, Delloye-Bourgeois C, and Chedotal A. Novel roles for Slits and netrins: axon guidance cues as anticancer targets? Nat Rev Cancer. 2011; 11(3):188-97.
Nasarre P, Potiron V, Drabkin H, and Roche J. Guidance molecules in lung cancer. Cell Adh Migr. 2010; 4(1):130-45.
Dammann R, Strunnikova M, Schagdarsurengin U, Rastetter M, Papritz M, Hattenhorst U E, Hofmann H S, Silber R E, Burdach S, and Hansen G. CpG island methylation and expression of tumour-associated genes in lung carcinoma. Eur J Cancer. 2005; 41(8):1223-36.
Dallol A, Da Silva N F, Viacava P, Minna J D, Bieche I, Maher E R, and Latif F. SLIT2, a human homologue of the Drosophila Slit2 gene, has tumor suppressor activity and is frequently inactivated in lung and breast cancers. Cancer Res. 2002; 62(20):5874-80.
Kim H K, Zhang H, Li H, Wu T T, Swisher S, He D, Wu L, Xu J, Elmets C A, Athar M, et al. Slit2 inhibits growth and metastasis of fibrosarcoma and squamous cell carcinoma. Neoplasia. 2008; 10(12):1411-20.
Tseng R C, Lee S H, Hsu H S, Chen B H, Tsai W C, Tzao C, and Wang Y C. SLIT2 attenuation during lung cancer progression deregulates beta-catenin and E-cadherin and associates with poor prognosis. Cancer Res. 2010; 70(2):543-51.
Yu J, Cao Q, Yu J, Wu L, Dallol A, Li J, Chen G, Grasso C, Cao X, Lonigro R J, et al. The neuronal repellent SLIT2 is a target for repression by EZH2 in prostate cancer. Oncogene. 2010; 29(39):5370-80.
Liao W, Elfrink K, and Bahler M. Head of myosin IX binds calmodulin and moves processively toward the plus-end of actin filaments. J Biol Chem. 2010; 285(32):24933-42.
van den Boom F, Dussmann H, Uhlenbrock K, Abouhamed M, and Bahler M. The Myosin IXb motor activity targets the myosin IXb RhoGAP domain as cargo to sites of actin polymerization. Mol Biol Cell. 2007; 18(4):1507-18.
Abouhamed M, Grobe K, San I V, Thelen S, Honnert U, Balda M S, Matter K, and Bahler M. Myosin IXa regulates epithelial differentiation and its deficiency results in hydrocephalus. Mol Biol Cell. 2009; 20(24):5074-85.
Hanley P J, Xu Y, Kronlage M, Grobe K, Schon P, Song J, Sorokin L, Schwab A, and Bahler M. Motorized RhoGAP myosin IXb (Myo9b) controls cell shape and motility. Proc Natl Acad Sci USA. 2010; 107(27):12145-50.
Xu Y, Pektor S, Balkow S, Hemkemeyer S A, Liu Z, Grobe K, Hanley P J, Shen L, Bros M, Schmidt T, et al. Dendritic cell motility and T cell activation requires regulation of Rho-cofilin signaling by the Rho-GTPase activating protein myosin IXb. J Immunol. 2014; 192(8):3559-68.
Post P L, Bokoch G M, and Mooseker M S. Human myosin-IXb is a mechanochemically active motor and a GAP for rho. J Cell Sci. 1998; 111(Pt 7):941-50.
Saeki N, Tokuo H, and Ikebe M. BIG1 is a binding partner of myosin IXb and regulates its Rho-GTPase activating protein activity. J Biol Chem. 2005; 280(11):10128-34.
Wirth J A, Jensen K A, Post P L, Bement W M, and Mooseker M S. Human myosin-IXb, an unconventional myosin with a chimerin-like rho/rac GTPase-activating protein domain in its tail. J Cell Sci. 1996; 109(Pt 3):653-61.
Heasman S J, and Ridley A J. Mammalian Rho GTPases: new insights into their functions from in vivo studies. Nat Rev Mol Cell Biol. 2008; 9(9):690-701.
Parsons J T, Horwitz A R, and Schwartz M A. Cell adhesion: integrating cytoskeletal dynamics and cellular tension. Nat Rev Mol Cell Biol. 2010; 11(9):633-43.
Yuasa-Kawada J, Kinoshita-Kawada M, Rao Y, and Wu J Y. Deubiquitinating enzyme USP33/VDU1 is required for Slit signaling in inhibiting breast cancer cell migration. Proc Natl Acad Sci USA. 2009; 106(34):14530-5.
Wong K, Ren X R, Huang Y Z, Xie Y, Liu G, Saito H, Tang H, Wen L, Brady-Kalnay S M, Mei L, et al. Signal transduction in neuronal migration: roles of GTPase activating proteins and the small GTPase Cdc42 in the Slit-Robo pathway. Cell. 2001; 107(2):209-21.
Jaffe A B, and Hall A. Rho GTPases: biochemistry and biology. Annu Rev Cell Dev Biol. 2005; 21:247-69.
Yuasa-Kawada J, Kinoshita-Kawada M, Wu G, Rao Y, and Wu J Y. Midline crossing and Slit responsiveness of commissural axons require USP33. Nat Neurosci. 2009; 12(9):1087-9.
Rhodes D R, Yu J, Shanker K, Deshpande N, Varambally R, Ghosh D, Barrette T, Pandey A, and Chinnaiyan A M. ONCOMINE: a cancer microarray database and integrated data-mining platform. Neoplasia. 2004; 6(1):1-6.
Gyorffy B, Surowiak P, Budczies J, and Lanczky A. Online survival analysis software to assess the prognostic value of biomarkers using transcriptomic data in non-small-cell lung cancer. PLoS One. 2013; 8(12):e82241.
Gao J, Aksoy B A, Dogrusoz U, Dresdner G, Gross B, Sumer S O, Sun Y, Jacobsen A, Sinha R, Larsson E, et al. Integrative analysis of complex cancer genomics and clinical profiles using the cBioPortal. Sci Signal. 2013; 6(269):pl 1.
Spiro S G, and Silvestri G A. One hundred years of lung cancer. Am J Respir Crit Care Med. 2005; 172(5):523-9.
Sanchez-Cespedes M. The role of LKB1 in lung cancer. Familial cancer. 2011; 10(3):447-53.
Vaahtomeri K, and Makela T P. Molecular mechanisms of tumor suppression by LKB1. FEBS Lett. 2011; 585(7):944-51.
Yiin J J, Hu B, Jarzynka M J, Feng H, Liu K W, Wu J Y, Ma H I, and Cheng S Y. Slit2 inhibits cancer cell invasion in the brain by suppression of Cdc42 activity. Neuro Oncol. 2009; 11(6):779-89.
Werbowetski-Ogilvie T E, Seyed Sadr M, Jabado N, Angers-Loustau A, Agar N Y, Wu J, Bjerkvig R, Antel J P, Faury D, Rao Y, et al. Inhibition of medulloblastoma cell invasion by Slit. Oncogene. 2006; 25(37):5103-12.
Greenberg J M, Thompson F Y, Brooks S K, Shannon J M, Akeson A L. Slit and robo expression in the developing mouse lung. Dev Dyn. 2004; 230(2):350-60
Muller R T, Honnert U, Reinhard J, and Bahler M. The rat myosin myr 5 is a GTPase-activating protein for Rho in vivo: essential role of arginine 1695. Mol Biol Cell. 1997; 8(10):2039-53.
Jelen F, Lachowicz P, Apostoluk W, Mateja A, Derewenda Z S, and Otlewski J. Dissecting the thermodynamics of GAP-RhoA interactions. Journal of structural biology. 2009; 165(1):10-8.
Ridley A J. Life at the leading edge. Cell. 2011; 145(7):1012-22.
Besson A, Gurian-West M, Schmidt A, Hall A, and Roberts J M. p27Kip1 modulates cell migration through the regulation of RhoA activation. Genes Dev. 2004; 18(8):862-76.
Kurokawa K, and Matsuda M. Localized RhoA activation as a requirement for the induction of membrane ruffling. Mol Biol Cell. 2005; 16(9):4294-303.
Wu X, Liu T, Fang O, Leach L J, Hu X, and Luo Z. miR-194 suppresses metastasis of non-small cell lung cancer through regulating expression of BMP1 and p27(kip1). Oncogene. 2014; 33(12):1506-14.
Liu Y, Li Q C, Miao Y, Xu H T, Dai S D, Wei Q, Dong Q Z, Dong X J, Zhao Y, Zhao C, et al. Ablation of p120-catenin enhances invasion and metastasis of human lung cancer cells. Cancer Sci. 2009; 100(3):441-8.
Cools J. RHOA mutations in peripheral T cell lymphoma. Nat Genet. 2014; 46(4):320-1.
Kakiuchi M, Nishizawa T, Ueda H, Gotoh K, Tanaka A, Hayashi A, Yamamoto S, Tatsuno K, Katoh H, Watanabe Y, et al. Recurrent gain-of-function mutations of RHOA in diffuse-type gastric carcinoma. Nat Genet. 2014; 46(6):583-7.
Palomero T, Couronne L, Khiabanian H, Kim M Y, Ambesi-Impiombato A, Perez-Garcia A, Carpenter Z, Abate F, Allegretta M, Haydu J E, et al. Recurrent mutations in epigenetic regulators, RHOA and FYN kinase in peripheral T cell lymphomas. Nat Genet. 2014; 46(2):166-70.
Sakata-Yanagimoto M, Enami T, Yoshida K, Shiraishi Y, Ishii R, Miyake Y, Muto H, Tsuyama N, Sato-Otsubo A, Okuno Y, et al. Somatic RHOA mutation in angioimmunoblastic T cell lymphoma. Nat Genet. 2014; 46(2):171-5.
Wang K, Yuen S T, Xu J, Lee S P, Yan H H, Shi S T, Siu H C, Deng S, Chu K M, Law S, et al. Whole-genome sequencing and comprehensive molecular profiling identify new driver mutations in gastric cancer. Nat Genet. 2014; 46(6):573-82.
Yoo H Y, Sung M K, Lee S H, Kim S, Lee H, Park S, Kim S C, Lee B, Rho K, Lee J E, et al. A recurrent inactivating mutation in RHOA GTPase in angioimmunoblastic T cell lymphoma. Nat Genet. 2014; 46(4):371-5.
Zhou J, Hayakawa Y, Wang T C, and Bass A J. RhoA mutations identified in diffuse gastric cancer. Cancer Cell. 2014; 26(1):9-11.
Battye T G, Kontogiannis L, Johnson O, Powell H R, and Leslie A G. iMOSFLM: a new graphical interface for diffraction-image processing with MOSFLM. Acta crystallographica Section D, Biological crystallography. 2011; 67(Pt 4):271-81.
Dodson E J, Winn M, and Ralph A. Collaborative Computational Project, number 4: providing programs for protein crystallography. Methods Enzymol. 1997; 277:620-33.
McCoy A J. Solving structures of protein complexes by molecular replacement with Phaser. Acta crystallographica Section D, Biological crystallography. 2007; 63(Pt 1):32-41.
Emsley P, and Cowtan K. Coot: model-building tools for molecular graphics. Acta crystallographica Section D, Biological crystallography. 2004; 60(Pt 12 Pt 1):2126-32.
Adams P D, Afonine P V, Bunkoczi G, Chen V B, Davis I W, Echols N, Headd J J, Hung L W, Kapral G J, Grosse-Kunstleve R W, et al. PHENIX: a comprehensive Python-based system for macromolecular structure solution. Acta crystallographica Section D, Biological crystallography. 2010; 66(Pt 2):213-21.
Laskowski R A, Macarthur M W, Moss D S, and Thornton J M. Procheck—a Program to Check the Stereochemical Quality of Protein Structures. J Appl Cryst. 1993; 26(2): 283-91.
Phillips J C, Braun R, Wang W, Gumbart J, Tajkhorshid E, Villa E, Chipot C, Skeel R D, Kale L, and Schulten K. Scalable molecular dynamics with NAMD. Journal of computational chemistry. 2005; 26(16):1781-802.
MacKerell A D, Bashford D, Bellott M, Dunbrack R L, Evanseck J D, Field M J, Fischer S, Gao J, Guo H, Ha S, et al. All-atom empirical potential for molecular modeling and dynamics studies of proteins. The journal of physical chemistry B. 1998; 102(18):3586-616.
Humphrey W, Dalke A, and Schulten K. VIVID: visual molecular dynamics. Journal of molecular graphics. 1996; 14(1):33-8, 27-8.
Tong X, Li K, Luo Z, Lu B, Liu X, Wang T, Pang M, Liang B, Tan M, Wu M, et al. Decreased TIP30 expression promotes tumor metastasis in lung cancer. Am J Pathol. 2009; 174(5): 1931-9.
Almoguera, C. et al. (1988) Most human carcinomas of the exocrine pancreas contain mutant c-K-ras genes. Cell 53, 549-554 PMCID: PMC1032821
Amit M, Na'ara S, Gil Z. (2016) Mechanisms of cancer dissemination along nerves. Nat Rev Cancer. 2016 May 6. doi: 10.1038/nrc.2016.38
Bailey P, Chang D K, Nones K, Johns A L, et al (2016). Genomic analyses identify molecular subtypes of pancreatic cancer. Nature 531(7592):47-52. PMID: 26909576
Ballard M S, Hinck L. (2012) A roundabout way to cancer. Adv Cancer Res. 2012; 114:187-235. PMCID: PMC4121377
Bakst R L, Wong R J. (2016) Mechanisms of Perineural Invasion. J Neurol Surg B Skull Base. 77(2):96-106
Bapat A A, Hostetter G, Von Hoff D D, Han H. (2011). Perineural invasion and associated pain in pancreatic cancer. Nat Rev Cancer 11:695-707.
Bedell V M, Yeo S Y, Park K W, Chung J, Seth P, Shivalingappa V, et al. (2005) Roundabout4 is essential for angiogenesis in vivo. Proc Natl Acad Sci USA 102:6373-8. PMCID: PMC1088354
Biankin A V, Waddell N, Kassahn K S, Gingras M C, Muthuswamy L B, Johns A L, et al. (2012) Pancreatic cancer genomes reveal aberrations in axon guidance pathway genes. Nature 491:399-405 PMCID: PMC3032824
Birchmeier C, Bennett D L (2016) Neuregulin/ErbB Signaling in Developmental Myelin Formation and Nerve
Repair. Curr Top Dev Biol. 116:45-64
Bockman D E, Buehler M, Beger H G. (1994) Interaction of pancreatic ductal carcinoma with nerves leads to nerve damage. Gastroenterology.; 107(1):219-30.
Brantley-Sieders D M, Dunaway C M, Rao M, Short S, Hwang Y, Gao Y, Li D, Jiang A, Shyr Y, Wu J Y, Chen J. (2011). Angiocrine factors modulate tumor proliferation and motility through EphA2 repression of Slit2 tumor suppressor function in endothelium. Cancer Res. 71(3):976-8. PMCID: PMC3032824
Brantley-Sieders D M. (2011). Clinical relevance of Ephs and ephrins in cancer: Lessons from breast, colorectal, and lung cancer profiling. Semin Cell Dev Biol. 23(1):102-8. PMCID: PMC3288332.
Breindel J L, Haskins J W, Cowell E P, Zhao M, Nguyen D X, Stern D F. (2013) EGF receptor activates MET through MAPK to enhance non-small cell lung carcinoma invasion and brain metastasis. Cancer Res. 73(16):5053-65. PMCID: PMC3745527
Brose K, Bland K S, Wang K H, Arnott D, Henzel W, Goodman C S, et al. (1999) Slit proteins bind Robo receptors and have an evolutionarily conserved role in repulsive axon guidance. Cell 96:795-806.
Caldas, C. et al. Frequent somaticmutations and homozygous deletions of the p16 (MTS1) gene in pancreatic adenocarcinoma. Nature Genet. 8, 27-32 (1994).
Carmeliet P, Tessier-Lavigne M. (2005) Common mechanisms of nerve and blood vessel wiring. Nature 436:193-200.
Carro M S, Lim W K, Alvarez M J, Bollo R J, Zhao X, Snyder E Y, Sulman E P, Anne S L, Doetsch F, Colman H, Lasorella A, Aldape K, Califano A, Iavarone A. (2010) The transcriptional network for mesenchymal transformation of brain tumours. Nature. 463(7279):318-25. PMCID: PMC4011561
Celesti G, Di Caro G, Bianchi P, Grizzi F, Marchesi F, Basso G, Rahal D, Delconte G, Catalano M, Cappello P, Roncalli M, Zerbi A, Montorsi M, Novelli F, Mantovani A, Allavena P, Malesci A, Laghi L. (2013) Early expression of the fractalkine receptor CX3CR1 in pancreatic carcinogenesis. Br J Cancer. 109(9):2424-33. PMCID: PMC3817321
Deheeger M, Lesniak M S, Ahmed A U. (2014). Cellular plasticity regulated cancer stem cell niche: a possible new mechanism of chemoresistance. Cancer Cell Microenviron. 1(5). pii: e295. PMCID: PMC4494756
D'Arca D, Zhao X, Xu W, Ramirez-Martinez N C, Iavarone A, Lasorella A. (2010) Huwe1 ubiquitin ligase is essential to synchronize neuronal and glial differentiation in the developing cerebellum. Proc Natl Acad Sci USA. 107(13):5875-80. PMCID: PMC2851916
Demir I E, Friess H, Ceyhan G O. (2012) Nerve-cancer interactions in the stromal biology of pancreatic cancer. Front Physiol. 3:97 PMID: 22529816. PMCID: PMC3327893
Dey M, Chang A L, Wainwright D A, Ahmed A U, Balyasnikova I V, Pyter P, Han Y, Tobias A, Zhang L, Qiao J, and Lesniak M S. Dendritic cell based vaccines that utilize myeloid rather than plasmacytoid cells offer a superior survival advantage in malignant glioma. J Immunology, 195(1): 367-76, 2015
Ceyhan G O, Bergmann F, Kadihasanoglu M, Altintas B, Demir I E, Hinz U, et al. (2009) Pancreatic neuropathy and neuropathic pain—a comprehensive pathomorphological study of 546 cases. Gastroenterology 136:177-86 el.
Ceyhan G O, Demir I E, Altintas B, Rauch U, Thiel G, Muller M W, et al. (2008) Neural invasion in pancreatic cancer: a mutual tropism between neurons and cancer cells. Biochem Biophys Res Commun 374:442-7.
Chang P H, Hwang-Verslues W W, Chang Y C, Chen C C, Hsiao M, Jeng Y M, et al. (2012) Activation of Robo1 Signaling of Breast Cancer Cells by Slit2 from Stromal Fibroblast Restrains Tumorigenesis via Blocking PI3K/Akt/beta-Catenin Pathway. Cancer research 72:4652-61. PMCID: PMC3445732
Chatterjee D, Katz M R, Rashid A, Wang H, Iuga A C, Varadhachary G R, et al. (2012) Perineural and intraneural invasion in post-therapy pancreaticoduodenectomy specimens predicts poor prognosis in patients with pancreatic ductal adenocarcinoma. Am J Surg Pathol 36:409-17. PMCID: PMC3288807
Chedotal A. Slits and their receptors. (2007) Adv Exp Med Biol 621:65-80.
Chen H, Wang Q, Lei Q, Zhang L, Kang L. (2015) Ontogenic expression profiles and oxaliplatin regulation of leptin expression in mice dorsal root ganglion. Neuroreport. 2015 Sep. 30; 26(14):870-6. doi: 10.1097/WNR.0000000000000440.
Chen Y, Xie D, Yin Li W, Man Cheung C, Yao H, Chan C Y, Chan C Y, Xu F P, Liu Y H, Sung J J, Kung H F. (2010) RNAi targeting EZH2 inhibits tumor growth and liver metastasis of pancreatic cancer in vivo. Cancer Lett.297(1):109-16.
Chen L H, Sun Y T, Chen Y F, Lee M Y, Chang L Y, Chang J Y, Shen M R. (2015) Integrating Image-Based High-Content Screening with Mouse Models Identifies 5-Hydroxydecanoate as a Neuroprotective Drug for Paclitaxel-Induced Neuropathy. Mol Cancer Ther. 14(10):2206-14. doi: 10.1158/1535-7163.MCT-15-0268
Chen S H, Hung W C, Wang P, Paul C, Konstantopoulos K. (2013) Mesothelin binding to CA125/MUC16 promotes pancreatic cancer cell motility and invasion via MMP-7 activation. Sci Rep. 3:1870. PMCID: PMC3660778
Clark, M. J., Chen, R., Lam, H. Y., Karczewski, K. J., Euskirchen, G., Butte, A. J. and Snyder, M. (2011) Performance comparison of exome DNA sequencing technologies. Nat Biotechnol, 29, 908-14. PMCID: PMC4127531
Dallol A, Da Silva N F, Viacava P, Minna J D, Bieche I, Maher E R, et al. (2002) SLIT2, a human homologue of the Drosophila Slit2 gene, has tumor suppressor activity and is frequently inactivated in lung and breast cancers. Cancer Res 62:5874-80.
Dallol A, Morton D, Maher E R, Latif F. (2003) SLIT2 axon guidance molecule is frequently inactivated in colorectal cancer and suppresses growth of colorectal carcinoma cells. Cancer Res 63:1054-8.
De Bellard M E, Rao Y, Bronner-Fraser M. (2013) Dual function of Slit2 in repulsion and enhanced migration of trunk, but not vagal, neural crest cells. J Cell Biol 162:269-79. PMCID: PMC2172792
Demir I E, Friess H, Ceyhan G O. (2012). Nerve-cancer interactions in the stromal biology of pancreatic cancer. Front Physiol. 3:97-108. PMCID: PMC3327893
Denk A E, Braig S, Schubert T, Bosserhoff A K. (2011) Slit3 inhibits activator protein 1-mediated migration of malignant melanoma cells. Int J Mol Med. 28(5):721-6
Ding L, Getz G, Wheeler D A, Mardis E R, McLellan M D, Cibulskis K, et al. Somatic mutations affect key pathways in lung adenocarcinoma. Nature. 2008; 455:1069-75. PMCID: PMC2694412.
Ding, L., Ellis, M. J., Li, S., et al. (2010) Genome remodelling in a basal-like breast cancer metastasis and xenograft. Nature, 464, 999-1005. PMCID: PMC2872544
DiNorcia J, M. K. Lee, D. N. Moroziewicz, M. D. Winner, P. Suman, F. Bao, H. E. Remotti, Y. S. Zou, S. F. Yan, W. Qiu, G. H. Su, A. M. Schmidt, J. D. Allendorf. (2012) RAGE gene deletion inhibits the development and progression of ductal neoplasia and prolongs survival in a mouse model of pancreatic cancer. Journal of Gastrointestinal Surgery 16:104-12. PMCID: PMC4049447
Dunaway C M, Hwang Y, Lindsley C W, Cook R S, Wu J Y, Boothby M, et al. (2011) Cooperative signaling between Slit2 and Ephrin-A1 regulates a balance between angiogenesis and angiostasis. Mol Cell Biol 31:404-16. PMCID: PMC3028625
Fischer C, Jonckx B, Mazzone M, Zacchigna S, Loges S, Pattarini L, et al. (2007) Anti-PlGF Inhibits Growth of VEGF(R)-Inhibitor-Resistant Tumors without Affecting Healthy Vessels. Cell 131:463-75.
Fischer C, Sanchez-Ruderisch H, Welzel M, Wiedenmann B, Sakai T, Andre S, et al. (2005) Galectin-1 interacts with the {alpha}5{beta}1 fibronectin receptor to restrict carcinoma cell growth via induction of p21 and p27. J Biol Chem 280:37266-77.
Foley K, Rucki A A, Xiao Q, Zhou D, Leubner A, Mo G, Kleponis J, Wu A A, Sharma R, Jiang Q, Anders R A, Iacobuzio-Donahue C A, Hajjar K A, Maitra A, Jaffee E M, Zheng L. (2015) Semaphorin 3D autocrine signaling mediates the metastatic role of annexin A2 in pancreatic cancer. Science Signaling. 8(388):ra77. PMCID: PMC4811025
Gil Z, Rein A, Brader P, Li S, Shah J P, Fong Y, Wong R J. (2007) Nerve-sparing therapy with oncolytic herpes virus for cancers with neural invasion. Clin Cancer Res. 13(21):6479-85
Gil Z, Cavel O, Kelly K, Brader P, Rein A, Gao S P, et al. (2010) Paracrine regulation of pancreatic cancer cell invasion by peripheral nerves. J Natl Cancer Inst 102:107-18. PMCID: PMC2911041
Gara R K, Kumari S, Ganju A, Yallapu M M, Jaggi M, Chauhan S C. (2015) Slit/Robo pathway: a promising therapeutic target for cancer. Drug Discov Today. 20(1):156-6. PMCID: PMC4445861
Giovannone D, Reyes M, Reyes R, Correa L, Martinez D, Ra H, et al. (2012) Slits affect the timely migration of neural crest cells via Robo receptor. Dev Dyn 241:1274-88. PMCID: PMC3632352
Giridharan S S, Caplan S. (2014) MICAL-family proteins: Complex regulators of the actin cytoskeleton. Antioxid Redox Signal.; 20(13):2059-73. PMCID: PMC3993057.
Göhrig A, Detjen K M, Hilfenhaus G, Korner J, Arsenic R, Welzel M, Schmuck R, Bahra M, Wu J Y, Wiedenmann B, Fischer C (2014). The Axon Guidance Factor Slit2 inhibits Pancreatic Cancer Metastasis and Cell Movement along Neurites. Cancer Res. 74(5):1529-40
Govindan, R., Ding, L., Griffith, M., Subramanian, J., Dees, N. D., Kanchi, K. L., Maher, C. A., Fulton, R., Fulton, L., Wallis, J., et al. (2012). Genomic landscape of non-small cell lung cancer in smokers and never-smokers. Cell 150, 1121-1134. PMCID: PMC3656590
Grage-Griebenow E, Jerg E, Gorys A, Wicklein D, Wesch D, Freitag-Wolf S, Goebel L, Vogel I, Becker T, Ebsen M, Rocken C, Altevogt P, Schumacher U, Schäfer H, Sebens S. (2014) L1CAM promotes enrichment of immunosuppressive T cells in human pancreatic cancer correlating with malignant progression. Mol Oncol. 8(5):982-9
Grieshammer U, Le M, Plump A S, Wang F, Tessier-Lavigne M, Martin G R. (2004) SLIT2-mediated ROBO2 signaling restricts kidney induction to a single site. Dev Cell 6:709-17.
Gu D, H. Liu, G. H. Su, X. Zhang, H. Chin-Sinex, H. Hanenberg, M. Mendonca, H. E. Shannon, E. G. Chiorean, J. Xie. (2013) Combining hedgehog signaling inhibition with focal irradiation on reduction of pancreatic cancer metastasis. Molecular Cancer Therapeutics (Epub Mar. 6, 2013). PMCID: PMC3681871
Guan H, Zu G, Xie Y, Tang H, Johnson M, Xu X, Kevil C, Xiong W-C, Elmets C, Wu J Y, Xu H (2003). Neuronal repellent Slit2 inhibits dendritic cell migration and the development of immune responses. J. Immunol. 171:6519-6526.
Guo K, Ma Q, Li J, Wang Z, Shan T, Li W, Xu Q, Xie K. (2013) Interaction of the sympathetic nerve with pancreatic cancer cells promotes perineural invasion through the activation of STAT3 signaling. Mol Cancer Ther. 12(3):264-73
He S, Chen C H, Chernichenko N, He S, Bakst R L, Barajas F, Deborde S, Allen P J, Vakiani E, Yu Z, Wong R J. (2014) GFRα1 released by nerves enhances cancer cell perineural invasion through GDNF-RET signaling. Proc Natl Acad Sci USA. 111(19):E2008-17. PMCID: PMC4024863
He S, He S, Chen C H, Deborde S, Bakst R L, Chernichenko N, McNamara W F, Lee S Y, Barajas F, Yu Z, Al-Ahmadie H A, Wong R J. (2015) The chemokine (CCL2-CCR2) signaling axis mediates perineural invasion. Mol Cancer Res. 13(2):380-90. PMCID: PMC4336839
Heidenreich M, Zhang F. (2016) Applications of CRISPR-Cas systems in neuroscience. Nat Rev Neurosci. 17(1):36-44. PMCID: PMC4899966
Hertzer K M, Donald G W, Hines O J. (2013) CXCR2: a target for pancreatic cancer treatment? Expert Opin Ther Targets. 17(6):667-80. PMCID: PMC3686651
Hezel A F, Kimmelman A C, Stanger B Z, Bardeesy N, Depinho R A. (2006) Genetics and biology of pancreatic ductal adenocarcinoma. Genes Dev. 20(10):1218-49.
Hingorani, S. R. et al. Preinvasive and invasive ductal pancreatic cancer and its early detection in the mouse. Cancer Cell 4, 437-450 (2003).
Hruban R H, Goggins M, Parsons J, Kern S E (2000) Progression model for pancreatic cancer. Clin Cancer Res 6:2969-2972.
Huffmaster N E, Sollars P J2, Richards A L1, Pickard G E3, Smith G A (2015) Dynamic ubiquitination drives herpesvirus neuroinvasion Proc Natl Acad Sci USA. 112(41):12818-23. PMCID: PMC4611658
Huang P, Kishida S, Cao D, Murakami-Tonami Y, Mu P, Nakaguro M, Koide N, Takeuchi I, Onishi A, Kadomatsu K. (2011). The neuronal differentiation factor NeuroD1 downregulates the neuronal repellent factor Slit2 expression and promotes cell motility and tumor formation of neuroblastoma. Cancer Res. 71(8):2938-48. PMCID: PMC21349947.
Huang Z, Wen P, Kong R, Cheng H, Zhang B, Quan C, Bian Z, Chen M, Zhang Z, Chen X, Du X, Liu J, Zhu L, Fushimi K, Hua D, Wu J Y. (2014). USP33 mediates Slit-Robo signaling in inhibiting colorectal cancer cell migration. Int J Cancer. 2014 Sep. 20. doi: 10.1002/ijc.29226. PMCID: PMC4323690
Huang Z, Wen P, Kong R, Cheng H, Zhang B, Quan C, Bian Z, Chen M, Zhang Z, Chen X, Du X, Liu J, Zhu L, Fushimi K, Hua D, Wu J Y. (2015) USP33 mediates Slit-Robo signaling in inhibiting colorectal cancer cell migration. Int J Cancer. 136(8):1792-802.
Huangfu W C, Fuchs S Y.2010. Ubiquitination-dependent regulation of signaling receptors in cancer. Genes Cancer. 1(7):725-34. PMCID: PMC2994580.
Hung R J, Pak C W, Terman J R. (2011a) Direct redox regulation of F-actin assembly and disassembly by Mical. Science 334(6063):1710-3. PMCID: PMC3612955
Hung R J, Terman J R. (2011b) Extracellular inhibitors, repellents, and semaphorin/plexin/MICAL-mediated actin filament disassembly. Cytoskeleton (Hoboken). 68(8):415-33. PMCID: PMC3612987
Hung R J, Yazdani U, Yoon J, Wu H, Yang T, Gupta N, Huang Z, van Berkel W J, Terman J R. (2010) Mical links semaphorins to F-actin disassembly. Nature. 463(7282):823-7. PMCID: PMC3215588.
Huynh, A. S., et al. (2011). Development of an orthotopic human pancreatic cancer xenograft model using ultrasound guided injection of cells. PLoS One, 6(5), e20330. PMCID: PMC3103544
Imoto A, Mitsunaga S, Inagaki M, Aoyagi K, Sasaki H, Ikeda M, Nakachi K, Higuchi K, Ochiai A. (2012) Neural invasion induces cachexia via astrocytic activation of neural route in pancreatic cancer. Int J Cancer. 131(12):2795-807
Jackson E L, Willis N, Mercer K. et al (2001). Analysis of lung tumor initiation and progression using conditional expression of oncogenic K-ras. Genes Dev. 15:3243-3248. PMCID: PMC312845.
Jaworski A, Tessier-Lavigne M. (2012) Autocrine/juxtaparacrine regulation of axon fasciculation by Slit-Robo signaling. Nat Neurosci 15:367-9.
Jia L, Cheng L, Raper J. (2005) Slit/Robo signaling is necessary to confine early neural crest cells to the ventral migratory pathway in the trunk. Dev Biol 282:411-21.
Jones C A et al. Robo4 stabilizes the vascular network by inhibiting pathologic angiogenesis and endothelial hyperpermeability. Nature Med. 14, 448-453 (2008). PMCID: PMC2875252
Jones C A, Nishiya N, London N R, Zhu W, Sorensen L K, Chan A C, et al. (2009) Slit2-Robo4 signalling promotes vascular stability by blocking Arf6 activity. Nat Cell Biol 11:1325-31. PMCID: PMC2854659
Jones, S. et al. Core signaling pathways in human pancreatic cancers revealed by global genomic analyses. Science 321, 1801-1806 (2008). PMCID: PMC2848990
Kapoor A, Yao W, Ying H, Hua S, Liewen A, Wang Q, Zhong Y, Wu C J, Sadanandam A, Hu B, Chang Q, Chu G C, Al-Khalil R, Jiang S, Xia H, Fletcher-Sananikone E, Lim C, Horwitz G I, Viale A, Pettazzoni P, Sanchez N, Wang H, Protopopov A, Zhang J, Heffernan T, Johnson R L, Chin L, Wang Y A, Draetta G, DePinho R A. (2014). Yap1 activation enables bypass of oncogenic kras addiction in pancreatic cancer. Cell 158(1):185-97. PMCID: PMC4109295
Khan S, Jaggi M, Chauhan S C. (2015). Revisiting stroma in pancreatic cancer. Oncoscience. 2(10):819-20
Kienast, Y.; von Baumgarten, L.; Fuhrmann, M.; Klinkert, W. E.; Goldbrunner, R.; Herms, J.; Winkler, F. (2010) Real-time imaging reveals the single steps of brain metastasis formation. Nat. Med., 16, 116-122.
Kim H N, Choi D W, Lee K T, Lee J K, Heo J S, Choi S H, et al. (2007) Gene expression profiling in lymph node-positive and lymph node-negative pancreatic cancer. Pancreas 34:325-34.
Kim S, Alexander C M. (2014). Tumorsphere assay provides more accurate prediction of in vivo responses to chemotherapeutics. Biotechnol Lett. 2014 March; 36(3):481-8. PMCID: PMC3966961
Klein R S, Rubin J B, Gibson H D, DeHaan E N, Alvarez-Hernandez X, Segal R A, Luster A D. (2001) SDF-1 alpha induces chemotaxis and enhances Sonic hedgehog-induced proliferation of cerebellar granule cells. Development. 2001 June; 128(11): 1971-81.
Koboldt, D. C., Zhang, Q., Larson, D. E., Shen, D., McLellan, M. D., Lin, L., Miller, C. A., Mardis, E. R., Ding, L. and Wilson, R. K. (2012) VarScan 2: somatic mutation and copy number alteration discovery in cancer by exome sequencing. Genome Res, 22, 568-76. PMCID: PMC3290792.
Koide, N., Yamada, T., Shibata, R., Mori, T., Fukuma, M., Yamazaki, K., Aiura, K., Shimazu, M., Hirohashi, S., Nimura, Y., et al. (2006). Establishment of perineural invasion models and analysis of gene expression revealed an invariant chain (CD74) as a possible molecule involved in perineural invasion in pancreatic cancer. Clinical Cancer Research 12, 2419-2426.
Komander D, Clague M J, Urbé S. (2009) Breaking the chains: structure and function of the deubiquitinases. Nat Rev Mol Cell Biol. 10(8):550-63
Kuriyama S, Mayor R. Molecular analysis of neural crest migration. (2008) Philos Trans R Soc Lond B Biol Sci 363:1349-62. Göhrig et al. 18 PMCID: PMC2610123
Lahoz A, Hall A. (2013). A tumor suppressor role for srGAP3 in mammary epithelial cells. Oncogene. 32(40):4854-60
Lee, B. C.; Lee, T. H.; Avraham, S.; Avraham, H. K. Involvement of the chemokine receptor CXCR4 and its ligand stromal cell-derived factor 1alpha in breast cancer cell migration through human brain microvascular endothelial cells. Mol. Cancer Res. 2004, 2, 327-338.
Legg J A, Herbert J M, Clissold P, Bicknell R. (2008) Slits and Roundabouts in cancer, tumour angiogenesis and endothelial cell migration. Angiogenesis 11:13-21.
Li H S, Chen J H, Wu W, Fagaly T, Zhou L, Yuan W, et al. (1999) Vertebrate slit, a secreted ligand for the transmembrane protein roundabout, is a repellent for olfactory bulb axons. Cell 96:807-18.
Li X, Gao X, Liu G, Xiong W, Wu J, Rao Y. (2008). Netrin signal transduction and the guanine nucleotide exchange factor DOCK180 in attractive signaling Nature Neuroscience 11:28-35. PMCID: PMC2262939.
Li, F. P., J. F. Fraumeni, Jr., et al. (1988). A cancer family syndrome in twenty-four kindreds. Cancer Res 48(18): 5358-62.
Li, H. and Durbin, R. (2009a) Fast and accurate short read alignment with Burrows-Wheeler transform. Bioinformatics, 25, 1754-60. PMCID: PMC2705234.
Li, H., Handsaker, B., Wysoker, A., Fennell, T., Ruan, J., Homer, N., Marth, G., Abecasis, G. and Durbin, R. (2009b) The Sequence Alignment/Map format and SAMtools. Bioinformatics, 25, 2078-9. PMCID: PMC2723002.
Li, Y., Vinckenbosch, N., Tian, G., et al., (2010) Resequencing of 200 human exomes identifies an excess of low-frequency non-synonymous coding variants. Nat Genet, 42, 969-72.
Li Y, Yang M, Huang Z, Chen X, Maloney M T, Zhu L, Liu J, Yang Y, Du S, Jiang X, Wu J Y. (2014) AxonQuant: A Microfluidic Chamber Culture-Coupled Algorithm That Allows High-Throughput Quantification of Axonal Damage. Neurosignals. 22(1):14-29. PMCID: PMC4148468
Liang D, Shi S, Xu J, Zhang B, Qin Y, Ji S, Xu W, Liu J, Liu L, Liu C, Long J, Ni Q, Yu X. (2016) New insights into perineural invasion of pancreatic cancer: More than pain. Biochim Biophys Acta. 2016 Jan. 12
Liebig C, Ayala G, Wilks J A, Berger D H, Albo D (2009) Perineural invasion in cancer: A review of the literature. Cancer 115(15):3379-3391.
Lin Y Y, Yang C H, Sheu G T, Huang C Y, Wu Y C, Chuang S M, Fann M J, Chang H, Lee H, Chang J T (2011) A novel exon 15-deleted, splicing variant of Slit2 shows potential for growth inhibition in addition to invasion inhibition in lung cancer. Cancer. 117(15):3404-15.
Lin, S., A. Chakravarti, et al. (2004). Exhaustive allelic transmission disequilibrium tests as a new approach to genome-wide association studies. Nat Genet 36(11): 1181-8.
Liu G, Li W Q, Wang L, Kar A, Guan K-L, Rao Y, Wu J Y (2009). DSCAM Functions as a Netrin Receptor in Commissural Axon Pathfinding. Proc. Natl. Acad. Sci. USA 106(8): 2951-6. PMCID: PMC2650370.
Liu P, Morrison C, Wang L, Xiong D, Vedell P, et al. (2012) Identification of Somatic Mutations in Non-Small Cell Lung Carcinomas Using Whole-Exome Sequencing. Carcinogenesis. 33(7):1270-6 PMCID: PMC3499051
Liu, J. S., C. Sabatti, et al. (2001). Bayesian analysis of haplotypes for linkage disequilibrium mapping. Genome Res 11(10): 1716-24. PMCID: PMC311130.
Liu, P, Vikis H, et al. (2008). Familial Aggregation of Common Sequ65q.ence Variants on 15q24-25.1 in Lung Cancer J Natl Cancer Inst. 100(18):1326-30. PMCID: PMC2538550.
Liu, P., Y. Wang, et al. (2006). Candidate lung tumor susceptibility genes identified through whole-genome association analyses in inbred mice. Nat Genet 38(8): 888-95
Lukas R V, Lesniak M S, Salgia R. (2014). Brain metastases in non-small-cell lung cancer: better outcomes through current therapies and utilization of molecularly targeted approaches. CNS Oncol. 3(1):61-75.
Macias H, Moran A, Samara Y, Moreno M, Compton J E, Harburg G, et al. (2011) SLIT/ROBO1 signaling suppresses mammary branching morphogenesis by limiting basal cell number. Dev Cell 20:827-40. PMCID: PMC3129866
Majumder K, Arora N, Modi S, Chugh R, Nomura A, Giri B, Dawra R, Ramakrishnan S, Banerjee S, Saluja A, Dudeja V. (2016) A Novel Immunocompetent Mouse Model of Pancreatic Cancer with Robust Stroma: a Valuable Tool for Preclinical Evaluation of New Therapies. J Gastrointest Surg. 20(1):53-65
Marchesi F, Grizzi F, Laghi L, Mantovani A, Allavena P. (2012) Molecular mechanisms of pancreatic cancer dissemination: the role of the chemokine system. Curr Pharm Des. 18(17):2432-8. PMID: 22372498
Marlow R, Strickland P, Lee J S, Wu X, Pebenito M, Binnewies M, Le E K, Moran A, Macias H, Cardiff R D, Sukumar S, Hinck L. (2008) SLITs suppress tumor growth in vivo by silencing Sdf1/Cxcr4 within breast epithelium. Cancer Res. 68(19):7819-27. PMCID: PMC3075571.
McKenna, A., Hanna, M., Banks, E., Sivachenko, A., Cibulskis, K., Kernytsky, A., Garimella, K., Altshuler, D., Gabriel, S., Daly, M. and DePristo, M. A. (2010) The Genome Analysis Toolkit: a MapReduce framework for analyzing next-generation DNA sequencing data. Genome Res, 20, 1297-303. PMCID: PMC2928508.
McPeek, M. S. A. Strahs. (1999). Assessment of linkage disequilibrium by the decay of haplotype sharing, with application to fine-scale genetic mapping. Am J Hum Genet 65(3): 858-75. PMCID: PMC1378001
Mehlen P, Delloye-Bourgeois C, Chedotal A. (2011) Novel roles for Slits and netrins: axon guidance cues as anticancer targets? Nat Rev Cancer 11:188-97.
Mitsunaga S, Hasebe T, Kinoshita T, Konishi M, Takahashi S, Gotohda N, et al. (2007) Detail histologic analysis of nerve plexus invasion in invasive ductal carcinoma of the pancreas and its prognostic impact. Am J Surg Pathol 31:1636-44.
Morlot C, Thielens N M, Ravelli R B, Hemrika W, Romijn R A, Gros P, Cusack S, McCarthy A A. (2007). Structural insights into the Slit-Robo complex. Proc Natl Acad Sci USA. 104(38):14923-8. PMCID: PMC1975871.
Morton, J. P., et al. (2010). Mutant p53 drives metastasis and overcomes growth arrest/senescence in pancreatic cancer. Proc Natl Acad Sci USA, 107(1), 246-251. PMCID: PMC2806749
M [symbol] zes G, Sipos F. (2014). Relation of immune semaphorin/plexin signaling to carcinogenesis. Eur J Cancer Prey. 2014 Jun. 11
Nagakawa T, Mori K, Nakano T, et al. Perineural invasion of carcinoma of the pancreas and biliary tract. Br J Surg. 1993; 80:619-621.
Nakao A, Harada A, Nonami T, et al. Clinical significance of carcinoma invasion of the extrapancreatic nerve plexus in pancreatic cancer. Pancreas. 1996; 12:357-361.
Neagu M R, Gill C M, Batchelor T T, Brastianos P K. (2015). Genomic profiling of brain metastases: current knowledge and new frontiers. Chin Clin Oncol. 2015 June; 4(2):22.
Nelson M, Millican-Slater R, Forrest L C, Brackenbury W J. (2014) The sodium channel β1 subunit mediates outgrowth of neurite-like processes on breast cancer cells and promotes tumour growth and metastasis. Int J Cancer. 2014 Apr. 12. PMCID: PMC4200311
Nijmeijer R M, van Santvoort H C, Zhernakova A et al, Dutch Pancreatitis Study Group. (2013) Association analysis of genetic variants in the myosin IXB gene in acute pancreatitis. PLoS One. 8(12):e85870. PMCID: PMC3875581
Nomura A, McGinn O, Dudeja V, Sangwan V, Saluja A K, Banerjee S. (2015). Minnelide effectively eliminates CD133(+) side population in pancreatic cancer. Mol Cancer. 14(1):200; PMCID: PMC4657383
Nones K, Waddell N, Song S, Patch A M, Miller D, Johns A, Wu J, Kassahn K S, Wood D, Bailey P, Fink L, Manning S, Christ A N, Nourse C, Kazakoff S, Taylor D, Leonard C, Chang D K, Jones M D, Thomas M, Watson C, Pinese M, Cowley M, Rooman I, Pajic M; APGI, Butturini G, Malpaga A, Corbo V, Crippa S, Falconi M, Zamboni G, Castelli P, Lawlor R T, Gill A J, Scarpa A, Pearson J V, Biankin A V, Grimmond S M. (2014) Genome-wide DNA methylation patterns in pancreatic ductal adenocarcinoma reveal epigenetic deregulation of SLIT-ROBO, ITGA2 and MET signaling. Int J Cancer 135(5):1110-8 [Slit2, 3, Robo1, 3 hypermethylated, expression decreased in PDAC
Nguyen Ba-Charvet K T, Brose K, Ma L, Wang K H, Marillat V, Sotelo C, et al. (2001) Diversity and specificity of actions of Slit2 proteolytic fragments in axon guidance. J Neurosci 21:4281-9.
Oser M, Yamaguchi H, Mader C C, Bravo-Cordero J J, Arias M, Chen X, Desmarais V, van Rheenen J, Koleske A J, Condeelis J. (2009). Cortactin regulates cofilin and N-WASp activities to control the stages of invadopodium assembly and maturation. J Cell Biol. 186(4):571-87. PMCID: PMC2733743
Owonikoko T K, Arbiser J, Zelnak A, Shu H K, Shim H, Robin A M, Kalkanis S N, Whitsett T G, Salhia B, Tran N L, Ryken T, Moore M K, Egan K M, Olson J J. (2014). Current approaches to the treatment of metastatic brain tumours. Nat Rev Clin Oncol. 2014 April; 11(4):203-22. PMCID: PMC4041037
Panapakkam Giridharan S S, Rohn J L, Naslaysky N, Caplan S. (2012) Differential regulation of actin microfilaments by human MICAL proteins. J Cell Sci. 125(Pt 3):614-24. PMCID: PMC3367829
Park K W, Morrison C M, Sorensen L K, Jones C A, Rao Y, Chien C B, et al. (2003) Robo4 is a vascular-specific receptor that inhibits endothelial migration. Dev Biol 261:251-67.
Pasterkamp R J, Dai H N, Terman J R, Wahlin K J, Kim B, Bregman B S, Popovich P G, Kolodkin A L. (2006). MICAL flavoprotein monooxygenases: expression during neural development and following spinal cord injuries in the rat. Mol Cell Neurosci. 31(1):52-69.
Perini M V, Montagnini A L, Jukemura J, Penteado S, Abdo E E, Patzina R, et al. (2008) Clinical and pathologic prognostic factors for curative resection for pancreatic cancer. HPB(Oxford) 10:356-62. PMCID: PMC2575675
Pollack I F. (2013). Tumor-stromal interactions in medulloblastoma. N Engl J Med. 368(20):1942-3.
Prasad A, Paruchuri V, Preet A, Latif F, Ganju R K. (2008). Slit-2 induces a tumor-suppressive effect by regulating beta-catenin in breast cancer cells. J Biol Chem. 283(39):26624-33. PMCID: PMC2546548
Prasad A, Qamri Z, Wu J, Ganju R K. (2007). Pivotal Advance: Slit-2/Robo-1 modulates the CXCL12/CXCR4-induced chemotaxis of T cells. J Leukoc Biol. 82(3):465-76. PMCID: PMC2286829.
Qiao X, Zhang L, Gamper A M, Fujita T, Wan Y. (2010) APC/C-Cdh1: from cell cycle to cellular differentiation and genomic integrity. Cell Cycle. 9(19):3904-12. PMCID: PMC3047751
Qiu W and G. H. Su. (2013) Development of orthotopic pancreatic tumor mouse models. Methods Mol Biol. 2013; 980:215-23.
Qiu W, F. Sahin, C. A. Iacobuzio-Donahue, Dario Garcia-Carracedo, W. M. Wang, Chia-Yu Kuo, Dan E. Arking, A. M. Lowy, R. H. Hruban, H. E. Remotti, G. H. Su. (2011) Disruption of p16 and Activation of Kras in Pancreas Increases Ductal Adenocarcinoma Formation and Metastasis in vivo. Oncotarget, 2:862-73 PMCID: PMC3259996
Qu D, Johnson J, Chandrakesan P, Weygant N, May R, Aiello N, Rhim A, Zhao L, Zheng W, Lightfoot S, Pant S, Irvan J, Postier R, Hocker J, Hanas J S, Ali N, Sureban S M, An G, Schlosser M J, Stanger B, Houchen C W. (2015). Doublecortin-like kinase 1 is elevated serologically in pancreatic ductal adenocarcinoma and widely expressed on circulating tumor cells. PLoS One. 10(2):e0118933. PMCID: PMC4344195
Qiu W, Tang S M, Lee S, Turk A T, Sireci A N, Qiu A, Rose C, Xie C, Kitajewski J, Wen H J, Crawford H C, Sims P A, Hruban R H, Remotti H E, Su G H. (2016) Loss of Activin Receptor Type 1B Accelerates Development of Intraductal Papillary Mucinous Neoplasms in Mice With Activated KRAS. Gastroenterology. 150(1):218-228 PMID: 26408346
Rahajeng J, Giridharan S S, Cai B, Naslaysky N, Caplan S. (2010) Important relationships between Rab and MICAL proteins in endocytic trafficking. World J Biol Chem. 26; 1(8):254-64. PMCID: PMC3083971.
Rao Y, Wong K, Ward M, Jurgensen C, Wu J Y (2002) Neuronal Migration and molecular conservation with leukocyte chemotaxis. Genes & Development 16:2973-84. PMCID: PMC2064004.
Rao Y, Wu J Y (2001). Neuronal Migration and the Evolution of the Human Brain. Nature Neuroscience 4:860-862. PMCID: PMC2064000
Redston, M. S. et al. p53 mutations in pancreatic carcinoma and evidence of common involvement of homocopolymer tracts in DNA microdeletions. Cancer Res. 54, 3025-3033 (1994).
Reichert M, Rustgi A K. (2011) Pancreatic ductal cells in development, regeneration, and neoplasia. J Clin Invest. 121(12):4572-8 PMCID: PMC3225990
Rosenberger I, Strauss A, Dobiasch S, Weis C, Szanyi S, Gil-Iceta L, Alonso E, González Esparza M, Gómez-Vallejo V, Szczupak B, Plaza-Garcia S, Mirzaei S, Israel L L, Bianchessi S, Scanziani E, Lellouche J P, Knoll P, Werner J, Felix K, Grenacher L, Reese T, Kreuter J, Jiménez-González M. (2015) Targeted diagnostic magnetic nanoparticles for medical imaging of pancreatic cancer. J Control Release.; 214:76-84
Rubin J B, Kung A L, Klein R S, Chan J A, Sun Y, Schmidt K, Kieran M W, Luster A D, Segal R A. (2003) A small-molecule antagonist of CXCR4 inhibits intracranial growth of primary brain tumors. Proc Natl Acad Sci USA. 100(23):13513-8 PMCID: PMC4355991
Rubinson, D. A., C. P. Dillon, et al. (2003). A lentivirus-based system to functionally silence genes in primary mammalian cells, stem cells and transgenic mice by RNA interference. Nat Genet 33(3): 401-6
San Lucas, F. A., Wang, G., Scheet, P. Peng, B. (2012) Integrated annotation and analysis of genetic variants from next-generation sequencing studies with variant tools. Bioinformatics, 28, 421-2. PMCID: PMC3268240.
Saghin R and Xia Z J (2010) The entropy conjecture for partially hyperbolic diffeomorphisms with 1-d center, Topology and Its Applications, 157(1): 29-34.
Saghin R and Xia Z J (2009) Geometric expansion, Lyapunov exponents and foliations, Ann. Inst. H. Poincare Anal. Non Lineaire 26 (2): 689-704
Sakane A, Honda K, Sasaki T. (2010) Rab13 regulates neurite outgrowth in PC12 cells through its effector protein, JRAB/MICAL-L2. Mol Cell Biol. 2010 February; 30(4):1077-87. PMCID: PMC2815571
Sander E et al, (1998). Matrix-dependent Tiam1/Rac signaling in epithelial cells promotes either cell-cell adhesion or cell migration and is regulated by phosphatidylinositol 3-kinase. J Cell Biol 143:1385-1398. PMCID: PMC2133078
Schaid, D. J., C. M. Rowland, et al. (2002). Score tests for association between traits and haplotypes when linkage phase is ambiguous. Am J Hum Genet 70(2): 425-34. PMCID: PMC384917
Scheet P, Stephens M (2006). A fast and flexible statistical model for large-scale population genotype data: applications to inferring missing genotypes and haplotypic phase. Am J Hum Genet 78(4): 629-44. PMCID: PMC1424677.
Schmid B C, Rezniczek G A, Fabjani G, Yoneda T, Leodolter S, Zeillinger R. (2007) The neuronal guidance cue Slit2 induces targeted migration and may play a role in brain metastasis of breast cancer cells. Breast Cancer Res Treat 106:333-42.
Schmid B C, Rezniczek G A, Fabjani G, Yoneda T, Leodolter S, Zeillinger R (2007). The neuronal guidance cue Slit2 induces targeted migration and may play a role in brain metastasis of breast cancer cells. Breast Cancer Res. Treat 106, 333-342
Schulz P, Fischer C, Detj en K M, Rieke S, Hilfenhaus G, von Marschall Z, et al. (2011) Angiopoietin-2 drives lymphatic metastasis of pancreatic cancer. Faseb J 25:3325-35.
Schwab B, Rudolph A, Jost E, Klinge U. (2014) Collaborative network of predictive markers complicates formation of prognostic groups in patients with advanced lung cancer. Anticancer Res. 34(6):2745-54.
Siegel R L, Miller K D, Jemal A. (2016). Cancer statistics, 2016; C A Cancer J Clin.66(1):7-30; PMID: 26742998
Sharma M, Giridharan S S, Rahajeng J, Naslaysky N, Caplan S. (2009) MICAL-L1 links EHD1 to tubular recycling endosomes and regulates receptor recycling. Mol Biol Cell. 20(24):5181-94. PMCID: PMC2793294
Secq V, Leca J, Bressy C, Guillaumond F, Skrobuk P, Nigri J, Lac S, Lavaut M N, Bui T T, Thakur A K, Callizot N, Steinschneider R, Berthezene P, Dusetti N, Ouaissi M, Moutardier V, Calvo E, Bousquet C, Garcia S, Bidaut G, Vasseur S, Iovanna J L, Tomasini R. (2015) Stromal SLIT2 impacts on pancreatic cancer-associated neural remodeling. Cell Death Dis.; 6:e1592. PMCID: PMC4669755
Shimada K, Nara S, Esaki M, Sakamoto Y, Kosuge T, Hiraoka N. (2011) Intrapancreatic nerve invasion as a predictor for recurrence after pancreaticoduodenectomy in patients with invasive ductal carcinoma of the pancreas. Pancreas 40:464-8.
Sowa M E, Bennett E J, Gygi S P, Harper J W. Defining the human deubiquitinating enzyme interaction landscape. Cell 2009; 138:389-403. PMCID: PMC2716422
Stathis A, Moore M J. (2010) Advanced pancreatic carcinoma: current treatment and future challenges. Nature reviews 7:163-72.
Stella M C, Trusolino L, Comoglio P M. (2009) The Slit/Robo system suppresses hepatocyte growth factor-dependent invasion and morphogenesis. Mol Biol Cell 20:642-57. PMCID: PMC2626568
Stoll E A, Homer P J, Rostomily R C. (2013) The impact of age on oncogenic potential: tumor-initiating cells and the brain microenvironment. Aging Cell. 12(5):733-41. PMCID: PMC4199664
Saukkonen K, Hagstrom J, Mustonen H, Juuti A, Nordling S, Fermér C, Nilsson O, Seppänen H, Haglund C. (2015). Podocalyxin Is a Marker of Poor Prognosis in Pancreatic Ductal Adenocarcinoma. PLoS One. 10(6):e0129012. PMCID: PMC4459962
Svokos K A, Salhia B, Toms S A (2014). Molecular biology of brain metastasis. Int J Mol Sci. 15(6):9519-30.
Tang H, Wei P, Duell E J, Risch H A, Olson S H, Bueno-de-Mesquita H B, Gallinger S, Holly E A, Petersen G, Bracci P M, McWilliams R R, Jenab M, Riboli E, Tjonneland A, Boutron-Ruault M C, Kaaks R, Trichopoulos D, Panico S, Sund M, Peeters P H, Khaw K T, Amos C I, Li D. (2014) Axonal guidance signaling pathway interacting with smoking in modifying the risk of pancreatic cancer: a gene- and pathway-based interaction analysis of GWAS data. Carcinogenesis. 2014 May; 35(5):1039-45. PMCID: PMC4004205
Terman J R, Mao T, Pasterkamp R J, Yu H H, Kolodkin A L. (2002). MICALs, a family of conserved flavoprotein oxidoreductases, function in plexin-mediated axonal repulsion. Cell. 109(7):887-900.
Termini J, Neman J, Jandial R. (2014). Role of the Neural Niche in Brain Metastatic Cancer. Cancer Res.
Thomas A M, Seidlits S K, Goodman A G, Kukushliev T V, Hassani D M, Cummings B J, Anderson A J, Shea L D. (2014) Sonic hedgehog and neurotrophin-3 increase oligodendrocyte numbers and myelination after spinal cord injury. Integr Biol 24; 6(7):694-705. PMCID: PMC4086750
Timpson P, McGhee E J, Morton J P, von Kriegsheim A, Schwarz J P, Karim S A, Doyle B, Quinn J A, Carragher N O, Edward M, Olson M F, Frame M C, Brunton V G, Sansom O J, Anderson K I. (2011). Spatial regulation of RhoA activity during pancreatic cancer cell invasion driven by mutant p53. Cancer Res. 71(3):747-57107. PMCID: PMC3033324
Thomas, J. K. et al. (2014). Pancreatic Cancer Database: An integrative resource for pancreatic cancer. Cancer Biology and Therapy. 15, 8. PMCID: PMC4119079
Thomas, R. K., E. Nickerson, et al. (2006). Sensitive mutation detection in heterogeneous cancer specimens by massively parallel picoliter reactor sequencing. Nat Med 12(7): 852-5
Tseng, W. W., et al. (2010). Development of an orthotopic model of invasive pancreatic cancer in an immunocompetent murine host. Clinical Cancer Research, 16(14), 3684-3695. PMCID: PMC3085509
Tsukamoto N, Egawa S, Akada M, Abe K, Saiki Y, Kaneko N, Yokoyama S, Shima K, Yamamura A, Motoi F, Abe H, Hayashi H, Ishida K, Moriya T, Tabata T, Kondo E, Kanai N, Gu Z, Sunamura M, Unno M and Horii (2013) The expression of S100A4 in human pancreatic cancer is associated with invasion. Pancreas 42 (6), 1027-1033 (2013).
Xia Z J (2000) Homoclinic points and intersections of Lagrangian submanifolds, Discrete Contin. Dynam. Systems 6 (1): 243-253.
Xia Z J (2000) C1 connecting lemmas, Trans. Amer. Math. Soc. 352 (1): 5213-5230.
Xia Z J (1998) Arnold diffusion: a variational construction, Proceedings of the International Congress of Mathematicians, Vol. I I (Berlin, 1998). Doc. Math. Extra Vol. II, 867-877.
Xia Z J (1996) Homoclinic points in symplectic and volume-preserving diffeomorphisms, Commun. Math. Phys. 177:435-449.
Xie D, Xie K. (2015) Pancreatic cancer stromal biology and therapy. Genes Dis.2(2):133-143. PMCID: PMC4476547
Wainwright D A, Chang A L, Dey M, Balyasnikova I V, Kim C K, Tobias A, Cheng Y, Kim J W, Qiao J, Zhang L, Han Y, Lesniak M S. (2014) Durable therapeutic efficacy utilizing combinatorial blockade against IDO, CTLA-4, and PD-L1 in mice with brain tumors. Clin Cancer Res. 20(20):5290-301. PMCID: PMC4182350
Wainwright D A, Dey M, Chang A, and Lesniak M S. (2013) Targeting Tregs in malignant brain cancer: Overcoming IDO. Front Immunol 15; 4:116, PMCID: PMC3654236
Wang B, Xiao Y, Ding B B, Zhang N, Yuan X, Gui L, et al. (2003) Induction of tumor angiogenesis by Slit-Robo signaling and inhibition of cancer growth by blocking Robo activity. Cancer Cell 4:19-29.
Wang L, Zhi X, Zhu Y, Zhang Q, Wang W, Li Z, Tang J, Wang J, Wei S, Li B, Zhou J, Jiang J, Yang L, Xu H, Xu Z. (2015). MUC4-promoted neural invasion is mediated by the axon guidance factor Netrin-1 in PDAC. Oncotarget. 6(32):33805-22. PMCID: PMC4741804
Ward M, McCann C M, DeWulf M, Wu J Y*, Rao Y (2003). Distinguishing Between Directional Guidance and Motility Regulation in Neuronal Migration. J. Neurosci 23(12):5170-7. PMCID: PMC2041933.
Ward M E, Wu J Y, Rao Y (2004). Visualization of spatially and temporally regulated N-WASP activity during cytoskeletal reorganization in living cells. Proc. Natl. Acad. Sci. USA 101:970-974 [* co-corresponding author]. PMCID: PMC327126.
Wei D, Wang L, Yan Y, Jia Z, Gagea M, Li Z, Zuo X, Kong X, Huang S, Xie K. (2016) KLF4 Is Essential for Induction of Cellular Identity Change and Acinar-to-Ductal Reprogramming during Early Pancreatic Carcinogenesis. Cancer Cell. 29(3):324-38
Whitsett T G, Fortin Ensign S P, Dhruv H D, Inge L J, Kurywchak P, Wolf K K, LoBello J, Kingsley C B, Allen J W, Weiss G J, Tran N L. (2014). FN14 expression correlates with MET in NSCLC and promotes MET-driven cell invasion. Clin Exp Metastasis. 31(6):613-23
Williams S A, Maecker H L, French D M, Liu J, Gregg A, Silverstein L B, Cao T C, Carano R A, Dixit V M. (2011) USP1 deubiquitinates I D proteins to preserve a mesenchymal stem cell program in osteosarcoma. Cell. 146(6):918-30.
Wong K, Ren X-R, Huang Y-Z, Xie Y, Liu G, Saito H, Tang H, Wen L, Brady-Kalnay S M, Mei, L, Wu J Y, Xiong W-C., and Rao Y. (2001) Signal Transduction in Neuronal Migration: Roles of GTPase Activating Proteins and the Small GTPase Cdc42 in the Slit-Robo Pathway. Cell 107:209-221.
Worthylake R A, Lemoine S, Watson J M, et al.2001 RhoA is required for monocyte tail retraction during transendothelial migration. J Cell Biol 154:147-160. PMCID: PMC2196864.
Wu J Y*, Feng L, Park, H-T, Havlioglu N, Wen L, Bacon K B, Jiang Z, Tang H, Zhang X-C, and Rao Y (2001). The neuronal repellent Slit also inhibits leukocyte chemotaxis induced by chemotactic factors. Nature 410:948-952 [* co-corresponding author]. PMCID: PMC2072862
Wu W, Wong K, Chen J H, Jiang Z H, Dupuis S, *Wu J Y, and Rao Y (1999). Directional guidance of neuronal migration in the olfactory system by the secreted protein Slit. Nature 400:331-326. PMCID: PMC2041931.
Xian J, Aitchison A, Bobrow L, Corbett G, Pannell R, Rabbitts T, Rabbitts P. (2004). Targeted disruption of the 3p12 gene, Dutt1/Robo1, predisposes mice to lung adenocarcinomas and lymphomas with methylation of the gene promoter. Cancer Res.64(18):6432-7
Xian J, Clark K J, Fordham R, Pannell R, Rabbitts T H, Rabbitts P H. (2001) Inadequate lung development and bronchial hyperplasia in mice with a targeted deletion in the Dutt1/Robo1 gene. Proc Natl Acad Sci USA 98:15062-6. PMCID: PMC64983
Xu X, Wang Y, Chen J, Ma H, Shao Z, Chen H, Jin G. (2012) High expression of CX3CL1/CX3CR1 axis predicts a poor prognosis of pancreatic ductal adenocarcinoma. J Gastrointest Surg. 16(8):1493-8
Yan F, Liu J J, Ip V, Jamieson S M, McKeage M J. (2015) Role of platinum DNA damage induced transcriptional inhibition in chemotherapy-induced neuronal atrophy and peripheral neurotoxicity. J Neurochem. doi: 10.1111/jnc.13355
Yao J, Li W Y, Li S G, Feng X S, Gao S G. (2014). Midkine promotes perineural invasion in human pancreatic cancer. World J Gastroenterol. 20(11):3018-24. PMCID: PMC3960408
Yiin J J, Hu B, Jarzynka M J, Feng H, Liu K W, Wu J Y, Ma H I, Cheng S Y. (2009) Slit2 inhibits glioma cell invasion in the brain by suppression of Cdc42 activity. Neuro Oncol. 11(6):779-89. PMCID: PMC2802398.
Ypsilanti A R, Zagar Y, Chedotal A. (2010) Moving away from the midline: new developments for Slit and Robo. Development 137:1939-52.
Yu J D, Cao Q, Yu J J, Wu L T, Dallol A, Li J, Guoan Chen G A, Grasso C, CaoXH, Lonigro R J, Varambally S, Mehra R, Palanisamy N, Wu J Y, Latif F, Chinnaiyan A M (2010) The neuronal repellent SLIT2 is a target for repression by EZH2 in prostate cancer Oncogene 29(39):5370-80. PMCID: PMC2948081.
Yuan J, Luo K, Zhang L, Cheville J C, Lou Z. (2010) USP10 regulates p53 localization and stability by deubiquitinating p53. Cell. 140(3):384-96. PMCID: PMC2820153
Yuan W, Zhou L, Chen J H, et al. (1999) The mouse SLIT family: secreted ligands for ROBO expressed in patterns that suggest a role in morphogenesis and axon guidance. Dev Biol 212:290-306.
Yuan W L, Rao Y, Babiuk R P, Greer J, Wu J Y, and Ornitz D M (2003). A genetic model for a central (septum transversum) congenital diaphragmatic hernia in mice lacking Slit3. Proc. Natl. Acad. Sci. USA. 100(9):5217-22. PMCID: PMC154325.
Yuasa-Kawada J, Kinoshita-Kawada M, Rao Y, Wu J Y. (2009) Deubiquitinating enzyme USP33/VDU1 is required for Slit signaling in inhibiting breast cancer cell migration. Proc Natl Acad Sci USA 106:14530-5. PMCID: PMC2732860
Yuasa-Kawada J, Kinoshita-Kawada M, Rao Y, Wu J Y* (2009) Deubiquitinating enzyme USP33NDU1 is required for Slit signaling in inhibiting breast cancer cell migration. Proc Natl Acad Sci USA 106:14530-14535. PMCID: PMC2732860.
Yuasa-Kawada J, Kinoshita-Kawada M, Wu G, Rao Y, Wu J Y. (2009) Midline crossing and Slit responsiveness of commissural axons require USP33. Nat Neurosci 12:1087-9. PMCID: PMC3893063
Zhang J F, Hua R, Liu D J, Liu W, Huo Y M, Sun Y W. (2014) Effect of CD74 on the prognosis of patients with resectable pancreatic cancer. Hepatobiliary Pancreat Dis Int. 13(1):81-6.
Zhou Y, Gunput R A, Adolfs Y, Pasterkamp R J. (2011) MICALs in control of the cytoskeleton, exocytosis, and cell death. Cell Mol Life Sci.; 68(24):4033-44. PMCID: PMC3221843
Zhu Y, Yu T, Zhang X, Nagasawa T, Wu J Y*, Rao Y (2002) Role of the Chemokine SDF-1 as the meningeal attractant for embryonic cerebellar neurons. Nature Neuroscience, 5:719-720 [* co-corresponding author]. PMCID: PMC2072873.
Zhu Y, Knolhoff B L, Meyer M A, Nywening T M, West B L, Luo J, Wang-Gillam A, Goedegebuure S P, Linehan D C, DeNardo D G. (2014) CSF1/CSF1R blockade reprograms tumor-infiltrating macrophages and improves response to T-cell checkpoint immunotherapy in pancreatic cancer models. Cancer Res. 15; 74(18):5057-69. PMCID: PMC4182950

1. A method for treating a subject having cancer, the method comprising inhibiting the activity of Myo9 in the subject.

2. The method of claim 1 wherein inhibiting the activity of Myo9 in the subject comprises administering a therapeutically effective amount of a Myo9 inhibitor to the subject.

3. The method of claim 2 wherein the inhibitor of Myo9 is an antibody specific for the Myo9 protein or an antigen-binding antibody fragment specific for the Myo9 protein.

4. The method of claim 2 wherein the inhibitor of Myo9 inhibits expression of a gene encoding the Myo9 protein.

5. The method of claim 4 wherein the inhibitor of Myo9 inhibits transcription of a Myo9 gene or inhibits the translation of a Myo9 messenger ribonucleic acid.

6. The method of claim 2 wherein the inhibitor of Myo9 is a monoclonal antibody specific for the Myo9 protein.

7. The method of claim 2 wherein the inhibitor of Myo9 is a humanized antibody specific for the Myo9 protein.

8. The method of claim 1 wherein the Myo9 is Myo9a or Myo9b.

9. The method of claim 1 wherein the cancer is lung cancer or pancreatic cancer.

10. The method of claim 1 further comprising detecting the level of Myo9 expression or Myo9 activity in the subject.

11. The method of claim 10 further comprising comparing the level of Myo9 expression or Myo9 activity in the subject to a level of Myo9 expression or Myo9 activity in a cancer-free subject.

12. The method of claim 2 wherein the therapeutically effective amount of an inhibitor of Myo9 is administered by intratumor injection, intravenous systemic administration, intranasal administration, or using an adenovirus.

13. The method of claim 1 wherein the inhibitor of Myo9 is an immunotherapeutic antibody conjugate.

14. The method of claim 1 wherein inhibiting the activity of Myo9 comprises increasing the activity of Slit or Robo in the subject.

15. A method for identifying a sample comprising a cancer cell, the method comprising detecting the level of Myo9 expression or activity in the sample and identifying the sample as a sample comprising a cancer cell by determining that the level of Myo9 expression or activity measured for the sample is greater than the level of Myo9 expression or activity for a non-cancer sample.

16. The method of claim 15 wherein determining the level of Myo9 expression or activity in the sample comprises a method selected from quantitative nucleic acid amplification or immunostaining.

17. The method of claim 15 comprising detecting the level of Myo9b expression or activity in the sample.

18. A composition for treating a subject having a cancer, the composition comprising an inhibitor of Myo9.

19. The composition of claim 18 wherein the inhibitor of Myo9 is an antibody specific for Myo9, an antigen-binding antibody fragment specific for Myo9, or a nucleic acid complementary to the Myo9 gene or Myo9 messenger ribonucleic acid.

20. The composition of claim 18 formulated for administration to the subject.
